SEQUENCE LISTING (64 sequences)

SEQ ID NO: 1 - protein, 870 aa, synthetic construct
  REGION 1..870: note = WWP2-FL (UniProtKB: O00308-1, v2)
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 2 - protein, 81 aa, synthetic construct
  REGION 1..81: note = WWP2 C2 domain
LTLKVVSAKPKVHNRQPRINSYVEVAVDGLPSETKKTGKRIGSSELLWNEIIILNVTAQSHLDLKVWSCHTLRNELLGTAS

SEQ ID NO: 3 - protein, 34 aa, synthetic construct
  REGION 1..34: note = WWP2 WW1 domain
DALPAGWEQRELPNGRVYYVDHNTKTTTWERPLP

SEQ ID NO: 4 - protein, 34 aa, synthetic construct
  REGION 1..34: note = WWP2 WW2 domain
RPLPPGWEKRTDPRGRFYYVDHNTRTTTWQRPTA

SEQ ID NO: 5 - protein, 33 aa, synthetic construct
  REGION 1..33: note = WWP2 WW3 domain
GPLPPGWEKRQDNGRVYYVNHNTRTTQWEDPRT

SEQ ID NO: 6 - protein, 34 aa, synthetic construct
  REGION 1..34: note = WWP2 WW4 domain
PALPPGWEMKYTSEGVRYFVDHNTRTTTFKDPRP

SEQ ID NO: 7 - protein, 335 aa, synthetic construct
  REGION 1..335: note = WWP2 HECT domain
KPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 8 - protein, 335 aa, synthetic construct
  REGION 1..335: note = WWP2-N (UniProtKB: O00308-4)
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPG

SEQ ID NO: 9 - protein, 431 aa, synthetic construct
  REGION 1..431: note = WWP2-C (UniProtKB: O00308-3)
MIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 10 - protein, 754 aa, synthetic construct
  REGION 1..754: note = WWP2-2 (UniProtKB: O00308-2)
MQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 11 - protein, 870 aa, synthetic construct
  REGION 1..870: note = Mouse WWP2 (UniProtKB: Q9DBH0-1, v1)
MASASSSRAGVALPFEKSQLTLKVVSAKPKVHNRQPRINSYVEVAVDGLPSETKKTGKRIGSSELLWNEIIVLNVTAQSHLDLKVWSCHTLRNELLGTASVNLSNVLKNNGGKMENTQLTLNLQTENKGSVVSGGELTIFLDGPTVDLGSVPNGSAVTDGSQPPSRESSGTAIAPETRHQPPSTNCFGGRSRTHRHSGGSARTATAASEQSPGARNRHRQPVKNSSSSGLANGTVNEEPTPASEPEESSVVGVTSLPAAALSVSSNPNTTSLPAQSTPAEGEEASTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELFFIQDMEILGKVTTHELKEGGENIRVTEENKEEYIMLLTDWRFTRGVEEQTKAFLDGFNEVAPLEWLRYFDEKELELMLCGMQEIDMSDWQKNAIYRHYTKSSKQIQWFWQVVKEMDNEKRIRLLQFVTGTCRLPVGGFAELIGSNGPQKFCIDRVGKETWLPRSHTCFNRLDLPPYKSYEQLKEKLLYAIEETEGFGQE

SEQ ID NO: 12 - DNA, 4608 bp, synthetic construct
  misc_feature 1..4608: note = WWP2-FL mRNA (NCBI NM_007014.4)
  source 1..4608: mol_type = other DNA
ctcttccgggtcaggtgaccgcgcgcggtcacgtgacccggcccgagtgcgggcggtggaaggcggaagtaggagaggagttcggcgccgcttctgtggccacggcagatgaaacagaaaggctaaagagggctggagtcaggggacttctcttccaccagcttcacggtgatgatatggcatctgccagctctagccgggcaggagtggccctgccttttgagaagtctcagctcactttgaaagtggtgtccgcaaagcccaaggtgcataatcgtcaacctcgaattaactcctacgtggaggtggcggtggatggactccccagtgagaccaagaagactgggaagcgcattgggagctctgagcttctctggaatgagatcatcattttgaatgtcacggcacagagtcatttagatttaaaggtctggagctgccataccttgagaaatgaactgctaggcaccgcatctgtcaacctctccaacgtcttgaagaacaatgggggcaaaatggagaacatgcagctgaccctgaacctgcagacggagaacaaaggcagcgttgtctcaggcggagagctgacaattttcctggacgggccaactgttgatctgggaaatgtgcctaatggcagtgccctgacagatggatcacagctgccttcgagagactccagtggaacagcagtagctccagagaaccggcaccagccccccagcacaaactgctttggtggaagatcccggacgcacagacattcgggtgcttcagccagaacaaccccagcaaccggcgagcaaagccccggtgctcggagccggcaccgccagcccgtcaagaactcaggccacagtggcttggccaatggcacagtgaatgatgaacccacaacagccactgatcccgaagaaccttccgttgttggtgtgacgtccccacctgctgcacccttgagtgtgaccccgaatcccaacacgacttctctccctgccccagccacaccggctgaaggagaggaacccagcacttcgggtacacagcagctcccagcggctgcccaggcccccgacgctctgcctgctggatgggaacagcgagagctgcccaacggacgtgtctattatgttgaccacaataccaagaccaccacctgggagcggccccttcctccaggctgggaaaaacgcacagatccccgaggcaggttttactatgtggatcacaatactcggaccaccacctggcagcgtccgaccgcggagtacgtgcgcaactatgagcagtggcagtcgcagcggaatcagctccagggggccatgcagcacttcagccaaagattcctctaccagtcttcgagtgcttcgactgaccatgatcccctgggccccctccctcctggctgggagaagagacaggacaatggacgggtgtattacgtgaaccataacactcgcacgacccagtgggaggatccccggacccaggggatgatccaggaaccagctctgcccccaggatgggagatgaaatacaccagcgagggggtgcgatactttgtggaccacaatacccgcaccaccacctttaaggatcctcgcccggggtttgagtcggggacgaagcaaggttcccctggtgcttatgaccgcagttttcggtggaagtatcaccagttccgtttcctctgccattcaaatgccctacctagccacgtgaagatcagcgtttccaggcagacgcttttcgaagattccttccaacagatcatgaacatgaaaccctatgacctgcgccgccggctctacatcatcatgcgtggcgaggagggcctggactatgggggcatcgccagagagtggtttttcctcctgtctcatgaggtgctcaaccctatgtattgtttatttgaatatgccggaaagaacaattactgcctgcagatcaaccccgcctcctccatcaacccggaccacctcacctactttcgctttataggcagattcatcgccatggcgctgtaccatggaaagttcatcgacacgggcttcaccctccctttctacaagcggatgctcaataagagaccaaccctgaaagacctggagtccattgaccctgagttctacaactccattgtctggatcaaagagaacaacctggaagaatgtggcctggagctgtacttcatccaggacatggagatactgggcaaggtgacgacccacgagctgaaggagggcggcgagagcatccgggtcacagaggagaacaaggaagagtacatcatgctgctgactgactggcgtttcacccgaggcgtggaagagcagaccaaagccttcctggatggcttcaacgaggtggccccgctggagtggctgcgctactttgacgagaaagagctggagctgatgctgtgcggcatgcaggagatagacatgagcgactggcagaagagcaccatctaccggcactacaccaagaacagcaagcagatccagtggttctggcaggtggtgaaggagatggacaacgagaagaggatccggctgctgcagtttgtcaccggtacctgccgcctgcccgtcgggggatttgccgaactcatcggtagcaacggaccacagaagttttgcattgacaaagttggcaaggaaacctggctgcccagaagccacacctgcttcaaccgtctggatcttccaccctacaagagctacgaacagctgagagagaagctgctgtatgccattgaggagaccgagggctttggacaggagtaaccgaggccgcccctcccacgccccccagcgcacatgtagtcctgagtcctccctgcctgagaggccactggccccgcagcccttgggaggcccccgtggatgtggccctgtgtgggaccacactgtcatctcgctgctggcagaaaagcctgatcccaggaggccctgcagttcccccgacccgcggatggcagtctggaataaagccccctagttgcctttggccccacctttgcaaagttccagagggctgaccctctctgcaaaactctcccctgtcctctagaccccaccctgggtgtatgtgagtgtgcaagggaaggtgttgcatccccaggggctgccgcagaggccggagacctcctggactagttcggcgaggagactggccactgggggtggctgttcgggactgagagcgccaagggtctttgccagcaaaggaggttctgcctgtaattgagcctctctgatgatggagatgaagtgaaggtctgagggagcgggccctggggcgaggccatctctgcctgcctccctagcaggcgccagcggtggaggctgagtcgcaggacacatgccggccagttaattcattctcagccaatgaaggtttgtctaagctgcctgggtatccacgggacaaaaacagcaaactccctcagactttgtccatgtataaacttgaagtggttgtgttgtagggttgcaggttttttgttacgctgctgtcactttctgtccaggagctggcaccccaggtgttctgagaccttgagggacccagacctttgggtccaagagtttcccaaacagccacgcctctcaggaacccacctggcggttccgtgagctcaggcaggcctgacccggcggcacagcctggcagggacctcgtccccaagcctggcagaatgagaggggttgaggtcccgagcgccactcctagccttgccgccttcaatagagaagaaatccctttgctagatagggtcccccaggcagtccccagtggcgggacacaggggtccggctgtggagctcccctgccagcccctggagctccaggagggcctgttggtcccctgttcagaatggagtgcagcccgccagcggaaagtgttcattctgcataggtgtgaggctttatctgcacacaggacatgaaaaccagcagaaaggccctgagctgctgcatagccccatctgatttctgcagttcccgccagcctccaacacggggactctgccgtaactggaatcttcataggtcatattgaaatcttcaaggtgaccatgccccaccggggtgctggggcagtagtcatggcagactcccggcctgggcccccaggattctaggacccccaggcagccccttggactggtcccgggtgccttccaagcacagtctccatgctcccagattctcgaccttcccccggcccgggaggtgcagcctgcgtctgcctctgtcgtgtgtgctgatttgagtggcttagcttgccacagcgcagcctcttctgtccctttcagtcatttgctgtacttccctgtggcacgttaccatggaagccgctccagggtgggtcagggtgcaagctgctggtgaggtttggaagcatcaggctcacgggtgttcatgtgtgttcgtgcgtgtgtgtgcgtacgtgtatataactgaagtgtctgtacggaatgccctttgctagccatgggctggtcaccagattgttttgtaatgcccgccccttgcctcgatattgccagtttcttgtgcaataaacaatcagcagctgtgaaaaaaaaaaaaaaaaaa

SEQ ID NO: 13 - DNA, 2682 bp, synthetic construct
  misc_feature 1..2682: note = WWP2-N mRNA (NCBI NM_001270455.1)
  source 1..2682: mol_type = other DNA
ctcttccgggtcaggtgaccgcgcgcggtcacgtgacccggcccgagtgcgggcggtggaaggcggaagtaggagaggagttcggcgccgcttctgtggccacggcagcttcacggtgatgatatggcatctgccagctctagccgggcaggagtggccctgccttttgagaagtctcagctcactttgaaagtggtgtccgcaaagcccaaggtgcataatcgtcaacctcgaattaactcctacgtggaggtggcggtggatggactccccagtgagaccaagaagactgggaagcgcattgggagctctgagcttctctggaatgagatcatcattttgaatgtcacggcacagagtcatttagatttaaaggtctggagctgccataccttgagaaatgaactgctaggcaccgcatctgtcaacctctccaacgtcttgaagaacaatgggggcaaaatggagaacatgcagctgaccctgaacctgcagacggagaacaaaggcagcgttgtctcaggcggagagctgacaattttcctggacgggccaactgttgatctgggaaatgtgcctaatggcagtgccctgacagatggatcacagctgccttcgagagactccagtggaacagcagtagctccagagaaccggcaccagccccccagcacaaactgctttggtggaagatcccggacgcacagacattcgggtgcttcagccagaacaaccccagcaaccggcgagcaaagccccggtgctcggagccggcaccgccagcccgtcaagaactcaggccacagtggcttggccaatggcacagtgaatgatgaacccacaacagccactgatcccgaagaaccttccgttgttggtgtgacgtccccacctgctgcacccttgagtgtgaccccgaatcccaacacgacttctctccctgccccagccacaccggctgaaggagaggaacccagcacttcgggtacacagcagctcccagcggctgcccaggcccccgacgctctgcctgctggatgggaacagcgagagctgcccaacggacgtgtctattatgttgaccacaataccaagaccaccacctgggagcggccccttcctccagggtaggtcatcaactgagaagacctgagactctggaactgacaccatgagtcacccaatggcttcttgaaacggtccctttctgcggaggtagcatagcacagtgacgtttattccgggtcacttgattgcttttcctatccacttaccttaatattgctcccatgtcttaggacatattagaattattagaagatctctgggaaacaaaaggcgcatgggaggccaagatttagagtgttgatgcattccgccgtaccctatgcccagcctgtccctaaaggcagagtgcttcttattcggcagggcttcgtgagaatgccgccaagttatccaaaaccaaaggatgagagaggcttccaagagagggaagactgtccaagttcaaggagaaggggtttggttccctgtctgccagggacagaaagttctttccacatgtgtctcctggtcccgagagctttcaaaaggggatgtattctgctccaagaaagaatcacctcccagtaaagtagccatgaagggctcctgtataaaatatgcacacttcccctgccctgagtttgtggagggaaaggctgtcggaagtagggactcttaggagagcccgtgtgccctgtttggttcagctgtcaggcgtgtgccacacacatcccacctcatcttctggtcccataattctccatggatgggctgcttttagctccagttttccagtagaatgcttgagttttccttttttccgtacctcctggaggatgaaactcataacctgtcatcatcagtactcactgtgttttccactttgacttgaagtcaaatgaagttaccaaagttaaacatggacttctcaaattagaaaaaaacaaacaacaaaaaaacaggttaagtgttttgttatgaaaattatgaaaatgttcttattattatggaaaatttcaaatagatatagagagaataatgttacaaacccccacatactcaacatttagcttcaaaaattatcaactcatggcgggttttgtttcatctgtacaaactagtcggcacactatgatggcctgtggacaaaatccggccctctgactgtttttgtaaataaggttttattggaacacagccacacatatttgttgacttattgtttttggctgtttttgtacttcattggcagagttgagtaattgcagcagactctatggcttgcaaaacctaaaataattactttgtgtagcgctttatagaaaaaatttgctaactcccgatctgtaacccccaacctatccgccgcaccctgatctaggtaattttgaagcaaatcccaaatgtcacatcatctatccatatttcactatgtatttctaaaacatattcttttattcaaaataaaactctaatatcattaacagtattttgtttatattatcaaatacccagtcaatatttgactttctataattgtgtcacaaattaaacattgactttaacagtatgtattacacacttgtactttgtgtactttataacatgtgcactttaaaacagcaggagctctgt

SEQ ID NO: 14 - DNA, 3700 bp, synthetic construct
  misc_feature 1..3700: note = WWP2-C mRNA (NCBI NM_199424.2)
  source 1..3700: mol_type = other DNA
attttttcccctcctgcgtgtggttcttggagaaagttggaggtggtggtgatttcagtcgccttggccgccttgagccggagctgagcggaggcactgggccgagcctgcttcccgggccttcctaccatgccagggctgctccctgcctccgccaccctggcacaccttcacccgcgtaccgcctcctccccgtcgctctgccttttccaaaactcacttgggccctccgtgcgcagggttcttttttggtttttctgtaaaaatcaaaacaaaaaacagagacttttgagaggagcagatgccacctaaagtcccactgcattccctgcaaagcgctcaaatgtggaagccagtcattggcatttttattttttattgattgattgattttttcaccagtggctttttgtaacctctgtgttctgctgtgtttcttgtgtttagtcttcgagtgcttcgactgaccatgatcccctgggccccctccctcctggctgggagaagagacaggacaatggacgggtgtattacgtgaaccataacactcgcacgacccagtgggaggatccccggacccaggggatgatccaggaaccagctctgcccccaggatgggagatgaaatacaccagcgagggggtgcgatactttgtggaccacaatacccgcaccaccacctttaaggatcctcgcccggggtttgagtcggggacgaagcaaggttcccctggtgcttatgaccgcagttttcggtggaagtatcaccagttccgtttcctctgccattcaaatgccctacctagccacgtgaagatcagcgtttccaggcagacgcttttcgaagattccttccaacagatcatgaacatgaaaccctatgacctgcgccgccggctctacatcatcatgcgtggcgaggagggcctggactatgggggcatcgccagagagtggtttttcctcctgtctcatgaggtgctcaaccctatgtattgtttatttgaatatgccggaaagaacaattactgcctgcagatcaaccccgcctcctccatcaacccggaccacctcacctactttcgctttataggcagattcatcgccatggcgctgtaccatggaaagttcatcgacacgggcttcaccctccctttctacaagcggatgctcaataagagaccaaccctgaaagacctggagtccattgaccctgagttctacaactccattgtctggatcaaagagaacaacctggaagaatgtggcctggagctgtacttcatccaggacatggagatactgggcaaggtgacgacccacgagctgaaggagggcggcgagagcatccgggtcacagaggagaacaaggaagagtacatcatgctgctgactgactggcgtttcacccgaggcgtggaagagcagaccaaagccttcctggatggcttcaacgaggtggccccgctggagtggctgcgctactttgacgagaaagagctggagctgatgctgtgcggcatgcaggagatagacatgagcgactggcagaagagcaccatctaccggcactacaccaagaacagcaagcagatccagtggttctggcaggtggtgaaggagatggacaacgagaagaggatccggctgctgcagtttgtcaccggtacctgccgcctgcccgtcgggggatttgccgaactcatcggtagcaacggaccacagaagttttgcattgacaaagttggcaaggaaacctggctgcccagaagccacacctgcttcaaccgtctggatcttccaccctacaagagctacgaacagctgagagagaagctgctgtatgccattgaggagaccgagggctttggacaggagtaaccgaggccgcccctcccacgccccccagcgcacatgtagtcctgagtcctccctgcctgagaggccactggccccgcagcccttgggaggcccccgtggatgtggccctgtgtgggaccacactgtcatctcgctgctggcagaaaagcctgatcccaggaggccctgcagttcccccgacccgcggatggcagtctggaataaagccccctagttgcctttggccccacctttgcaaagttccagagggctgaccctctctgcaaaactctcccctgtcctctagaccccaccctgggtgtatgtgagtgtgcaagggaaggtgttgcatccccaggggctgccgcagaggccggagacctcctggactagttcggcgaggagactggccactgggggtggctgttcgggactgagagcgccaagggtctttgccagcaaaggaggttctgcctgtaattgagcctctctgatgatggagatgaagtgaaggtctgagggagcgggccctggggcgaggccatctctgcctgcctccctagcaggcgccagcggtggaggctgagtcgcaggacacatgccggccagttaattcattctcagccaatgaaggtttgtctaagctgcctgggtatccacgggacaaaaacagcaaactccctcagactttgtccatgtataaacttgaagtggttgtgttgtagggttgcaggttttttgttacgctgctgtcactttctgtccaggagctggcaccccaggtgttctgagaccttgagggacccagacctttgggtccaagagtttcccaaacagccacgcctctcaggaacccacctggcggttccgtgagctcaggcaggcctgacccggcggcacagcctggcagggacctcgtccccaagcctggcagaatgagaggggttgaggtcccgagcgccactcctagccttgccgccttcaatagagaagaaatccctttgctagatagggtcccccaggcagtccccagtggcgggacacaggggtccggctgtggagctcccctgccagcccctggagctccaggagggcctgttggtcccctgttcagaatggagtgcagcccgccagcggaaagtgttcattctgcataggtgtgaggctttatctgcacacaggacatgaaaaccagcagaaaggccctgagctgctgcatagccccatctgatttctgcagttcccgccagcctccaacacggggactctgccgtaactggaatcttcataggtcatattgaaatcttcaaggtgaccatgccccaccggggtgctggggcagtagtcatggcagactcccggcctgggcccccaggattctaggacccccaggcagccccttggactggtcccgggtgccttccaagcacagtctccatgctcccagattctcgaccttcccccggcccgggaggtgcagcctgcgtctgcctctgtcgtgtgtgctgatttgagtggcttagcttgccacagcgcagcctcttctgtccctttcagtcatttgctgtacttccctgtggcacgttaccatggaagccgctccagggtgggtcagggtgcaagctgctggtgaggtttggaagcatcaggctcacgggtgttcatgtgtgttcgtgcgtgtgtgtgcgtacgtgtatataactgaagtgtctgtacggaatgccctttgctagccatgggctggtcaccagattgttttgtaatgcccgccccttgcctcgatattgccagtttcttgtgcaataaacaatcagcagctgtgaaaaaaaaaaaaaaaaaa

SEQ ID NO: 15 - DNA, 62 bp, synthetic construct
  misc_feature 1..62: note = Soond et al. primer for WWP2-FL specific shRNA (sense)
  source 1..62: mol_type = other DNA
gatctccggagtacgtgcgcaactatgatcaagagtcatagttgcgcacgtactccttttta

SEQ ID NO: 16 - DNA, 62 bp, synthetic construct
  misc_feature 1..62: note = Soond et al. primer for WWP2-FL specific shRNA (antisense)
  source 1..62: mol_type = other DNA
gatctaaaaaggagtacgtgcgcaactatgactcttgatcatagttgcgcacgtactccgga

SEQ ID NO: 17 - DNA, 62 bp, synthetic construct
  misc_feature 1..62: note = Soond et al. primer for WWP2-N specific shRNA (sense)
  source 1..62: mol_type = other DNA
gatctccgacgtttattccgggtcactttcaagagaagtgacccggaataaacgtcttttta

SEQ ID NO: 18 - DNA, 62 bp, synthetic construct
  misc_feature 1..62: note = Soond et al. primer for WWP2-N specific shRNA (antisense)
  source 1..62: mol_type = other DNA
gatctaaaaagacgtttattccgggtcacttctcttgaaagtgacccggaataaacgtcgga

SEQ ID NO: 19 - DNA, 62 bp, synthetic construct
  misc_feature 1..62: note = Soond et al. primer for WWP2-C specific shRNA (sense)
  source 1..62: mol_type = other DNA
gatctccggagcagatgccacctaaagttcaagagactttaggtggcatctgctccttttta

SEQ ID NO: 20 - DNA, 62 bp, synthetic construct
  misc_feature 1..62: note = Soond et al. primer for WWP2-C specific shRNA (antisense)
  source 1..62: mol_type = other DNA
gatctaaaaaggagcagatgccacctaaagtctcttgaactttaggtggcatctgctccgga

SEQ ID NO: 21 - RNA, 20 nt, synthetic construct
  misc_feature 1..20: note = Maddika et al. WWP2 siRNA 1
  source 1..20: mol_type = other RNA
tgacaaagttggaaggaatt

SEQ ID NO: 22 - RNA, 21 nt, synthetic construct
  misc_feature 1..21: note = Maddika et al. WWP2 siRNA 2
  source 1..21: mol_type = other RNA
gggagaagagacaggacaatt

SEQ ID NO: 23 - RNA, 21 nt, synthetic construct
  misc_feature 1..21: note = Maddika et al. WWP2 siRNA 3
  source 1..21: mol_type = other RNA
gggagaagagacaggacaatt

SEQ ID NO: 24 - RNA, 21 nt, synthetic construct
  misc_feature 1..21: note = Maddika et al. WWP2 siRNA 4
  source 1..21: mol_type = other RNA
acatggagatactgggcaatt

SEQ ID NO: 25 - RNA, 22 nt, synthetic construct
  misc_feature 1..22: note = Maddika et al. WWP2 shRNA 1
  source 1..22: mol_type = other RNA
agcacagagtcatttagattta

SEQ ID NO: 26 - RNA, 22 nt, synthetic construct
  misc_feature 1..22: note = Maddika et al. WWP2 shRNA 2
  source 1..22: mol_type = other RNA
acctatgtattgtttatttgaa

SEQ ID NO: 27 - DNA, 22 bp, synthetic construct
  misc_feature 1..22: note = Mouse Wwp2-P1 RT-qPCR forward primer
  source 1..22: mol_type = other DNA
tttgagaagtcccagcttaccc

SEQ ID NO: 28 - DNA, 23 bp, synthetic construct
  misc_feature 1..23: note = Mouse Wwp2-P1 RT-qPCR reverse primer
  source 1..23: mol_type = other DNA
ctccagaccttcagatccaaatg

SEQ ID NO: 29 - DNA, 20 bp, synthetic construct
  misc_feature 1..20: note = Mouse Wwp2-P2 RT-qPCR forward primer
  source 1..20: mol_type = other DNA
accaagagcagcaagcagat

SEQ ID NO: 30 - DNA, 20 bp, synthetic construct
  misc_feature 1..20: note = Mouse Wwp2-P2 RT-qPCR reverse primer
  source 1..20: mol_type = other DNA
gtctcctcgatggcgtacag

SEQ ID NO: 31 - DNA, 20 bp, synthetic construct
  misc_feature 1..20: note = Mouse 18S rRNA RT-qPCR forward primer
  source 1..20: mol_type = other DNA
gtaacccgttgaaccccatt

SEQ ID NO: 32 - DNA, 20 bp, synthetic construct
  misc_feature 1..20: note = Mouse 18S rRNA RT-qPCR reverse primer
  source 1..20: mol_type = other DNA
ccatccaatcggtagtagcg

SEQ ID NO: 33 - DNA, 19 bp, synthetic construct
  misc_feature 1..19: note = Mouse Tgfbr1 RT-qPCR forward primer
  source 1..19: mol_type = other DNA
ctcccgtggcttctagtgc

SEQ ID NO: 34 - DNA, 22 bp, synthetic construct
  misc_feature 1..22: note = Mouse Tgfbr1 rRNA RT-qPCR reverse primer
  source 1..22: mol_type = other DNA
gccttagtttggacaggatctg

SEQ ID NO: 35 - DNA, 18 bp, synthetic construct
  misc_feature 1..18: note = Mouse Tgfbr2 RT-qPCR forward primer
  source 1..18: mol_type = other DNA
catttggttccaaggtgc

SEQ ID NO: 36 - DNA, 17 bp, synthetic construct
  misc_feature 1..17: note = Mouse Tgfbr2 rRNA RT-qPCR reverse primer
  source 1..17: mol_type = other DNA
tggtagtgttcagcgag

SEQ ID NO: 37 - DNA, 21 bp, synthetic construct
  misc_feature 1..21: note = Mouse Acta2 RT-qPCR forward primer
  source 1..21: mol_type = other DNA
gtcccagacatcagggagtaa

SEQ ID NO: 38 - DNA, 21 bp, synthetic construct
  misc_feature 1..21: note = Mouse Acta2 RT-qPCR reverse primer
  source 1..21: mol_type = other DNA
tcggatacttcagcgtcagga

SEQ ID NO: 39 - DNA, 22 bp, synthetic construct
  misc_feature 1..22: note = Mouse Smad2 RT-qPCR forward primer
  source 1..22: mol_type = other DNA
aatacggtagatcagtgggaca

SEQ ID NO: 40 - DNA, 21 bp, synthetic construct
  misc_feature 1..21: note = Mouse Smad2 RT-qPCR reverse primer
  source 1..21: mol_type = other DNA
cagttttcgattgccttgagc

SEQ ID NO: 41 - DNA, 19 bp, synthetic construct
  misc_feature 1..19: note = Mouse Col1a1 RT-qPCR forward primer
  source 1..19: mol_type = other DNA
gctcctcttaggggccact

SEQ ID NO: 42 - DNA, 19 bp, synthetic construct
  misc_feature 1..19: note = Mouse Col1a1 RT-qPCR reverse primer
  source 1..19: mol_type = other DNA
ccacgtctcaccattgggg

SEQ ID NO: 43 - DNA, 21 bp, synthetic construct
  misc_feature 1..21: note = Mouse FN-EDA RT-qPCR forward primer
  source 1..21: mol_type = other DNA
atgtggacccctcctgatagt

SEQ ID NO: 44 - DNA, 21 bp, synthetic construct
  misc_feature 1..21: note = Mouse FN-EDA RT-qPCR reverse primer
  source 1..21: mol_type = other DNA
gcccagtgatttcagcaaagg

SEQ ID NO: 45 - DNA, 21 bp, synthetic construct
  misc_feature 1..21: note = Mouse TCF21 RT-qPCR forward primer
  source 1..21: mol_type = other DNA
cccactaagaaaagcccgctc

SEQ ID NO: 46 - DNA, 21 bp, synthetic construct
  misc_feature 1..21: note = Mouse TCF21 RT-qPCR reverse primer
  source 1..21: mol_type = other DNA
ccgttctcgtacttgtcgttg

SEQ ID NO: 47 - DNA, 21 bp, synthetic construct
  misc_feature 1..21: note = Mouse Postn RT-qPCR forward primer
  source 1..21: mol_type = other DNA
cctgcccttatatgctctgct

SEQ ID NO: 48 - DNA, 23 bp, synthetic construct
  misc_feature 1..23: note = Mouse Postn RT-qPCR reverse primer
  source 1..23: mol_type = other DNA
aaacatggtcaataggcatcact

SEQ ID NO: 49 - DNA, 21 bp, synthetic construct
  misc_feature 1..21: note = Mouse CTGF RT-qPCR forward primer
  source 1..21: mol_type = other DNA
cctgcccttatatgctctgct

SEQ ID NO: 50 - DNA, 23 bp, synthetic construct
  misc_feature 1..23: note = Mouse CTGF RT-qPCR reverse primer
  source 1..23: mol_type = other DNA
aaacatggtcaataggcatcact

SEQ ID NO: 51 - DNA, 21 bp, synthetic construct
  misc_feature 1..21: note = Human WWP2-P1 RT-qPCR forward primer
  source 1..21: mol_type = other DNA
gagaaactgtccgttcgggtg

SEQ ID NO: 52 - DNA, 21 bp, synthetic construct
  misc_feature 1..21: note = Human WWP2-P1 RT-qPCR reverse primer
  source 1..21: mol_type = other DNA
gacaagcgcgtcctcaatgat

SEQ ID NO: 53 - DNA, 20 bp, synthetic construct
  misc_feature 1..20: note = Human WWP2-P2 RT-qPCR forward primer
  source 1..20: mol_type = other DNA
cttgtcgtgtcgttccgctt

SEQ ID NO: 54 - DNA, 23 bp, synthetic construct
  misc_feature 1..23: note = Human WWP2-P2 RT-qPCR reverse primer
  source 1..23: mol_type = other DNA
accatttgcttcacacttgactt

SEQ ID NO: 55 - DNA, 21 bp, synthetic construct
  misc_feature 1..21: note = Human COL1A1 RT-qPCR forward primer
  source 1..21: mol_type = other DNA
gtgcgatgacgtgatctgtga

SEQ ID NO: 56 - DNA, 19 bp, synthetic construct
  misc_feature 1..19: note = Human COL1A1 RT-qPCR reverse primer
  source 1..19: mol_type = other DNA
cggtggtttcttggtcggt

SEQ ID NO: 57 - DNA, 20 bp, synthetic construct
  misc_feature 1..20: note = Human COL1A2 RT-qPCR forward primer
  source 1..20: mol_type = other DNA
gagcggtaacaagggtgagc

SEQ ID NO: 58 - DNA, 20 bp, synthetic construct
  misc_feature 1..20: note = Human COL1A2 RT-qPCR reverse primer
  source 1..20: mol_type = other DNA
cttccccattagggcctctc

SEQ ID NO: 59 - DNA, 23 bp, synthetic construct
  misc_feature 1..23: note = Human LUM RT-qPCR forward primer
  source 1..23: mol_type = other DNA
ctgcgtttatctcacaacgaact

SEQ ID NO: 60 - DNA, 20 bp, synthetic construct
  misc_feature 1..20: note = Human LUM RT-qPCR reverse primer
  source 1..20: mol_type = other DNA
cagatccagctcaaccaggg

SEQ ID NO: 61 - DNA, 22 bp, synthetic construct
  misc_feature 1..22: note = Human 18S rRNA RT-qPCR forward primer
  source 1..22: mol_type = other DNA
ggccctgtaattggaatgagtc

SEQ ID NO: 62 - DNA, 21 bp, synthetic construct
  misc_feature 1..21: note = Human 18S rRNA RT-qPCR reverse primer
  source 1..21: mol_type = other DNA
ccaagatccaactacgagctt

SEQ ID NO: 63 - DNA, 25 bp, synthetic construct
  misc_feature 1..25: note = Mouse Wwp2 genotyping forward primer
  source 1..25: mol_type = other DNA
gtggtgtcagcaaagcccaaggtgc

SEQ ID NO: 64 - DNA, 25 bp, synthetic construct
  misc_feature 1..25: note = Mouse Wwp2 genotyping reverse primer
  source 1..25: mol_type = other DNA
gagcaccatgggggccaagggcagc

(19) United States
(12) Patent Application Publication
(10) Pub. No.: US 2026/0201389 A1
(43) Pub. Date: Jul. 16, 2026

(54) TREATMENT AND PREVENTION OF DISEASE MEDIATED BY WWP2

(71) Applicant: National University of Singapore, Singapore (SG)

(72) Inventors: Enrico Giuseppe Petretto, Singapore (SG); Aida Moreno Moral, Jaén (ES)

(73) Assignee: National University of Singapore, Singapore (SG)

(21) Appl. No.: 19/415,695

(22) Filed: Dec. 10, 2025

Related U.S. Application Data
(63) Continuation of application No. 17/618,859, filed on Jun. 12, 2020, now abandoned.

(30) Foreign Application Priority Data
Jun. 14, 2019 (GB) 1908544.8

(51) Int. Cl. C12N 15/113; A61K 31/519; A61K 31/55; A61P 29/00

(52) U.S. Cl. CPC C12N 15/1137; A61K 31/519; A61K 31/55; A61P 29/00; C12N 2310/14

(57) ABSTRACT

Methods of treating and preventing fibrosis and pathological inflammation through inhibition of WWP2 are disclosed, as well as agents for use in such methods.

### RELATED APPLICATIONS

This application is a continuation of U.S. application Ser. No. 17/618,859, filed Dec. 13, 2021, which is a national stage filing under 35 U.S.C. 371 of International Patent Application Serial No. PCT/EP2020/066260, filed Jun. 12, 2020, which claims priority from GB Application Serial No. 1908544.8 filed Jun. 14, 2019, the contents of each of which are herein incorporated by reference in their entirety.

### REFERENCE TO AN ELECTRONIC SEQUENCE LISTING

The contents of the electronic sequence listing (M054670026US01-SEQ-AZW.xml; Size: 97,633 bytes; and Date of Creation: Dec. 10, 2025) are herein incorporated by reference in their entirety.

### FIELD OF THE INVENTION

The present invention relates to the treatment and prevention of disease, in particular diseases characterised by pathological inflammation and/or fibrosis.

### BACKGROUND TO THE INVENTION

Tissue fibrosis occurs when the natural healing process ongoing in a tissue is dysregulated, which as a consequence creates damaging extra scar tissue. Although substantial progress has been made in understanding the pathogenesis of excessive tissue fibrosis and fibrotic disorders, there is still no effective (and safe) cure. As fibrosis and resultant organ failure account for at least one third of natural deaths worldwide (Rockey et al., 2015), management of fibrotic diseases still present an important economic burden for human health. There are several common important fibrotic diseases (atherosclerosis, cirrhosis, scleroderma, pulmonary fibrosis, etc.) and since there are no effective treatments to prevent fibrotic tissue from forming, fibrosis is often considered to be an irreversible process. In the clinic, doctors typically try to slow down the fibrotic progress by anti-inflammatory and immunosuppressive drugs, which however lack specificity and present considerable toxicity. Thus, effective and adverse-side-effect free therapies for fibrotic diseases have not been achieved yet. Many fibrotic diseases share the common feature of disordered and exaggerated deposition of extracellular matrix (ECM) in affected tissues (e.g., kidney, lungs, heart), although the specific etiology and causative mechanisms might differ across diseases.

The fibrogenic response comprises several stages starting from the primary injury of the organ. This injury induces the activation of the local resident cells (such as endothelial cells, alveolar cells, etc.), which in turn will recruit and activate immune cells. These immune cells will then induce the activation of fibroblasts into myofibroblasts and these cells will produce ECM deposition (Leach et al., 2013). Fibroblast is a key effector cell in tissue fibrosis, responsible for homeostasis of the ECM. Fibroblasts are triggered by components of the innate and adaptive immune system under injury, when a low-grade and persistent inflammation promotes the triggering of the fibrogenic response. These molecular components induce a complex inflammatory response characterized by the recruitment, proliferation, and activation of several hematopoietic (and non-hematopoietic) cells, e.g., neutrophils, macrophages, innate lymphoid cells, natural killer cells, B cells, T cells, fibroblasts, epithelial cells, endothelial cells, and stem cells. Among these immune cells, macrophages have been shown to have a key regulatory and reparative role (DeBerge et al., 2019); for example, resident tissue macrophages have been found to be able to cloak excess tissue damage to preserve tissue homeostasis (Uderhardt et al., 2019). As such, suppression of inflammatory triggers and resolution of inflammation are significantly important to prevent fibroblasts' activation. Upon triggering of the fibrogenic response, activated fibroblasts (also known as myofibroblasts) increase the synthesis of ACTA2 and of other ECM components, reduce their proliferation and ultimately differentiate into myofibroblasts (Baum and Duffy, 2011). Interactions of fibroblasts with cells involved in innate and adaptive immunity contribute to the fibrogenic program. At the molecular level, the development of tissue fibrosis comprises of a complex signalling cascade, for which many regulators have been proposed such as angiotensin II (Ang II), connective tissue growth factor (CTGF), bone morphogenetic protein (BMP), WNT and cytokines such as interleukin 11 (IL-11) and the transforming growth factor beta (TGFβ) superfamily (Schafer et al., 2017; Leask, 2015; Wang et al., 2011; Piersma et al., 2015). In particular, TGFβ1 binds directly to receptors for signal transduction via downstream effector proteins known as SMADs, which leads to the transcription of ECM proteins (Ross and Hill, 2008; Moustakas et al., 2001).

Ubiquitin ligases are involved in a wide range of diseases and, more specifically, E3 ubiquitin ligases are thought to yield high specificity and less toxicity than other ubiquitins (Jia and Sun, 2011). E3 ubiquitin ligases can both degrade and activate specific substrates, and so may allow for the specific degradation of currently “undruggable” protein targets (Huang and Dixit, 2016). However, to date only a handful of small molecules for cancer therapy (e.g., targeting MDM2 (Carvajal et al., 2018) and SKP2 (Ding et al., 2017)) have been successfully developed for this type of enzyme. To date, several E3 ubiquitin ligases such as SMURF1, SMURF2, ARKADIA, SYNOVIOLIN, NEDD4 and PELLINO1 (reviewed in Huang et al., 2016) have been proposed as regulators of tissue fibrosis, through several mechanisms including direct regulation of TGFβ-signaling. The WW domain containing E3 ubiquitin protein ligase 2 (WWP2) gene, also known as atrophin-1-interacting protein 2 (AIP-2), is a multifunctional ubiquitin E3 ligase. It has been previously reported to regulate various and heterogeneous cellular processes, including palatogenesis (Nakamura et al., 2011), craniofacial development (Zou et al., 2011), TLRβ-mediated innate immune and inflammatory responses (Yang et al., 2013), cell death and tumorigenesis (Maddika et al., 2011) and tumor growth (Fukumoto et al., 2014). More recently, common genetic variation within the WWP2 locus has been associated with plantar fascial disorders and osteoarthritis (Styrkarsdottir et al., 2018; Kim et al., 2018), supporting the heterogeneous functions associated with the WWP2 gene. Moreover, working in cancer cell lines, Soond and colleagues (Soond and Chantry, 2011) implicated WWP2 in oncogenic TGFβ-induced epithelial-mesenchymal transition (EMT), reporting the selective and TGFβ-dependent targeting of SMADs by specific ectopic WWP2 isoforms. Specifically, the WWP2-N isoform was shown to enhance the activity of WWP2-FL, which in turn degrades SMAD2/3. Given the primary role of TGFβ-SMAD2/3 signaling in activating tissue-resident cardiac fibroblasts and the fibrotic response (Khalil et al., 2017), the data by Soond et al. (Soond and Chantry, 2011) might suggest that increased WWP2-N protein isoform expression with concurrent increased activity of WWP2-FL would lead to reduced fibrogenesis (although no data have ever been reported). However, any potential fibrotic or anti-fibrotic function of WWP2 has never been investigated, especially in organ pathophysiology.

### SUMMARY OF THE INVENTION

The present invention concerns the treatment and/or prevention of disease through inhibition of the pro-fibrotic and/or pro-inflammatory functions of WWP2.

The present invention provides methods for treating or preventing disease, comprising inhibiting the expression and/or activity of WWP2, and WWP2 inhibitors and compositions comprising such WWP2 inhibitors for use in such methods.

In particular, the present invention provides for the treatment/prevention of pathological inflammation, fibrosis, diseases characterised by pathological inflammation and/or diseases characterised fibrosis.

In one aspect the present invention provides a WWP2 inhibitor for use in a method of treating or preventing fibrosis, or a disease characterised by fibrosis.

Also provided is the use of a WWP2 inhibitor in the manufacture of a medicament for use in a method of treating or preventing fibrosis, or a disease characterised by fibrosis.

Also provided is a method of treating or preventing fibrosis, or a disease characterised by fibrosis, comprising administering a therapeutically or prophylactically effective amount of a WWP2 inhibitor to a subject.

In some embodiments, the fibrosis is of the heart, kidney, liver, lung, skeletal muscle, blood vessels, eye, skin, pancreas, bowel, small intestine, large intestine, colon, brain, and/or bone marrow.

Also provided is a WWP2 inhibitor for use in a method of treating or preventing pathological inflammation, or a disease characterised by pathological inflammation.

Also provided is the use of a WWP2 inhibitor in the manufacture of a medicament for use in a method of treating or pathological inflammation, or a disease characterised by pathological inflammation.

Also provided is a method of treating or pathological inflammation, or a disease characterised by pathological inflammation, comprising administering a therapeutically or prophylactically effective amount of a WWP2 inhibitor to a subject.

In some embodiments, the pathological inflammation is associated with a chronic infection, a cancer, an autoimmune disease, a degenerative disease or an allergic disease.

In some embodiments, the WWP2 inhibitor is an inhibitor of a WWP2 isoform comprising the C2 domain, optionally wherein the WWP2 inhibitor is an inhibitor of WWP2-FL and/or WWP2-N.

In some embodiments, the WWP2 inhibitor is a WWP2-binding molecule, a WWP2 target-binding molecule, or a molecule capable of reducing expression of WWP2.

In some embodiments, the WWP2 inhibitor is a small molecule.

In some embodiments, the method comprises administering the WWP2 inhibitor to a subject in which expression and/or activity of WWP2 is upregulated.

### DESCRIPTION

The present invention concerns the identification of WWP2 as a positive regulator of a pro-fibrotic gene network common to several chronic diseases upon onset of disease (e.g., in chronic dilated cardiomyopathy, DCM). Increased WWP2 expression in the human diseased heart is shown to lead to increased expression of this gene network of extracellular matrix proteins and pro-fibrotic genes (including collagens, MMPs, cytokines, etc.).

This positive association between expression of WWP2 and pro-fibrotic genes is found to be replicated in different organs associated with chronic diseases. Therefore, the WWP2-regulated molecular pathway captures the pathogenic fibrosis process that takes place across a wide range of fibrotic diseases occurring throughout the body.

Using several loss of function, gain of function and rescue experiments in primary cardiac fibroblasts, the inventors demonstrate that WWP2 regulates the TGFβ-induced transcriptional response of profibrotic genes (e.g., type 1 collagen). In addition, mice lacking the N-terminal region of WWP2 also show a reduction in the complement and coagulation cascade, which is responsible for the activation of immune cells during the fibrogenic response. WWP2 is shown to be involved in the activation of macrophages during fibrosis.

Inhibition of WWP2 is found to reduce proinflammatory macrophage accumulation and expression of proinflammatory factors by macrophages in the context of fibroinflammatory disease. Inhibition of WWP2 is also shown to improve survival and reduce correlates of inflammation and fibrosis in models of lung and kidney fibrosis.

Finally, WWP2 inhibitors are shown to reduce activation of fibroblasts to myofibroblasts, confirming inhibition of WWP2 as a useful strategy for the treatment/prevention of fibroinflammatory disease.

### WWP2

WW domain-containing protein 2 (WWP2; also known as NEDD4-like E3 ubiquitin-protein ligase WWP2, and atrophin-1-interacting protein 2 (AIP2)) is the E3 ubiquitin-protein ligase protein identified by UniProtKB O00308.

WWP2 structure and function is described e.g. in Chen et al., Pathol Oncol Res. (2014) 20(4):799-803 and Soond and Chantry, Oncogene (2011) 30 (21), 2451-2462, both of which are hereby incorporated by reference in their entirety.

WWP2 is a member of the NEDD4-like subgroup of HECT-domain E3 ligases, which are characterised by possessing three functional domains: an N-terminal Ca²⁺/phospholipid-binding C2 domain, a central region containing up to four WW (double-tryptophan) domains that govern target specificity via interaction with PY motifs in target proteins, and a C-terminal HECT domain for which has been shown to be involved in ubiquitin protein ligation.

Ubiquitination is a post-translational modification involved in a variety of cellular processes including protein degradation, membrane protein endocytosis, protein-protein interaction in signal transduction, cell-cycle progression, apoptosis, transcription and immune responses. Ubiquitination involves covalent attachment of ubiquitin protein to free amino groups of internal lysine residues of the target, a process which is catalysed by ubiquitin-activating (E1) ubiquitin-conjugating (E2) and ubiquitin ligase (E3) enzymes. HECT-domain E3 ubiquitin ligases such as WWP2 have intrinsic ligase activity, and bind to and directly mediate ubiquitination of their target substrates.

WWP2 has been shown to be expressed in a wide range of tissues and cell types, including the heart, lung, liver, kidney, peripheral leukocytes, pancreas, muscle (skeletal muscle), spleen, placenta, brain, thymus, liver, colon and intestine (Wood et al., Mol Cell Neurosci (1998) 11(3):149-160; Xu et al., Cell Res (2009) 19(5):561-573).

Several isoforms of WWP2 arise from the same gene. The full-length isoform (WWP2-FL) is an 870 amino acid protein having the sequence shown in SEQ ID NO:1. Positions 20-100 of SEQ ID NO:1 constitute the C2 domain (SEQ ID NO:2), positions 300-333 of SEQ ID NO:1 constitute the WW1 domain (SEQ ID NO:3), positions 330-363 of SEQ ID NO:1 constitute the WW2 domain (SEQ ID NO:4), positions 405-437 of SEQ ID NO:1 constitute the WW3 domain (SEQ ID NO:5), positions 444-477 of SEQ ID NO:1 constitute the WW4 domain (SEQ ID NO:6) and positions 536-870 of SEQ ID NO:1 constitute the HECT domain (SEQ ID NO:7).

The N-terminal isoform (WWP2-N) has the amino acid sequence shown in SEQ ID NO:8, and differs from the WWP2-FL in that it lacks the sequence corresponding to positions 336-870 of SEQ ID NO:1. It therefore comprises only the C2 and WW1 domains. WWP2-N may arise as a result of alternative splicing, through failure to remove the intron between exons 9 and 10.

The C-terminal isoform (WWP2-C) has the amino acid sequence shown in SEQ ID NO:9, and differs from the WWP2-FL in that it lacks the sequence corresponding to positions 1-439 of SEQ ID NO:1. It therefore comprises only the WW4 and HECT domains. WWP2-C may arise from a second internal promoter located in the intron between exons 10 and 11.

A further isoform has been identified which has the amino acid sequence shown in SEQ ID NO:10, and which differs from the WWP2-FL in that it lacks the sequence corresponding to positions 1-116 of SEQ ID NO:1. It therefore comprises the WW1, WW2, WW3, WW4 and HECT domains.

In this specification “WWP2” refers to WWP2 from any species and includes WWP2 isoforms, fragments, variants or homologues from any species. In some embodiments, “WWP2” may refer to a particular isoform or subgroup of isoforms of WWP2 (and/or variants/homologues thereof), e.g. WWP2-FL and/or WWP2-N.

Different WWP2 isoforms display binding to different target proteins, and have different functional properties. For example, WWP2-FL has been shown to bind to TGFβ receptor-regulated R-SMADs SMAD2 and SMAD3, and also to the inhibitory SMAD7. By contrast, WWP2-N only binds to SMAD2 and SMAD3, whilst WWP2-C only binds to SMAD7.

WWP2-N, which lacks a functional HECT ligase domain, has also been shown to interact with WWP2-FL to form a complex with WWP2-FL, and activate WWP2-FL-mediated polyubiqutination and degradation of unphosphorylated SMAD2 and SMAD3.

As used herein, a “fragment”, “variant” or “homologue” of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments, fragments, variants, isoforms and homologues of a reference protein may be characterised by ability to perform a function performed by the reference protein.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. Homologues include orthologues.

A “fragment” may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

In some embodiments, the WWP2 is WWP2 from a mammal (any species in the class Mammalia, e.g. a primate (rhesus, cynomolgous, non-human primate or human) and/or a rodent (e.g. rat or mouse) WWP2). Full-length mouse WWP2 has the amino acid sequence shown in SEQ ID NO:11.

Isoforms, fragments, variants or homologues of WWP2 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature WWP2 isoform from a given species, e.g. human.

Isoforms, fragments, variants or homologues may optionally be functional isoforms, fragments, variants or homologues, e.g. having a functional property/activity of the reference WWP2, as determined by analysis by a suitable assay for the functional property/activity. For example, an isoform, fragment, variant or homologue of WWP2 may e.g. display ubiquitin ligase activity, e.g. as determined by the ability to ubiquitinate a target substrate for WWP2.

In this specification, reference to “WWP2-FL” refers to the protein having the amino acid sequence shown in SEQ ID NO:1, and fragments, variants or homologues thereof. In some embodiments WWP2-FL comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1.

In this specification, reference to “WWP2-N” refers to the protein having the amino acid sequence shown in SEQ ID NO:8, and fragments, variants or homologues thereof. In some embodiments WWP2-N comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:8.

In this specification, reference to “WWP2-C” refers to the protein having the amino acid sequence shown in SEQ ID NO:9, and fragments, variants or homologues thereof. In some embodiments WWP2-C comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:9.

In this specification, reference to “WWP2-2” refers to the protein having the amino acid sequence shown in SEQ ID NO:10, and fragments, variants or homologues thereof. In some embodiments WWP2-2 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:10.

In some embodiments, the WWP2 is a WWP2 isoform selected from WWP2-FL, WWP2-N, WWP2-C, or WWP2-2. In some embodiments, the WWP2 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1, 8, 9 or 10.

In some embodiments, the WWP2 is a WWP2 isoform comprising the C2 domain. Herein, “C2 domain” refers to an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:2.

In some embodiments, the WWP2 comprises an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:2.

In some embodiments, the WWP2 is a WWP2 isoform comprising the WW1 domain. Herein, “WW1 domain” refers to an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:3.

In some embodiments, the WWP2 comprises an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:3.

In some embodiments, the WWP2 is a WWP2 isoform comprising the C2 domain and the WW1 domain.

In some embodiments, the WWP2 comprises an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:2, and an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:3.

In some embodiments, the WWP2 is a WWP2 isoform selected from WWP-FL or WWP2-N. In some embodiments, the WWP2 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1 or 8.

Targets for WWP2 include PTEN, SMAD proteins and OCT4, which have important roles in cell growth and survival. WWP2-mediated ubiquitination and consequent degradation of PTEN has been shown to result in increased AKT signalling and survival of prostate cancer cells, and overexpression of WWP2 has been shown to contribute to oncogenesis and tumorigenicity. WWP2 also interacts with SMAD proteins, which are transcription factor effectors of TGFβ-mediated signalling, which is involved in epithelial-mesenchymal cell transition (EMT), and thus tissue invasion and metastasis. Inhibition of WWP2-FL-mediated degradation of SMAD7 has been shown to reduce TGFβ-induced EMT. OCT4 is involved in maintaining pluripotency of stem cells. Some cancerous cells regain the ability to express OCT4, and reduced ubiquitination and degradation of OCT4 by WWP2 may promote expansion of tumor-initiating cells.

WWP2 has also been shown to be involved in the regulation of immune system function. Overexpression of WWP2 enhances proliferation, increases IL-2 production and suppresses apoptosis of primary mouse T cells. WWP2 may inhibit activation-induced cell death (AICD) through ubiquitin-mediated degradation of EGR, which has been shown to regulate FasL expression. Knockdown of WWP2 reduces TRIF degradation, and elevated TRIF enhances IFNB expression and TLR3 activation.

### Inhibition of WWP2

The present invention is concerned with inhibition of WWP2. That is, the invention is concerned with inhibition of the expression and/or activity of WWP2 and the downstream functional consequences thereof.

Inhibition of WWP2 encompasses decreased/reduced expression (gene and/or protein expression) of WWP2 and/or decreased/reduced activity of WWP2, relative to the level of expression/activity observed in the absence of inhibition. “Inhibition” may herein also be referred to as “antagonism”.

In some embodiments, inhibition is of a particular isoform or subgroup of isoforms of WWP2. In some embodiments, inhibition is of WWP2-FL, WWP2-N and/or WWP2-C. In some embodiments, inhibition is of WWP2 comprising the C2 domain. In some embodiments, inhibition is of WWP2-FL and/or WWP2-N.

In accordance with the present disclosure, inhibition of WWP2 may be characterised by one of more of the following (relative to the uninhibited state):
Reduced expression (e.g. gene and/or protein expression) of WWP2;
Reduced level of RNA encoding WWP2;
Reduced transcription of nucleic acid encoding WWP2;
Increased degradation of RNA encoding WWP2;
Reduced level of WWP2 protein;
Reduced post-transcriptional processing (e.g. splicing, translation, post-translational processing) of RNA encoding WWP2;
Increased degradation of WWP2 protein;
Reduced level of a WWP2 function;
Reduced nuclear translocation of WWP2;
Reduced proportion of WWP2 localised to the nucleus;
Increased proportion of WWP2 localised to the cytoplasm;
Reduced interaction between WWP2 and an interaction partner for WWP2;
Reduced ubiquitin ligase activity by WWP2;
Reduced ubiquitination of a target for WWP2 (e.g. a SMAD, PTEN);
Reduced degradation of a target for WWP2 (e.g. a SMAD, PTEN);
Reduced level of a correlate of WWP2 function;
Reduced gene and/or protein expression, and/or activity, of one or more proteins whose expression is directly/indirectly upregulated as a consequence of WWP2 function;
Increased gene and/or protein expression, and/or activity, of one or more proteins whose expression is directly/indirectly downregulated as a consequence of WWP2 function.

Gene expression can be determined by means well known to the skilled person. The level of RNA encoding WWP2 can be determined e.g. by techniques such as RT-qPCR, northern blot, etc. By way of illustration, in the experimental Examples of the present disclosure the inventors employ qRT-PCR to determine the level of RNA encoding different WWP2 isoforms using primers specific for the detection of regions of the transcripts present in some isoforms but not in others (see e.g. FIG. 6A).

A reduction in the level of RNA encoding WWP2 may e.g. be the result of reduced transcription of nucleic acid encoding WWP2, or increased degradation of RNA encoding WWP2.

Reduced transcription of nucleic acid encoding WWP2 may be a consequence of inhibition of assembly and/or activity of factors required for transcription of the DNA encoding WWP2. Increased degradation of RNA encoding WWP2 may be a consequence of increased enzymatic degradation of RNA encoding WWP2, e.g. as a consequence of RNA interference (RNAi), and/or reduced stability of RNA encoding WWP2.

Protein expression can be determined by means well known to the skilled person. The level of protein encoding WWP2 can be determined e.g. by antibody-based methods including western blot, immunohisto/cytochemistry, flow cytometry, ELISA, ELISPOT, or by reporter-based methods. By way of illustration, in the experimental Examples of the present disclosure the inventors employ western blot and immunocytochemistry techniques to detect and quantify different WWP2 isoforms.

A reduction in the level of protein encoding WWP2 may e.g. be the result of reduced level of RNA encoding WWP2, reduced post-transcriptional processing of RNA encoding WWP2, or increased degradation of WWP2 protein.

Reduced post-transcriptional processing of WWP2 may e.g. be reduced splicing of pre-mRNA encoding WWP2 to mature mRNA encoding WWP2, reduced translation of mRNA encoding WWP2, or reduced post-translational processing of WWP2.

Reduced splicing of pre-mRNA encoding WWP2 to mature mRNA encoding WWP2 may be a consequence of inhibition of assembly and/or activity of factors required for splicing. Reduced translation of mRNA encoding WWP2 may be a consequence of inhibition of assembly and/or activity of factors required for translation. Reduced post-translational processing (e.g. enzymatic processing, folding) of WWP2 may be a consequence of inhibition of assembly and/or activity of factors required for post-translational processing of WWP2. Increased degradation of WWP2 protein may be a consequence of increased enzymatic (e.g. protease-mediated) degradation of WWP2 protein.

In some embodiments, inhibition of WWP2 may be characterised by a reduced level of a WWP2 function. A WWP2 function may be any functional property of WWP2.

In some embodiments, the function is selected from: interaction with an interaction partner, ubiquitin ligase activity, ubiquitination of a target protein, degradation of a target protein, and nuclear translocation.

The subcellular localisation of WWP2 within cells can be analysed using techniques which are well known to the person skilled in the art. Such techniques include e.g. analysis by immunocytochemistry, and western blot of extracts prepared from different cellular fractions. By way of illustration, in the experimental Examples of the present disclosure the inventors employ western blot analysis of nuclear and cytoplasmic fractions in order to analyse the subcellular localisation of WWP2 isoforms.

Such techniques can be employed to analyse the level of nuclear translocation of WWP2, and/or the proportion of WWP2 isoforms in the nucleus and cytoplasm.

Reduced nuclear translocation of WWP2, a reduced proportion of WWP2 localised to the nucleus and/or an increased proportion of WWP2 localised to the cytoplasm may occur e.g. as a result of increased cytoplasmic retention and/or reduced nuclear import of WWP2.

An interaction partner may e.g. be a target protein. A target protein may be any protein which interacts with and/or is ubiquitinated by WWP2. A target protein may be e.g. a SMAD (e.g. SMAD2, SMAD3 and/or SMAD7), PTEN, OCT (e.g. OCT4) or EGR protein. An interaction partner may e.g. be a nucleic acid (e.g. a DNA sequence or an RNA sequence) with which WWP2 interacts.

An interaction partner may be a WWP2; for example, WWP2-FL and WWP-N are known to interact with one another.

The target protein may be e.g. a SMAD (e.g. SMAD2, SMAD3 and/or SMAD7), PTEN, OCT (e.g. OCT4) or EGR protein.

It will be appreciated that different WWP2 isoforms have different functional properties, and a WWP2 function may be selected accordingly. For example, where the WWP2 is WWP2-FL, a SMAD protein may be selected from SMAD2, SMAD3 and/or SMAD7. By contrast, where the WWP2 is WWP2-N a SMAD protein may be SMAD2 and/or SMAD3, and where the WWP2 is WWP2-C a SMAD protein may be SMAD7.

Functional properties of WWP2 can be analysed using appropriate assays, e.g. in vitro assays.

Inhibition of interaction between WWP2 and an interaction partner for WWP2 can be identified e.g. by detection of a reduction in the level of interaction between WWP2 and the interaction partner for WWP2, relative to a control, uninhibited condition. The ability of proteins to interact can be analysed by methods well known to the skilled person, such as co-immunoprecipitation, and resonance energy transfer (RET) assays.

Inhibition of ubiquitin ligase activity/ubiquitination of a target protein can be identified e.g. by detection of a reduction in the level of ubiquitin ligase activity/ubiquitination, relative to a control, uninhibited condition. An assay of ubiquitin ligase activity/ubiquitination of a target protein may comprise detection/quantification of ubiquitination of a target protein, and/or detection/quantification of ubiquitinated target protein.

Inhibition of degradation of a target protein can be identified e.g. by detection of an increase in the level of the target protein, and/or a reduction in the level of polyubiquitinated target protein, relative to a control, uninhibited condition. An assay of target protein degradation may comprise detection/quantification of the target protein, and/or detection/quantification of polyubiquitinated target protein.

Inhibition of nuclear translocation of WWP2 can be identified e.g. by detection of an increase in the level of nuclear translocation of WWP2, relative to a control, uninhibited condition. An assay of nuclear translocation of WWP2 may comprise detection/quantification of WWP2 in the nucleus and/or the cytoplasm, and/or determination of the proportion of WWP2 localised to the nucleus and/or the cytoplasm.

Inhibition of WWP2 function can also be evaluated by analysis of one or more correlates of WWP2 function. That is, WWP2 function can be evaluated by analysis of downstream functional consequences of WWP2 function. For example, inhibition of WWP2 function can be identified by detection of reduced expression (gene and/or protein expression) and/or activity of one or more proteins whose expression is directly/indirectly upregulated as a consequence of WWP2 function. Inhibition of WWP2 function can also be identified by detection of increased expression (gene and/or protein expression) and/or activity of one or more proteins whose expression is directly/indirectly downregulated as a consequence of WWP2 function.

### WWP2 Inhibitors

Aspects of the present invention comprise inhibition of WWP2 using an inhibitor of WWP2.

An “inhibitor of WWP2” refers to any agent capable of inhibiting WWP2 expression and/or function. Such agents may be effectors of (i.e. may directly or indirectly cause) inhibition of WWP2 as described hereinabove. Agents capable of inhibiting WWP2 may be referred to herein as WWP2 inhibitors. WWP2 inhibitors may also be referred to herein as antagonists of WWP2/WWP2 antagonists.

In some embodiments, an inhibitor of WWP2 may:
Reduce expression (e.g. gene and/or protein expression) of WWP2;
Reduce the level of RNA encoding WWP2;
Reduce transcription of nucleic acid encoding WWP2;
Increase degradation of RNA encoding WWP2;
Reduce the level of WWP2 protein;
Reduce post-transcriptional processing (e.g. splicing, translation, post-translational processing) of RNA encoding WWP2;
Increase degradation of WWP2 protein;
Reduce the level of a WWP2 function;
Reduce nuclear translocation of WWP2;
Reduce the proportion of WWP2 localised to the nucleus;
Increase the proportion of WWP2 localised to the cytoplasm;
Reduce interaction between WWP2 and an interaction partner for WWP2; and/or
Reduce ubiquitin ligase activity by WWP2.

A given agent may be evaluated for the properties recited in the preceding paragraph using suitable assays. The assays may be e.g. in vitro assays, optionally cell-based assays or cell-free assays.

Where assays are cell-based assays, they may comprise treating cells to upregulate WWP2 expression and/or activity, e.g. via stimulation with TGFβ1 (e.g. at a final concentration of 5 ng/ml), and treating cells with the test agent in order to determine whether the agent displays one or more of the recited properties.

Agents capable of reducing gene expression of WWP2 (e.g. reducing the level of RNA encoding WWP2, reducing transcription of nucleic acid encoding WWP2 and/or increasing degradation of RNA encoding WWP2) may be identified using assays comprising detecting the level of RNA encoding WWP2, e.g. by RT-qPCR. Such assays may comprise treating cells/tissue with the agent, and subsequently comparing the level of RNA encoding WWP2 in such cells/tissue to the level of RNA encoding WWP2 in cells/tissue of an appropriate control condition (e.g. untreated/vehicle-treated cells/tissue).

Agents capable of reducing protein expression of WWP2 (e.g. reducing the level of WWP2 protein, increasing degradation of WWP2 protein) may be identified using assays comprising detecting the level of WWP2 protein, e.g. using antibody/reporter-based methods (western blot, ELISA, immunohisto/cytochemistry, etc.). Such assays may comprise treating cells/tissue with the agent, and subsequently comparing the level of WWP2 protein in such cells/tissue to the level of WWP2 protein in cells/tissue of an appropriate control condition (e.g. untreated/vehicle-treated cells/tissue).

Agents capable of reducing nuclear translocation of WWP2, reducing the proportion of WWP2 localised to the nucleus and/or increasing the proportion of WWP2 localised to the cytoplasm may be identified using assays comprising detecting the level/proportion of WWP2 in the nucleus/cytoplasm, e.g. using antibody/reporter-based methods. The level/proportion of WWP2 in the nucleus/cytoplasm of cells/tissue may be determined e.g. by immunocytochemistry, or western blot of extracts prepared from different cellular fractions. Assays may comprise treating cells/tissue with the agent, and subsequently detecting the level/proportion of WWP2 in the nucleus and/or cytoplasm of such cells/tissue to the level/proportion of WWP2 in the nucleus and/or cytoplasm of cells/tissue of an appropriate control condition (e.g. untreated/vehicle-treated cells/tissue).

Agents capable of reducing interaction between WWP2 and an interaction partner for WWP2 may be identified using assays comprising detecting the level of interaction between WWP2 and an interaction partner for WWP2, e.g. using antibody/reporter-based methods. The level of interaction between WWP2 and an interaction partner for WWP2 can be analysed e.g. using resonance energy transfer techniques (e.g. FRET, BRET), or methods analysing a correlate of interaction between WWP2 and the interaction partner. Assays may comprise treating cells/tissue with the agent, and subsequently comparing the level of interaction between WWP2 and an interaction partner for WWP2 in such cells/tissue to the level of interaction between WWP2 and the interaction partner for WWP2 in cells/tissue of an appropriate control condition (e.g. untreated/vehicle-treated cells/tissue). The level of interaction between WWP2 and an interaction partner for WWP2 can also be analysed e.g. using techniques such as ELISA, surface plasmon resonance or biolayer interferometry analysis. Assays may comprise comparing the level of interaction between WWP2 and an interaction partner for WWP2 in the presence of the agent to the level of interaction between WWP2 and the interaction partner for WWP2 in an appropriate control condition (e.g. the absence of the agent).

Agents capable of reducing WWP2 ubiquitin ligase activity may be identified using assays comprising detecting the level of WWP2 ubiquitin ligase activity, e.g. using antibody/reporter-based methods. The level of ubiquitin ligase activity by WWP2 e.g. using an in vitro ubiquitination assays, e.g. as described in Example 11.2 herein. Assays may comprise treating cells/tissue with the agent, and subsequently comparing the level of WWP2 ubiquitin ligase activity in such cells/tissue to the level of WWP2 ubiquitin ligase activity in cells/tissue of an appropriate control condition (e.g. untreated/vehicle-treated cells/tissue). The level of WWP2 ubiquitin ligase activity can also be analysed in cell-free assays, which may comprise comparing the level of WWP2 ubiquitin ligase activity in the presence of the agent to the level of WWP2 ubiquitin ligase activity in an appropriate control condition (e.g. the absence of the agent).

Agents capable of inhibiting WWP2 function may be identified using assays comprising detecting the level of a correlate of WWP2 function (e.g. the gene and/or protein expression, and/or activity, of one or more proteins whose expression is directly/indirectly upregulated or downregulated as a consequence of WWP2 function), e.g. using antibody/reporter-based methods. For example, agents capable of inhibiting WWP2 function can be investigated using an in vitro assay of cellular fibrosis as described in Example 11.3 herein. Such assays may comprise treating cells/tissue with the agent, and subsequently comparing the level of the correlate of WWP2 function in such cells/tissue to the level of the correlate of WWP2 function in cells/tissue of an appropriate control condition (e.g. untreated/vehicle-treated cells/tissue).

In some embodiments, a WWP2 inhibitor is an inhibitor of a WWP2 isoform selected from WWP-FL, WWP2-N, WWP2-C, or WWP2-2. In some embodiments, a WWP2 inhibitor is an inhibitor of WWP2 comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1, 8, 9 or 10.

In some embodiments, a WWP2 inhibitor is an inhibitor of a WWP2 isoform comprising the C2 domain. In some embodiments, a WWP2 inhibitor is an inhibitor of WWP2 comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:2.

In some embodiments, a WWP2 inhibitor is an inhibitor of a WWP2 isoform comprising the WW1 domain. In some embodiments, a WWP2 inhibitor is an inhibitor of WWP2 comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:3.

In some embodiments, a WWP2 inhibitor is an inhibitor of a WWP2 isoform comprising the C2 domain and the WW1 domain. In some embodiments, a WWP2 inhibitor is an inhibitor of WWP2 comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:2, and an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:3.

In some embodiments, a WWP2 inhibitor is an inhibitor of WWP-FL or WWP2-N. In some embodiments, a WWP2 inhibitor is an inhibitor of WWP2 comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1 or 8.

In some embodiments, a WWP2 inhibitor is not an inhibitor of WWP2-C. In some embodiments, a WWP2 inhibitor is not an inhibitor of WWP2 consisting of the amino acid sequence of SEQ ID NO:9.

WWP2 inhibitors according to the present disclosure may be any kind of agent possessing the appropriate inhibitory activity.

In some embodiments, a WWP2 inhibitor is selected from: a WWP2-binding molecule, a WWP2 target-binding molecule, or a molecule capable of reducing expression of WWP2.

A “WWP2-binding molecule” refers to a molecule which is capable of binding to WWP2. In some embodiments a WWP2-binding molecule binds to a polypeptide according to SEQ ID NO:1, 8 and/or 9.

A “WWP2 target-binding molecule” refers to a molecule which is capable of binding to an interaction partner for WWP2 (e.g. an interaction partner for WWP2 as described herein, such as SMAD2, PTEN etc.).

Such binding molecules can be identified using any suitable assay for detecting binding of a molecule to the relevant factor (i.e. WWP2, or the interaction partner for WWP2). Such assays may comprise detecting the formation of a complex between the relevant factor and the molecule. For example, a WWP2-binding molecule may be determined as described in Example 11.1 herein.

WWP2-binding molecules and WWP2 target-binding molecules include small molecules. WWP2-binding small molecules and WWP2 target-binding small molecules can be identified by screening of small molecule libraries.

As used herein, a “small molecule” refers to a low molecular weight (<1000 daltons, typically between ~300-700 daltons) organic compound.

Small molecule inhibitors of WWP2 are described e.g. in Watt et al., Chemistry. (2018) 24(67):17677-17680, which is hereby incorporated by reference in its entirety.

Small molecule inhibitors of WWP2 can be identified e.g. using the methods described in Watt et al., Chemistry. (2018) 24(67):17677-17680. In some embodiments the WWP2 inhibitor according to the present invention is a small molecule described in Watt et al., Chemistry. (2018) 24(67):17677-17680, e.g. a compound shown in Table S1 of the Supporting Information.

In some embodiments, a small molecule is selected from: 4,4′-Dimethyl-[1,1′-biphenyl]-2,2′,5,5′-tetraol (NSC 2805), N-((1Z)-2-(2,5-dihydroxyphenyl) ethenyl formamide (NSC 650438), 7-Nitro-4-(1-oxidopyridin-1-ium-2-yl)sulfanyl-2,1,3-benzoxadiazole (NSC 228155), 3,5-Bis(2,5-dioxopyrrol-1-yl)benzoic acid (NSC 44750), 2-(3-Nitrophenyl)-3-nitrosoimidazo[1,2-a]pyrimidine (NSC 369066), 7,8-Dimethyl-10-(2′-acetoxyethyl)isoalloxazine (NSC 3064), 4-[[(1-Amino-2-sulfosulfanylethylidene)amino]methyl]-6,6-dimethylbicyclo[3.1.1]heptane (NSC 194308), 4-Nitro-7-(p-tolylsulfinyl)benzofurazan (NSC 228150), 10-(2-Methoxyethyl)-3-phenylbenzo[g]pteridine-2,4-dione (NSC 288387) and Ethyl violet (NSC 8675).

Small molecule inhibitors of WWP2 can also be identified e.g. using the methods described in Examples 11 and 16 herein.

In some embodiments, a small molecule is 3-(2-chloro-5,6-dihydrobenzo[b][1]benzazepin-11-yl)-N,N-dimethylpropan-1-amine hydrochloride (Clomipramine hydrochloride). In some embodiments, a small molecule is 2-[11-(2,4-dimethoxyphenyl)-10,12-dioxo-7-thia-9,11-diazatricyclo[6.4.0.02,6]dodeca-1(8),2(6)-dien-9-yl]-N-(furan-2-ylmethyl)acetamide.

WWP2-binding molecules and WWP2 target-binding molecules include peptides/polypeptides, e.g. peptide aptamers, thioredoxins, monobodies, anticalin, Kunitz domains, avimers, knottins, fynomers, atrimers, DARPins, affibodies, nanobodies (i.e. single-domain antibodies (sdAbs)) affilins, armadillo repeat proteins (ArmRPs), OBodies and fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48). WWP2-binding molecules and WWP2 target-binding molecules include peptides/polypeptides that can be identified by screening of libraries of the relevant peptides/polypeptides.

WWP2-binding peptides/polypeptides and WWP2 target-binding peptides/polypeptides also include e.g. peptide/polypeptide interaction partners for the relevant factor.

WWP2-binding peptide/polypeptide interaction partners may be based on an interaction partner for WWP2, and may e.g. comprise a WWP2-binding fragment of an interaction partner for WWP2. WWP2 target-binding peptide/polypeptide interaction partners may be based on WWP2, and may e.g. comprise a WWP2 target-binding fragment of WWP2. Such agents may behave as ‘decoy’ molecules, and preferably display competitive inhibition of interaction between WWP2 and an interaction partner for WWP2 and/or WWP2-mediated function.

In some embodiments, a WWP2-binding peptide/polypeptide may comprise or consist of an amino acid sequence having at least 60%, e.g. one of at least 65%, 70%, 75%, 80%, 85%, 90%, 91%, 92%, 93%, 94%, 95%, 96%, 97%, 98% or 99% sequence identity to the amino acid sequence of an interaction partner for WWP2, or the amino acid sequence of a WWP2-binding fragment thereof.

In some embodiments, a WWP2 target-binding peptide/polypeptide may comprise or consist of an amino acid sequence having at least 60%, e.g. one of at least 65%, 70%, 75%, 80%, 85%, 90%, 91%, 92%, 93%, 94%, 95%, 96%, 97%, 98% or 99% sequence identity to the amino acid sequence of WWP2, or the amino acid sequence of a WWP2 target-binding fragment thereof. In such embodiments it will be appreciated that the WWP2 target-binding peptide/polypeptide will lack WWP2 activity and/or have reduced WWP2 activity. For example, in some embodiments a WWP2 target-binding peptide/polypeptide may be a variant (e.g. mutant) WWP2 having reduced function relative to wildtype WWP2.

WWP2-binding molecules and WWP2 target-binding molecules include aptamers. Nucleic acid aptamers are reviewed e.g. in Zhou and Rossi Nat Rev Drug Discov. 2017 16(3):181-202, and may be identified and/or produced by the method of Systematic Evolution of Ligands by EXponential enrichment (SELEX), or by developing SOMAmers (slow off-rate modified aptamers) (Gold L et al. (2010) PLoS ONE 5(12):e15004). Aptamers and SELEX are described in Tuerk and Gold, Science (1990) 249(4968):505-10, and in WO 91/19813. Nucleic acid aptamers may comprise DNA and/or RNA, and may be single stranded or double stranded. They may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose. Nucleic acid aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H. Nucleic Acids Res. 1984, 12, 4539; and Beaucage, S. L.; Lyer, R. P. (1992). Tetrahedron 48 (12): 2223). Peptide aptamers and methods for their generation and identification are reviewed in Reverdatto et al., Curr Top Med Chem. (2015) 15(12):1082-101, which is hereby incorporated by reference in its entirety.

WWP2-binding peptides/polypeptides and WWP2 target-binding peptides/polypeptides also include antibodies (immunoglobulins) such as monoclonal antibodies, polyclonal antibodies, monospecific antibodies, multispecific antibodies (e.g., bispecific antibodies), and fragments and derivatives thereof (e.g. Fv, scFv, Fab, scFab, F(ab′)₂, Fab₂, diabodies, triabodies, scFv-Fc, minibodies, single domain antibodies (e.g. VhH), etc.).

WWP2-binding molecules and WWP2 target-binding molecules may display specific binding to the relevant factor (i.e. WWP2, or the interaction partner for WWP2). As used herein, “specific binding” refers to binding which is selective, and which can be discriminated from non-specific binding to non-target molecules. A WWP2-binding molecule that specifically binds to WWP2 preferably binds to WWP2 with greater affinity, and/or with greater duration than it binds to other, non-target molecules; such binding molecules may be described as being “specific for” WWP2. A WWP2 target-binding molecule that specifically binds to an interaction partner for WWP2 preferably binds to the interaction partner for WWP2 with greater affinity, and/or with greater duration than it binds to other, non-target molecules; such binding molecules may be described as being “specific for” the interaction partner for WWP2.

In some embodiments a WWP2-binding molecule/WWP2 target-binding molecule inhibits the ability of WWP2 to bind to an interaction partner for WWP2. In some embodiments a WWP2-binding molecule/WWP2 target-binding molecule behaves as a competitive inhibitor of interaction between WWP2 and an interaction partner for WWP2. The binding molecule may occupy, or otherwise reduce access to, a region of WWP2 required for binding to an interaction partner for WWP2, or may occupy, or otherwise reduce access to, a region of an interaction partner for WWP2 required for binding to WWP2.

The ability of a WWP2-binding molecule/WWP2 target-binding molecule to inhibit interaction between WWP2 and an interaction partner for WWP2 can be evaluated e.g. by analysis of interaction in the presence of, or following incubation of one or both of the interaction partners with, the WWP2-binding molecule/WWP2 target-binding molecule. An example of a suitable assay to determine whether a given binding agent is capable of inhibiting interaction between WWP2 and an interaction partner for WWP2 is a competition ELISA.

In some embodiments a WWP2-binding molecule/WWP2 target-binding molecule inhibits the ability of WWP2 to perform ubiquitin ligase activity. In some embodiments a WWP2-binding molecule binds to a region of WWP2 required for ubiquitin ligase activity. In some embodiments a WWP2 target-binding molecule binds to a region of an interaction partner for WWP2 required for ubiquitin ligase activity. An example of a suitable assay to determine whether a given binding agent is capable of inhibiting ubiquitin the ability of WWP2 to perform ubiquitin ligase activity is an in vitro ubiquitination assay, which may be performed as described in Example 11.2 herein.

A “molecule capable of reducing expression of WWP2” refers to a molecule which is capable of reducing gene and/or protein expression of WWP2. In some embodiments the molecule reduces or prevents the expression of a polypeptide according to SEQ ID NO:1, 8 and/or 9. In some embodiments the molecule reduces or prevents the expression of a polypeptide from a sequence according to SEQ ID NO:12, 13 and/or 14.

Repression of expression of WWP2 or an isoform thereof will preferably result in a decrease in the quantity of WWP2 expressed by a cell/tissue/organ/organ system/subject. For example, in a given cell the repression of WWP2 by administration of a suitable nucleic acid will result in a decrease in the level of expression relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function. Gene and protein expression may be determined as described herein or by methods in the art that are well known to a skilled person.

Such agents may be of any kind. In some embodiments a molecule capable of reducing expression of WWP2 is an inhibitory nucleic acid.

In some embodiments, the inhibitory nucleic acid is an antisense nucleic acid. In some embodiments the inhibitory nucleic acid is an antisense oligonucleotide (ASO). Antisense oligonucleotides are preferably single-stranded, and bind by complementary sequence binding, to a target oligonucleotide, e.g. mRNA.

In view of the known nucleic acid sequences for WWP2, oligonucleotides may be designed to repress or silence the expression of WWP2, or particular isoforms thereof. The mRNA sequence for human WWP-FL is shown in NCBI Reference Sequence NM_007014.4 (SEQ ID NO:12). The mRNA sequence for human WWP-N is shown in NCBI Reference Sequence NM_001270455.1 (SEQ ID NO:13). The mRNA sequence for human WWP-C is shown in NCBI Reference Sequence NM_199424.2 (SEQ ID NO:14). Oligonucleotides designed to repress or silence the expression of WWP2, or particular isoforms thereof, may have substantial sequence identity to a portion of WWP2/the relevant isoform, or the complementary sequence thereto.

In some embodiments, the inhibitory nucleic acid reduces WWP2 expression by RNA interference (RNAi). RNAi involves inhibition of gene expression and translation by targeted neutralisation of mRNA molecules. In some embodiments, the inhibitory nucleic acid is small interfering RNA (siRNA), a short hairpin RNA (shRNA), or a micro RNA (miRNA).

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase 1, 11 and Ill clinical trials for a number of indications (Nature 2009 Jan. 22; 457(7228):426-433).

In the art, such RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNAs are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences. siRNAs are typically double stranded and, in order to optimise the effectiveness of RNA mediated downregulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response. miRNAs are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of WWP2. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

Targeted inhibition of WWP2 (and particular isoforms) using shRNA is described e.g. Soond et al., Biochimica et Biophysica Acta (2013) 1832 (12): 2127-2135, which is hereby incorporated by reference in its entirety. In some embodiments the WWP2 inhibitor according to the present invention comprises WWP2-targeted shRNA described in Soond et al., Biochimica et Biophysica Acta (2013) 1832 (12): 2127-2135. The primers used to prepare shRNA-pTER plasmids employed in Soond et al., Biochimica et Biophysica Acta (2013) 1832 (12): 2127-2135 for the shRNA-mediated knockdown of specific WWP2 isoforms are shown in SEQ ID NOs:15 to 20.

Targeted inhibition of WWP2 using siRNA and shRNA is described e.g. in Maddika et al., Nat Cell Biol. (2011) 13(6): 728-733, which is hereby incorporated by reference in its entirety. In some embodiments the WWP2 inhibitor according to the present invention comprises WWP2-targeted siRNA described in Maddika et al., Nat Cell Biol. (2011) 13(6): 728-733. In some embodiments the WWP2 inhibitor according to the present invention comprises WWP2-targeted shRNA described in Maddika et al., Nat Cell Biol. (2011) 13(6): 728-733. The siRNAs against WWP2 employed in Maddika et al., Nat Cell Biol. (2011) 13(6): 728-733 for the siRNA-mediated knockdown of WWP2 are shown in SEQ ID NOs:21 to 24. The shRNA against WWP2 employed in Maddika et al., Nat Cell Biol. (2011) 13(6): 728-733 for the shRNA-mediated knockdown of WWP2 are shown in SEQ ID NOs:25 and 26.

Lentiviral vector pLKO.1 encoding shRNA targeting WWP2 designed by The RNAi Consortium is also available from Open Biosystems (Cat. No. RHS4533-EG11060).

In some embodiments, the inhibitory nucleic acid is a splice-switching oligonucleotide (SSO). Splice switching oligonucleotides are reviewed e.g. in Haves and Hastings, Nucleic Acids Res. (2016) 44(14): 6549-6563, which is hereby incorporated by reference in its entirety. SSOs disrupt the normal splicing of target RNA transcripts by blocking the RNA—RNA base-pairing and/or protein—RNA binding interactions that occur between components of the splicing machinery and pre-mRNA. SSOs may be employed to reduce the number/proportion of mature mRNA transcripts encoding the WWP2 isoform that it is intended to inhibit. SSOs generally comprise alterations to oligonucleotide sugar-phosphate backbones to prevent RNAse H degradation, and may comprise include e.g. phosphorodiamidate morpholino (PMOs), peptide nucleic acid (PNA), locked nucleic acid (LNA), and/or 2′O-methyl (2′OMe) and 2′-O-methoxyethyl (MOE) ribose modifications.

Inhibitory nucleic acids may be made recombinantly by transcription of a nucleic acid sequence, preferably contained within a vector. In some embodiments inhibitory nucleic acids are produced within a cell, e.g. by transcription from a vector. Vectors encoding such molecules may be introduced into cells in any of the ways known in the art. Optionally, expression of the RNA sequence can be regulated using a tissue specific (e.g. heart, liver, or kidney specific) promoter.

Inhibitory nucleic acids may also be synthesized using standard solid or solution phase synthesis techniques which are known in the art.

In some embodiments, inhibition of WWP2 may comprise modification of a cell(s) to reduce or prevent expression of WWP2. In some embodiments inhibition of WWP2 comprises modifying nucleic acid encoding WWP2. The modification causes the cell to have a reduced level of gene and/or protein expression of WWP2 as compared to an unmodified cell.

In some embodiments inhibition of WWP2 may comprise modifying a gene encoding WWP2.

In some embodiments inhibition of WWP2 comprises introducing an insertion, substitution or deletion into a nucleic acid sequence encoding WWP2.

In some embodiments inhibition of WWP2 comprises introducing a modification which reduces or prevents the expression of a polypeptide according to SEQ ID NO:1, 8 and/or 9 from the modified nucleic acid sequence. In some embodiments inhibition of WWP2 comprises modifying a cell to comprise a WWP2 allele which does not encode an amino acid sequence according to SEQ ID NO:1, 8 and/or 9. In some embodiments inhibition of WWP2 comprises modifying a cell to lack nucleic acid encoding a polypeptide according to SEQ ID NO:1, 8 and/or 9.

In some embodiments inhibition of WWP2 comprises modifying WWP2 to introduce a premature stop codon in the sequence transcribed from WWP2. In some embodiments inhibition of WWP2 comprises modifying WWP2 to encode a truncated and/or non-functional WWP2 polypeptide. In some embodiments inhibition of WWP2 comprises modifying WWP2 to encode a WWP2 polypeptide which is misfolded and/or degraded.

Modification of a nucleic acid encoding WWP2 in accordance with the methods of the present invention can be achieved in a variety of ways known to the skilled person, including modification of the target nucleic acid by homologous recombination, and target nucleic acid editing using site-specific nucleases (SSNs). Disruption of mouse Wwp2 using a CRISPR/Cas9 system is describe in Example 1.8 herein.

Suitable methods may employ targeting by homologous recombination, which is reviewed, for example, in Mortensen Curr Protoc Neurosci. (2007) Chapter 4:Unit 4.29 and Vasquez et al., PNAS 2001, 98(15): 8403-8410 both of which are hereby incorporated by reference in their entirety. Targeting by homologous recombination involves the exchange of nucleic acid sequence through crossover events guided by homologous sequences.

The methods employ target nucleic acid editing using SSNs. Gene editing using SSNs is reviewed e.g. in Eid and Mahfouz, Exp Mol Med. 2016 October; 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively, DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence.

TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (Moscou and Bogdanove, Science (2009) 326(5959):1501.).

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some embodiments, inhibition of WWP2 employs a site-specific nuclease (SSN) system targeting WWP2. The SSN system may be a ZFN system, a TALEN system, CRISPR/Cas9 system, a CRISPR/Cpf1 system, a CRISPR/C2c1 system, a CRISPR/C2c2 system or a CRISPR/C2c3 system.

For example, inhibition of WWP2 may employ nucleic acid(s) encoding a CRISPR/Cas9 system. The nucleic acid(s) may encode a CRISPR RNA (crRNA) targeting an exon of WWP2 and a trans-activating crRNA (tracrRNA) for processing the crRNA to its mature form.

Treatment/prevention of diseases through inhibition of WWP2 The present invention provides methods and articles (agents and compositions) for the treatment and/or prevention of diseases through inhibition of WWP2. Treatment/prevention of disease is achieved by inhibition of WWP2 in e.g. a cell, tissue/organ/organ system/subject.

The invention is concerned with the treatment and/or prevention of diseases which are caused and/or exacerbated by an increase in the expression/activity of a factor whose expression and/or activity is upregulated by WWP2, and diseases which are caused and/or exacerbated by a decrease in the level/activity of a factor whose expression and/or activity is downregulated by WWP2.

The utility of the present invention extends to the treatment/prevention of any disease that would derive therapeutic/prophylactic benefit from a reduction in the level of WWP2 expression and/or activity.

In some embodiments, a disease to be treated/prevented may be characterised by an increase in the expression/activity of WWP2 (or a correlate thereof) in an organ/tissue/subject affected by the disease e.g. as compared to normal organ/tissue/subject (i.e. in the absence of the disease).

Treatment/prevention may be of a disease that is associated with an upregulation in the expression/activity of WWP2 (or a correlate thereof) in cells/tissue/an organ in which the symptoms of the disease manifest.

The experimental examples of the present disclosure identify WWP2 as a regulator of fibroinflammatory processes, which are moreover conserved between different tissue types.

In particular, the experimental examples demonstrate that inhibition of WWP2 inhibits expression of proinflammatory factors by inflammatory cells (e.g. macrophages, Ly6C-expressing monocytes), and that inhibition of WWP2 also inhibits activation of fibroblasts to myofibroblasts. Inflammatory cells and myofibroblasts are effectors of fibroinflammatory disease affecting various different tissues.

Thus, the present disclosure establishes inhibition of WWP2 as being useful for the treatment/prevention of diseases in which inflammatory cells and/or myofibroblasts are pathologically-implicated, e.g. diseases characterised by inflammation and/or fibrosis.

Aspects of the present invention are concerned with the treatment/prevention of diseases in which profibrotic processes are pathologically implicated.

The inventors have identified WWP2 as a central regulator of the transcription of a network of profibrotic factors, which is highly conserved between different tissues. Accordingly, in some embodiments the disease is fibrosis, or a disease characterised by fibrosis.

As used herein, “fibrosis” refers to the formation of excess fibrous connective tissue as a result of the excess deposition of extracellular matrix components, for example collagen. Fibrous connective tissue is characterised by having extracellular matrix (ECM) with a high collagen content. The collagen may be provided in strands or fibers, which may be arranged irregularly or aligned. The ECM of fibrous connective tissue may also include glycosaminoglycans.

As used herein, “excess fibrous connective tissue” refers to an amount of connective tissue at a given location (e.g. a given tissue or organ, or part of a given tissue or organ) which is greater than the amount of connective tissue present at that location in the absence of fibrosis, e.g. under normal, non-pathological conditions. As used herein, “excess deposition of ECM components” refers to a level of deposition of one or more ECM components which is greater than the level of deposition in the absence of fibrosis, e.g. under normal, non-pathological conditions.

The cellular and molecular mechanisms of fibrosis are described in Wynn, J. Pathol. (2008) 214(2): 199-210, and Wynn and Ramalingam, Nature Medicine (2012) 18:1028-1040, which are hereby incorporated by reference in their entirety.

Damage to tissues can result from various stimuli, including infections, autoimmune reactions, toxins, radiation and mechanical injury. Repair typically involves replacement of injured cells by cells of the same type, and replacement of normal parenchymal tissue with connective tissue. Repair processes become pathogenic when they are not controlled properly, resulting in substantial deposition of ECM components in which normal tissue is replaced with permanent scar tissue. In diseases such as idiopathic pulmonary fibrosis, liver cirrhosis, cardiovascular fibrosis, systemic sclerosis and nephritis, extensive tissue remodelling and fibrosis can ultimately lead to organ failure and death.

The main cellular effectors of fibrosis are myofibroblasts, which produce a collagen-rich ECM. In response to tissue injury, damaged cells and leukocytes produce pro-fibrotic factors such as TGFβ, IL-13 and PDGF, which activate fibroblasts to αSMA-expressing myofibroblasts, and recruit myofibroblasts to the site of injury. Myofibroblasts produce a large amount of ECM, and are important mediators in aiding contracture and closure of the wound. However, under conditions of persistent infection or during chronic inflammation there can be overactivation and recruitment of myofibroblasts, and thus over-production of ECM components, resulting in the formation of excess fibrous connective tissue.

Inflammatory reactions play an important part in triggering fibrosis in many different organ systems. Inflammation can lead to excess in deposition of ECM components in the affected tissues. Low-grade but persistent inflammation is also thought to contribute to the progression of fibrosis in cardiovascular disease and hypertension. In many fibrotic disorders, a persistent inflammatory trigger is crucial to upregulation of production of growth factors, proteolytic enzymes, angiogenic factors and fibrogenic cytokines, which stimulate the deposition of connective tissue elements that progressively remodel and destroy normal tissue architecture.

In some embodiments fibrosis may be triggered by pathological conditions, e.g. conditions, infections or disease states that lead to production of pro-fibrotic factors such as TGFβ1. In some embodiments, fibrosis may be caused by physical injury/stimuli, chemical injury/stimuli or environmental injury/stimuli. Physical injury/stimuli may occur during surgery, e.g. iatrogenic causes. Chemical injury/stimuli may include drug induced fibrosis, e.g. following chronic administration of drugs such as bleomycin, cyclophosphamide, amiodarone, procainamide, penicillamine, gold and nitrofurantoin (Daba et al., Saudi Med J 2004 June; 25(6): 700-6). Environmental injury/stimuli may include exposure to asbestos fibres or silica.

Fibrosis can be of any tissue/organ of the body. In some embodiments, fibrosis is of the heart, kidney, liver, lung, skeletal muscle, blood vessels, eye, skin, pancreas, bowel, small intestine, large intestine, colon, brain, or bone marrow. In some embodiments, the fibrosis is of the heart, lung or kidney. Fibrosis may also occur in multiple tissues/organs at once.

In embodiments, fibrosis may be of an organ of the cardiovascular system, e.g. of the heart or blood vessels. In embodiments herein, fibrosis may be of an organ of the gastrointestinal system, e.g. of the liver, bowel, small intestine, large intestine, colon, or pancreas. In some embodiments, fibrosis may be of an organ of the respiratory system, e.g. the lung. In some embodiments, fibrosis may be of the skin. In some embodiments, fibrosis may be of an organ of the nervous system, e.g. the brain. In some embodiments, fibrosis may be of an organ of the urinary system, e.g. the kidneys. In some embodiments, fibrosis may be of an organ of the musculoskeletal system, e.g. muscle tissue.

In some preferred embodiments, the fibrosis is cardiac or myocardial fibrosis, or renal fibrosis. In some embodiments cardiac or myocardial fibrosis is associated with dysfunction of the musculature or electrical properties of the heart, or thickening of the walls of valves of the heart. In some embodiments fibrosis is of the atrium and/or ventricles of the heart. Treatment or prevention of atrial or ventricular fibrosis may help reduce risk or onset of atrial fibrillation, ventricular fibrillation, or myocardial infarction.

Diseases characterised by fibrosis include but are not limited to: respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, cirrhosis, non-alcoholic fatty liver disease (NAFLD), non-alcoholic steatohepatitis (NASH), primary biliary cirrhosis (PBC), schistosomal liver disease, cardiovascular conditions such as hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Crohn's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's opthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy).

It will be appreciated that many of the diseases/conditions recited in the preceding paragraph are interrelated. For example, fibrosis of the ventricle may occur post myocardial infarction, and is associated with DCM, HCM and myocarditis.

Fibrosis can lead directly or indirectly to, and/or increase susceptibility to development of, diseases. For example, more than 80% of hepatocellular carcinomas (HCCs) develop in fibrotic or cirrhotic livers (Affo et al. 2016, Annu Rev Pathol.), suggesting an important role for liver fibrosis in the premalignant environment (PME) of the liver.

Accordingly, the present invention also finds use in methods for the treatment and prevention of diseases associated with fibrosis, and/or for which fibrosis is a risk factor. In some embodiments, the disease associated with fibrosis, or for which fibrosis is a risk factor, is a cancer, e.g. cancer of the liver (e.g. hepatocellular carcinoma).

In some embodiments, the fibrosis to be treated/prevented according to the present invention may be of fibrosis that is associated with an upregulation of WWP2 expression and/or activity, e.g. in cells/tissue/an organ in which the fibrosis occurs or may occur.

The therapy may be effective to inhibit development (delay/prevent) of the fibrosis, or of progression (e.g. worsening) of the fibrosis. In some embodiments therapy may lead to an improvement in the disease, e.g. a reduction in the symptoms of fibrosis. Prevention of fibrosis may refer to prevention of a worsening of the condition or prevention of the development of fibrosis, e.g. preventing an early stage fibrosis developing to a later stage.

Aspects of the present invention are concerned with the treatment/prevention of diseases in which proinflammatory processes are pathologically implicated.

The inventors have also identified WWP2 as a factor promoting the inflammatory response. In particular, WWP2 expression is shown to be correlated with the expression of complement genes by tissue-resident immune cells (in particular, tissue-resident macrophages), and reduced expression of WWP2 is shown to inhibit upregulation of the expression of proinflammatory factors.

Accordingly, in some embodiments the disease to be treated/prevented in connection with the present invention is a disease characterised by pathological inflammation.

Inflammation is reviewed e.g. in Chen et al., Oncotarget. (2018) 9(6): 7204-7218, which is hereby incorporated by reference in its entirety. Inflammation refers to the bodily response to cellular/tissue injury, and is characterised by edema, erythema (redness), heat, pain, and loss of function (stiffness and immobility) resulting from local immune, vascular and inflammatory cell responses to infection or injury. The injury may result from e.g. of physical (e.g. mechanical) or chemical insult, trauma, infection, cancer or overactive/aberrant immune responses (e.g. autoimmune disease). Inflammation forms part of the innate immune response, and plays an important physiological role in wound healing and the control of infection, and contributes to the restoration of tissue homeostasis.

However, many diseases are associated with an overactive inflammatory response (i.e. excessive inflammation and/or aberrantly activated inflammation), and/or chronic (prolonged) inflammation. Herein, excessive and/or chronic inflammation may be referred to as “pathological inflammation”. Pathological inflammation may refer to inflammation which is implicated in (i.e. which positively contributes to) the pathology of a disease.

In some embodiments, the disease to be treated/prevented in accordance with the present invention is a disease characterised by chronic inflammation. In some embodiments, the disease to be treated/prevented is a disease characterised by an overactive inflammatory response.

In some embodiments, the disease to be treated/prevented is a disease characterised by an increase in the number/proportion of macrophages in a tissue/organ affected by the disease (i.e. a tissue/organ in which symptoms of the disease manifest). In some embodiments, the disease to be treated/prevented is a disease characterised by an increase in the number/proportion of monocytes (e.g. Ly6C-expressing monocytes, e.g. Ly6C^{high} monocytes) in a tissue/organ affected by the disease.

In some embodiments, the treatment/prevention of chronic inflammation or an overactive inflammatory immune response associated with a chronic infection, cancer, autoimmune disease, degenerative disease or allergic disease is contemplated.

Pathological inflammation which is “associated with” a given disease (e.g. a chronic infection, a cancer, an autoimmune disease, a degenerative disease or an allergic disease) may refer to pathological inflammation caused by, initiated by and/or which is a consequence of the disease. Pathological inflammation associated with a given disease may be concurrent with the disease.

Chronic inflammation generally refers to inflammation lasting for prolonged periods of time, e.g. from months to years. Chronic inflammation can result e.g. from failure to properly control/eliminate an infectious agent causing inflammation (i.e. chronic infection), prolonged/repeated exposure to physical/chemical insult, prolonged/repeated exposure to an allergen (allergy), and autoimmune disease.

The chronic inflammation, overactive inflammatory immune response, chronic infection, cancer, autoimmune disease, degenerative disease or allergic disease may affect any tissue/organ of the body, e.g. the heart, kidney, liver, lung, skeletal muscle, blood vessels, eye, skin, pancreas, bowel, small intestine, large intestine, colon, brain, or bone marrow, or multiple tissues/organs at once.

An overactive inflammatory immune response generally refers to an inflammatory immune response that is excessive, and/or which has been activated inappropriately (i.e. an inflammatory immune response which is aberrant). An excessive inflammatory immune response refers to an inflammatory immune response which is greater than the response required for restoration of tissue homeostasis following injury to tissue (e.g. as a result of physical or chemical insult or infection). Aberrant inflammatory immune responses include inflammatory immune responses resulting from autoimmunity and allergy.

Chronic infections include persistent/unresolved infection by any infectious agent, e.g. chronic viral, bacterial, fungal and protozoal infections. Chronic viral infections may be caused e.g. by infection with human immunodeficiency viruses (HIVs), hepatitis B virus (HBV), hepatitis C virus (HCV), Epstein-Barr Virus (EBV), measles virus (MV), cytomegalovirus (CMV), human T-cell leukemia viruses (HTLVs), human herpesviruses (HHVs), herpes simplex viruses (HSVs), Varicella-Zoster virus (VZV), human papovaviruses (e.g. JC virus, BK virus), adenoviruses (AdVs), parvoviruses or human papillomaviruses (HPVs). Chronic bacterial infections may be caused e.g. by infection with Mycobacterium tuberculosis Helicobacter pylori, Salmonella Typhi, Treponema pallidum, Pseudomonas aeruginosa, Escherichia coli, Staphylococcus aureus, Hemophilus influenza or Mycobacterium leprae. Chronic fungal infections may be caused e.g. by infection with Candida spp or Aspergillus. Chronic protozoal infections may be caused e.g. by infection with Plasmodium spp., Babesia spp., Giardia spp., Leishmania spp., Trypanosoma spp. or Toxoplasma spp.

A cancer may be any cancer. As used herein, cancers include any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

An autoimmune disease may be selected from: diabetes mellitus type 1, diabetes mellitus type 2, coeliac disease, Graves' disease, inflammatory bowel disease (e.g. Crohn's disease), multiple sclerosis, psoriasis, rheumatoid arthritis, and systemic lupus erythematosus.

Degenerative diseases are characterised by deterioration of cell/tissue/organ condition or function over time. Proinflammatory and profibrotic processes are implicated in the pathology of many degenerative diseases.

Degenerative disease include e.g. Alzheimer's disease, amyotrophic lateral sclerosis, cancers, Charcot-Marie-Tooth disease, chronic traumatic encephalopathy, cystic fibrosis, degenerative Leigh syndrome, Ehlers-Danlos syndrome, fibrodysplasia ossificans progressiva, Friedreich's ataxia, frontotemporal dementia, cardiovascular diseases (e.g. atherosclerotic cardiovascular disease (e.g. coronary artery disease, aortic stenosis), myocardial infarction, pulmonary arterial hypertension), Huntington's disease, infantile neuroaxonal dystrophy, keratoconus, keratoglobus, leukodystrophies, macular degeneration, Marfan's syndrome, mitochondrial myopathies, mitochondrial DNA depletion syndrome, multiple sclerosis, multiple system atrophy, muscular dystrophies, neuronal ceroid lipofuscinosis, Niemann-Pick disease, osteoarthritis, osteoporosis, Parkinson's disease, pulmonary arterial hypertension, all prion diseases (Creutzfeldt-Jakob disease, fatal familial insomnia etc.), progressive supranuclear palsy, retinitis pigmentosa, rheumatoid arthritis, Sandhoff Disease, spinal muscular atrophy, subacute sclerosing panencephalitis, Tay-Sachs disease and vascular dementia.

An allergic disease may be selected from allergic asthma, allergic rhinitis, food allergy and atopic dermatitis.

In some embodiments the chronic inflammation, overactive inflammatory immune response, chronic infection, cancer, autoimmune disease or allergic disease may be of: an organ of the cardiovascular system, e.g. of the heart or blood vessels; an organ of the gastrointestinal system, e.g. of the liver, bowel, small intestine, large intestine, colon, or pancreas; an organ of the respiratory system, e.g. the lung; the skin; an organ of the nervous system, e.g. the brain; an organ of the urinary system, e.g. the kidneys; or an organ of the musculoskeletal system, e.g. muscle tissue.

Pathological inflammation often leads to fibrosis—see e.g. Mack, Matrix Biol. (2018) 68-69:106-121 and Suthahar et al., Curr Heart Fail Rep. (2017) 14(4): 235-250, both of which are hereby incorporated by reference in their entirety.

The present invention also finds use in methods for the treatment and prevention of diseases associated with pathological inflammation, and/or for which pathological inflammation is a risk factor. In some embodiments, the disease associated with pathological inflammation, or for which pathological inflammation is a risk factor, is fibrosis or a disease characterised by fibrosis.

In some embodiments, the pathological inflammation to be treated/prevented according to the present invention may be of pathological inflammation that is associated with an upregulation of WWP2 expression and/or activity, e.g. in cells/tissue/an organ in which the pathological inflammation occurs or may occur.

The therapy may be effective to inhibit development (delay/prevent) of the pathological inflammation, or of progression (e.g. worsening) of the pathological inflammation. In some embodiments therapy may lead to an improvement in the disease, e.g. a reduction in the symptoms of pathological inflammation. Prevention of pathological inflammation may refer to prevention of a worsening of the condition or prevention of the development of pathological inflammation, e.g. preventing an early stage pathological inflammation developing to a later stage.

Therapeutic/prophylactic intervention in accordance with the present invention may be employed in the context of additional treatment for the relevant disease. That is, WWP2 expression/activity may be inhibited in a subject (e.g. by treatment with a WWP2 inhibitor) that is also receiving/has received/will receive further therapeutic/prophylactic intervention for the treatment/prevention of the disease.

In accordance with various aspects of the present invention, a method of treating and/or preventing a disease according to the present invention may comprise one or more of the following:
Reducing the level of gene/protein expression of WWP2 (e.g. WWP2-FL, WWP-N and/or WWP-C);
Reducing the level of activity of WWP2 (e.g. WWP2-FL, WWP-N and/or WWP-C);
Reducing the level of SMAD (e.g. SMAD2) ubiquitination (e.g. monoubiquitination);
Reducing the level of a correlate of fibrosis (e.g. a collagen, αSMA, periostin, fibronectin, CTGF, vimentin or lumican);
Reducing gene/protein expression of a pro-fibrotic factor (e.g. a collagen, αSMA, periostin, fibronectin, CTGF, vimentin or lumican);
Reducing the number/proportion of myofibroblasts;
Reducing the level of a correlate of pathological inflammation (e.g. a complement pathway protein, C5aR, IL-6, CD206);
Reducing gene/protein expression of a pro-inflammatory factor (e.g. a complement pathway protein, C5aR, IL-6, CD206);
Reducing the number/proportion of C5aR-expressing cells;
Reducing the number/proportion of myofibroblasts;
Reducing the gene/protein expression of ACTA2;
Increasing the function of an organ/tissue affected by the disease;
Increasing the survival of a subject having the disease;
Reducing the number/proportion of macrophages in an organ/tissue affected by the disease;
Reducing the number/proportion of monocytes (e.g. Ly6C-expressing monocytes, e.g. Ly6C^{high} monocytes) in an organ/tissue affected by the disease;
Reducing gene/protein expression of a group 1 or group 3 gene shown in FIG. 22E (e.g. S100A9, IF12712A or IRF7), e.g. by a macrophage;
Increasing gene/protein expression of a group 2 gene shown in FIG. 22E, e.g. by a macrophage;
Reducing gene/protein expression or the level of a proinflammatory factor (e.g. S100A9, CCL12, CCL5, CCL2, TNFα, S100A8, IFNγ, IL-6 or iNOS), e.g. by a macrophage;
Reducing CCL5-mediated signalling, e.g. by a macrophage.

### Administration

Inhibition of WWP2 or administration of a WWP2 inhibitor is preferably in a “therapeutically effective” or “prophylactically effective” amount, this being sufficient to show therapeutic/prophylactic benefit to the subject.

The actual amount administered, and rate and time-course of administration, will depend on the nature and severity of the disease to be treated/prevented, and the nature of the agent. Prescription of treatment, e.g. decisions on dosage etc., is within the responsibility of general practitioners and other medical doctors, and typically takes account of the disease/condition to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of the agent may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In therapeutic applications, WWP2 inhibitors are preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio.

Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulations may be prepared for topical, parenteral, systemic, intravenous, intra-arterial, intramuscular, intrathecal, intraocular, intra-conjunctival, subcutaneous, oral or transdermal routes of administration which may include injection. Injectable formulations may comprise the selected agent in a sterile or isotonic medium. The formulation and mode of administration may be selected according to the agent and disease to be treated/prevented.

Aspects and embodiments of the present invention concern detection of WWP2 expression (gene and/or protein expression) and/or activity in a cell/tissue/organ of a subject, e.g. as determined by analysis of a cell/tissue/organ of a subject, e.g. in a sample obtained from the subject.

Upregulated WWP2 expression and/or activity may identify a subject as a subject to be treated with a WWP2 inhibitor in accordance with the present invention.

Upregulated WWP2 expression/activity refers to a level of expression/activity that is greater than would be expected for a cell/tissue of a given type. WWP2 gene or protein expression and WWP2 activity can be analysed as described herein.

Upregulation may be determined by measuring the level of expression/activity of WWP2 in a cell/tissue. Comparison may be made between the level of expression/activity in a cell or tissue sample from a subject and a reference level of expression/activity, e.g. a value/range of values representing a normal level of expression/activity for the same or corresponding cell/tissue type. In some embodiments reference levels may be determined by detecting expression/activity of WWP2 in a control sample, e.g. in corresponding cells or tissue from a healthy subject or from healthy tissue of the same subject. In some embodiments reference levels may be obtained from a standard curve or data set.

A sample obtained from a subject may be of any kind. A biological sample may be taken from any tissue or bodily fluid, e.g. a blood sample, blood-derived sample, serum sample, lymph sample, semen sample, saliva sample, synovial fluid sample. A blood-derived sample may be a selected fraction of a patient's blood, e.g. a selected cell-containing fraction or a plasma or serum fraction. A sample may comprise a tissue sample or biopsy; or cells isolated from a subject. Samples may be collected by known techniques, such as biopsy or needle aspirate. Samples may be stored and/or processed for subsequent determination of the level of WWP2 expression/activity.

In some preferred embodiments a sample may be a tissue sample, e.g. biopsy, taken from a tissue/organ affected by a disease described herein. A sample may contain cells.

A subject may be selected for therapy/prophylaxis in accordance with the present invention based on determination that the subject has an upregulated level WWP2 expression/activity. Upregulated WWP2 expression/activity may serve as a marker of a disease suitable for treatment in accordance with the present invention.

Following selection, a subject may be treated to inhibit WWP2 expression and/or activity, e.g. by administration of a WWP2 inhibitor.

### Diagnosis and Prognosis

Detection of upregulation of WWP2 expression/activity may also be used in a method of diagnosing a disease described herein, identifying a subject at risk of developing a disease described herein, and in methods of prognosing a subject's response to inhibition of WWP2 inhibition (e.g. via treatment with a WWP2 inhibitor).

In some embodiments a subject may be suspected of having or suffering from a disease, e.g. based on the presence of other symptoms indicative of the disease in the subject's body or in selected cells/tissues of the subject's body, or be considered at risk of developing the disease, e.g. because of genetic predisposition or exposure to environmental conditions, known to be risk factors for the disease. Determination of upregulation of WWP2 expression/activity may confirm a diagnosis or suspected diagnosis, or may confirm that the subject is at risk of developing the disease. The determination may also diagnose a disease or predisposition as one suitable for treatment with a WWP2 inhibitor.

As such, a method of providing a prognosis for a subject having, or suspected of having a disease may be provided, the method comprising determining whether WWP2 expression/activity is upregulated in a sample obtained from the subject and, based on the determination, providing a prognosis for treatment of the subject with a WWP2 inhibitor.

In some aspects, methods of diagnosis or methods of prognosing or predicting a subject's response to treatment with a WWP2 inhibitor may not require determination of WWP2 expression/activity, but may be based on determining genetic factors in the subject that are predictive of upregulation of expression/activity. Such genetic factors may include the determination of genetic variation such as single nucleotide polymorphisms (SNPs) which are correlated with and/or predictive of upregulation of WWP2 expression/activity. The use of genetic factors to predict predisposition to a disease state or response to treatment is known in the art, e.g. see Peter Stärkel Gut 2008; 57:440-442; Wright et al., Mol. Cell. Biol. March 2010 vol. 30 no. 6 1411-1420.

Genetic factors may be assayed by methods known to those of ordinary skill in the art, including PCR based assays. By determining the presence of genetic factors, e.g. in a sample obtained from a subject, a diagnosis may be confirmed, and/or a subject may be classified as being at risk of developing a disease described herein, and/or a subject may be identified as being suitable for treatment with WWP2 inhibitor.

Some methods may comprise determination of the presence of one or more SNPs linked to WWP2 expression/activity, or susceptibility to development of a disease described herein. SNPs are usually bi-allelic and therefore can be readily determined using one of a number of conventional assays known to those of skill in the art (e.g. see Anthony J. Brookes. The essence of SNPs. Gene Volume 234, Issue 2, 8 Jul. 1999, 177-186; Fan et al., Highly Parallel SNP Genotyping. Cold Spring Harb Symp Quant Biol 2003. 68: 69-78; Matsuzaki et al., Parallel Genotyping of Over 10,000 SNPs using a one-primer assay on a high-density oligonucleotide array. Genome Res. 2004. 14: 414-425).

In some embodiments, the methods comprise determining which allele is present at the polymorphic nucleotide position of rs9936589 or a SNP in linkage disequilibrium rs9936589 with an R²≥0.8.

The methods may comprise determining which SNP allele is present in a sample obtained from a subject. In some embodiments determining the presence of the minor allele may be associated with WWP2 expression/activity or susceptibility to development of a disease described herein.

The determining step may comprise determining whether the minor allele is present in the sample at the selected polymorphic nucleotide position. The screening method may be, or form part of, a method for determining susceptibility of the subject to development of a disease described herein, or a method of diagnosis or prognosis as described herein.

The method may further comprise the step of identifying the subject as having susceptibility to, or an increased risk of, developing a disease described herein, e.g. if the subject is determined to have a minor allele at the polymorphic nucleotide position. The method may further comprise the step of selecting the subject for treatment with a WWP2 inhibitor and/or administering WWP2 inhibitor to the subject in order to provide a treatment for a disease described herein in the subject or to prevent development or progression of a disease described herein in the subject.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body. The sample obtained from a subject may be of any kind, as described herein above.

Other diagnostic or prognostic tests may be used in conjunction with those described here to enhance the accuracy of the diagnosis or prognosis or to confirm a result obtained using the tests described herein.

### Subjects

Subjects may be animal or human. Subjects are preferably mammalian, more preferably human. The subject may be a non-human mammal, but is more preferably human. The subject may be male or female. The subject may be a patient.

The patient may have a disease described herein. A subject may have been diagnosed with a disease requiring treatment, may be suspected of having such a disease, or may be at risk from developing a disease.

In embodiments according to the present invention the subject is preferably a human subject. In embodiments according to the present invention, a subject may be selected for treatment according to the methods based on characterisation for certain markers of a disease described herein.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

Sequences

| SEQ ID | | |
|---|---|---|
| NO: | DESCRIPTION | SEQUENCE |
| 1 | WWP2-FL (UniProtKB: | MASASSSRAGVALPFEKSQLTLKVVSAKPKVHNRQPRINSYVEVAVDGLPSETKKTGKRI |
| | O00308-1, v2) | GSSELLWNEIIILNVTAQSHLDLKVWSCHTLRNELLGTASVNLSNVLKNNGGKMENMQLT |
| | | LNLQTENKGSVVSGGELTIFLDGPTVDLGNVPNGSALTDGSQLPSRDSSGTAVAPENRH |
| | | QPPSTNCFGGRSRTHRHSGASARTTPATGEQSPGARSRHRQPVKNSGHSGLANGTVN |
| | | DEPTTATDPEEPSVVGVTSPPAAPLSVTPNPNTTSLPAPATPAEGEEPSTSGTQQLPAA |
| | | AQAPDALPAGWEQRELPNGRVYYVDHNTKTTTWERPLPPGWEKRTDPRGRFYYVDHN |
| | | TRTTTWQRPTAEYVRNYEQWQSQRNQLQGAMQHFSQRFLYQSSSASTDHDPLGPLPP |
| | | GWEKRQDNGRVYYVNHNTRTTQWEDPRTQGMIQEPALPPGWEMKYTSEGVRYFVDH |
| | | NTRTTTFKDPRPGFESGTKQGSPGAYDRSFRWKYHQFRFLCHSNALPSHVKISVSRQT |
| | | LFEDSFQQIMNMKPYDLRRRLYIIMRGEEGLDYGGIAREWFFLLSHEVLNPMYCLFEYAG |
| | | KNNYCLQINPASSINPDHLTYFRFIGRFIAMALYHGKFIDTGFTLPFYKRMLNKRPTLKDLE |
| | | SIDPEFYNSIVWIKENNLEECGLELYFIQDMEILGKVTTHELKEGGESIRVTEENKEEYIML |
| | | LTDWRFTRGVEEQTKAFLDGFNEVAPLEWLRYFDEKELELMLCGMQEIDMSDWQKSTI |
| | | YRHYTKNSKQIQWFWQVVKEMDNEKRIRLLQFVTGTCRLPVGGFAELIGSNGPQKFCID |
| | | KVGKETWLPRSHTCFNRLDLPPYKSYEQLREKLLYAIEETEGFGQE |
| 2 | WWP2 C2 domain | LTLKVVSAKPKVHNRQPRINSYVEVAVDGLPSETKKTGKRIGSSELLWNEIIILNVTAQSH |
| | | LDLKVWSCHTLRNELLGTAS |
| 3 | WWP2 WW1 domain | DALPAGWEQRELPNGRVYYVDHNTKTTTWERPLP |
| 4 | WWP2 WW2 domain | RPLPPGWEKRTDPRGRFYYVDHNTRTTTWQRPTA |
| 5 | WWP2 WW3 domain | GPLPPGWEKRQDNGRVYYVNHNTRTTQWEDPRT |
| 6 | WWP2 WW4 domain | PALPPGWEMKYTSEGVRYFVDHNTRTTTFKDPRP |
| 7 | WWP2 HECT domain | KPYDLRRRLYIIMRGEEGLDYGGIAREWFFLLSHEVLNPMYCLFEYAGKNNYCLQINPAS |
| | | SINPDHLTYFRFIGRFIAMALYHGKFIDTGFTLPFYKRMLNKRPTLKDLESIDPEFYNSIVWI |
| | | KENNLEECGLELYFIQDMEILGKVTTHELKEGGESIRVTEENKEEYIMLLTDWRFTRGVE |
| | | EQTKAFLDGFNEVAPLEWLRYFDEKELELMLCGMQEIDMSDWQKSTIYRHYTKNSKQIQ |
| | | WFWQVVKEMDNEKRIRLLQFVTGTCRLPVGGFAELIGSNGPQKFCIDKVGKETWLPRS |
| | | HTCFNRLDLPPYKSYEQLREKLLYAIEETEGFGQE |
| 8 | WWP2-N | MASASSSRAGVALPFEKSQLTLKVVSAKPKVHNRQPRINSYVEVAVDGLPSETKKTGKRI |
| | (UniProtKB: O00308-4) | GSSELLWNEIIILNVTAQSHLDLKVWSCHTLRNELLGTASVNLSNVLKNNGGKMENMQLT |
| | | LNLQTENKGSVVSGGELTIFLDGPTVDLGNVPNGSALTDGSQLPSRDSSGTAVAPENRH |
| | | QPPSTNCFGGRSRTHRHSGASARTTPATGEQSPGARSRHRQPVKNSGHSGLANGTVN |
| | | DEPTTATDPEEPSVVGVTSPPAAPLSVTPNPNTTSLPAPATPAEGEEPSTSGTQQLPAA |
| | | AQAPDALPAGWEQRELPNGRVYYVDHNTKTTTWERPLPPG |
| 9 | WWP2-C | MIQEPALPPGWEMKYTSEGVRYFVDHNTRTTTFKDPRPGFESGTKQGSPGAYDRSFR |
| | (UniProtKB: O00308-3) | WKYHQFRFLCHSNALPSHVKISVSRQTLFEDSFQQIMNMKPYDLRRRLYIIMRGEEGLD |
| | | YGGIAREWFFLLSHEVLNPMYCLFEYAGKNNYCLQINPASSINPDHLTYFRFIGRFIAMAL |
| | | YHGKFIDTGFTLPFYKRMLNKRPTLKDLESIDPEFYNSIVWIKENNLEECGLELYFIQDMEI |
| | | LGKVTTHELKEGGESIRVTEENKEEYIMLLTDWRFTRGVEEQTKAFLDGFNEVAPLEWL |
| | | RYFDEKELELMLCGMQEIDMSDWQKSTIYRHYTKNSKQIQWFWQVVKEMDNEKRIRLL |
| | | QFVTGTCRLPVGGFAELIGSNGPQKFCIDKVGKETWLPRSHTCFNRLDLPPYKSYEQLR |
| | | EKLLYAIEETEGFGQE |
| 10 | WWP2-2 | MQLTLNLQTENKGSVVSGGELTIFLDGPTVDLGNVPNGSALTDGSQLPSRDSSGTAVAP |
| | (UniProtKB: O00308-2) | ENRHQPPSTNCFGGRSRTHRHSGASARTTPATGEQSPGARSRHRQPVKNSGHSGLAN |
| | | GTVNDEPTTATDPEEPSVVGVTSPPAAPLSVTPNPNTTSLPAPATPAEGEEPSTSGTQQ |
| | | LPAAAQAPDALPAGWEQRELPNGRVYYVDHNTKTTTWERPLPPGWEKRTDPRGRFYY |
| | | VDHNTRTTTWQRPTAEYVRNYEQWQSQRNQLQGAMQHFSQRFLYQSSSASTDHDPL |
| | | GPLPPGWEKRQDNGRVYYVNHNTRTTQWEDPRTQGMIQEPALPPGWEMKYTSEGVR |
| | | YFVDHNTRTTTFKDPRPGFESGTKQGSPGAYDRSFRWKYHQFRFLCHSNALPSHVKIS |
| | | VSRQTLFEDSFQQIMNMKPYDLRRRLYIIMRGEEGLDYGGIAREWFFLLSHEVLNPMYCL |
| | | FEYAGKNNYCLQINPASSINPDHLTYFRFIGRFIAMALYHGKFIDTGFTLPFYKRMLNKRP |
| | | TLKDLESIDPEFYNSIVWIKENNLEECGLELYFIQDMEILGKVTTHELKEGGESIRVTEENK |
| | | EEYIMLLTDWRFTRGVEEQTKAFLDGFNEVAPLEWLRYFDEKELELMLCGMQEIDMSD |
| | | WQKSTIYRHYTKNSKQIQWFWQVVKEMDNEKRIRLLQFVTGTCRLPVGGFAELIGSNGP |
| | | QKFCIDKVGKETWLPRSHTCFNRLDLPPYKSYEQLREKLLYAIEETEGFGQE |
| 11 | Mouse WWP2 | MASASSSRAGVALPFEKSQLTLKVVSAKPKVHNRQPRINSYVEVAVDGLPSETKKTGKRI |
| | (UniProtKB: Q9DBH0-1, | GSSELLWNEIIVLNVTAQSHLDLKVWSCHTLRNELLGTASVNLSNVLKNNGGKMENTQL |
| | v1) | TLNLQTENKGSVVSGGELTIFLDGPTVDLGSVPNGSAVTDGSQPPSRESSGTAIAPETR |
| | | HQPPSTNCFGGRSRTHRHSGGSARTATAASEQSPGARNRHRQPVKNSSSSGLANGTV |
| | | NEEPTPASEPEESSVVGVTSLPAAALSVSSNPNTTSLPAQSTPAEGEEASTSGTQQLPA |
| | | AAQAPDALPAGWEQRELPNGRVYYVDHNTKTTTWERPLPPGWEKRTDPRGRFYYVDH |
| | | NTRTTTWQRPTAEYVRNYEQWQSQRNQLQGAMQHFSQRFLYQSSSASTDHDPLGPLP |
| | | PGWEKRQDNGRVYYVNHNTRTTQWEDPRTQGMIQEPALPPGWEMKYTSEGVRYFVD |
| | | HNTRTTTFKDPRPGFESGTKQGSPGAYDRSFRWKYHQFRFLCHSNALPSHVKISVSRQ |
| | | TLFEDSFQQIMNMKPYDLRRRLYIIMRGEEGLDYGGIAREWFFLLSHEVLNPMYCLFEYA |
| | | GKNNYCLQINPASSINPDHLTYFRFIGRFIAMALYHGKFIDTGFTLPFYKRMLNKRPTLKD |
| | | LESIDPEFYNSIVWIKENNLEECGLELFFIQDMEILGKVTTHELKEGGENIRVTEENKEEYI |
| | | MLLTDWRFTRGVEEQTKAFLDGFNEVAPLEWLRYFDEKELELMLCGMQEIDMSDWQK |
| | | NAIYRHYTKSSKQIQWFWQVVKEMDNEKRIRLLQFVTGTCRLPVGGFAELIGSNGPQKF |
| | | CIDRVGKETWLPRSHTCFNRLDLPPYKSYEQLKEKLLYAIEETEGFGQE |
| 12 | WWP2-FL mRNA | CTCTTCCGGGTCAGGTGACCGCGCGCGGTCACGTGACCCGGCCCGAGTGCGGGC |
| | (NCBI NM_007014.4) | GGTGGAAGGCGGAAGTAGGAGAGGAGTTCGGCGCCGCTTCTGTGGCCACGGCAGA |
| | | TGAAACAGAAAGGCTAAAGAGGGCTGGAGTCAGGGGACTTCTCTTCCACCAGCTTC |
| | | ACGGTGATGATATGGCATCTGCCAGCTCTAGCCGGGCAGGAGTGGCCCTGCCTTTT |
| | | GAGAAGTCTCAGCTCACTTTGAAAGTGGTGTCCGCAAAGCCCAAGGTGCATAATCGT |
| | | CAACCTCGAATTAACTCCTACGTGGAGGTGGCGGTGGATGGACTCCCCAGTGAGAC |
| | | CAAGAAGACTGGGAAGCGCATTGGGAGCTCTGAGCTTCTCTGGAATGAGATCATCA |
| | | TTTTGAATGTCACGGCACAGAGTCATTTAGATTTAAAGGTCTGGAGCTGCCATACCTT |
| | | GAGAAATGAACTGCTAGGCACCGCATCTGTCAACCTCTCCAACGTCTTGAAGAACAA |
| | | TGGGGGCAAAATGGAGAACATGCAGCTGACCCTGAACCTGCAGACGGAGAACAAAG |
| | | GCAGCGTTGTCTCAGGCGGAGAGCTGACAATTTTCCTGGACGGGCCAACTGTTGAT |
| | | CTGGGAAATGTGCCTAATGGCAGTGCCCTGACAGATGGATCACAGCTGCCTTCGAG |
| | | AGACTCCAGTGGAACAGCAGTAGCTCCAGAGAACCGGCACCAGCCCCCCAGCACAA |
| | | ACTGCTTTGGTGGAAGATCCCGGACGCACAGACATTCGGGTGCTTCAGCCAGAACA |
| | | ACCCCAGCAACCGGCGAGCAAAGCCCCGGTGCTCGGAGCCGGCACCGCCAGCCC |
| | | GTCAAGAACTCAGGCCACAGTGGCTTGGCCAATGGCACAGTGAATGATGAACCCAC |
| | | AACAGCCACTGATCCCGAAGAACCTTCCGTTGTTGGTGTGACGTCCCCACCTGCTG |
| | | CACCCTTGAGTGTGACCCCGAATCCCAACACGACTTCTCTCCCTGCCCCAGCCACA |
| | | CCGGCTGAAGGAGAGGAACCCAGCACTTCGGGTACACAGCAGCTCCCAGCGGCTG |
| | | CCCAGGCCCCCGACGCTCTGCCTGCTGGATGGGAACAGCGAGAGCTGCCCAACGG |
| | | ACGTGTCTATTATGTTGACCACAATACCAAGACCACCACCTGGGAGCGGCCCCTTCC |
| | | TCCAGGCTGGGAAAAACGCACAGATCCCCGAGGCAGGTTTTACTATGTGGATCACA |
| | | ATACTCGGACCACCACCTGGCAGCGTCCGACCGCGGAGTACGTGCGCAACTATGAG |
| | | CAGTGGCAGTCGCAGCGGAATCAGCTCCAGGGGGCCATGCAGCACTTCAGCCAAA |
| | | GATTCCTCTACCAGTCTTCGAGTGCTTCGACTGACCATGATCCCCTGGGCCCCCTCC |
| | | CTCCTGGCTGGGAGAAGAGACAGGACAATGGACGGGTGTATTACGTGAACCATAAC |
| | | ACTCGCACGACCCAGTGGGAGGATCCCCGGACCCAGGGGATGATCCAGGAACCAG |
| | | CTCTGCCCCCAGGATGGGAGATGAAATACACCAGCGAGGGGGTGCGATACTTTGTG |
| | | GACCACAATACCCGCACCACCACCTTTAAGGATCCTCGCCCGGGGTTTGAGTCGGG |
| | | GACGAAGCAAGGTTCCCCTGGTGCTTATGACCGCAGTTTTCGGTGGAAGTATCACC |
| | | AGTTCCGTTTCCTCTGCCATTCAAATGCCCTACCTAGCCACGTGAAGATCAGCGTTT |
| | | CCAGGCAGACGCTTTTCGAAGATTCCTTCCAACAGATCATGAACATGAAACCCTATG |
| | | ACCTGCGCCGCCGGCTCTACATCATCATGCGTGGCGAGGAGGGCCTGGACTATGG |
| | | GGGCATCGCCAGAGAGTGGTTTTTCCTCCTGTCTCATGAGGTGCTCAACCCTATGTA |
| | | TTGTTTATTTGAATATGCCGGAAAGAACAATTACTGCCTGCAGATCAACCCCGCCTC |
| | | CTCCATCAACCCGGACCACCTCACCTACTTTCGCTTTATAGGCAGATTCATCGCCAT |
| | | GGCGCTGTACCATGGAAAGTTCATCGACACGGGCTTCACCCTCCCTTTCTACAAGC |
| | | GGATGCTCAATAAGAGACCAACCCTGAAAGACCTGGAGTCCATTGACCCTGAGTTCT |
| | | ACAACTCCATTGTCTGGATCAAAGAGAACAACCTGGAAGAATGTGGCCTGGAGCTGT |
| | | ACTTCATCCAGGACATGGAGATACTGGGCAAGGTGACGACCCACGAGCTGAAGGAG |
| | | GGCGGCGAGAGCATCCGGGTCACAGAGGAGAACAAGGAAGAGTACATCATGCTGC |
| | | TGACTGACTGGCGTTTCACCCGAGGCGTGGAAGAGCAGACCAAAGCCTTCCTGGAT |
| | | GGCTTCAACGAGGTGGCCCCGCTGGAGTGGCTGCGCTACTTTGACGAGAAAGAGCT |
| | | GGAGCTGATGCTGTGCGGCATGCAGGAGATAGACATGAGCGACTGGCAGAAGAGC |
| | | ACCATCTACCGGCACTACACCAAGAACAGCAAGCAGATCCAGTGGTTCTGGCAGGT |
| | | GGTGAAGGAGATGGACAACGAGAAGAGGATCCGGCTGCTGCAGTTTGTCACCGGTA |
| | | CCTGCCGCCTGCCCGTCGGGGGATTTGCCGAACTCATCGGTAGCAACGGACCACA |
| | | GAAGTTTTGCATTGACAAAGTTGGCAAGGAAACCTGGCTGCCCAGAAGCCACACCT |
| | | GCTTCAACCGTCTGGATCTTCCACCCTACAAGAGCTACGAACAGCTGAGAGAGAAG |
| | | CTGCTGTATGCCATTGAGGAGACCGAGGGCTTTGGACAGGAGTAACCGAGGCCGC |
| | | CCCTCCCACGCCCCCCAGCGCACATGTAGTCCTGAGTCCTCCCTGCCTGAGAGGCC |
| | | ACTGGCCCCGCAGCCCTTGGGAGGCCCCCGTGGATGTGGCCCTGTGTGGGACCAC |
| | | ACTGTCATCTCGCTGCTGGCAGAAAAGCCTGATCCCAGGAGGCCCTGCAGTTCCCC |
| | | CGACCCGCGGATGGCAGTCTGGAATAAAGCCCCCTAGTTGCCTTTGGCCCCACCTT |
| | | TGCAAAGTTCCAGAGGGCTGACCCTCTCTGCAAAACTCTCCCCTGTCCTCTAGACCC |
| | | CACCCTGGGTGTATGTGAGTGTGCAAGGGAAGGTGTTGCATCCCCAGGGGCTGCC |
| | | GCAGAGGCCGGAGACCTCCTGGACTAGTTCGGCGAGGAGACTGGCCACTGGGGGT |
| | | GGCTGTTCGGGACTGAGAGCGCCAAGGGTCTTTGCCAGCAAAGGAGGTTCTGCCTG |
| | | TAATTGAGCCTCTCTGATGATGGAGATGAAGTGAAGGTCTGAGGGAGCGGGCCCTG |
| | | GGGCGAGGCCATCTCTGCCTGCCTCCCTAGCAGGCGCCAGCGGTGGAGGCTGAGT |
| | | CGCAGGACACATGCCGGCCAGTTAATTCATTCTCAGCCAATGAAGGTTTGTCTAAGC |
| | | TGCCTGGGTATCCACGGGACAAAAACAGCAAACTCCCTCAGACTTTGTCCATGTATA |
| | | AACTTGAAGTGGTTGTGTTGTAGGGTTGCAGGTTTTTTGTTACGCTGCTGTCACTTTC |
| | | TGTCCAGGAGCTGGCACCCCAGGTGTTCTGAGACCTTGAGGGACCCAGACCTTTGG |
| | | GTCCAAGAGTTTCCCAAACAGCCACGCCTCTCAGGAACCCACCTGGCGGTTCCGTG |
| | | AGCTCAGGCAGGCCTGACCCGGCGGCACAGCCTGGCAGGGACCTCGTCCCCAAGC |
| | | CTGGCAGAATGAGAGGGGTTGAGGTCCCGAGCGCCACTCCTAGCCTTGCCGCCTTC |
| | | AATAGAGAAGAAATCCCTTTGCTAGATAGGGTCCCCCAGGCAGTCCCCAGTGGCGG |
| | | GACACAGGGGTCCGGCTGTGGAGCTCCCCTGCCAGCCCCTGGAGCTCCAGGAGGG |
| | | CCTGTTGGTCCCCTGTTCAGAATGGAGTGCAGCCCGCCAGCGGAAAGTGTTCATTC |
| | | TGCATAGGTGTGAGGCTTTATCTGCACACAGGACATGAAAACCAGCAGAAAGGCCC |
| | | TGAGCTGCTGCATAGCCCCATCTGATTTCTGCAGTTCCCGCCAGCCTCCAACACGG |
| | | GGACTCTGCCGTAACTGGAATCTTCATAGGTCATATTGAAATCTTCAAGGTGACCAT |
| | | GCCCCACCGGGGTGCTGGGGCAGTAGTCATGGCAGACTCCCGGCCTGGGCCCCCA |
| | | GGATTCTAGGACCCCCAGGCAGCCCCTTGGACTGGTCCCGGGTGCCTTCCAAGCAC |
| | | AGTCTCCATGCTCCCAGATTCTCGACCTTCCCCCGGCCCGGGAGGTGCAGCCTGCG |
| | | TCTGCCTCTGTCGTGTGTGCTGATTTGAGTGGCTTAGCTTGCCACAGCGCAGCCTCT |
| | | TCTGTCCCTTTCAGTCATTTGCTGTACTTCCCTGTGGCACGTTACCATGGAAGCCGC |
| | | TCCAGGGTGGGTCAGGGTGCAAGCTGCTGGTGAGGTTTGGAAGCATCAGGCTCAC |
| | | GGGTGTTCATGTGTGTTCGTGCGTGTGTGTGCGTACGTGTATATAACTGAAGTGTCT |
| | | GTACGGAATGCCCTTTGCTAGCCATGGGCTGGTCACCAGATTGTTTTGTAATGCCCG |
| | | CCCCTTGCCTCGATATTGCCAGTTTCTTGTGCAATAAACAATCAGCAGCTGTGAAAAA |
| | | AAAAAAAAAAAAA |
| 13 | WWP2-N mRNA (NCBI | CTCTTCCGGGTCAGGTGACCGCGCGCGGTCACGTGACCCGGCCCGAGTGCGGGC |
| | NM_001270455.1) | GGTGGAAGGCGGAAGTAGGAGAGGAGTTCGGCGCCGCTTCTGTGGCCACGGCAGC |
| | | TTCACGGTGATGATATGGCATCTGCCAGCTCTAGCCGGGCAGGAGTGGCCCTGCCT |
| | | TTTGAGAAGTCTCAGCTCACTTTGAAAGTGGTGTCCGCAAAGCCCAAGGTGCATAAT |
| | | CGTCAACCTCGAATTAACTCCTACGTGGAGGTGGCGGTGGATGGACTCCCCAGTGA |
| | | GACCAAGAAGACTGGGAAGCGCATTGGGAGCTCTGAGCTTCTCTGGAATGAGATCA |
| | | TCATTTTGAATGTCACGGCACAGAGTCATTTAGATTTAAAGGTCTGGAGCTGCCATAC |
| | | CTTGAGAAATGAACTGCTAGGCACCGCATCTGTCAACCTCTCCAACGTCTTGAAGAA |
| | | CAATGGGGGCAAAATGGAGAACATGCAGCTGACCCTGAACCTGCAGACGGAGAACA |
| | | AAGGCAGCGTTGTCTCAGGCGGAGAGCTGACAATTTTCCTGGACGGGCCAACTGTT |
| | | GATCTGGGAAATGTGCCTAATGGCAGTGCCCTGACAGATGGATCACAGCTGCCTTC |
| | | GAGAGACTCCAGTGGAACAGCAGTAGCTCCAGAGAACCGGCACCAGCCCCCCAGC |
| | | ACAAACTGCTTTGGTGGAAGATCCCGGACGCACAGACATTCGGGTGCTTCAGCCAG |
| | | AACAACCCCAGCAACCGGCGAGCAAAGCCCCGGTGCTCGGAGCCGGCACCGCCAG |
| | | CCCGTCAAGAACTCAGGCCACAGTGGCTTGGCCAATGGCACAGTGAATGATGAACC |
| | | CACAACAGCCACTGATCCCGAAGAACCTTCCGTTGTTGGTGTGACGTCCCCACCTG |
| | | CTGCACCCTTGAGTGTGACCCCGAATCCCAACACGACTTCTCTCCCTGCCCCAGCC |
| | | ACACCGGCTGAAGGAGAGGAACCCAGCACTTCGGGTACACAGCAGCTCCCAGCGG |
| | | CTGCCCAGGCCCCCGACGCTCTGCCTGCTGGATGGGAACAGCGAGAGCTGCCCAA |
| | | CGGACGTGTCTATTATGTTGACCACAATACCAAGACCACCACCTGGGAGCGGCCCC |
| | | TTCCTCCAGGGTAGGTCATCAACTGAGAAGACCTGAGACTCTGGAACTGACACCATG |
| | | AGTCACCCAATGGCTTCTTGAAACGGTCCCTTTCTGCGGAGGTAGCATAGCACAGTG |
| | | ACGTTTATTCCGGGTCACTTGATTGCTTTTCCTATCCACTTACCTTAATATTGCTCCCA |
| | | TGTCTTAGGACATATTAGAATTATTAGAAGATCTCTGGGAAACAAAAGGCGCATGGG |
| | | AGGCCAAGATTTAGAGTGTTGATGCATTCCGCCGTACCCTATGCCCAGCCTGTCCCT |
| | | AAAGGCAGAGTGCTTCTTATTCGGCAGGGCTTCGTGAGAATGCCGCCAAGTTATCCA |
| | | AAACCAAAGGATGAGAGAGGCTTCCAAGAGAGGGAAGACTGTCCAAGTTCAAGGAG |
| | | AAGGGGTTTGGTTCCCTGTCTGCCAGGGACAGAAAGTTCTTTCCACATGTGTCTCCT |
| | | GGTCCCGAGAGCTTTCAAAAGGGGATGTATTCTGCTCCAAGAAAGAATCACCTCCCA |
| | | GTAAAGTAGCCATGAAGGGCTCCTGTATAAAATATGCACACTTCCCCTGCCCTGAGT |
| | | TTGTGGAGGGAAAGGCTGTCGGAAGTAGGGACTCTTAGGAGAGCCCGTGTGCCCT |
| | | GTTTGGTTCAGCTGTCAGGCGTGTGCCACACACATCCCACCTCATCTTCTGGTCCCA |
| | | TAATTCTCCATGGATGGGCTGCTTTTAGCTCCAGTTTTCCAGTAGAATGCTTGAGTTT |
| | | TCCTTTTTTCCGTACCTCCTGGAGGATGAAACTCATAACCTGTCATCATCAGTACTCA |
| | | CTGTGTTTTCCACTTTGACTTGAAGTCAAATGAAGTTACCAAAGTTAAACATGGACTT |
| | | CTCAAATTAGAAAAAAACAAACAACAAAAAAACAGGTTAAGTGTTTTGTTATGAAAATT |
| | | ATGAAAATGTTCTTATTATTATGGAAAATTTCAAATAGATATAGAGAGAATAATGTTAC |
| | | AAACCCCCACATACTCAACATTTAGCTTCAAAAATTATCAACTCATGGCGGGTTTTGT |
| | | TTCATCTGTACAAACTAGTCGGCACACTATGATGGCCTGTGGACAAAATCCGGCCCT |
| | | CTGACTGTTTTTGTAAATAAGGTTTTATTGGAACACAGCCACACATATTTGTTGACTTA |
| | | TTGTTTTTGGCTGTTTTTGTACTTCATTGGCAGAGTTGAGTAATTGCAGCAGACTCTA |
| | | TGGCTTGCAAAACCTAAAATAATTACTTTGTGTAGCGCTTTATAGAAAAAATTTGCTAA |
| | | CTCCCGATCTGTAACCCCCAACCTATCCGCCGCACCCTGATCTAGGTAATTTTGAAG |
| | | CAAATCCCAAATGTCACATCATCTATCCATATTTCACTATGTATTTCTAAAACATATTC |
| | | TTTTATTCAAAATAAAACTCTAATATCATTAACAGTATTTTGTTTATATTATCAAATACC |
| | | CAGTCAATATTTGACTTTCTATAATTGTGTCACAAATTAAACATTGACTTTAACAGTAT |
| | | GTATTACACACTTGTACTTTGTGTACTTTATAACATGTGCACTTTAAAACAGCAGGAG |
| | | CTCTGT |
| 14 | WWP2-C mRNA (NCBI | ATTTTTTCCCCTCCTGCGTGTGGTTCTTGGAGAAAGTTGGAGGTGGTGGTGATTTCA |
| | NM_199424.2) | GTCGCCTTGGCCGCCTTGAGCCGGAGCTGAGCGGAGGCACTGGGCCGAGCCTGCT |
| | | TCCCGGGCCTTCCTACCATGCCAGGGCTGCTCCCTGCCTCCGCCACCCTGGCACAC |
| | | CTTCACCCGCGTACCGCCTCCTCCCCGTCGCTCTGCCTTTTCCAAAACTCACTTGGG |
| | | CCCTCCGTGCGCAGGGTTCTTTTTTGGTTTTTCTGTAAAAATCAAAACAAAAAACAGA |
| | | GACTTTTGAGAGGAGCAGATGCCACCTAAAGTCCCACTGCATTCCCTGCAAAGCGCT |
| | | CAAATGTGGAAGCCAGTCATTGGCATTTTTATTTTTTATTGATTGATTGATTTTTTCAC |
| | | CAGTGGCTTTTTGTAACCTCTGTGTTCTGCTGTGTTTCTTGTGTTTAGTCTTCGAGTG |
| | | CTTCGACTGACCATGATCCCCTGGGCCCCCTCCCTCCTGGCTGGGAGAAGAGACAG |
| | | GACAATGGACGGGTGTATTACGTGAACCATAACACTCGCACGACCCAGTGGGAGGA |
| | | TCCCCGGACCCAGGGGATGATCCAGGAACCAGCTCTGCCCCCAGGATGGGAGATG |
| | | AAATACACCAGCGAGGGGGTGCGATACTTTGTGGACCACAATACCCGCACCACCAC |
| | | CTTTAAGGATCCTCGCCCGGGGTTTGAGTCGGGGACGAAGCAAGGTTCCCCTGGTG |
| | | CTTATGACCGCAGTTTTCGGTGGAAGTATCACCAGTTCCGTTTCCTCTGCCATTCAAA |
| | | TGCCCTACCTAGCCACGTGAAGATCAGCGTTTCCAGGCAGACGCTTTTCGAAGATTC |
| | | CTTCCAACAGATCATGAACATGAAACCCTATGACCTGCGCCGCCGGCTCTACATCAT |
| | | CATGCGTGGCGAGGAGGGCCTGGACTATGGGGGCATCGCCAGAGAGTGGTTTTTC |
| | | CTCCTGTCTCATGAGGTGCTCAACCCTATGTATTGTTTATTTGAATATGCCGGAAAGA |
| | | ACAATTACTGCCTGCAGATCAACCCCGCCTCCTCCATCAACCCGGACCACCTCACCT |
| | | ACTTTCGCTTTATAGGCAGATTCATCGCCATGGCGCTGTACCATGGAAAGTTCATCG |
| | | ACACGGGCTTCACCCTCCCTTTCTACAAGCGGATGCTCAATAAGAGACCAACCCTGA |
| | | AAGACCTGGAGTCCATTGACCCTGAGTTCTACAACTCCATTGTCTGGATCAAAGAGA |
| | | ACAACCTGGAAGAATGTGGCCTGGAGCTGTACTTCATCCAGGACATGGAGATACTG |
| | | GGCAAGGTGACGACCCACGAGCTGAAGGAGGGCGGCGAGAGCATCCGGGTCACA |
| | | GAGGAGAACAAGGAAGAGTACATCATGCTGCTGACTGACTGGCGTTTCACCCGAGG |
| | | CGTGGAAGAGCAGACCAAAGCCTTCCTGGATGGCTTCAACGAGGTGGCCCCGCTG |
| | | GAGTGGCTGCGCTACTTTGACGAGAAAGAGCTGGAGCTGATGCTGTGCGGCATGCA |
| | | GGAGATAGACATGAGCGACTGGCAGAAGAGCACCATCTACCGGCACTACACCAAGA |
| | | ACAGCAAGCAGATCCAGTGGTTCTGGCAGGTGGTGAAGGAGATGGACAACGAGAAG |
| | | AGGATCCGGCTGCTGCAGTTTGTCACCGGTACCTGCCGCCTGCCCGTCGGGGGATT |
| | | TGCCGAACTCATCGGTAGCAACGGACCACAGAAGTTTTGCATTGACAAAGTTGGCAA |
| | | GGAAACCTGGCTGCCCAGAAGCCACACCTGCTTCAACCGTCTGGATCTTCCACCCT |
| | | ACAAGAGCTACGAACAGCTGAGAGAGAAGCTGCTGTATGCCATTGAGGAGACCGAG |
| | | GGCTTTGGACAGGAGTAACCGAGGCCGCCCCTCCCACGCCCCCCAGCGCACATGT |
| | | AGTCCTGAGTCCTCCCTGCCTGAGAGGCCACTGGCCCCGCAGCCCTTGGGAGGCC |
| | | CCCGTGGATGTGGCCCTGTGTGGGACCACACTGTCATCTCGCTGCTGGCAGAAAAG |
| | | CCTGATCCCAGGAGGCCCTGCAGTTCCCCCGACCCGCGGATGGCAGTCTGGAATAA |
| | | AGCCCCCTAGTTGCCTTTGGCCCCACCTTTGCAAAGTTCCAGAGGGCTGACCCTCT |
| | | CTGCAAAACTCTCCCCTGTCCTCTAGACCCCACCCTGGGTGTATGTGAGTGTGCAAG |
| | | GGAAGGTGTTGCATCCCCAGGGGCTGCCGCAGAGGCCGGAGACCTCCTGGACTAG |
| | | TTCGGCGAGGAGACTGGCCACTGGGGGTGGCTGTTCGGGACTGAGAGCGCCAAGG |
| | | GTCTTTGCCAGCAAAGGAGGTTCTGCCTGTAATTGAGCCTCTCTGATGATGGAGATG |
| | | AAGTGAAGGTCTGAGGGAGCGGGCCCTGGGGCGAGGCCATCTCTGCCTGCCTCCC |
| | | TAGCAGGCGCCAGCGGTGGAGGCTGAGTCGCAGGACACATGCCGGCCAGTTAATT |
| | | CATTCTCAGCCAATGAAGGTTTGTCTAAGCTGCCTGGGTATCCACGGGACAAAAACA |
| | | GCAAACTCCCTCAGACTTTGTCCATGTATAAACTTGAAGTGGTTGTGTTGTAGGGTT |
| | | GCAGGTTTTTTGTTACGCTGCTGTCACTTTCTGTCCAGGAGCTGGCACCCCAGGTGT |
| | | TCTGAGACCTTGAGGGACCCAGACCTTTGGGTCCAAGAGTTTCCCAAACAGCCACG |
| | | CCTCTCAGGAACCCACCTGGCGGTTCCGTGAGCTCAGGCAGGCCTGACCCGGCGG |
| | | CACAGCCTGGCAGGGACCTCGTCCCCAAGCCTGGCAGAATGAGAGGGGTTGAGGT |
| | | CCCGAGCGCCACTCCTAGCCTTGCCGCCTTCAATAGAGAAGAAATCCCTTTGCTAGA |
| | | TAGGGTCCCCCAGGCAGTCCCCAGTGGCGGGACACAGGGGTCCGGCTGTGGAGCT |
| | | CCCCTGCCAGCCCCTGGAGCTCCAGGAGGGCCTGTTGGTCCCCTGTTCAGAATGGA |
| | | GTGCAGCCCGCCAGCGGAAAGTGTTCATTCTGCATAGGTGTGAGGCTTTATCTGCA |
| | | CACAGGACATGAAAACCAGCAGAAAGGCCCTGAGCTGCTGCATAGCCCCATCTGAT |
| | | TTCTGCAGTTCCCGCCAGCCTCCAACACGGGGACTCTGCCGTAACTGGAATCTTCAT |
| | | AGGTCATATTGAAATCTTCAAGGTGACCATGCCCCACCGGGGTGCTGGGGCAGTAG |
| | | TCATGGCAGACTCCCGGCCTGGGCCCCCAGGATTCTAGGACCCCCAGGCAGCCCC |
| | | TTGGACTGGTCCCGGGTGCCTTCCAAGCACAGTCTCCATGCTCCCAGATTCTCGAC |
| | | CTTCCCCCGGCCCGGGAGGTGCAGCCTGCGTCTGCCTCTGTCGTGTGTGCTGATTT |
| | | GAGTGGCTTAGCTTGCCACAGCGCAGCCTCTTCTGTCCCTTTCAGTCATTTGCTGTA |
| | | CTTCCCTGTGGCACGTTACCATGGAAGCCGCTCCAGGGTGGGTCAGGGTGCAAGCT |
| | | GCTGGTGAGGTTTGGAAGCATCAGGCTCACGGGTGTTCATGTGTGTTCGTGCGTGT |
| | | GTGTGCGTACGTGTATATAACTGAAGTGTCTGTACGGAATGCCCTTTGCTAGCCATG |
| | | GGCTGGTCACCAGATTGTTTTGTAATGCCCGCCCCTTGCCTCGATATTGCCAGTTTC |
| | | TTGTGCAATAAACAATCAGCAGCTGTGAAAAAAAAAAAAAAAAAA |
| 15 | Soond et al. primer | GATCTCCGGAGTACGTGCGCAACTATGATCAAGAGTCATAGTTGCGCACGTACTCCT |
| | for WWP2-FL specific | TTTTA |
| | shRNA (sense) | |
| 16 | Soond et al. primer | GATCTAAAAAGGAGTACGTGCGCAACTATGACTCTTGATCATAGTTGCGCACGTACT |
| | for WWP2-FL specific | CCGGA |
| | shRNA (antisense) | |
| 17 | Soond et al. primer | GATCTCCGACGTTTATTCCGGGTCACTTTCAAGAGAAGTGACCCGGAATAAACGTCT |
| | for WWP2-N specific | TTTTA |
| | shRNA (sense) | |
| 18 | Soond et al. primer | GATCTAAAAAGACGTTTATTCCGGGTCACTTCTCTTGAAAGTGACCCGGAATAAACG |
| | for WWP2-N specific | TCGGA |
| | shRNA (antisense) | |
| 19 | Soond et al. primer | GATCTCCGGAGCAGATGCCACCTAAAGTTCAAGAGACTTTAGGTGGCATCTGCTCCT |
| | for WWP2-C specific | TTTTA |
| | shRNA (sense) | |
| 20 | Soond et al. primer | GATCTAAAAAGGAGCAGATGCCACCTAAAGTCTCTTGAACTTTAGGTGGCATCTGCT |
| | for WWP2-C specific | CCGGA |
| | shRNA (antisense) | |
| 21 | Maddika et al. | UGACAAAGUUGGAAGGAAUU |
| | WWP2 siRNA 1 | |
| 22 | Maddika et al. | GGGAGAAGAGACAGGACAAUU |
| | WWP2 siRNA 2 | |
| 23 | Maddika et al. | GGGAGAAGAGACAGGACAAUU |
| | WWP2 siRNA 3 | |
| 24 | Maddika et al. | ACAUGGAGAUACUGGGCAAUU |
| | WWP2 siRNA 4 | |
| 25 | Maddika et al. | AGCACAGAGUCAUUUAGAUUUA |
| | WWP2 shRNA 1 | |
| 26 | Maddika et al. | ACCUAUGUAUUGUUUAUUUGAA |
| | WWP2 shRNA 2 | |
| 27 | Mouse Wwp2-P1 RT- | TTTGAGAAGTCCCAGCTTACCC |
| | qPCR forward primer | |
| 28 | Mouse Wwp2-P1 RT- | CTCCAGACCTTCAGATCCAAATG |
| | qPCR reverse primer | |
| 29 | Mouse Wwp2-P2 RT- | ACCAAGAGCAGCAAGCAGAT |
| | qPCR forward primer | |
| 30 | Mouse Wwp2-P2 RT- | GTCTCCTCGATGGCGTACAG |
| | qPCR reverse primer | |
| 31 | Mouse 18S rRNA RT- | GTAACCCGTTGAACCCCATT |
| | qPCR forward primer | |
| 32 | Mouse 18S rRNA RT- | CCATCCAATCGGTAGTAGCG |
| | qPCR reverse primer | |
| 33 | Mouse Tgfbr1 RT- | CTCCCGTGGCTTCTAGTGC |
| | qPCR forward primer | |
| 34 | Mouse Tgfbr1 rRNA | GCCTTAGTTTGGACAGGATCTG |
| | RT-qPCR reverse | |
| | primer | |
| 35 | Mouse Tgfbr2 RT- | CATTTGGTTCCAAGGTGC |
| | qPCR forward primer | |
| 36 | Mouse Tgfbr2 rRNA | TGGTAGTGTTCAGCGAG |
| | RT-qPCR reverse | |
| | primer | |
| 37 | Mouse Acta2 RT-qPCR | GTCCCAGACATCAGGGAGTAA |
| | forward primer | |
| 38 | Mouse Acta2 RT-qPCR | TCGGATACTTCAGCGTCAGGA |
| | reverse primer | |
| 39 | Mouse Smad2 RT- | AATACGGTAGATCAGTGGGACA |
| | qPCR forward primer | |
| 40 | Mouse Smad2 RT- | CAGTTTTCGATTGCCTTGAGC |
| | qPCR reverse primer | |
| 41 | Mouse Col1a1 RT- | GCTCCTCTTAGGGGCCACT |
| | qPCR forward primer | |
| 42 | Mouse Col1a1 RT- | CCACGTCTCACCATTGGGG |
| | qPCR reverse primer | |
| 43 | Mouse FN-EDA RT- | ATGTGGACCCCTCCTGATAGT |
| | qPCR forward primer | |
| 44 | Mouse FN-EDA RT- | GCCCAGTGATTTCAGCAAAGG |
| | qPCR reverse primer | |
| 45 | Mouse TCF21 RT- | CCCACTAAGAAAAGCCCGCTC |
| | qPCR forward primer | |
| 46 | Mouse TCF21 RT- | CCGTTCTCGTACTTGTCGTTG |
| | qPCR reverse primer | |
| 47 | Mouse Postn RT-qPCR | CCTGCCCTTATATGCTCTGCT |
| | forward primer | |
| 48 | Mouse Postn RT-qPCR | AAACATGGTCAATAGGCATCACT |
| | reverse primer | |
| 49 | Mouse CTGF RT- | CCTGCCCTTATATGCTCTGCT |
| | qPCR forward primer | |
| 50 | Mouse CTGF RT- | AAACATGGTCAATAGGCATCACT |
| | qPCR reverse primer | |
| 51 | Human WWP2-P1 RT- | GAGAAACTGTCCGTTCGGGTG |
| | qPCR forward primer | |
| 52 | Human WWP2-P1 RT- | GACAAGCGCGTCCTCAATGAT |
| | qPCR reverse primer | |
| 53 | Human WWP2-P2 RT- | CTTGTCGTGTCGTTCCGCTT |
| | qPCR forward primer | |
| 54 | Human WWP2-P2 RT- | ACCATTTGCTTCACACTTGACTT |
| | qPCR reverse primer | |
| 55 | Human COL1A1 RT- | GTGCGATGACGTGATCTGTGA |
| | qPCR forward primer | |
| 56 | Human COL1A1 RT- | CGGTGGTTTCTTGGTCGGT |
| | qPCR reverse primer | |
| 57 | Human COL1A2 RT- | GAGCGGTAACAAGGGTGAGC |
| | qPCR forward primer | |
| 58 | Human COL1A2 RT- | CTTCCCCATTAGGGCCTCTC |
| | qPCR reverse primer | |
| 59 | Human LUM RT-qPCR | CTGCGTTTATCTCACAACGAACT |
| | forward primer | |
| 60 | Human LUM RT-qPCR | CAGATCCAGCTCAACCAGGG |
| | reverse primer | |
| 61 | Human 18S rRNA RT- | GGCCCTGTAATTGGAATGAGTC |
| | qPCR forward primer | |
| 62 | Human 18S rRNA RT- | CCAAGATCCAACTACGAGCTT |
| | qPCR reverse primer | |
| 63 | Mouse Wwp2 | GTGGTGTCAGCAAAGCCCAAGGTGC |
| | genotyping forward | |
| | primer | |
| 64 | Mouse Wwp2 | GAGCACCATGGGGGCCAAGGGCAGC |
| | genotyping reverse | |
| | primer | |

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably performed in vitro. The term “in vitro” is intended to encompass experiments with cells in culture whereas the term “in vivo” is intended to encompass experiments with intact multi-cellular organisms.

### BRIEF DESCRIPTION OF THE FIGURES

Embodiments and experiments illustrating the principles of the invention will now be discussed with reference to the accompanying figures.

FIGS. 1A and 1B. Histograms showing the distribution of the Spearman's pairwise correlations (p) between WWP2 expression levels and each of the genes in the hECM-network in (1A) DCM patients and control samples, and (1B) rTOF and control samples. P-values were calculated using Mann-Whitney U Test.

FIG. 2. Network graph showing the core set of hECM-network genes with strongest significant correlation (FDR<0.01) to WWP2 in both DCM and rTOF patients, out of 326 genes here only the connected component (40 genes) is displayed. Nodes represent individual genes and edges denote protein-protein interaction or co-expression in STRING protein database. Node colour is mapped to the average correlation of each gene with WWP2 in DCM and rTOF patients. Genes that are annotated in the Gene Ontology Cellular Component database as “extracellular matrix” (ECM) are highlighted with thicker border.

FIG. 3. Boxplot showing the median expression level of the hECM-network genes (683 genes) in the DCM patients stratified by genotype at the regulatory SNP rs9936589. Kruskal-Wallis test p-value is shown.

FIG. 4. Schematic representation of the three main WWP2 protein isoforms and their constituent domains.

FIG. 5. Boxplots showing WWP2-FL, WWP2-N and WWP2-C isoform expression levels measured in the DCM patients by RNA-seq data, stratified by genotype at the regulatory SNP rs9936589 (FDR of Kruskal-Wallis test p-value after correcting for the number of WWP2 isoforms detected in the DCM heart, 18 gene isoforms). NS denotes non-significant (i.e. FDR>0.05), and graphs showing the results of Bayesian cis-eQTL mapping of WWP2 isoform at the WWP2 regulatory locus (1 Mb region centred on WWP2). The strength of eQTL mapping is reported as marginal posterior probability of association (MPPI, y-axis) between each SNP and WWP2 isoform.

FIGS. 6A to 6C. Schematic, bar chart and images relating to the generation of Wwp2 mutant mice. (6A) Schematic representation of Wwp2 isoform open reading frames (ORFs), primer pairs and PCR products for detecting transcripts for the different isoforms by qPCR (P1 detects Wwp2-FL and Wwp2-N, P2 detects Wwp2-FL only, and P3 detects Wwp2-FL and Wwp2-C), and the site of the 4 bp insertion disrupting expression of the Wwp2-FL and Wwp2-N isoforms in Wwp2^{Mut/Mut} mice. (6B) Bar chart showing the result of analysis of Wwp2 transcript levels by qPCR in heart from WT and Wwp2^{Mut/Mut} mice, as detected using the indicated primer pairs. (6C) Shows the results of analysis of protein expression of Wwp2-FL in cardiac fibroblasts from WT and Wwp2^{Mut/Mut} mice, performed using two different anti-WWP2 antibodies recognising the N-terminal region of Wwp2.

FIGS. 7A to 7F. Images, bar charts and graphs showing the effects of Ang II infusion in wildtype (i.e. Wwp2^{Wt/Wt}) mice. (7A) Representative Sirius Red-stained sections from Ang II-infused hearts reveals increased fibrosis as compared to control, saline-infused hearts. Scale bar: 0.5 mm. (7B) Representative WGA-stained section of Ang II-infused hearts showing hypertrophic myocytes with higher mean cell volume as compared to saline-infused hearts (6 biological replicates, 8 images each, Mann-Whitney U test, means±SD). Scale bar: 20 μm. (7C) Graphs showing increased left ventricular mass index (LVMI), and decreased ejection fraction (EF %) and fractional shortening (FS %) in Ang II-infused mice as compared to saline-infused controls. (Mann-Whitney U test, means±SD). (7D) Bar chart showing expression Wwp2 transcripts corresponding to different isoforms in LV tissue (see FIG. 6A), as determined by RT-qPCR; Wwp2 expression is upregulated in Ang II-infused mice as compared to saline-infused controls. Mann-Whitney U test, n=6 for each genotype; means±SD. (7E) Immunofluorescence images showing more WWP2-positive cells in heart sections from Ang II-infused mice as compared to saline-infused controls. Scale bar: 100 μm. (7F) Western blot analysis showing the expression of different isoforms, with increased expression WWP2-FL, WWP2-N and similar levels of WWP2-C in the Ang II infused mice, as comparted with saline-infused control mice.

FIGS. 8A to 8J. Schematic, images, bar charts and graphs showing the effects of Ang II infusion in Wwp2^{Mut/Mut} mice as compared to wildtype mice. (8A) Schematic of the experiment in which WT and Wwp2^{Mut/Mut} mice are subjected to Ang II infusion, representative Sirius red and Masson's Trichrome staining of short-axis sections in left ventricle taken from WT and Wwp2^{Mut/Mut} mice following Saline (Control) and Angiotensin II (Ang II) infusion (scale bar: 0.5 mm), and quantification of the area of fibrosis in transverse histological sections with Sirius red staining at the mid-ventricular level. (8B) Representative M-mode echocardiograms (parasternal short-axis, middle) in WT and Wwp2^{Mut/Mut} mice following Ang II infusion. (8C) Cardiac echocardiogram-based analysis of left ventricular posterior wall thickness during end diastole in Wwp2^{Mut/Mut} mice and WT mice after Ang II infusion. Left ventricular posterior wall thickness during end diastole was reduced in the Wwp2^{Mut/Mut} mice (Mann-Whitney U test; means±SD). (8D) Left ventricular mass index (LVMI) and echocardiogram-based quantification of left ventricular ejection fraction (EF %) and fractional shortening (FS %) in WT and Wwp2^{Mut/Mut} mice after saline- (Control) or Ang II infusion. (8E) Biological processes differentially expressed in the LV transcriptome of the Wwp2^{Mut/Mut} relative to WT mice after 28 days Ang II infusion (only terms from the Hallmark gene sets database are shown). NES, represents the strength of the downregulation by the GSEA algorithm (lower NES corresponds to stronger downregulation). (8F) Representative western blot showing ACTA2 in heart from both WT and Wwp2^{Mut/Mut} mice following Ang II infusion. (8G) Bar chart showing collagen content (as determined by HPA assay) in heart of WT and Wwp2^{Mut/Mut} mice following Ang II infusion. (8H) Representative western blot showing Fibronectin extracellular domain A (FN-EDA, ~220 kD) and Periostin (POSTN, ~94 kD) expression in the heart of WT and Wwp2^{Mut/Mut} mice following Ang II infusion. (8I) Representative immunostaining showing ACTA2 expression in both WT and Wwp2^{Mut/Mut} mice following AngII-infusion. Scale bar: 400 μm. (8J) Relative mRNA expression of Acta2 and Col1a1 in left ventricle from WT and Wwp2^{Mut/Mut} mice, following AngII or saline infusion. mRNA expression was normalized to the level of 18S rRNA. (Mann-Whitney U test, n=5 for each group; means±SD).

FIGS. 9A to 9C. Schematic, images and graphs showing the effects of Myocardial Infarction in Wwp2^{Mut/Mut} mice as compared to wildtype mice. (9A) Schematic of the experiment in which WT and Wwp2^{Mut/Mut} mice are subjected to myocardial infarction (MI), permanent ligation of the left coronary artery with a 8-0 nylon monofilament suture, thorax was closed with 6-0 coated vicryl suture and mice were followed for 4 weeks after surgery. Representative Sirius red (top panels) and Masson's Trichrome staining (bottom panels) of short-axis sections in left ventricle taken from WT and Wwp2^{Mut/Mut} mice following MI (scale bar: 0.5 mm). Quantification of the area of fibrosis in transverse histological sections with Sirius red staining at the infarct site. (9B) Representative M-mode echocardiograms (Middle LV long-axis) in WT and Wwp2^{Mut/Mut} mice following MI. (9C) Cardiac echocardiogram-based analysis of left ventricular inner diameter (LVIDd), left ventricular ejection fraction (EF %) and fractional shortening (FS %) of WT and Wwp2^{Mut/Mut} mice after MI. p-values were calculated by the Mann-Whitney U test and data reported as mean±SD.

FIGS. 10A to 10D. Images and scatterplot relating to characterisation of WWP2-expressing cardiac cells. (10A) Immunofluorescence images of left ventricle (LV) section staining from WT mouse following Ang II infusion showed that WWP2 (arrow) is expressed in non-myocytes (Left), and co-localized with part of the FSP1-positive cells (Right). Far left: WGA; inner left and inset: WGA/Wwp2/DAPI merge; inner right: FSP1/DAPI merge; inner right and inset: FSP1/Wwp2/DAPI merge. Scale bar: 40 μm. (10B) Immunofluorescence images of LV section staining from WT LV after Ang II infusion showed that WWP2 (arrows) is expressed in non-myocytes (top), and co-localized with some of the FSP1 positive cells (bottom). Top left: WGA; top right: WGA/Wwp2/DAPI merge; bottom left: FSP1/DAPI merge; bottom right: FSP1/Wwp2/DAPI merge. Scale bar: 40 μm. (10C) Immunofluorescence images showing both FSP1 (top) and WWP2 (bottom) positive staining in primary WT cardiac fibroblasts. Scale bar: 10 μm. (10D) t-SNE displaying single-cell RNA-seq data in the LV of WT mice after Ang II infusion. A total of 508 cells were detected. Each dot corresponds to a single cell, Wwp2 expression level is indicated (Log₂ normalized counts). Cells belonging to the different subpopulations identified in these mouse heart data are indicated.

FIGS. 11A to 11J. Bar charts, images and box plots relating to the effects of TGFβ1 stimulation. (11A) Relative mRNA expression targeting different parts of Wwp2 isoforms (see FIG. 6A) in WT cardiac fibroblasts treated with TGFβ1 (5 ng/ml) for 24, 48 and 72 hrs. mRNA expression was normalized to 18S level. (Mann-Whitney U test, n=4 for each group; means±SD). (11B) Relative mRNA expression of Acta1 and Coil al in WT cardiac fibroblast after TGFβ1 treatment (5 ng/ml) for 24, 48 and 72 hrs. mRNA expression was normalized to 18S level. (Mann-Whitney U test, n=4 for each group; means±SD). (11C) Relative mRNA expression of genes referred to ECM and fibroblast markers in primary cardiac fibroblasts, including Acta2, Coil al, Tcf21, Ctgf, Postn and Fn1. mRNA expression was normalized to the level of 18S. Mann-Whitney U test, n=5-6 for each group; means±SD. (11D) Western blot of ACTA2 and POSTN protein expression reveals that WWP2 deficiency reduces the pro-fibrotic response induced by TGFβ1 in cardiac fibroblasts. (11E) Representative microscopy images and (11F) quantification analysis with immunostaining for ACTA2 and COL1A1 after stimulation with TGFβ1 in cardiac fibroblasts from both WT and Wwp2^{Mut/Mut} mice (5 biological replicates, 9 images each; Mann-Whitney U test; box-and-whisker plots). (11G) Western blot of vimentin in primary cardiac fibroblasts revealed that TGFβ1 treatment (5 ng/ml, 72h) increased protein levels of vimentin in WT cells, but not in Wwp2^{Mut/Mut} cells. (Mann-Whitney U test, n=3 for each group; means±SD). (11H) Representative immunostaining showing vimentin in cardiac fibroblasts (WT and Wwp2^{Mut/Mut}) after TGFβ1 treatment (5 ng/ml, 72h). (11I) Relative mRNA expression of vimentin in cardiac fibroblasts (WT and Wwp2^{Mut/Mu}) after TGFβ1 treatment (5 ng/ml, 72h). mRNA expression was normalized to 18S level. (Mann-Whitney U test, n=5 for each group; means±SD). (11J) Relative mRNA expression of genes encoding TGFβ receptors in primary cardiac fibroblasts. RNA expression was normalized to 18S level. Mann-Whitney U test, n=6 for each group; means±SD.

FIGS. 12A and 12B. Images and graphs showing the effects of WWP2 deficiency on proliferation and migration of cardiac fibroblasts. (12A) Representative image and quantification of wound closure scratch assay in a monolayer of WT and Wwp2^{Mut/Mut} cardiac fibroblast cells (Mann-Whitney U test, n=8 for each group; means±SD) (12B) MTS assay on primary cardiac fibroblast from both WT and Wwp2^{Mut/Mut} and absorbance value (490 nm) used to indicate cell number. (Mann-Whitney U test, n=8 for each group; means±SD).

FIGS. 13A to 13D. Images and bar charts showing the effects of WWP2 knockdown. (13A to 13D) WT cardiac fibroblasts were incubated with TGFβ1 (72 hrs) and siRNA pools (SiRNA-Wwp2-N′ and SiRNA-Wwp2-C′) against 5′- or 3′-region of Wwp2 mRNA, or scramble siRNA pools (Scr). (13A) RNA transcript levels of different parts of Wwp2 isoforms (see FIG. 6A) by qPCR (Mann-Whitney U test, n=5-6 for each group; means±SD). (13B) Western blot showing expression levels of different proteins by cardiac fibroblasts. (13C) RNA transcript levels of Acta2, Col1a1 and TGFβ receptors by qPCR (Mann-Whitney U test, n=5-6 for each group; means±SD). (13D) RNA transcript levels of different parts of Wwp2 isoforms, COL1A1, COL1A2 and LUM by cultured human right ventricle primary cardiac fibroblasts following siRNA-Wwp2-N′ treatment (Mann-Whitney U test, n=3 for each group; means±SD).

FIGS. 14A to 14C. Images and bar charts showing the effects of WWP2 rescue in Wwp2^{Mut/Mut} fibroblasts. Wwp2^{Mut/Mut} cardiac fibroblasts were transfected with plasmid encoding Wwp2-FL or Wwp2-N, and incubated with TGFβ1 for 72 hrs. (14A) RNA transcript levels of different parts of Wwp2 isoforms (see FIG. 6A) by qPCR (Mann-Whitney U test, n=6 for each genotype; means±SD). (14B) RNA transcript levels of Acta2, Col1a1 and TGFβ receptors by qPCR (Mann-Whitney U test, n=5 for each group; means±SD). (14C) Western blot showing expression levels of different proteins.

FIGS. 15A to 15C. Images relating to the subcellular localisation of WWP2. (15A) Immunofluorescence analysis of WWP2 expression in WT primary fibroblasts showing nuclear localization after 16 hrs TGFβ1 stimulation. Scale bar: 20 μm. (15B) Western blot for WWP2 isoforms in whole cell lysates (WCL), cytoplasmic (Cyto) and nuclear (Nuc) extracts from WT fibroblasts after 16 hrs TGFβ1 stimulation. (15C) Representative immunofluorescence images showing the subcellular localisation of FLAG-tagged WWP2 isoforms in NIH-3T3 fibroblast cells, as determined using anti-FLAG antibody. Scale bar, 40 μm.

FIGS. 16A to 16H. Bar charts and images relating to interaction of WWP2 with SMAD proteins. (16A) Smad2 expression in WT or Wwp2^{Mut/Mut} fibroblast cells after 16 hrs TGFβ1 stimulation (16B) Smad2 expression in murine cardiac fibroblasts after transfection with plasmids encoding Wwp2-FL or Wwp2-N, or after transfection with SiRNA-Wwp2-N′ or SiRNA-Wwp2-C′. (Mann-Whitney U test, n=6 for each genotype; means±SD). (16C) Western blot showing direct interaction of SMAD2 or SMAD7 with WWP2 isoforms by co-immunoprecipitation analysis of lysates NIH-3T3 cells transfected with WWP2-Flag isoforms, using anti-FLAG antibody. (16D) Western blot showing direct interaction of SMAD2 with WWP2 isoforms by co-immunoprecipitation analysis of lysates of TGFβ1 (5 ng/ml, 16 hr)-stimulated WT or Wwp2^{Mut/Mut} fibroblasts, using anti-SMAD2 antibody. (16E) Western blot showing direct interaction of SMAD7 with WWP2 isoforms by co-immunoprecipitation analysis of lysates of TGFβ1 (5 ng/ml, 16 hr)-stimulated WT or Wwp2^{Mut/Mut} fibroblasts, using anti-SMAD7 antibody. (16F) Representative western blot showing SMAD7 protein levels in WT and Wwp2^{Mut/Mut} fibroblasts with/without TGFβ1 stimulation. (16G) Western blot showing p-SMAD2 with WWP2-FL by co-immunoprecipitation analysis of lysates of TGFβ1 (5 ng/ml, 16 hr)-stimulated NIH-3T3 cells transfected with WWP2-FL. (16H) Representative western blot showing p-SMAD2 and SMAD2 protein levels in WT and Wwp2^{Mut/Mut} fibroblasts with or without TGFβ1 stimulation.

FIGS. 17A to 17H. Images, graph and schematic relating to WWP2-mediated ubiquitination and nucleocytoplasmic shuttling of SMAD proteins. (17A) Western blot showing in-cell ubiquitylation of SMAD2 by immunoprecipitation in NIH-3T3 fibroblasts. Cells were treated with TGFβ1 (5 ng/ml, 16 hr) and then MG132 (10 μM, 4 hr), then lysates were prepared and subjected to immunoprecipitation with anti-SMAD antibody or IgG control, followed by western blot with anti-ubiquitin antibody. (17B) Western blot showing in-cell ubiquitylation of SMAD2 by immunoprecipitation in primary fibroblasts from WT and Wwp2^{Mut/Mut} mice. Cells were treated with MG132 (10 μM, 3 hr) followed by TGFβ1 (5 ng/ml, 6 hr), then lysates were prepared and subjected to immunoprecipitation with anti-SMAD2 antibody, followed by western blot with anti-ubiquitin antibody. (17C) Quantification of TGFβ1-induced SMAD2 luciferase reporter activity in WT and Wwp2^{Mut/Mut} fibroblasts (Mann-Whitney U test; n=4 for each group; means±SD). (17D) Western blot showing SMAD2 and SMAD4 protein distribution in cytoplasmic and nuclear fractions of WT and Wwp2^{Mut/Mut} fibroblasts after TGFβ1 stimulation (16 hrs). WCL, whole cell lysis; Cyto, cytoplasmic; Nuc, nuclear. (17E) Representative immunostaining of SMAD2 in WT and Wwp2^{Mut/Mut} fibroblasts treated with TGFβ1 for 1 hr and then with SB431542 for 5 hr. Scale bar: 50 μm. (17F) Quantification analysis shows delayed exportation of SMAD2 from the nucleus in the Wwp2^{Mut/Mut} cells. (5 biological replicates, 8 images each; Mann-Whitney U test; means±SD). (17G) Western blot of p-SMAD2 and SMAD2 in WT and Wwp2^{Mut/Mut} fibroblasts treated with TGFβ1 for 1 hr and then with SB431542 for 5 hr. (17H) Schematic of the proposed model for the WWP2-mediated regulation of SMAD2 nucleocytoplasmic shuttling in cardiac fibrosis.

FIGS. 18A to 18D. Graph and images relating to C5aR expression by cardiac cells in response to pro-fibrotic stimuli. (18A) Frequency of C5aR positive cells in cardiac tissue of WT and Wwp2^{Mut/Mut} mice after saline- (Control) or Ang II infusion, as determined by quantitative analysis of immunofluorescence images. Data represent means±s.d. (n=6 per group). (18B) Immunofluorescence images showing that C5aR+ cells from Ang II infused WT mice also express FSP1. Left: C5aR/DAPI merge; right: FSP1/C5aR/DAPI merge. Scale bar: 100 um. (18C) Immunofluorescence images showing that C5aR+ cells from primarily cultured cardiac fibroblasts also express FSP1. Left: FSP1/DAPI merge; middle: C5aR; right: FSP1/C5aR/DAPI merge. Scale bar: 15 um. (18D) Immunofluorescence images increased C5aR and α-SMA expression in cardiac fibroblasts following treatment with C5a.

FIGS. 19A and 19B. Scatterplot and images showing expression of WWP2 and complement genes in immune cells. (19A) t-SNE visualization of a preliminary single-cell sequencing (SCS) in the mouse heart of WT mice. Cell type clusters were inferred and annotated to the corresponding cell type by projection to the Fantom Consortia mouse dataset. M1/M2 macrophages subpopulations were identified based on the expression of CD206 (Mrc1) gene (M2). (19B) Immunofluorescence images showing that CD68+/Arg-1+ cells (M2 macrophages) express WWP2. Left: CD68/Arg-1 merge; middle: CD68/Arg-1/WWP2 merge; right: CD68/Arg-1/WWP2/DAPI merge.

FIGS. 20A to 20C. Images and bar chart relating to the effect of WWP2 on macrophage phenotype. (20A) Western blot showing expression of WWP2 isoforms containing the N-terminal region in BMDMs following M1 (left) and M2 (right) polarity stimulation. (20B) Transcriptional levels of IL-6 and CD206 in BMDMs from WT and Wwp2 LOF mice (n=3 in each group). (20C) Decreased pro-fibrotic M2 response and active TGFβ1 production by BMDMs from Wwp2 LOF mice.

FIGS. 21A to 21D. Images and graphs relating to WWP2 expression in kidney in a UUO model of renal injury. (21A) Increased expression of WWP2 isoforms in the kidney following UUO (14 days). (21B) Representative Sirius red and Masson's Trichrome staining of kidney sections from WT and Wwp2^{Mut/Mut} mice following UUO, or sham treatment. (21C) Percentage of cortical fibrosis and (21D) HPA collagen quantification of kidney sections from WT and Wwp2^{Mut/Mut} mice following UUO, or sham treatment.

FIGS. 22A to 22G. Schematic, heatmap, images, bar charts and graphs showing the effects of Wwp2 on immune function of cardiac macrophages in cardiac fibrosis. (22A and 22B) WT and Wwp2^{Mut/Mut} mice subjected to Ang II infusion show lower percentage of macrophages (22A) and inflammatory macrophages (22B; referring to LY6C^{high} macrophages) in Wwp2 LOF hearts compared with WT mice. (22C) Cellphone DB analysis showing significant Ligand-Receptor interactions in the cardiac macrophages. The CCL5 signalling pathways were inhibited in Wwp2 LOF inflammatory macrophages (rectangle). (22D) Relative expression levels of CCL5 in sorted WT and Wwp2 LOF inflammatory macrophages (upper panel) and in cultured bone marrow-derived macrophages (lower panel). (22E) Heatmap showing scaled, differentially expressed genes between WT and Wwp2 LOF inflammatory macrophages. WT Control: negative scaled expression=Ctss, H2K1, B2m, Bst2, Lst1, Ly6e, H3f3b, Rpl38, Rpl36, Rps29, Plac8, Atox1, Ccl12, S100a8, Gm47283, Ifi2712a, Ifitm3, Rtp4, Atp5e, S100a9, Irf7, H2-Q7, Oas2, Isg15, Ifi213, Slfn4, Slfn1, Ly6a, Lgals3 bp, Tet3, Cd83, Tpcn1, Actb, Gdi2, Kctd12, Fosb, Cx3cr1, Pim1; ~0 scaled expression=Gm26917, Ahnak; positive scaled expression=Malat1, Mrc1, F13a1, Cdc7, Gm43936, Ccl24, Retnla. Mut/Mut Control (Wwp2^{Mut/Mut} mice no treatment): negative scaled expression=Ctss, H2K1, B2m, Bst2, Lst1, Ly6e, H3f3b, Plac8, Atox1, Ccl12, S100a8, Gm47283, Ifi2712a, Rtp4, S100a9, Irf7, H2-Q7, Oas2, Isg15, Ifi213, Slfn4, Slfn1, Ly6a, Lgals3 bp, Tet3, Tpcn1, Gdi2, Kctd12, Fosb, Cx3cr1, Pim1, Malat1, Cdc7, Gm43936; ~0 scaled expression=Gm26917, Ifitm3, Atp5e, Cd83, Ahnak; positive scaled expression=Rpl38, Rpl36, Rps29, Mrc1, F13a1, Actb, Ccl24, Retnla. WT AngII: negative scaled expression=Mrc1, F13a1, Tet3, Cd83, Tpcn1, Actb, Ahnak, Gdi2, Kctd12, Fosb, Cx3cr1, Pim1, Cdc7, Gm43936, Ccl24, Retnla; ~0 scaled expression=Rpl38, Rpl36, Rps29, Plac8, Gm26917, Atp5e, Malat1, Isg15, Lgals3 bp; positive scaled expression=Ctss, H2K1, B2m, Bst2, Lst1, Ly6e, H3f3b, Atox1, Cc112, S100a8, Gm47283, Ifi2712a, Ifitm3, Rtp4, S100a9, Irf7, H2-Q7, Oas2, Ifi213, Slfn4, Slfn1, Ly6a. Mut/Mut AngII (Wwp2^{Mut/Mut} mice treated with AngII): negative scaled expression=Ctss, H2K1, B2m, Bst2, Lst1, Ly6e, H3f3b, Rpl38, Rpl36, Rps29, Plac8, Gm26917, Atox1, Ccl12, S100a8, Gm47283, Ifi2712a, Ifitm3, Rtp4, Atp5e, Malat1, S100a9, Irf7, H2-Q7, Oas2, Isg15, Ifi213, Slfn4, Slfn1, Ly6a, Lgals3 bp, Cdc7, Gm43936, Ccl24, Retnla; ~0 scaled expression=F13a1, Actb, Ahnak; positive scaled expression=Mrc1, Tet3, Cd83, Tpcn1, Gdi2, Kctd12, Fosb, Cx3cr1, Pim1. Darker grey=more intense up/downregulation of expression, as indicated by scaled expression bar. (22F) Relative mRNA expression of genes involved in interferon signalling in cardiac macrophages under Ang II infusion (7 days). For each gene, from left to right bars correspond to WT control; Wwp2^{Mut/Mut} control; WT AngII; and Wwp2^{Mut/Mut} AngII; (22G) Expression of inflammatory markers in BMDM following LPS/INFγ stimulation (4 hrs).

FIGS. 23A to 23D. Images, bar chart and graph showing the protective effects of WWP2 against lung fibrosis. (23A) WT and Wwp2^{Mut/Mut} mice subjected to bleomycin-induction show higher percentage of survival in Wwp2 LOF compared to WT mice. (23B) Representative Sirius red staining of lung section 21 days after bleomycin administration. (23C) Semi-quantitative analysis of lung lesions according to modified Ashcroft score. (23D) Using the same experimental setting, lung sections were analysed by Masson's Trichrome staining for collagen I deposition.

FIGS. 24A to 24G. Images, bar chart and graphs showing WWP2 upregulation in chronic kidney disease in humans, and protective effects against renal fibrosis associated with Wwp2 LOF in mice. (24A) Representative immune-histological staining of WWP2 showing upregulation of WWP2 expression in human kidney tissue in patients with CKD relative to healthy controls. (24B) Representative Sirius red and Masson's Trichrome staining of short-axis sections of WT and Wwp2^{Mut/Mut} mice subjected to UUO model (14 days). (24C and 24D) Quantification of fibrosis and collagen content (HPA assay) in kidney of WT and Wwp2^{Mut/Mut} mice following UUO (14 days). (24E) Representative microscopy images with immunostaining for ACTA2 after TGFβ1 stimulation (72 hrs) of renal fibroblasts from WT and Wwp2^{Mut/Mut} mice. (24F) Relative mRNA expression (normalised to 18S) after TGFβ1 stimulation of renal fibroblasts from WT and Wwp2^{Mut/Mut} mice. For each gene, from left to right bars correspond to WT control; Wwp2^{Mut/Mut} control; WT TGFβ1 stimulation; and Wwp2^{Mut/Mut} TGFβ1 stimulation (72 hrs). (24G) Western Blot showing ECM and pro-fibrotic markers in primary renal (myo)fibroblasts.

FIGS. 25A and 25B. Schematic representations of the chemical structures of (25A) EP1, and (25B) Clomipramine hydrochloride.

FIGS. 26A and 26B. Representative images showing ACTA2 staining of primary renal fibroblasts following 72 hours of stimulation with TGFβ1, (26A) without prior treatment, or (26B) with prior treatment of the cells with EP1 at a concentration of 0.3 mM, for 1 hour. Images show ACTA2 staining and DAPI staining (for visualisation of nuclei).

### EXAMPLES

In the following Examples, the inventors describe the identification of WWP2 as a positive regulator of a pro-fibrotic gene network common to a range of chronic diseases. They show that increased WWP2 expression in the human diseased heart leads to increased expression of this gene network of extracellular matrix proteins and pro-fibrotic genes (including collagens, MMPs, cytokines, etc.), and that the positive association between expression of WWP2 and pro-fibrotic gene expression exists in tissues of various organs in the context of chronic disease.

The inventors further demonstrate that WWP2 regulates the TGFβ-induced transcriptional response of pro-fibrotic genes, and that transgenic mice lacking the N-terminal region of WWP2 also show a reduction in the complement and coagulation cascade, which is responsible for the activation of immune cells during the fibrogenic response.

The inventors further demonstrate that inhibition of WWP2 expression reduces macrophage accumulation and expression of proinflammatory factors by macrophages in a fibroinflammatory disease setting.

The inventors further demonstrate that inhibition of WWP2 expression reduces mortality and tissue fibrosis in a model of lung fibrosis, and that inhibition of WWP2 expression reduces tissue fibrosis in a model of kidney fibrosis, that WWP2 expression is upregulated in renal tissue of human subjects with chronic kidney disease, and that inhibition of WWP2 expression abrogates TGFβ-induced upregulation of ACTA2 expression in fibroblasts.

The inventors further demonstrate that inhibition of WWP2 using a small molecule inhibitor targeting the WWP2 N-terminal isoform (WWP2-N) comprising the C2 domain abrogates TGFβ-induced upregulation of ACTA2 expression in fibroblasts, confirming that WWP2 inhibitors are useful for the treatment/prevention of fibroinflammatory disease.

### Example 1: Materials and Methods

### 1.1 Description of Rat Segregating Population and Human Cohorts (RNA-Seq, Phenotypic and Genetic Data) and Data Processing

### Rat Recombinant Inbred (RI) Strains Data

### Rat RI Left Ventricle (LV) Expression Data

This expression dataset consists of the heart LV RNA-sequencing in the 30 RI strains. This data was published in [3]. TopHat 1.2.0 [4] was used to map the reads to the BN reference genome RGSC 3.4 (known splice junctions in the Ensembl reference database [5] were supplied, de novo splice junction detection was also enabled). After mapping the reads to the reference genome, Cufflinks 1.0.2 was used to assemble the aligned RNA-seq reads into transcripts by using the Ensembl annotation of the rat transcriptome. Cufflinks is able to reconstruct the set of transcripts that “explains” the reads observed in our RNA-seq experiment, in our case, by using a transcriptome reference annotation. The underlying reason for using a reference-based transcriptome assembly approach is that, comparative transcriptome assembly analyses have shown that, overall, reference-based transcriptome assembly approaches have a better performance than de novo approaches, in both sensitivity and the ability to discover splicing patterns [6]. Cufflinks measures transcripts abundances in Fragments Per Kilobase of transcript per Million mapped reads (FPKM). One of the assumptions of reference-based transcriptome assembly approaches is that all the isoforms of all the genes are known. However, transcriptomic reference sets are still incomplete, more specially in the case of the rat transcriptome [6]. Hence, the transcriptomic assembly is not very accurate in rat. Therefore, all the RNA-seq expression data analyses in the rat have been carried out by summarising expression at the gene level.

Steps followed for gene level FPKM computation and filtering of the data: 1) Replace the values of all the isoforms with isoform quantification status not OK by missing values. The isoform quantification status is part of the output from Cufflinks and it measures the successful deconvolution of each isoform. 2) Remove all isoforms with more than 10 missing values across all samples. 3) Impute all the missing values by using R function pca from the pcaMethods R package 1.56.0 [7]. The initial number of isoforms was 39,492. After removing those with more than 10 missing values across all samples, the number of isoforms got reduced to 39,181. 4) Sum up all the FPKM values of all the isoforms of each gene to get the FPKM value at the gene level (Number of genes: 29,469). 5) Filter out genes that did not have FPKM >1 in at least a 5% of the samples (in at least 2 samples). 6) Replace 0s in the data by the minimum number in the data set higher than 0. Afterwards, Log2 transform the data. By following these steps, we ended up with a filtered data set of 12,061.

The inventors also inspected the presence of unmeasured confounding factors (i.e. potential sources of expression variation that are not being measured in the study and they may be affecting gene expression) [8]. We computed the variability explained by each of the PCs of the transcriptomic data. We found out that the 1st PC was explaining a large proportion of the variance present in the data (38%) and it was significantly correlated to the library concentration of the Bioanalyser, (a library quantification measure, Spearman's ranked correlation=0.70, p-value=1.5×10⁻⁵). To account for possible confounding effects introduced by the differences in library concentration, we adjusted the gene expression data for this 1st PC using a linear regression model from which the residuals were computed. These residuals obtained from the regression model represent the variability present in the data after removing the effect of this 1st PC. These adjusted expression levels were the ones considered in all analyses.

### Rat Histomorphometric Cardiac Fibrosis

Histomorphometric measures of both interstitial and perivascular fibrosis were collected in the LV of the 30 RI strains (total n=180; n=5-7 per strain, males at 30 weeks of age). After fixation, short-axis heart slices were processed for paraffin embedding. Multiple 4 μm thick sections were de-paraffinized, rehydrated and picrosirius red-stained sections were prepared for evaluating fibrosis. The presence, type and extent of both interstitial and perivascular fibrosis was quantified via thresholding automated analysis using ImageJ 1.43 [9]. Blood pressure measurements were published in [10]. Indwelling aortic radiotelemetry transducers (Data Sciences International) at 8 weeks of age were implanted to measure arterial pressure in conscious, unrestrained rats. Radiotelemetry pressure was collected in 5-s bursts every 10 min and recorded over a period of 8 days, using 6-12 rats for each RI strain. The obtained blood pressure measurements were averaged within each RI strain and across eight sequential readings. Median blood pressure effects were removed from the interstitial and perivascular fibrosis measurements by performing standard multiple linear regression and then taking the residuals of the model.

### Genetic Data in the Rat RI Strains

Within the rat RI panel, SNPs are a comprehensive source of genetic diversity available for genetic association studies. The genetic map of the rat BXH/HXB RI strains was generated by the STAR Consortium as described in [10]. This genetic map was generated from over 13,000 SNP that led to 1,384 unique blocks of adjacent SNPs with identical strain distribution patterns.

Human Dilated Cardiomyopathy (DCM) and Healthy Expression Data from Left Ventricle (LV) and Genetic Data

Human LV RNA-seq data was collected in 128 DCM patients and 106 controls. In addition, genotyping data was collected in 96 DCM and 91 controls samples. We performed a quality control step (QC) in the RNA-seq data and removed outlier samples (see section “b. RNA extraction, sequencing and RNA-seq data processing”). After QC, the final cohort size used for co-expression analyses was 126 DCM patients and 92 control samples. The number of samples used for genetic mapping was 96 DCM and 91 controls (i.e. samples for which we have both RNA-seq and genotyping data). This data were published by Heinig et al [11] and are available at the European Genome-phenome Archive under the accession number EGAS00001002454.

### DCM/Controls LV Cohort Description

This data represents a retrospective cohort of heart patients diagnosed with DCM (obtained from the Royal Brompton and Harefield NHS Foundation Trust Tissue bank: EC Ref: 09/H0504/104+5) and healthy LV donors (healthy with respect to myocardial diseases such as DCM and HCM). Control left ventricular samples (healthy LV donors) were collected from healthy human hearts of non-related organ donors whose hearts were explanted to obtain pulmonary and aortic valves for transplant, valve replacement surgery or explanted for transplantation but not used due to logistical reasons. Control LV tissue was collected in four different centres: University of Szeged (Hungary), Vanderbilt University (Nashville, USA), University of Miami (USA), and the University of Sydney (Australia) [12]. In both DCM patients (“cases”) and controls only adult subjects (≥16 years old) were selected.

### RNA Extraction, Sequencing and RNA-Seq Data Processing

Reads were mapped to the human genome with TopHat 1.4.1 [4]. TopHat was run using annotation from Gencode release 19 (GRCh37.p13) annotation and allowing only 2 mismatches per 100 bp. In addition, TopHat was run with option −r 0, which specifies as zero the expected mate inner distance (as fragment size was 200 bp and read length was 100 bp). Option −M was also set, which removes multimapping reads before aligning to the transcriptome. Default options were chosen for the rest of parameters. TopHat mapping resulted in a mean of 173 M reads per sample being uniquely mappable (a mean of 92% of the total number of reads with all the samples having a mapping percentage higher than 60%) in the samples from DCM patients. Controls samples resulted in a mean of 185 M reads per sample being uniquely mappable (a mean of 90% of the total number of reads with all the samples had a mapping percentage higher than 60%).

RNA-seq read gene counts were computed with HTSeq software 0.5.3p3 [13]. In HTSeq the mode ‘intersection-nonempty’ was selected (mode suitable to quantify overlapping transcripts on different strands). To compute gene counts with HTSeq, we used the Gencode human annotation version 19 with a custom TTN annotation [11].

Steps followed for processing of the data: 1) Gene selection: From HTSeq output, only “protein coding” genes with “known” status in Ensembl genes (GRCh37.p13 data set) were consider in the analyses (n=19,456). 2) Filtering of low expressed genes: Transcript lengths were downloaded from Biomart Ensembl genes (GRCh37.p13 data set). FPKM was computed with DESeq2 1.6.3 R package [14] by considering for each gene, the average length of all the transcripts. A FPKM-based gene filtering was then applied (we only kept genes with FPKM >1 in at least 5% of the samples, considering DCM and controls samples together). This yielded a final number of 14,281 genes that were considered to be expressed in the samples cohort. 3) Normalisation and data transformation: After the FPKM filtering, gene raw counts were normalised and variance-stabilized transformed (VST) by using DESeq2 1.6.3 R package [14]. 4) Covariates adjustment: VST data was split into DCM patients and healthy controls. Then, the data was adjusted separately for relevant technical (“RIN score” and “library preparation day”) and clinical (“sex” and “age at tissue collection”) covariates by using a multivariate linear model. In the case of the healthy controls, the data was also adjusted for the centre from which the tissue had been collected by adding this information as an additional covariate. 5) Outlier samples removal (samples QC): Outliers samples were inspected by clustering the resulting samples gene expression levels. The DCM and control samples were independently clustered by hierarchical clustering with the R function flashClust from flashClust 1.01-2 R package [15], the agglomeration method used was set to “average”). The obtained dendrograms were cut at the 99th percentile of the dendrogram height distribution removing all the samples assigned to small clusters: 2 DCM patients and 14 healthy donors. By following these steps, we ended up with two LV adult cohorts of 126 DCM patients (cases) and 92 healthy donors (controls). Within the 126 DCM patients, 106 were males and 20 were females. In the Healthy Donors group there were 55 male and 37 female. The mean age in the DCM patients was 41.3±13.3 years (16 to 64), whereas the mean age in the controls was 42.6±13.6 years (17 to 72).

### Genetic Data

A subset of the DCM and control LV samples from the CEU population (96 DCM samples and 91 control samples [11]), were typed by using genotyping arrays. After imputation and quality control, the final number of SNPs in the DCM patients and healthy controls LV samples was 1,309,892 SNPs.

In order to run genetic mapping, we carried out a pairwise linkage disequilibrium (LD) filtering (with an R² threshold of 0.8) based on the 1,000 Genomes pilot 1 CEU population. Pairwise SNPs LD was computed using SNAP 2.2 [16] using as input the SNP identifiers. The SNP dataset chosen to run SNAP was the 1000 Genomes pilot 1 in the CEU reference population with an R2 threshold of 0.8 and a distance limit of 500 bp. From SNAP output, SNPs returning a warning message as not present in the SNAP database were removed and not considered further analyses (the number of missing SNPs was 101,020 SNPs).

SNAP web tool outputs the input SNPs clustered in LD blocks (a total number of 126,922 LD blocks were returned by SNAP). To obtain a genomic map filtered by LD, we selected one single SNP for each of the LD blocks (R²=0.8). SNPs that were not included in any of the LD blocks (not clustered by SNAP) were added at the end. For each LD block we followed these steps: 1) Get all the SNPs in the LD block and compute their pairwise Spearman's ranked correlation. 2) As representative of the LD block, take the SNP that has the highest average Spearman's ranked correlation with the rest of SNPs included in the LD block.

With this procedure we obtained a set of 126,922 SNPs, each of them representing a SNPLD block. Finally, we added to these SNPs the ones that were not included by SNAP in any of the LD blocks (41,315 SNPs). Then, the final number of SNPs in our LD pruned (R²=0.8) genetic map was 168,237 SNPs.

### Human Right Ventricle (RV) Cohort Data

This data is a cohort of RV RNA-seq samples with repaired Tetralogy of Fallot (rTOF) patients (fibrotic RV) and control samples. Tetralogy of Fallot (TOF) is a congenital heart disease (i.e. problem in the structure of the heart) characterized by pulmonary artery stenosis, ventricular septal defect (a hole between the LV and RV and an overriding aorta which allows blood from both ventricles to enter the aorta (leading to cyanosis) and RV hypertrophy. TOF needs used to be treated surgically in the first year of life to increase the size of the pulmonary valve and arteries and repairing the septal defect. However, surgical repaired TOF is usually followed by cardiac fibrosis in both ventricles [17]. Additionally, TOF cases are operated several times during heart post-natal development because the prostatic material used at surgeries is unable to grow with the growing of the heart and valve. Therefore, surgery leftover samples can be collected at different ages of the patients.

rTOF Cohort Description

In this cohort there are 27 patients with rTOF (mean age 32.0±10.6 years, 78% male). All the patients aged ≥16 years old and were scheduled for elective pulmonary valve replacement. They were also under the care of the Adult Congenital Heart Disease service at the Royal Brompton Hospital, UK. For the rTOF patients, the clinical data from noninvasive investigations performed as part of the surgical work-up was available. This clinical information included electrocardiography, chest radiograph, echocardiography and CMR. RV myocardial tissue samples from the 27 patients were snap-frozen in liquid nitrogen at the time of tissue sampling intra-operatively.

RV tissue from 11 structurally normal hearts donated for cardiac transplantation was also collected (age donors: 34.0±13.0, Sex, Male/Female: 6/5). RV myocardial biopsies were made available via the Cardiovascular Biomedical Research Unit Biobank of the Royal Brompton & Harefield NHS Foundation Trust. Donor RV control tissue was collected and stored at the time of surgery following similar procedures as with the TOF tissue samples.

### RNA Extraction, Sequencing and RNA-Seq Data Processing

TRIzol (Life Technologies) was used for total RNA extraction from the frozen samples by following the manufacturer's protocol. RNA was quantified by ultraviolet spectrophotometry and RNA quality was assessed on the Agilent 2100 bioanalyzer. RINs ranged from 6.3 to 9.1 (mean 8.2±0.6). 1 μg of total RNA was used to prepare the RNA-Seq libraries. RNA-Seq libraries were prepared with Illumina TruSeq RNA sample preparation kits by using the protocol for poly-A enriched mRNA. To avoid batch effects, samples were pooled (4-5 samples/pool, 2 lanes per pool). Finally, paired-end 2×100 bp sequencing was performed on the Illumina Hi-Seq platform (mean sequencing depth of 196M). TopHat 2.0.12 with Bowtie2 2.2.3 and Samtools 0.1.18 [4] was run by using human genome version GRch38 (hg38.78) reference genome. RNA-seq read counts were computed with HTSeq 0.6.1 [13]. The percentage of reads mapping to the human genome was higher that 80% (above 70% is considered an acceptable mapping percentage for paired-end sequenced reads).

Steps followed for the normalization, filtering and adjustment of the data: 1) Gene selection: From HTSeqoutput, only “protein coding” genes with status “known” in Ensembl genes (GRCh37.p13 data set) were selected (18,964 genes). 2) Filtering of low expressed genes: FPKMs were computed with DESeq2 1.6.3 R package [14] using the average transcript length of each gene, which were retrieved from Ensembl Biomart (GRCh37 version). A FKPM-based filtering criteria was applied by keeping only those genes with a value of FPKM >1 in at least 5% of the samples (in this case 2 samples). Following these criteria, the number of genes got reduced from 18,964 genes to 13,936 genes. 3) Data transformation: Size factors normalisation and VST was applied to the raw gene counts by using DESeq2 R package 1.6.3. Then, the gene counts that passed the filtering criteria described in the previous step were selected. 4) Covariates adjustment: After normalisation and filtering, the data was split into TOF patients and controls. Gene expression counts of TOF patients were adjusted for: 1) age at which the tissue was collected (age of operation) and 2) gender. This adjustment was performed by taking the residuals of a multivariate linear model in which both age and gender were added as predictors. Among the control samples, some had with missing values for age. More specifically, three samples had missing age. The gender of the sample with missing value was imputed by clustering the expression levels of selected sex specific genes (as described in [18]). Therefore, the gene expression counts of the control samples were adjusted for only sex by taking the residuals of a linear model in which gender was added as a predictor.

### 1.2 Co-Expression Network Analysis in LV

Co-expression network inference in rat RI strains and human DCM LV tissues Co-expression networks were inferred by using WGCNA 1.42 [19]. Two independent WGCNA runs were carried out in the processed RNA-seq LV data (as described in previous sections): one in rat (RI panel, n=30) and one in the DCM cohort (n=126). In both rat and human runs, WGCNA was run using Turkey's biweight midcorrelation. Biweight midcorrelation estimates are more robust to outliers than the standard Pearson correlation as it assigns lower weights to points further from the centre of the distribution [20]. In the rat run, amongst all the soft threshold values (β) with R²>0.8, we chose the β that presented the highest mean connectivity (β=8). For the human run, the automatic value of β returned by the WGCNA function pickSoftThreshold was selected (β=6). In both cases, a network merge height of 0.25 was chosen as suggested in the original WGCNA guidelines. For the rest of WGCNA parameters, default settings were used. Once the WGCNA networks were obtained, they were alphabetically sorted by name and renamed as M1-M (number last network, Hs-M in the case of human networks) removing the grey cluster (the grey cluster contains all the non-clustered genes).

Gene-annotation enrichment analysis of the rat (n=41) and human (n=48) networks was performed by carrying out functional gene list enrichment analysis using the R DAVID Web Service 1.4.0 [21]. This tool provides an R interface to DAVID [22]. Rat networks were queried independently from human networks for Gene Ontology (Biological process, BP, Molecular Function, MF and Cellular Component, CC independently) [23] and KEGG [24] gene annotation categories. The gene reference background was set to the group of genes from which the gene co-expression networks were inferred. In the rat data this background was constituted by the genes robustly expressed in the rat LV data (i.e. after the filtering procedure described in previous sections, n=12,061), whereas in the human data the background used was the set of genes robustly expressed in the human LV RNA-seq cohort (i.e. after the filtering procedure previously described, n=14,281). In this analysis results were deemed significant if DAVID FDR/100<0.05 (i.e. % FDR).

Association of the Rat Co-Expression Networks with Fibrosis

Genome-wide gene expression levels in the RI strains LV were correlated with both interstitial and perivascular fibrosis using Spearman's ranked correlation (after correcting for average blood pressure effects as described previous sections). Here we used the measurements of interstitial and perivascular fibrosis in the rat after normal transformation (qqnorm R function used) and adjustment for mean blood pressure. Student p-values were obtained for each correlation estimate by using the function corAndPvalue from the R package WGCNA 1.42 [19].

All the rat co-expression networks were tested for enrichment of genes varying with fibrosis in the rat heart by carrying out GSEA. In this analysis, each of the rat co-expression networks was considered as a gene set. The corresponding Student p-values of the Spearman's ranked correlation between each of the robustly expressed rat genes and each of the fibrosis measurements were used for ranking all the rat genes (n=12,061) and run GSEA. GSEA 2.1.0 [25] was run in classic, pre-ranked mode with 10,000 iterations. To consider all the co-expression networks, maximum gene set size was set to 5,000 and minimum gene set size was set to 10. As the genes were ranked by p-value, in this GSEA test, significant negative normalised enrichment score (NES, i.e. FDR<0.05) denotes significant association.

### Conservation of Rat and Human Co-Expression Networks

To assess whether the co-expression networks inferred in the rat were also inferred in the DCM patients, we computed the intersection between rat and human co-expression networks by carrying out Fisher's exact tests. The gene background used in these tests was composed by the genes with one-to-one human-rat ortholog relationship that were both robustly expressed in rat and human LV. For the generation of this gene background, rat human one-to-one orthologs relationships were downloaded from Ensembl archive (Ensembl 69). The common set of genes in both the rat LV expressed genes set (n=12,061) and human LV expressed genes (n=14,281), yielded a common set of 8,840 genes (this will be the rat-human orthologs background). Genes in the rat and human networks that were not included in this rat-human orthologs background were removed.

For each rat (n=41), human (n=48) network pair, a Fisher's exact test was computed with the R function fisher.test and setting the “alternative” parameter to “greater” (as we are interested in overrepresentation). The gene background used for computing the contingency table was the rat-human orthologs background (8,840 genes). Nominal Fisher's exact test pvalues were adjusted for the number of tests carried out (number of rat networks times number of human networks=1,968 tests) by using the R function p.adjust and the B&Y method.

### Test for Differential Co-Expression of Human DCM LV Networks Between DCM Patients and Controls

Differentially co-expressed networks (i.e. gene networks where the genes as a whole present divergent patterns of co-expression between cases and controls) can point to disease response for example. We carried out the following empirical differential coexpression test to assess the differential co-expression of the human DCM networks between DCM patients and controls samples. First, we computed Tukey's biweight pairwise gene-gene correlations [20] from the DCM and controls expression matrices. Then, for each of the DCM co-expression networks these steps were carried out: 1) Compute the network's dispersion value (DCM versus controls samples) as described in the section 3 of supplementary material of the reference [26]. The dispersion value quantifies the difference between the co-expression of the network in cases and controls; 2) Generate a null distribution of dispersion values for the network by randomly sampling networks with the same number of genes as the network being tested and then, compute the corresponding dispersion value of each randomly sampled network as described in step 1. 3) Compute the empirical p-value for the network under testing as [27]: P=(r+1)/(n+1), where n is the number of simulated dispersion values (number of permutations) and r is the number of simulated dispersion values that are higher than the actual dispersion value of the network of interest.

This differential co-expression test was run with 100,000 permutations, which yields a minimum nominal significance level of 1×10⁻⁵. Bonferroni-adjusted p-values were computed by correcting the nominal empirical p-values for the number of DCM co-expression networks tested (n=48). p.adjust R function was used to adjust the p-values for multiple testing.

This differential co-expression test was also carried out for the hECM-network in the rTOF/controls RV cohort. In this case the input was the sex-adjusted VST counts for the genes in the hECM-network. The test was also performed for 100,000 permutations.

### 1.3 Analysis of the Human Networks

### Enrichment of Human Networks for Cardiac Fibroblast and Myocyte Expression

### Description of the Cardiac Data Used

This RNA-Seq data set consists of primary mouse cardiac fibroblasts (n=2) and cardiomyocytes (n=2). Neonatal cardiomyocytes and cardiac fibroblasts were isolated from 0-1 day old C57bl/6 mice using the procedure described in [28]. Cells were cultured at 37° C. for 48 h in the presence of 10% fetal calf serum in DMEM medium. Total RNA was purified using the RNeasy kit (Qiagen) and sequenced with the Illumina Genome Analyzer IIx, 2 replicates per condition at 75 bp, paired-end library, one lane per replicate (sequencing depth of roughly 40M reads).

TopHat2 (version 2.0.13) [29] was used to align the paired-end reads to the GRCm38 mouse genome using available transcript annotations from Ensembl release 76. Mean insert size and standard deviation were computed empirically from uniquely mapping, perfect matching mate pairs via a preliminary alignment with Bowtie2 (version 2.2.3) [30]. These were supplied as input parameters to TopHat2. Default parameterisations were used for the rest of options. Read counts for each gene were retrieved using HTSeq (version 0.6.1p1) with default parameters [13].

### Enrichment Test

When inferring gene co-expression networks from complex tissue (i.e. LV), the obtained gene networks could be capturing transcriptional programs coming from specific cell types. We set out to explore this by analysing gene expression data from the two most abundant cell types in the heart: cardiac fibroblasts and myocytes [31]. We ran a test to assess whether any of the human DCM networks were overrepresented for genes with higher or lower expression levels in cardiac fibroblasts when comparing to cardiomyocytes. More specifically, we carried out a GSEA analysis of cell type gene expression data from the mouse cardiac fibroblasts and myocytes described hereinabove.

Mouse-human orthologs relationships was downloaded from Ensembl archive (Ensembl version GRCh37). Using this annotation, all mouse genes were mapped to their corresponding human ortholog by taking only those human genes with one-to-one (ortholog_one2one) human-mouse relationship. This resulted in 11,862 genes. The cardiac fibroblast-to-myocyte fold change was computed for each of these 11,862 genes by dividing the average expression counts in cardiac fibroblasts by the average expression counts in myocytes. This leads to a cardiac fibroblast-tomyocyte fold change value for each gene representing how many times is higher or lower the expression of that gene in fibroblasts in comparison to myocytes. Afterwards, all the human genes (11,862 genes) were ranked by their corresponding value of fibroblast-to-myocyte fold change. Then, this information was used as ranked list to run GSEA [25]. In this GSEA analysis we used the human DCM clusters (considering only the genes present in these 11,862 genes) as gene sets. We run GSEA (version 2.1.0) [25] in classic, pre-ranked mode with 10,000 iterations by setting the maximum gene set size to 5,000 and the minimum gene set size to 10.

Test for Conservation of the Human Networks in rTOF RV Tissue

To explore the conservation in rTOF RV patients of the prioritised human network inferred in human DCM LV, we implemented an empirical permutation test that assesses how likely is it to find by chance a set of genes of the same size as the network with a similar degree of average absolute correlation in the data set under testing. In this empirical we assess the conservation of each human network in rTOF expression data set as following: 1) From the background to test (i.e. expression data set in which we want to see whether our network is conserved), randomly sample without replacement a set of genes as the same size of the co-expression network under testing. Compute the mean absolute correlation of the sampled genes without considering self-correlations. We repeat this step for a number of times (e.g. 100,000) to generate a null distribution of simulated mean correlations. 2) Compute the mean absolute correlation (without considering self-correlations) of the genes in each network in the data set in which we are testing for conservation (rTOF). 3) Compute the corresponding empirical p-value by using the values obtained in steps 1 and 2 as in [27]: P=(r+1)/(n+1), where n is the number of simulations (number of permutations) and r is the number of simulated mean correlation values higher than the actual mean correlation value of the network of interest. We ran this test using Turkey's biweight midcorrelation and 100,000 permutations, which yields a minimum nominal significance level of 1×10⁻⁵. The pvalue obtained for the test of the hECM-network in rTOF RV patients was 1×10⁻⁵.

### Test for Overrepresentation of Genes Differentially Expressed by TGFβ1 and TGFβ2 (24 Hrs) in the Human Co-Expression Networks

### Description of the Cells

Human ventricular cardiac fibroblasts (passage 5) were grown to ~90% confluence in 1% fetal bovine serum medium for 24 h. Then, they were incubated in triplicates (9 samples in total) in 1% fetal bovine serum medium for 24 h with +/−: human TGFβ1 (R&D cat100B, 5 ng/ml), or human recombinant TGFβ2 (Millipore cat GF113, 5 ng/ml). Supplier of ventricular human cardiac fibroblasts: Lonza (Catalogue number: CC-2904), product name: NHCF-V human cardiac fibroblasts-ventricular. Media kit: Fibroblast Growth Medium-3 BulletKit™ Kit. Catalogue number: CC-4526.

### RNA Extraction, Sequencing and RNA-Seq Data Processing

Total RNA was extracted and then sequenced with the Solexa platform (2×100 bp pairedend). Reads were mapped to the human genome (Homo sapiens GRCh37.73, human genome 19 version) using TopHat 2.0.6 [4] and gene counts were computed with HTSeq 0.5.4p3 [13].

### Differential Expression Analysis

Raw human counts were used as input to run the R package DESeq2 1.6.3. [14]. DESeq R function was run, considering the following two treatments in the primary human cardiac fibroblasts data: 24 h induction with TGFβ1 (n=3) and 24 h induction with TGFβ2 (n=3). In both cases, as baseline, 24 h control human cardiac fibroblasts (n=3) were used. Due to the low number of samples, to obtain the adjusted DESeq2 DE p-values, the outlier correction parameter cooksCutoff from the DESeq2 package function results was set to false.

### Enrichment Test for 24 Hrs TGFβ DE Genes

In this analysis only genes expressed in human cardiac fibroblasts that were also robustly expressed in the DCM LV (i.e. WGCNA input set of genes) were considered for both the ranked list and human networks. Genes were ranked by the output statistic of the differential expression test (Wald statistic, as we were using default parameterizations). After this, GSEA was run twice using as gene set the co-expression networks built in human DCM LV, and as ranked list all the genes ranked by the statistic of the differential expression test after 24 h induction with either TGFβ1 or with TGFβ2. We run GSEA 2.1.0 in classic, pre-ranked mode with 10,000 iterations by setting the maximum gene set size to 5,000 and the minimum gene set size to 10.

hECM-Network Enrichment for Differentially Expressed Genes in DCM with Progression to Heart Failure

We carried out an enrichment analysis of the hECM-network by using the results of the study published by Burke et al [32]. In this study they use a mouse genetic model of DCM and perform RNA-seq and differentially expression analysis in both cardiomyocytes and non-myocytes from hearts of control mice, mice with DCM and heart failure. Two lists of nonmyocyte differentially expressed genes were downloaded from the Supplementary Table 3 from the study published by Burke et al (comparisons “nonmyocyte DCM vs WT” and “nonmyocyte HF vs WT”) [32]. As this study was carried out in mouse, to test for overrepresentation of the hECM-network genes in these lists of differentially expressed genes, we first mapped the genes included in the hECM-network (683 human genes) to their one-to-one mouse orthologs that were also expressed in the mouse heart. For this we downloaded all the human-to-mouse one-to-one orthologs from Biomart and also only considered genes expressed in the mouse left ventricle with FPKM>1 in at least two mice (as described in section “Analysis of Wwp2^{Mut/Mut} mouse RNA-seq data”). This resulted in a reduction of the 683 hECM-network genes to 415 genes as present in the network, expressed in the mouse heart left ventricle and with one-to-one human-mouse ortholog relationship. We then generated a background for this test composed by the set of mouse genes expressed in the mouse heart left ventricle also with one-to-one human-mouse ortholog relationship (10,271 genes). In the next step we selected only those genes in the differentially expressed gene lists provided by Burke et al that were present in these 10,271 mouse genes background. Finally, we carried out a Fisher's exact test testing for significant overlap between the hECM-network (now mapped to mouse, 415 genes) and the set of genes present in our 10,271 mouse gene background that were upregulated in noncardiomyocytes in DCM and HF differentially expressed gene lists separately (2,005 and 2,162 genes respectively). We used R function used fisher test and we set the alternative parameter to “greater” (as we tested for overrepresentation). This analysis resulted in 226 genes present in the hECM-network (now mapped to mouse) that were upregulated in DCM in non-myocytes and 221 genes upregulated in HF in non-myocytes (these represent 54% and 53% of the 415 network genes).

Enrichments of Experimentally Validated SMAD Targets in the hECM-Network

The ChEA TF curated database of experimental TF DNA binding mammalian data [33] was queried for experimentally validated SMAD1-8 targets. SMAD targets available for SMAD1-4 were also coming from the following references [34-37]. Overrepresentation of SMAD targets within the hECM-network was assessed by Fisher's exact test. Steps followed: 1) Remove SMAD targets that were not robustly expressed in DCM heart (here we considered the background from where the DCM network were inferred, n=14,281 genes). By applying this filtering, the initial number of SMAD targets (SMAD1=610, SMAD2=1936, SMAD3=3233 and SMAD4=5012) got reduced to SMAD1=386, SMAD2=1396, SMAD3=2076 and SMAD4=3164. 2) For each SMAD1-4 a Fisher's exact test was computed with the R function fisher.test and setting the alternative parameter to “greater” (as we are testing for overrepresentation). The gene background used for computing the contingency table was the set of genes from which the human DCM clusters were inferred (robustly expressed in the LV human cohort n=14,281).

### 1.4 Network Quantitative Trait Locus (Network-QTL) Analysis

Co-expression networks suggest coordinated genetic regulation, which can be exploited to uncover genetic regulators of these transcriptional programs. Moreover, conserved genetic regulation can be driving fundamental biological mechanisms. In keeping with previous studies in the rat, where the BXH/HXB rat panel yielded increased power to carry out genetic mapping of gene networks [38]. We used multivariate Bayesian genetic mapping approaches to map the rat and human ECM-networks to the rat and human genomes. We first considered the expression of the rat ECM-network genes as a multivariate quantitative trait and jointly mapped this to the rat genome. Then, we inspected whether the regulatory locus identified in the rat was independently replicated in human DCM heart by joint mapping of the genes in the ortholog human network (e.g. hECM-network), to the human locus that is syntenic to the rat regulatory loci. This two-step strategy (mapping in rats first followed by mapping in humans) has been previously used to identify trans-acting genetic regulators of transcriptional networks underlying complex disease [1].

The mapping of the rat and human networks was carried out by using HESS [39]. HESS is a sparse Bayesian multiple linear regression method in which mRNA expression levels for multiple genes are regressed against all SNPs to identify the minimum (non-redundant) set of SNPs that predicts the mRNA expression variability. This method has the following features: 1) It takes into account the LD structure of the genotype data (the dependence of the genetic determinants or predictors). This allows to reduce the number of tests to be carried out and pinpoint the putative causal genetic variant. 2) It makes possible to map several responses in one single test. Therefore, with HESS is possible to map to the genome (i.e. all genome-wide genetic markers) expression levels of several genes jointly (for instance, map the expression levels of genes included in a co-expression network, without having to summarise their variability by PC analysis). 3) It exploits multidimensional dependencies within the responses (i.e. correlation of the gene expression levels). This can be used to boost moderate associations. The output of this method is a marginal posterior probability of inclusion (MPPI) for each gene-SNP pair tested, which represents the posterior probability of association of each SNP given the data. From this MPPI, the Bayes Factor (BF) can be computed. BF represents the evidence of genetic regulation versus no genetic control and it is defined as the ratio between the posterior odds and the prior odds or ratio between the strengths of these models [38]. In our case, the prior probability (π) for the jth SNP associated with the gth gene is defined as: π=E(p_{g})/p, where p is the number of SNPs we are testing and E(p_{g}) is the a priori expected number of control points for the gth gene, in our case we fix E(p_{g})=2. For instance, in the case of the rat RI strains, as the number of genome wide SNPs we are testing is 1,394, p=1,384 and the prior probability becomes π=1.4×10⁻³. The BF is defined as the ratio between the posterior odds and the prior odds: BF=(MPPI_{gj}/(1−MPPI_{gj}))/(π/(1−π), where MPPI_{gj} represents the marginal posterior probability of inclusion for the g th gene and the jth SNP. By using this BF formula, we can compute the BF for each response-predictor pair (i.e. gene and SNP under testing) from the output MPPIs of HESS.

### Input Data for Genome-Wide Genetic Mapping of the Rat Networks in the RI Strains

The gene expression levels of the genes included in each of the rat co-expression networks built in the LV of interest were jointly mapped to the rat genome with HESS, i.e. rat networks conserved in human, overrepresented for genes correlating with fibrosis and with a pattern of co-expression not present in human control LV tissue: M1, M2 and M12 rat networks. The expression data used was the RNA-seq data in the RI strains (log 2 transformed FPKM adjusted for the 1^{st} PC). These runs were carried out with 1,384 genome-wide SNPs markers in 29 RI strains (instead of 30, as there is one RI strain with RNA-seq expression but no available genotype information).

### Input Data to Carry Out Fine Mapping at the Syntenic Human Regulatory Loci

For each of the rat networks that had regulatory loci with median BF of the genes in the rat networks >100 (i.e. rat networks M1 and M2), the rat regulatory SNPs were selected. Two independent runs were carried out, one for the DCM patients and one for controls. The human expression data input to HESS was the expression level of the genes in the human networks that were significantly intersecting the rat modules of interest (RNA-seq data in the DCM/controls after the processing described above). This was done in the n=96 DCM and n=91 control samples for which we had both gene expression and genotype information. The genetic data input to HESS was the set of SNPs tagging the human locus syntenic to the identified rat locus in each case. To identify each the human syntenic locus, we follow these steps: 1) Obtain the start and end positions of the rat haplotype that contained the regulatory SNP (rat Ensembl version 69). 2) Compute the central genetic coordinate of the rat haplotype as: centre haplotype=start haplotype+(end haplotype-start haplotype)/2. 3) Get the closest rat gene to that coordinate, then get the human start/end coordinates of the human ortholog gene. 4) In human (human Ensembl version GRCh37), compute the centre of the selected gene: centre gene=start gene+(end gene-start gene)/2. Take a window of 10 Mb (±5 Mb) around the centre gene, which in the case of the hECM-network yielded the region Hs-chr16: 64415969..74415969 (human Ensembl version GRCh37). This was the region that we mapped in the human DCM data (region comprising 475 SNPs from our LDpruned genetic map).

### Description of HESS Runs

HESS runs were performed with 20,000 sweeps, 5,000 burn ins and default parameterisations. From the output MPPIs, BFs were computed by using the formula described above. For each SNP, the interquantile range of the network genes BFs distribution was plotted by using the function errbar from the R package Hmisc 4.1-1.

### 1.5 Correlation Analyses Between WWP2 and hECM-Network Genes

WWP2 transcript levels in the LV cohorts (DCM/control) and RV cohorts (rTOF/control) were correlated separately in patients and controls to the rest of the genes included in the hECM network. We computed the Spearman's ranked correlation and p-value using the WGCNA R package function corAndPvalue WGCNA 1.42 [19]. The resulting correlation distributions were plotted as a density plot and tested for differences by using a two-sample nonparametric Mann-Whitney U test by using the R function wilcox.test. Nominal WWP2-gene correlation p-values were corrected for multiple testing by using the R function p.adjust (method “fdr”). In this multiple testing correction, we corrected for the number of genes in the hECM-network, 683. A core set of genes correlated to WWP2 was extracted by taking all the genes with correlation FDR<0.01 in both DCM and rTOF patients (326 genes). STRING protein-protein interaction database 10.0 [40] was queried with this set of genes on the May 9, 2018, here only experimental and co-expression connections were retrieved, confidence level 0.4. To obtain a network visualisation graph from STRING database, we only considered experimental connections and co-expression interactions with a minimum interaction score of 0.4. In the network graph, each node corresponds to a gene and colour was mapped to the correlation between WWP2 and each gene (the average correlation in DCM and rTOF patients). Genes annotated in String with the Gene Ontology CC term “extracellular matrix” were also retrieved and highlighted with thicker node border.

### 1.6 eQTL Mapping of WWP2 Isoforms in the DCM Cohort

WWP2 isoform expression levels were quantified in the DCM patients from the TopHat output with Sailfish 0.6.3 [42]. Sailfish was run with default parameterisations. The data was adjusted for relevant technical (“RIN score” and “library preparation day”) and clinical (“sex” and “age at tissue collection”) covariates by using a multivariate linear model and then mapped to the regulatory locus identified in human chromosome 16 (1 Mb centred around WWP2). In this case we mapped the 27 SNPs tagging this region in our genetic map. From the output MPPIs, BFs were computed as described above.

Non-parametric Kruskal-Wallis Tests were computed by using the R function kruskal.test. The obtained p-values were corrected for the number tests carried out (i.e. the number of WWP2 isoforms inferred in the DCM heart with an average RPKM level higher than 0, 18 isoforms). P-values were corrected by using the R function p.adjust and the correction method “fdr”.

### 1.7 Differential Expression Analysis Between rTOF RV and Control RV Tissue

Differentially expressed genes between rTOF patients (n=27) and controls samples (n=11), were computed with the R package DESeq2 1.6.3 [14] adding gender as covariate in the model (age was not added as there were several RV control samples with missing age values). CooksCutoff DeSeq2 parameter was set to False. The rest of parameters were left to default.

### 1.8 Animal Studies

Mice were bred and maintained in mice in a specific pathogen-free (SPF) environment and used according to guidelines issued by the National Advisory Committee on Laboratory Animal Research. Steps were taken to minimize animal suffering.

### Wwp2^{Mut/Mut} Mice

Wwp2^{mut/wt} mice were generated based on C57BL/6J background using CRISPR/Cas9 technology as previously reported [43, 44]. Briefly, mutant animals were generated by coinjection of Cas9 mRNA and individual gRNAs into one-cell mouse embryos. Founder animals carrying the indel mutations were identified first by PCR and T7 endonuclease I assay, and then by deep sequencing of the PCR products. Founders carrying the desired reading frame shift mutations were used to generate mutation-segregated heterozygous F1 animals by crossing with the wild type animals. Homozygous mutant animals were generated by heterozygote crossing and used for experiments in comparison with the wild type littermates. To look into specific function of individual Wwp2 isoforms, three gRNAs were designed to targeting coding Exon 2, aiming to introduce mutations in individual domains of the protein, which in humans have different functions and are encoded by the three different gene isoforms. Here, a reading frame shift mutation in Exon 2 would render Wwp2-FL and Wwp2-N to functionally null, but is unlikely to affect Wwp2-C function. The Wwp2^{Wt/Wt} mice were crossbred to generate Wwp2^{Mut/Mut} and Wwp2^{Wt/Wt} (WT) mice in vivarium. The primers used for genotyping mice for Wwp2 are shown in SEQ ID NOs:63 and 64.

### AngII (Angiotensin II) Infusion Model

Alzet mini osmotic pump (Model No. 1004, Durect Corporation) was subcutaneously implanted in eight-weeks old mice anaesthetized with 2% isoflurane. Miniosmotic pumps loaded with saline or Angiotensin II (Sigma Aldrich, #A9525) were implanted to deliver Ang II at 500 ng/kg/min for a period of 4 weeks [45].

### Myocardial Infarction (MI) Model

MI was induced in 8-10 week-old mice after anaesthetizing with Ketamine and Xylazine and intubated with 22G×1″ SURFLO Flash I.V. catheter (TERUMO) which was connected to an artificial rodent ventilator MINI VENT type 845 (Harvard Apparatus, USA). After exposing the heart via thoracotomy at the fourth left intercostal space, the left coronary artery (LAD) was permanently ligated with a 8-0 nylon monofilament suture [46]. The thorax was closed with 6-0 coated vicryl suture and mice were followed for 4 weeks after surgery.

For both models, mice were sacrificed after weighing them at indicated time points. Hearts were harvested for weight measurement, histological studies, collagen determination and molecular biology analyses. Samples sizes were determined by power analysis and n ≥8 mice per group were used to account for the inherent variability in the fibrotic response of mice. Mice died undergoing surgery before the sample collection were excluded from statistical analysis. Data from the animal studies were collected in a blinded manner.

### 1.9 Cell Culture and Treatment

Murine cardiac fibroblasts were taken from the left ventricle (LV) of mice. Minced LV pieces (1-3 mm³) were placed in 6 cm dishes with DMEM supplemented with 20% fetal bovine serum for less than 10 days to generate mice cardiac fibroblasts (P0) and passaged to P1 and P2 DMEM supplemented with 10% fetal bovine serum for experiments. Each experiment, all the cells from Wwp2^{Mut/Mut} and Wwp2^{Wt/Wt} (WT) hear were cultured at the same time with same generation. Human cardiac fibroblasts were isolated from right atrium (RA) appendage obtained from patients on cardiopulmonary-by-pass during cardiac surgery operations by digesting the tissue with Collagenase II. Cardiac fibroblasts are obtained by growing the homogenized tissue suspended in DMEM supplemented with 20% fetal bovine serum in a humidified atmosphere. C2C12 mouse myoblast cell line was grown in DMEM medium supplemented with 10% fetal bovine serum. The cells were passaged twice before being used for experiments.

### In Vitro Studies

To mimic the in vivo cardiac fibroblasts activity, cells were treated with transforming growth factor-b human (Sigma Aldrich, #T7039) at a concentration of 5 ng/μl for 16-72 hours. For siRNA and plasmid transfection, primary cardiac fibroblasts were seeded on a 6-well plate (~70%) and were transiently transfected with siRNA duplexes (20 nM) designed for targeting 5′ or 3′ in WWP2 mRNA (Qiagen) using Lipofectamine RNAiMAX (Life technologies holdings, #13778075) in a serum free medium for 48-72 hours according to the manufacturer's instructions. In parallel, WWP2-FL and N plasmids were transiently transfected using Lipofectamine 2000 (Life Technologies Holdings, #11668019) for 48-72 hours according to the manufacturer's instructions. For siRNA transfection in human cardiac fibroblasts, primary human cardiac fibroblasts were seeded on a 12 well plate (70000 cells/well) and were transiently transfected with siRNA duplexes (20 nM) designed for targeting 5′ or 3′ in WWP2 mRNA (Qiagen) using Lipofectamine RNAiMAX (Life technologies holdings, #13778075) in a serum free medium for 24 hours according to the manufacturer's instructions. For ubiquitination analysis, cells were treated with proteosomal inhibitor MG132 (Sigma Aldrich, #M7449) following stimulation with TGFβ1 for 4 h before harvesting. SB430542 (Stem Cell Technologies, #72234) was used to inhibit TGFβ1 effect for respective time points. Clomipramine hydrochloride (Sigma Aldrich, #C7291) was used to block the activity of HECT domain 1 hour before TGFβ1 treatment.

### 1.10 Echocardiography

Transthoracic echocardiography was performed on day 28 after Ang II infusion and MI model using Vevo 2100 (VisualSonics, VSI, Toronto, Canada) and a MS400 linear array transducer, 18- to 38-MHz under anesthetized condition. An average of 10 cardiac cycles of standard 2 dimensional (2D) were acquired and stored for subsequent analysis using Vevo Imaging Workstation version 1.7.2 (VisualSonics, VSI, Toronto, Canada). All images acquisition and analysis were performed by a blinded operator according to a previously described method [47].

For Ang II-infusion model, 2D guided M-mode of parasternal short-axis short (middle) were selected for visualization of the papillarymuscle during end systole and end diastole For MI model, the parasternal long-axis were analyzed at 3 levels (basal, mid and apical) and all measurements were averaged over three consecutive cardiac cycles. Left ventricular ejection fraction (EF) and fractional shortening (FS) were calculated using modified Quinone method, using the following formulas: LVEF=(LVIDed²−LVIDes²)/LVIDed2; FS=(LVIDed −LVIDes)/LVIDes; where LVIDed is Left ventricular internal diameter at end diastole and LVIDes is Left ventricular internal diameter at end systole.

### 1.11 Analysis of Single-Cell RNA-Sequencina (scRNA-Seq) Data from WT Mouse Heart with Anaiotensin II Infusion

### Mice Description and Experimental Protocol

Single cell suspension was prepared from the adult left ventricle of one mice with Angiotensin II infusion for 28 days, as previously described [48]. After removal of dead cells with MACS dead cell removal kit (Miltenyi Biotec, #130-090-101), cells were lysed and subsequently RNA was reverse-transcribed and converted into cDNA libraries for RNA-seq analysis using a Chromium Controller and a Chromium Single Cell 3′ v2 Reagent kit (Genomics 10×) following the manufacturer's protocol. The library was sequenced using the Illumina Hi-Seq3000 sequencing platform.

### Single-Cell Data Analysis

The reads were mapped to the mouse genome (m38, Ensembl version 89) and quantified using Cell Ranger 2.1.1 (10× Genomics). We provided to Cell Ranger a custom built reference transcriptome generated by filtering the Ensembl transcriptome (Ensembl file: Mus_musculus.GRCm38.89.gtf) for the gene biotypes: protein coding, lincRNA and antisense. Cell Ranger was run with the expect number of cells parameter (expect-cells) set to 3000. Cell Ranger out filtered matrices (i.e. genes.tsv and barcodes.tsv) were then input into R and genes with zero counts in all cells were discarded.

Three cell quality control filtering steps were implemented. We removed: (a) cells with less than the 50th percentile of the distribution of the total cells library size, (b) cells with less than the 50th percentile of the distribution of total of number of detected genes, (c) cells with more than 50% of their total gene count coming from mitochondrial genes. This resulted in a final number of 508 cells.

After applying these cell filtering steps, we carried out five gene quality control steps on the remaining cells: (a) we only kept “detectable” genes, defined as genes detected with more than one transcript in at least two cells, (b) we removed genes with low average expression in the data (i.e. genes with an average expression below 0.01, this cutoff was set based on the total distribution of average gene expression across all cells and all genes), (c) We removed genes encoded on the mitochondrial genome and the gene “Malati” as it was an outlier in the gene expression distribution. After all the gene quality control steps, the resulting number of genes was 6,728.

Gene counts were normalized with scran 1.8.4 R package [49]. Scran size factors were computed from cell pools by doing a pre-clustering of the data with the quickCluster function 45 (the output object of this function was provided to the computeSumFactors function and then run the normalize function was run, all of them with default parameterizations). t-SNE was computed by providing the Log2 scran-normalized data to the function plotTSNE from scatter 1.8.4 R package [50]. Two tSNE components were computed setting a random seed of 123456 using automatic perplexity (after removing an imposed minimum of 50, i.e. floor(number cells/5)). In the t-SNE graph, each cell was coloured by Wwp2 expression level (Log2 scran-normalized gene counts). Heart cell subpopulations were identified by using known cell marker genes. Specifically, we used: Aplnr and Pecami (endothelial cells); Lum (fibroblasts); Ttn (cardiomyocytes); Hbb-bs (eritrocytes); Ccr2, Cd163 and Ptprc (immune cells). 12.

### 1.12 Analysis of Wwp2^{Mut/Mut} Mouse RNA-Seq Data

### Generation of RNA-Seq Data

Total RNA from tissue was isolated from left ventricles of 9 Wwp2^{Mut/Mut} and 9 WT mice with 28 days of Angiotensin II infusion. mRNA libraries were constructed from poly(A)-selected RNA using the NEBNext Ultra Directional RNA library prep kit (Illumina, New England BioLabs) and sequenced on Illumina HiSeq 2500.

### Data Processing, Differential Expression, Functional Enrichment Test and Test for Overrepresentation of Human Co-Expression Networks in the Differentially Expressed Genes

RNA-seq reads were assessed for quality, aligned to m38 (Ensembl Gene annotation build 89) using STAR 2.5.2b [51] and quantified with RSEM 1.2.31 [52]. The average mapping rate (unique and multimapping) was 94.5%. Gene annotation was retrieved from Ensembl version 89 (m38) using the R library biomaRt 2.30.0 [53]. Ribosomal genes (Ensembl gene biotype “rRNA”) and mitochondrial genes were removed (391 genes). Gene counts were rounded using the R function round and differential expression analysis was performed with DESeq2 1.14.1 [14] with a pre-filtering step in which we considered only genes with more than 1 count when summing up across all samples.

DESeq2 was run pairwise comparing Wwp2^{Mut/Mut} with Angiotensin II against WT mice with Angiotensin II using Wald test, with the outlier correction parameter cooksCutoff set to false (default parameterizations for the rest of parameters). In the DESeq2 model, we added RNA concentration and sequencing lane as covariates. Functional enrichment analysis of the differential expression results was performed with Gene Set Enrichment Analysis (GSEA) software 2-2.2.2 [25]. From all genes included in DESeq2 output, we selected those with one-to-one mouse-human ortholog relationships (as downloaded from Biomart, 14820 genes) and then we mapped them to human gene symbols. Then ranked all the genes by the corresponding DESeq2 output Wald statistic (i.e. the estimate of the log 2 fold change divided by its standard error). GSEA was run assessing overrepresentation of the following gene sets and pathways derived from the Molecular Signatures Database gene sets 5.1 [54], (gene sets were queried using gene symbols): Hallmark gene sets (i.e. coherently expressed gene signatures derived from the aggregation of many MSigDB gene sets to represent well-defined biological states or processes), Gene Ontology and Reactome databases. GSEA was run in classic pre-rank mode with 10,000 permutations to assess false discovery rate (FDR). In the GSEA runs, maximum gene set size was set to 5,000 and minimum gene set size was set to 10. In this test, upregulated processes and pathways in the Wwp2^{Mut/Mut} will be positively enriched, whereas downregulated ones will be negatively enriched. Gene sets were deemed as enriched if FDR<0.05.

In addition, GSEA was run a second time using the same parameterization but this time testing for overrepresentation of all the human co-expression networks. In this last run, we further reduced the background to the common set of genes with one-one mouse-human ortholog relationship that were also present in both the DESeq2 mouse output and in the initial set of human genes considered for network inference in the human data. This background reduction was applied to both the ranked list and the human networks, resulting in a total number of 10,449 genes.

Differential Co-Expression Analysis of the hECM-Network in Wwp2^{Mut/Mut} Mice

We tested whether the hECM-network genes displayed differential co-expression upon Angiotensin II infusion when comparing the Wwp2^{Mut/Mut} mouse with control mice. To this aim, we followed the same procedure as we previously did to compute differential coexpression of the hECM-network genes in the two human cohorts (DCM/controls and rTOF/controls see previous sections). We applied a filtering and removed lowly expressed genes (i.e. we only took genes with an FPKM >1 in at least 2 out of the 18 samples), this led to 12,659 genes. Then, as the hECM-network was inferred in human, out of these 12,659 genes, we only considered the ones with one-to-one ortholog relationship to human (10,271 genes). From these genes we took the set of genes included in the hECM-network (415 genes). We added an offset of 1 and computed the Log₂ FPKM. Then we computed genegene pair Turkey's biweight midcorrelation separately in the WT-Angiotensin II and Wwp2^{Mut/Mut}-Angiotensin II mice and carried out the same test for differential co-expression as explain in the section “Test for differential co-expression of human DCM LV networks 47 between DCM patients and controls”.

### 1.13 Histology and Immunofluorescence

Left ventricles harvested from the mice were fixed in 10% Neutral buffered formalin (NBF) for 24 hours at RT, processed with Leica automatic tissue processor, paraffin-embedded and sectioned with thickness of 5 μm. After dewaxing and rehydration, slides were stained with Sirius Red Collagen kit (Chondrex, Inc, #9046) and Masson's Trichrome staining kit (Sigma-Aldrich, #HT15) as per manufacturer's instructions. Sections were stained using anti-ACTA2 (1:100), anti-S100A4 (1:100) to identify cell and biochemical features. Bovine Anti Rabbit IgG-CFL 488 (Santacruz Biotechnology, #sc-362260) and Bovine Anti Mouse IgG-CFL 488 (Santacruz Biotechnology, #sc-362256) were used as secondary antibodies for immunofluorescence. Rhodamine Wheat Germ Agglutinin (WGA, Vector laboratories, #RL-1022) was used to stain the myocytes.

Cells were grown in 8-well chamber slide with a removable silicone chamber (ibidi) up to 70% confluence. After fixation with ice cold acetone and blocking with 1% BSA for 30 min at RT, the slides were incubated with primary antibodies anti-WWP2-FL/N(1:100), anti-ACTA2 (1:100), anti-S100A4(1:100), anti-Vimentin(1:100) and anti-FLAG(1:100) overnight at 4° C. Following washing steps, the slides were incubated with Bovine Anti Rabbit IgG-CFL 488 (Santacruz Biotechnology, #sc-362260) and Bovine Anti Mouse IgG-CFL 488 (Santacruz Biotechnology, #sc-362256) for 2 hours at RT. VectaShield Mounting Medium (Vector laboratories, #H-1200) with DAPI was used to stain the nuclei and the slides were covered by coverslip.

Slides were imaged on Leica fluorescence microscope and image was processed using ImageJ software with the Fiji package. With merge function, the positive Sirius red staining in the whole section of the left ventricle was quantified using custom semiautomated image analysis routine. To measure the fluorescence intensity of ACTA2 and COL1A1 in different cellular sections, images were taken using 20× Plan Fluor objective. Fluorescence intensity was measured by taking the integrated intensity of a region of interest and subtracting the background intensity, and normalized to cell number. For each group, at least six fields were analyzed per section. To measure fluorescence intensities of SMAD2, 1-μm Z-stacks through cells of fields interested were acquired [55]. A region was drawn around each cell and nucleus to be measured, and background without fluorescence was subtracted. The nuclear/cellular fluorescence intensity ratio was calculated. Each field represented around 8-10 cells and at least 4 fields were analyzed for each section.

### 1.14 Hydroxyproline Assay

The amount of total collagen in the left ventricle was quantified using the Quickzyme Total Collagen assay kit (Quickzyme Biosciences). The assays were performed according to the manufacturer's protocol.

### 1.15 Luciferase Assay

Cells were transfected with a luciferase reporter gene plasmid with SMAD binding sites (Yeasen, SMAD-Luc, #11543ES03), and co-transfected with pGMLR-TK (Yeasen, #1 1557ES03) as a normalization control. 30 hours after transfection, cells were treated with vehicle or TGFβ1 for 16 hours and harvested. Luciferase assays were performed using the Dual-Luciferase Reporter Assay System (Yeasen, #11402ES60).

### 1.16 Cell Proliferation and Migration Assay

Cell proliferation was quantified by MTS assay (Promega) according to the manufacturers' protocol. For migration assay, cells were seeded at a density of 10,000 cells/well in a 96-well plate. A uniform, reproducible wound was created using Incucyte, Essen Bioscience (USA). The 96-well plate was placed in the Incucyte ZOOM apparatus and the images of cell migration was captured every 2 hours for up to a total of 48 hours [56].

### 1.17 RT-qPCR

Total RNA was extracted from snap-frozen fibrotic cardiac tissue and primary cardiac fibroblasts using the RNeasy mini kit (Qiagen, #74106) and cDNA was prepared using iScript cDNA synthesis kit (primer specific, BIORAD, #170-8897) according to the manufacturer's instructions. Fast SYBR-Green master mix (BIORAD, #170-8880AP) was used for the analysis of gene expression using the BIORAD CFX RT-PCR system. 18S was used to normalize the relative gene expression and 2^{-ΔΔct} method was used to measure the fold change.

The primers used for RT-qPCR analysis are shown in SEQ ID NOs: 27 to 62.

### 1.18 Western Blotting

Protein extracts were isolated from heart tissue and cells using RIPA buffer (Thermofischer, #89900) supplemented with protease (Sigma Aldrich, #11836170001) and phosphatase inhibitors cocktails (ROCHE, #PHOSS-RO). Nuclear and cytoplasmic extracts were obtained using NE-PER kit (Pierce, #78833) according to the manufacturer's instructions.

Co-Immunoprecipitation was performed with the cell lysates subjected to different treatment conditions with Pierce Direct Magnetic IP/CO-IP kit (Pierce, #88828) according to manufacturer's protocol.

Immunoprecipitates were washed from conjugated beads and boiled in 4×SDS-PAGE buffer for further WB analysis.

After quantification with Bradford method, protein lysates were loaded onto a 4-12% acrylamide gel subjected to SDS-PAGE and then transferred onto a nitrocellulose membrane. After blocking in 5% nonfat dry milk, blotting was performed with anti-WWP2 targeting N-terminal region (Santa Cruz biotechnology, #sc30052,1:500), anti-WWP2 targeting C-terminal region (Aviva Systems Biology, #ARP43089_P050,1:500), anti-TGFb1 (Santa Cruz biotechnology, #sc52893,1:500), anti-ACTA2 (Sigma-Aldrich, #A5228,1:10,000), anti-S100A4 (Abcam, #ab41532, 1:500), anti-Vimentin (Abcam, #ab45939,1:500), anti-Periostin (Novus bio, #NBP1-30042, 1:500), anti-Fibronectin (Sigma, #SAB4500974, 1:500), anti-pSMAD2 (CST, #18338,1:500), anti-SMAD 2/3 (CST, #3102, 1:500), anti-SMAD-4 (Santa Cruz biotechnology, #sc-7966, 1:500), anti-Ubiquitin (CST, #3933, 1:500), anti-FLAG (Sigma-Aldrich, #F7425, 1:1000). Loading control was blotted with Anti-tubulin (Sigma-Aldrich, #T5168, 1:5000) and anti-GAPDH (Abcam, #ab8245, 1:5000) anti-Lamin (abcam, #ab8984, 1:5000) and anti-PARP (abcam, #ab6079,1:5000) were used as nuclear controls. Blots were visualized by labeling with anti-Rabbit HRP (Bethyl laboratories, #A120-101P, 1:5000 or Thermo Fisher #101023, 1:1000) and anti-Mouse HRP (Bethyl laboratories, #A90-116P, 1:5000) and developed on a Kodak automated developer with the ECL and Femto Detection Systems (Pierce) and quantified using densitometry with Image J (version 2.0.0-rc-43).

### 1.19 Statistical Analysis

All data were analyzed using the appropriate statistical analysis methods with SPSS software (version 21.0), and the data are expressed as the mean±s.d. mean (95% CI or ±SD). The applied tests were dependent on the number of groups being compared and the study design. A two-tailed Mann-Whitney U test was used to compare two groups, with * denoting P<0.05, and ** denoting P<0.01. When comparing mice with different genotypes, male littermate mice were assigned to the WT and Mut/Mut groups according to the results of genotyping, and mice with the same genotype were randomly assigned to the control, AngII infusion or MI group using a simple random-sampling approach. All experiments requiring the use of animals, directly or as a source of cells, were subjected to randomization. The experimenters were blinded to the grouping information. All in vitro experiments were replicated at least three independent times.

### 1.20 References to Example 1

1. Moreno-Moral, A. and E. Petretto, From integrative genomics to systems genetics in the rat to link genotypes to phenotypes. Dis Model Mech, 2016. 9(10): p. 1097-1110.
2. https://amstat.tandfonline.com/doi/abs/10.1080/01621459.1995.10476572-.XD70SM8zb2K
3. Rintisch, C., et al., Natural variation of histone modification and its impact on gene expression in the rat genome. Genome Res, 2014. 24(6): p. 942-53.
4. Trapnell, C., L. Pachter, and S. L. Salzberg, TopHat: discovering splice junctions with RNA-Seq. Bioinformatics, 2009. 25(9): p. 1105-11.
5. Flicek, P., et al., Ensembl 2012. Nucleic Acids Res, 2012. 40(Database issue): p. D84-90.
6. Martin, J. A. and Z. Wang, Next-generation transcriptome assembly. Nat Rev Genet, 2011. 12(10): p. 671-82.
7. Stacklies, W., et al., pcaMethods—a bioconductor package providing PCA methods for incomplete data. Bioinformatics, 2007. 23(9): p. 1164-7.
8. Leek, J. T. and J. D. Storey, Capturing heterogeneity in gene expression studies by surrogate variable analysis. PLoS Genet, 2007. 3(9): p. 1724-35.
9. Mancini, M., et al., Mapping genetic determinants of coronary microvascular remodeling in the spontaneously hypertensive rat. Basic Res Cardiol, 2013. 108(1): p. 316.
10. Petretto, E., et al., Integrated genomic approaches implicate osteoglycin (Ogn) in the regulation of left ventricular mass. Nat Genet, 2008. 40(5): p. 546-52.
11. Heinig, M., et al., Natural genetic variation of the cardiac transcriptome in nondiseased donors and patients with dilated cardiomyopathy. Genome Biol, 2017. 18(1): p. 170.
12. Koopmann, T. T., et al., Genome-wide identification of expression quantitative trait loci (eQTLs) in human heart. PLoS One, 2014. 9(5): p. e97380.
13. Anders, S., P. T. Pyl, and W. Huber, HTSeq—a Python framework to work with highthroughput sequencing data. Bioinformatics, 2015. 31(2): p. 166-9.
14. Love, M. I., W. Huber, and S. Anders, Moderated estimation of fold change and dispersion for RNA-seq data with DESeq2. Genome Biol, 2014. 15(12): p. 550.
15. Langfelder, P. and S. Horvath, Fast R Functions for Robust Correlations and Hierarchical Clustering. J Stat Softw, 2012. 46(11).
16. Johnson, A. D., et al., SNAP: a web-based tool for identification and annotation of proxy SNPs using HapMap. Bioinformatics, 2008. 24(24): p. 2938-9.
17. Wald, R. M., A. M. Valente, and A. Marelli, Heart failure in adult congenital heart disease: Emerging concepts with a focus on tetralogy of Fallot. Trends Cardiovasc Med, 2015. 25(5): p. 422-32.
18. Heidecker, B., et al., The gene expression profile of patients with new-onset heart failure reveals important gender-specific differences. Eur Heart J, 2010. 31(10): p. 1188-96.
19. Zhang, B. and S. Horvath, A general framework for weighted gene co-expression network analysis. Stat Appl Genet Mol Biol, 2005. 4: p. Article 17.
20. Hardin, J., et al., A robust measure of correlation between two genes on a microarray. BMC Bioinformatics, 2007. 8: p. 220.
21. Fresno, C. and E. A. Fernandez, RDAVIDWebService: a versatile R interface to DAVID. Bioinformatics, 2013. 29(21): p. 2810-1.
22. Huang da, W., B. T. Sherman, and R. A. Lempicki, Systematic and integrative analysis of large gene lists using DAVID bioinformatics resources. Nat Protoc, 2009. 4(1): p. 44-57.
23. Ashburner, M., et al., Gene ontology: tool for the unification of biology. The Gene Ontology Consortium. Nat Genet, 2000. 25(1): p. 25-9.
24. Kanehisa, M., et al., KEGG for integration and interpretation of large-scale molecular data sets. Nucleic Acids Res, 2012. 40(Database issue): p. D109-14.
25. Subramanian, A., et al., Gene set enrichment analysis: a knowledge-based approach for interpreting genome-wide expression profiles. Proc Natl Acad Sci USA, 2005. 102(43): p. 15545-50.
26. Choi, Y. and C. Kendziorski, Statistical methods for gene set co-expression analysis. Bioinformatics, 2009. 25(21): p. 2780-6.
27. North, B. V., D. Curtis, and P. C. Sham, A note on the calculation of empirical P values from Monte Carlo procedures. Am J Hum Genet, 2002. 71(2): p. 439-41.
28. Brand, N. J., et al., Analysis of cardiac myocyte biology in transgenic mice: a protocol for preparation of neonatal mouse cardiac myocyte cultures. Methods Mol Biol, 2010. 633: p. 113-24.
29. Kim, D., et al., TopHat2: accurate alignment of transcriptomes in the presence of insertions, deletions and gene fusions. Genome Biol, 2013. 14(4): p. R36.
30. Langmead, B. and S. L. Salzberg, Fast gapped-read alignment with Bowtie 2. Nat Methods, 2012. 9(4): p. 357-9.
31. Souders, C. A., S. L. Bowers, and T. A. Baudino, Cardiac fibroblast: the renaissance cell. Circ Res, 2009. 105(12): p. 1164-76.
32. Burke, M. A., et al., Molecular profiling of dilated cardiomyopathy that progresses to heart failure. JCI Insight, 2016. 1(6).
33. Lachmann, A., et al., ChEA: transcription factor regulation inferred from integrating genome-wide ChIP-X experiments. Bioinformatics, 2010. 26(19): p. 2438-44.
34. Chen, X., et al., Integration of external signaling pathways with the core transcriptional network in embryonic stem cells. Cell, 2008. 133(6): p. 1106-17.
35. Koinuma, D., et al., Chromatin immunoprecipitation on microarray analysis of Smad2/3 binding sites reveals roles of ETS1 and TFAP2A in transforming growth factor beta signaling. Mol Cell Biol, 2009. 29(1): p. 172-86.
36. Kim, S. W., et al., Chromatin and transcriptional signatures for Nodal signaling during endoderm formation in hESCs. Dev Biol, 2011. 357(2): p. 492-504.
37. Kennedy, B. A., et al., ChIP-seq defined genome-wide map of TGFbeta/SMAD4 targets: implications with clinical outcome of ovarian cancer. PLoS One, 2011. 6(7): p. e22606.
38. Heinig, M., et al., A trans-acting locus regulates an anti-viral expression network and type 1 diabetes risk. Nature, 2010. 467(7314): p. 460-4.
39. Bottolo, L., et al., Bayesian detection of expression quantitative trait loci hot spots. Genetics, 2011. 189(4): p. 1449-59.
40. Szklarczyk, D., et al., STRING v10: protein-protein interaction networks, integrated over the tree of life. Nucleic Acids Res, 2015. 43(Database issue): p. D447-52.
41. Shannon, P., et al., Cytoscape: a software environment for integrated models of biomolecular interaction networks. Genome Res, 2003. 13(11): p. 2498-504.
42. Patro, R., S. M. Mount, and C. Kingsford, Sailfish enables alignment-free isoform quantification from RNA-seq reads using lightweight algorithms. Nat Biotechnol, 2014. 32(5): p. 462-4.
43. Zine-EddineKherraf, et al., Creation of knock out and knock in mice by CRISPR/Cas9 to validate candidate genes for human male infertility, interest, difficulties and feasibility. Molecular and Cell Endocrinology, 2018. 468: p. 70-80.
44. Bao, Q., et al., Publisher Correction: Utf1 contributes to intergenerational epigenetic inheritance of pluripotency. Sci Rep, 2018. 8(1): p. 1664.
45. Lu, H., et al., Subcutaneous Angiotensin II Infusion using Osmotic Pumps Induces Aortic Aneurysms in Mice. J Vis Exp, 2015(103).
46. Ye, L., et al., Thymosin beta4 increases the potency of transplanted mesenchymal stem cells for myocardial repair. Circulation, 2013. 128(11 Suppl 1): p. S32-41.
47. Fard, A., et al., Noninvasive assessment and necropsy validation of changes in left ventricular mass in ascending aortic banded mice. J Am Soc Echocardiogr, 2000. 13(6): p. 582-7.
48. Yen-Rei A. Yu, E. G. O. K., Danielle F. Hotten, Matthew J. Kan, David Kopin, Erik R. Nelson, Loretta Que, Michael D. Gunn, A Protocol for the Comprehensive Flow Cytometric Analysis of Immune Cells in Normal and Inflamed Murine Non-Lymphoid Tissues. PLoS One, 2016.
49. Lun, A. T., D. J. McCarthy, and J. C. Marioni, A step-by-step workflow for low-level analysis of single-cell RNA-seq data with Bioconductor. F1000 Res, 2016. 5: p. 2122.
50. McCarthy, D. J., et al., Scater: pre-processing, quality control, normalization and visualization of single-cell RNA-seq data in R. Bioinformatics, 2017. 33(8): p. 1179-1186.
51. Dobin, A., et al., STAR: ultrafast universal RNA-seq aligner. Bioinformatics, 2013. 29(1): p. 15-21.
52. Li, B. and C. N. Dewey, RSEM: accurate transcript quantification from RNA-Seq data with or without a reference genome. BMC Bioinformatics, 2011. 12: p. 323.
53. Durinck, S., et al., Mapping identifiers for the integration of genomic datasets with the R/Bioconductor package biomaRt. Nat Protoc, 2009. 4(8): p. 1184-91.
54. Liberzon, A., et al., The Molecular Signatures Database (MSigDB) hallmark gene set collection. Cell Syst, 2015. 1(6): p. 417-425.
55. Potapova, T. A., et al., Mitotic progression becomes irreversible in prometaphase and collapses when Wee1 and Cdc25 are inhibited. Mol Biol Cell, 2011. 22(8): p. 1191-206.
56. Stuart T. Johnston, E. T. S., Lisa K. Chopin, D. L. Sean McElwaina nd Matthew J. Simpson, Estimating cell diffusivity and cell proliferation rate by interpreting IncuCyteZOOM assay data using the Fisher-Kolmogorov model. BMC Systems Biology, 2015.

### Example 2: Identification of WWP2 as a Positive Regulator of a Network of ECM Genes Constituting a Pathological Fibrosis Program Conserved Across Fibrotic Diseases Affecting Different Organs

### 2.1 Identification of the hECM-Network

The inventors carried out a system genetics study that enabled the identification of pathological fibrosis programs (i.e. gene co-expression networks) conserved across species and common to several diseases. The analysis identified WWP2 gene as a regulator of the identified pathological fibrosis program.

The inventors set out to identify transcriptional programs conserved across species and associated to cardiac fibrosis, using a panel of 30 rat recombinant inbred (RI) strains (Hubner et al., 2005), allowing integrative analyses of cardiac gene expression with quantitative pathophysiological traits (e.g. cardiac fibrosis) and genome-wide genetic data (Petretto et al., 2008; Mancini et al., 2013; Langley et al., 2013). In addition, this panel of RI strains is an established model for cardiovascular traits and disease, including cardiac hypertrophy (Petretto et al., 2008), blood pressure (Pravenec et al., 2008) and heart remodeling (Mancini et al., 2013).

The inventors performed gene co-expression network inference in the rat RI strains left ventricle (LV) transcriptome using RNA-sequencing (RNA-seq) data. This identified 41 distinct gene co-expression networks, each with a different number of genes. The inventors then tested the association of these gene co-expression networks with quantitative histopathologic measurements of interstitial and perivascular fibrosis in the rat heart. Using Gene Set Enrichment Analysis (GSEA) (Subramanian et al., 2005), five gene co-expression networks were determined to be associated with both interstitial and perivascular cardiac fibrosis in rat LV tissue (adjusted P-value <0.05).

To identify transcriptional programs relevant to the fibrogenic processes in human heart disease, separate gene network analysis was also performed using LV RNA-seq data generated from a cohort of patients with DCM (n=126) and a cohort of control heart samples (n=92 organ donors whose hearts were explanted for transplantation, described in Heinig et al., 2017). Applying the same network methodology that was used for the rat analysis, the inventors inferred 48 genome-wide gene co-expression networks in the human DCM LV transcriptome.

The inventors next assessed which of the human DCM gene networks were conserved in the rat LV using a Fisher test. 14 human DCM networks were determined to have some degree of conservation with the rat networks (adjusted P-value <0.05).

In order to obtain additional evidence in support of a role for these networks in human heart disease, the inventors tested which gene co-expression patterns were present only in the LV from human DCM patients and not in LV from controls (i.e. they tested for differential co-expression between DCM and control hearts). The differential co-expression paradigm assumes that the disease state is linked to perturbations of the structure of the regulatory network itself, and might reflect the dysregulation of the underlying transcription factors in disease (Rotival and Petretto, 2014). Here, the inventors found that 8 human gene networks were both conserved across species and differentially co-expressed between human DCM and control LV tissues (adjusted P-value <0.05). Despite the fact that several networks were significantly conserved between rat and human DCM heart, only one human network (referred to hereafter as the hECM-network), containing 683 genes, was (i) significantly conserved in the rat (sharing 72 common genes with the rat ECM network (adjusted P-value=8.4×10⁻⁴⁵), (ii) correlated with both interstitial and perivascular fibrosis levels in the rat heart, and (iii) differentially co-expressed in human DCM heart. The hECM-network was also functionally relevant for ECM regulation, and was enriched (False Discovery Rate (FDR)<0.05) for genes belonging to the specific biological pathways and processes: “ECM-receptor interaction”, “TGFβ signaling pathway” and “focal adhesion”, as determined by KEGG annotation analysis.

The hECM-network contains 237 co-expressed genes annotated to encode extracellular ECM region proteins; 21 encode collagens, several encode focal adhesion molecules such as ITGB5, COMP, MAPK10 and THBS4; several encode extracellular genes involved in TGFβ-signalling (e.g., DCN, CHRD, TGFβ3); 3 encode members of the BMP family (BMP4, BMP6 and BMP8B), and genes encoding other important matricellular proteins are also represented, such as CTGF and PDGFD, which contribute to the fibrogenic response (Wang et al., 2011; Zhao et al., 2013).

The full list of hECM-network 237 genes is as follows: ABI3BP, COL12A1, EPHB3, LRRC4C, PRCD, ACHE, COL14A1, EXT2, LSP1, PRCP, AEBP1, COL15A1, F2R, LTBP2, PSMB2, AGRN, COL16A1, F2RL2, LTBP3, PSMD11, AKR1C1, COL1A1, FAM26E, LTBP4, PTGS1, AKR1C3, COL1A2, FAP, LUM, PTHLH, ALDH1A1, COL22A1, FAT4, LYPD1, PTN, ANGPT1, COL24A1, FBLN1, MATN2, PTPN13, ANO1, COL3A1, FBLN2, MDK, PTPRA, ANTXR1, COL4A1, FBLN7, MFAP2, PTPRR, ANXA1, COL4A2, FBN1, MFAP4, PXDN, ANXA4, COL4A3, FGL2, MFAP5, QPCT, ART4, COL4A4, FLRT2, MGP, RECK, ASPN, COL4A5, FMOD, MMP14, S100A4, BEND7, COL5A1, FN1, MMP2, SCG2, BGN, COL5A2, FRMD4B, MMP28, SCRG1, BMP4, COL6A1, FRZB, MOXD1, SCUBE2, BMP6, COL6A2, FSCN1, MTMR 11, SEPT11, BMP8B, COL8A1, FUT8, MXRA5, SEPT2, BST1, COL8A2, GAL3ST4, MXRA8, SERPINE2, C90RF72, COL9A2, GDF10, MYH10, SERPINF1, CAPN5, COLEC11, GDF6, MYO1D, SFRP4, CBR3, COMP, GDNF, MYO1E, SH3BGRL, CCL28, CPE, GLIPR2, NAGLU, SLIT2, CD34, CPXM2, GNB1, NBL1, SLIT3, CD44, CRABP2, GNG2, NCS1, SOD2, CD9, CRISPLD1, GOLM1, NOTCH2, SOD3, CDH 11, CTGF, GPM6A, NOTCH3, SOSTDC1, CDH6, CTHRC1, GPX3, NT5E, SPARC, CDK5RAP2, CTSK, GSN, NTM, SPON2, CHRD, CTSO, HAAO, OAF, SPTBN2, CHRDL1, CUL4B, HAPLN1, OGN, SSC5D, CHST14, CYBRD1, HMCN1, OLFML3, SULF1, CILP, CYTL1, HNMT, OMD, SUSD2, CLDN11, DCN, IGFBP6, PCDHGC3, TARS, CLEC11A, DDR2, IGFBP7, PCDHGC5, TGFB3, CNTN4, DNM1, IGSF10, PCOLCE, TGFBI, DPT, IL16, PCOLCE2, THBS3, DPYSL3, IL33, PCSK5, THBS4, DUOX2, ISLR, PCYOX1L, THY1, ECM1, ITGB5, PDGFD, TIMP2, ECM2, KIRREL, PDXK, TNXB, EFEMP2, LASP1, PENK, TOM1, EHD2, LEFTY2, P116, TPPP3, ELN, LINGO1, PLA2R1, TRABD2B, EMILIN3, LOX, PLAT, TSPAN8, ENPP2, LOXL1, PLSCR4, UCHL1, ENTPD1, LOXL2, PLXDC2, VCAN, EPB41L2, LOXL3, PODN, WNT10B, EPHB2, LRRC17, POSTN and ZDHHC1.

Because the hECM-network was identified in DCM LV, the inventors further investigated whether the network was specific to the ECM remodelling processes taking place in DCM and/or in LV tissue. They considered a separate heart condition, specifically repaired tetralogy of Fallot (rTOF) (Villafañe et al., 2013), which has a very different etiology from DCM but that is characterized by the presence of cardiac dysfunction and diffuse and pathologic myocardial fibrosis of both the right ventricle (RV) and LV (Pradegan et al., 2016).

RNA-seq data were analysed from RV in a cohort of adult patients with rTOF (n=27) who underwent surgery for pulmonary valve replacement and age-matched control donor samples (n=11). The hECM gene co-expression network identified in DCM LV was found to be significantly conserved in rTOF RV (empirical P-value <1×10⁻⁵) but not in control RV (empirical P-value=1). The inventors next determined that the pairwise correlation pattern of the hECM-network genes in rTOF RV mirrored the pattern observed in DCM LV (i.e., strongest gene-gene correlation in disease), and this was significantly different between rTOF patients and controls.

The consistent differential co-expression in dilated cardiomyopathy (DCM) and rTOF heart suggests that the hECM-network is capturing common ECM remodeling processes taking place across a range of human cardiac fibrotic diseases, irrespective of the specific disease etiology and the heart tissue (i.e. LV/RV).

In addition, using published longitudinal cardiac transcriptome data from a mouse genetic model of DCM that progresses to heart failure (HF; Burke et al., 2016), the inventors determined that this hECM-network is enriched for genes upregulated in DCM that progresses to HF. Specifically, 54% of the hECM-network genes are upregulated in DCM compared with control heart (enrichment P-value=5.4×10⁻⁵⁹) and 53% are upregulated in HF compared with control heart (enrichment P-value=2.4×10⁻⁴⁹). These results in human and mouse DCM/HF suggest that the identified hECM-network recapitulates the maladaptive fibrotic remodeling that promotes heart failure and adverse cardiovascular outcomes.

Since the hECM-network was enriched for genes involved in the TGFβ-signalling (as determined by KEGG annotation analysis), the inventors investigated whether the hECM-network was downstream of TGFβ-signalling activation. RNA-seq data were analysed from three sets of primary cultures of human atrial cardiac fibroblasts exposed to TGFβ1 for 24 h, TGFβ2 for 24 h, or control media.

A significantly large proportion of the genes belonging to the hECM-network were transcriptionally regulated after 24 h of stimulation with TGFβ1 or TGFβ2, which induced the differential expression of 46% and 49% of the genes in the hECM-network (315 and 335 genes), respectively.

Given the role of SMAD transcription factors downstream of TGFβ receptor activation (Hu et al., 2018; Massague and Wotton, 2000), the inventors also investigated whether the hECM-network was enriched for SMAD target genes. To this aim, published ChIP-seq data (Lachmann et al., 2010) were analysed, and the hECM-network was found to be significantly enriched for SMAD-regulated genes (291 genes, 43% of the network), especially for SMAD2, SMAD3 and SMAD4.

Together, these results revealed a cross-species conserved gene network, which might recapitulate the pathological ECM remodeling undergoing downstream of TGFβ/SMAD signalling activation in the diseased heart.

Injury in any organ triggers multicellular molecular responses that lead to tissue fibrosis. There is a wide range of complex diseases that affect different organs and are associated with fibrosis. This suggests that there may be shared pathways involved in this pathological process. The hECM-network identified in the heart could be capturing transcriptional responses that also act in other fibrotic diseases.

In order to investigate this, the inventors evaluated whether the hECM-network is conserved in other fibrotic tissues, by analysing additional transcriptomic data sets with/without fibrosis of right ventricle (rTOF), lung (pulmonary hypertension-associated lung fibrosis vs. controls, GEO database entry GDS4549) and liver (hepatitis C virus-infected subjects with liver fibrosis vs. controls, GEO database entries GSE33650 and GSE61260 respectively).

An empirical permutation test that assesses whether it is likely to find by chance a network of genes in an independent data set (how likely is it to find by chance a set of genes of the same size as the network with the same value of average absolute correlation in the data set under testing) was employed. In this test: i) expression of a random set of genes of the same size of each of the human LV DCM networks is sampled, ii) the mean correlation of both the network being tested and the randomly sampled network is computed, iii) after repeating steps 1 and 2 at least 10,000 times, an empirical p-value is computed for each network and adjusted for the number of networks present in LV (n=48). Significant results in this test imply that the hECM-network is specifically conserved in that tissue and condition.

Expression of the hECM-network was found to be conserved in heart right ventricle, lung and liver, only in fibrotic disease and not in control tissue (FDR <0.05 indicates that the hECM-network is conserved in that condition):

| | Heart (Right | | Lung | | | |
|---|---|---|---|---|---|---|
| | Ventricle) | | PH | | Liver | |
| | rTOF | Controls | fibrosis | Controls | Fibrosis | Controls |
| | (n = 27) | (n = 11) | (n = 62) | (n = 22) | (n = 72) | (n = 38) |
| hECM- | 0.0005 | 1.0000 | 0.0005 | 1.0000 | 0.0005 | 1.0000 |
| network | | | | | | |

The hECM-network was found to be conserved in fibrotic diseases such as repaired tetralogy of fallot (fibrosis of the right ventricle), liver and pulmonary hypertension-associated lung fibrosis.

Co-expression networks suggest coordinated genetic regulation, and this was exploited by the inventors to identify key genetic regulators of the hECM transcriptional program in the rat and DCM heart.

Multivariate Bayesian genetic mapping (Bottolo et al., 2011; Lewin et al., 2016) of the ECM-network was performed in the rat and then in humans. In this analysis, in order to identify loci regulating the whole ECM-network the expression of the rat (or human) genes in the ECM-networks were treated as multivariate quantitative traits, and it was investigated whether the joint expression levels of the network genes are associated with genome-wide genetic variants (i.e. single nucleotide polymorphisms, SNPs).

This Bayesian network-expression QTL (network-eQTL) mapping approach has previously been used to identify trans-acting genetic regulators of networks in several diseases, including type 1 diabetes (Heinig et al., 2010), epilepsy (Johnson et al., 2015) and inflammatory disease (Kang et al., 2014).

The rat ECM-network was mapped to the rat genome, and identified a single locus on rat chromosome 19 regulating 219 genes in the rat ECM-network (median Bayes Factor (BF)=181.7, where the Bayes Factor represents the strength of genetic regulation versus no genetic control).

### 2.2 Identification of WWP2 as a Regulator of the hECM-Network

It was investigated whether this regulatory locus for the rat ECM-network was replicated and conserved in human DCM heart. The cohort of DCM patients in which the hECM-network was strongly co-expressed, was analysed to understand whether the genes in the hECM-network were jointly mapping to the human locus syntenic to the regulatory locus found in the rat (the human syntenic locus is located on chromosome 16). Network-eQTL mapping in human DCM heart detected a single regulatory SNP (rs9936589) located within an intron of the WWP2 gene, an E3 ubiquitin ligase. This SNP was strongly associated with the expression of the hECM-network in the DCM heart (median BF=2004 for the 683 genes in the hECM-network). Moreover, the network-eQTL was not detectable in the heart from control organ donors, suggesting that the genetic regulation of the hECM-network is present only in diseased heart. This regulatory SNP (rs9936589) and the WWP2 gene have not previously been associated with fibrotic disease or heart disease by GWAS or other genetic and eQTL mapping approaches.

Systems genetics analyses revealed a coordinated pro-fibrotic and disease-associated ECM transcriptional program in the diseased heart, whose expression was associated with a genetic variant within the WWP2 locus. The inventors therefore, investigated whether WWP2 was a potential transcriptional regulator of the hECM-network in human fibrotic heart disease.

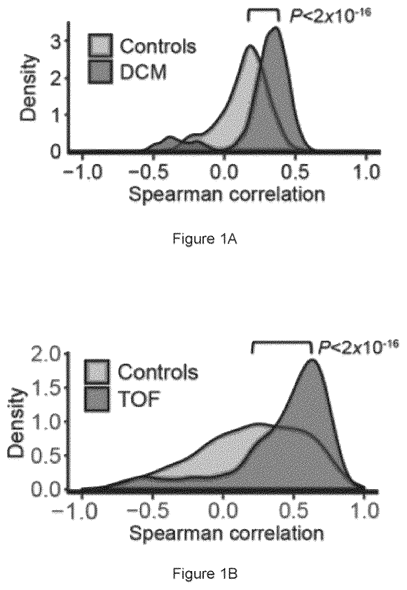

Expression levels of WWP2 were correlated to the expression levels of the 683 genes forming the hECM-network in LV and RV fibrotic disease hearts (i.e., in DCM and rTOF) and controls separately. In both LV (DCM) and RV (rTOF) fibrotic heart, a positive and significant shift in the distribution of the correlations between WWP2 expression and the expression of the hECM-network genes was observed (FIGS. 1A and 1B), suggesting a positive association between WWP2 expression and the hECM-network genes in disease. However, WWP2 expression was only moderately increased in the diseased heart: human rTOF, fold change (FC)=1.23 (False Discovery Rate, FDR=0.03); human DCM FC=1.02 (FDR=0.09) (Heinig et al., 2017) and mouse HF vs WT, FC=1.86 (P-value=9.66×10⁻¹³) (Burke et al., 2016).

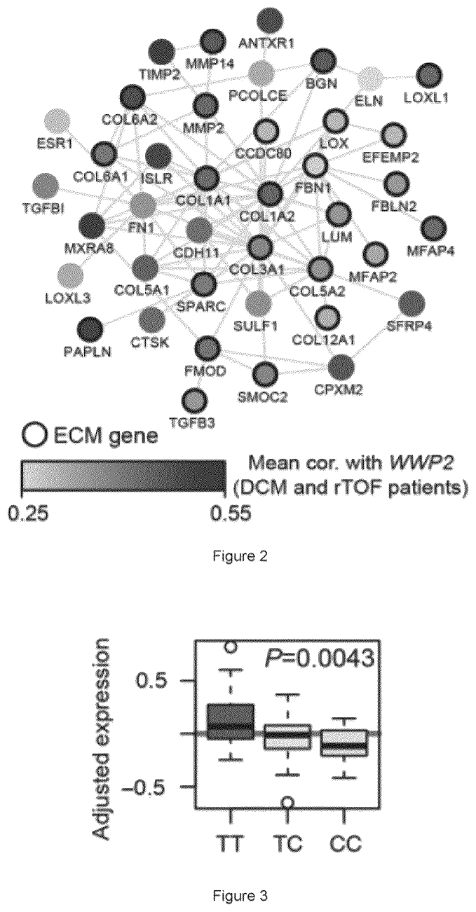

A core set of genes in the hECM-network was also identified that were positively and consistently correlated with WWP2 expression in the heart (FDR<1%) irrespective of heart tissue of origin (i.e., LV or RV) or heart condition (i.e., rTOF or DCM), FIG. 2). This core gene set includes genes encoding known regulators of the pathological ECM remodelling, such as matrix metalloproteinases (e.g., MMP2, MMP14, (Matsumura et al., 2005)) and their tissue inhibitors (e.g., TIMP2, (Peterson et al., 2000; Arpino et al., 2015)), several collagens and their binding partners (e.g., TGFβ1, (Schwanekamp et al., 2017)), microfibrillar-associated proteins and profibrotic cytokines (e.g., TGFβ3, (Burke et al., 2016)). Specifically, the top-correlated genes with WWP2 expression in the DCM heart included LTBP3 (member of the TGFβ-signaling pathway) and PTPRA, which has been shown to promote pro-fibrotic signalling in lung fibroblasts (Aschner et al., 2014). Examples of genes with highest correlation to WWP2 expression in the rTOF patients include BGN, which has recently been suggested as a serum marker of liver fibrosis (Ciftciler et al., 2017), and numerous collagens (COL1A1, COL5A1, COL1A2, COL8A2, COL6A2 and COL14A1).

These findings suggest that increased expression of WWP2 is associated with elevated expression of pro-fibrotic genes in the diseased heart. Analysis of the expression levels of hECM-network genes stratified by the genotypes of the regulatory SNP rs9936589, showed increased expression of the hECM-network associated with the TT genotype (FIG. 3).

To further analyse whether the regulatory effect of rs9936589 on the hECM-network was mediated by WWP2, the inventors analysed whether cardiac WWP2 expression was similarly regulated by this SNP (i.e., the inventors investigated whether expression of WWP2 was regulated in cis).

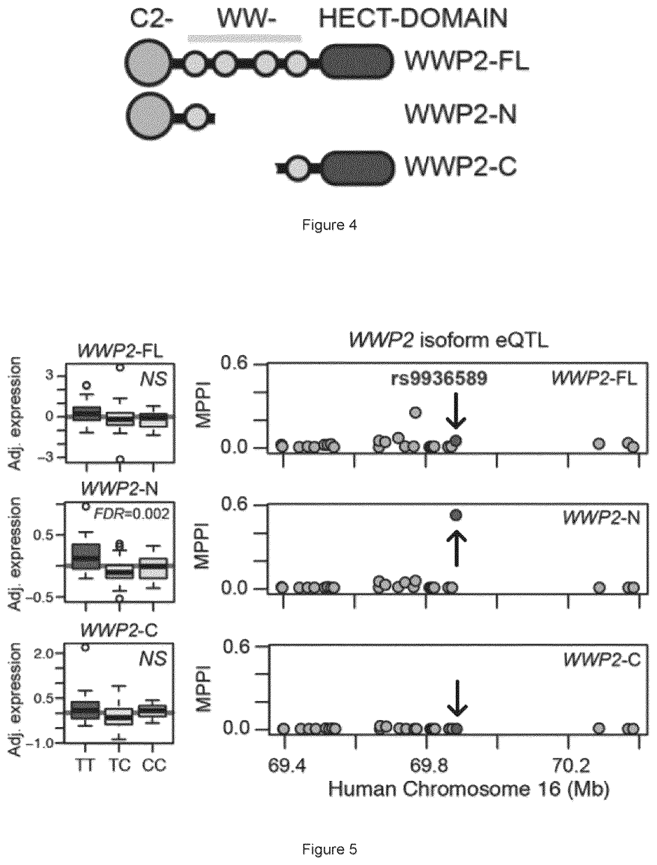

Three main WWP2 gene isoforms have been characterised, containing different protein domains: a full-length isoform (WWP2-FL, covering the entire gene), an N-terminal isoform (WWP2-N, corresponding to the 3′ end of the protein) and a C-terminal isoform (WWP2-C, containing the 5′ end of the protein) (Soond and Chantry, 2011)—see FIG. 4.

All these three isoforms showed robust expression in the hearts of DCM patients. WWP2 isoform-specific cis-QTL mapping was performed, and showed that only the WWP2-N was regulated by the SNP rs9936589 (FIG. 5). Increased cardiac expression of WWP2-N was associated with the TT genotype of SNP rs9936589, indicating genotype-dependent WWP2-N expression levels in DCM heart. Consistent with the hECM-gene network regulation by SNP rs9936589, the TT genotype yielded was associated with expression of WWP2-N, suggesting that this WWP2-N isoform, containing the N-terminal C2 and WW1 domains of WWP2, could be a positive regulator of the hECM-network in the diseased heart.

### Example 3: WWP2 Regulates Cardiac Fibrosis In Vivo

### 3.1 Generation and Characterisation of Mice Lacking Expression of WWP2-FL and WWP2-N

In order to identify pathophysiological processes regulated by the N-terminal region of WWP2, the inventors generated Wwp2 mutant mice (Wwp2^{Mut/Mut}) using CRISPR/Cas9 technology to introduce a 4-bp deletion (CTAC) in exon 2 of Wwp2, leading to disruption of Wwp2-FL and Wwp2-N isoforms. Consistent to the phenotype that was previously reported in global Wwp2-null mice (Zou et al., 2011), the Wwp2^{Mut/Mut} mice showed reduced body weight relative to wildtype (P<0.001 at 10 weeks of age, repeated-measures t-test n=8 mice per group), abnormal craniofacial development and elongated teeth.

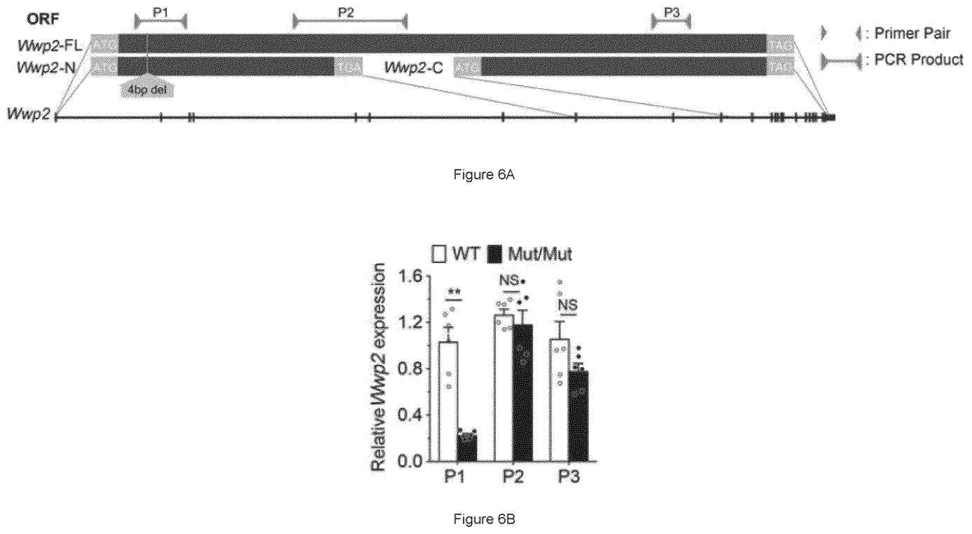

The 4-bp deletion in Wwp2 resulted in ablation of WWP2 isoforms containing the Wwp2 N-terminal region (that is, Wwp2-FL and Wwp2-N isoforms), as detected at the mRNA level by isoform-specific primer pair 1 (P1; FIGS. 6A and 6B).

Western blot analysis confirmed lack of Wwp2-FL expression at the protein level of protein extracts prepared from cardiac fibroblasts obtained from cardiac tissue of Wwp2^{Mut/Mut} mice (FIG. 6C), using anti-Wwp2 antibodies targeting the N-terminal region of the protein (Santa Cruz biotechnology, #sc30052, or antibody obtained from Bethyl Laboratories). However, the cardiac expression of WWP2-C protein was not affected.

### 3.2 Analysis of the Effects of Loss of Wwp2-N/FL Isoform Expression in Mouse Models of Fibrotic Disease

### Ang II Infusion Model

Systems genetics analysis indicated that the WWP2-N isoform positively regulates a pro-fibrotic transcriptional network in diseased heart (Example 2.2), and so the inventors hypothesised that loss of function (LOF) of WWP2-N/FL protein isoforms might inhibit the in vivo fibrogenic response.

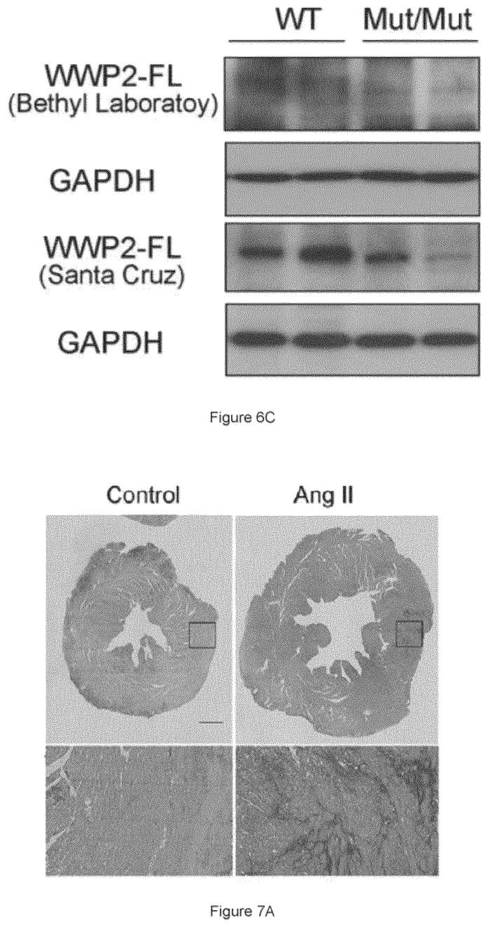

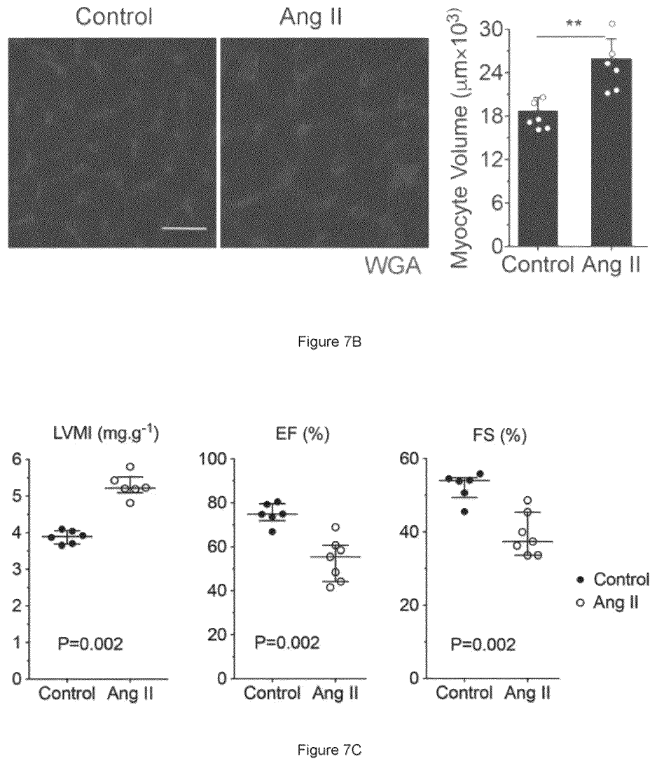

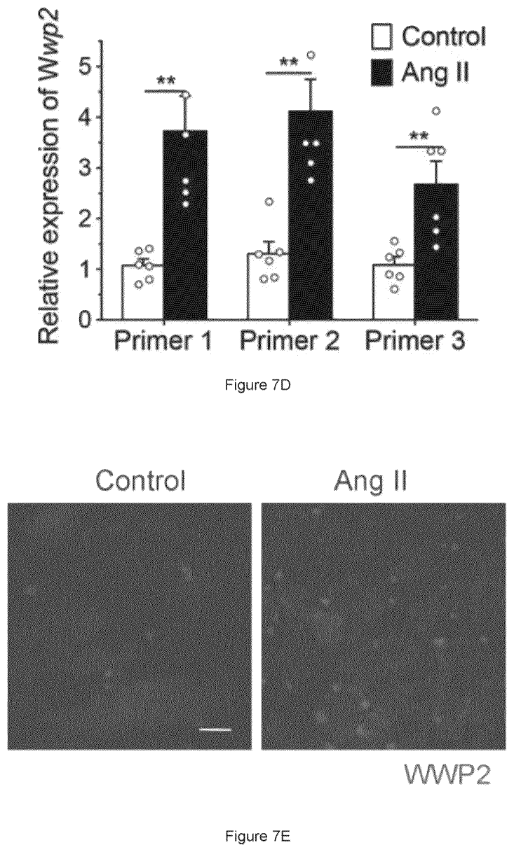

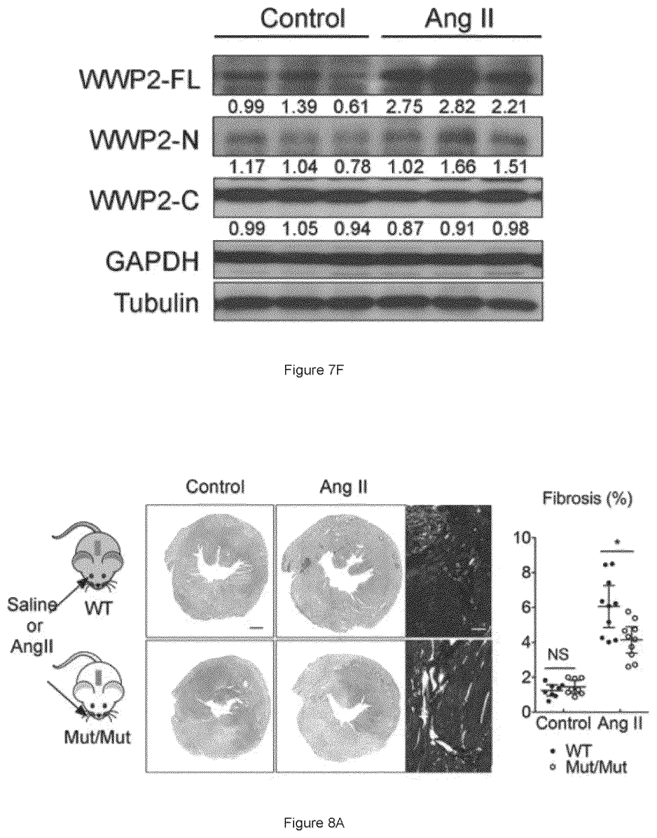

Sections from the hearts of WT mice infused with Ang II had increased collagen content, as determined by increased Sirius Red staining relative to controls infused with saline (FIG. 7A). Sections from Ang II-infused hearts also contained more hypertrophic myocytes, with increased mean cell volume relative to saline-infused controls (FIG. 7B).

Ang II infusion was also associated with ventricular remodelling and impaired cardiac function. Increased left ventricular mass index (LVMI), and decreased ejection fraction, and fractional shortening was observed in WT mice subjected to Ang II infusion relative to saline-infused controls (FIG. 7C).

RT-qPCR analysis also detected increased Wwp2 transcript levels (FIG. 7D) and protein levels (FIGS. 7E and 7F) in LV tissue in WT mice subjected to Ang II infusion relative to saline-infused controls.

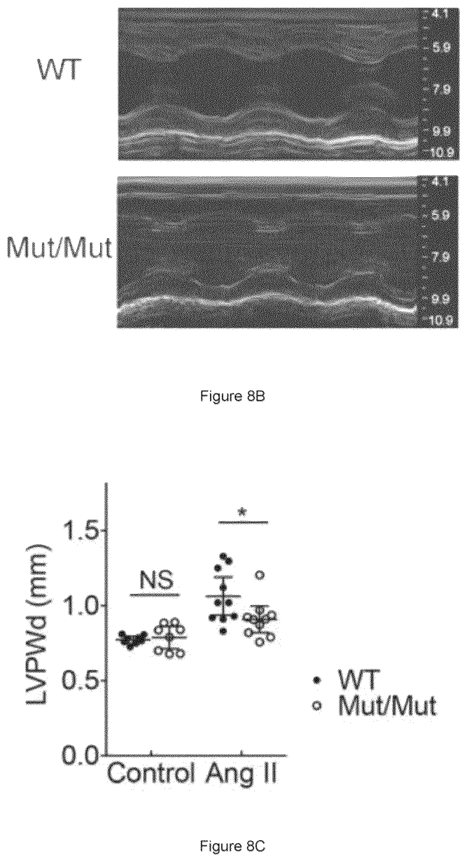

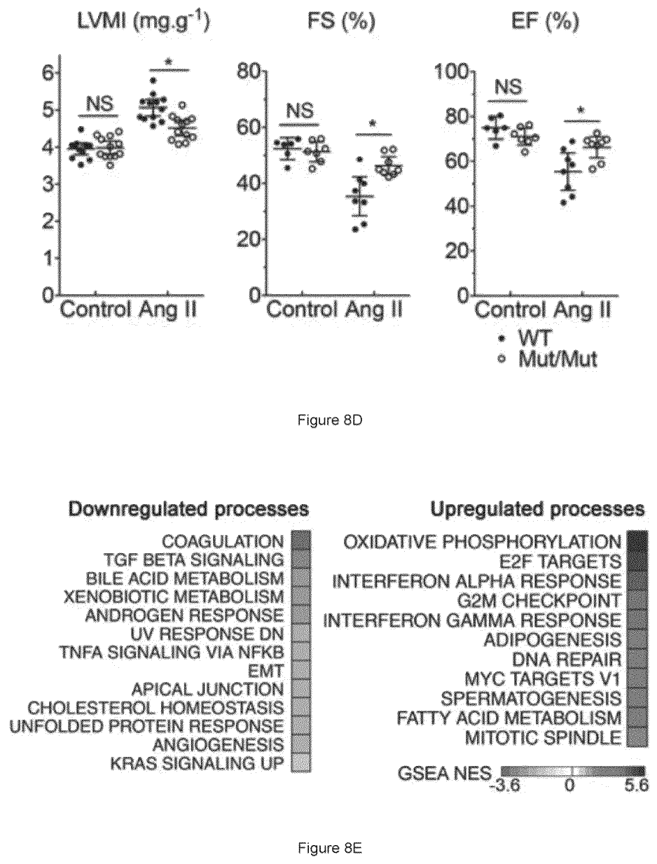

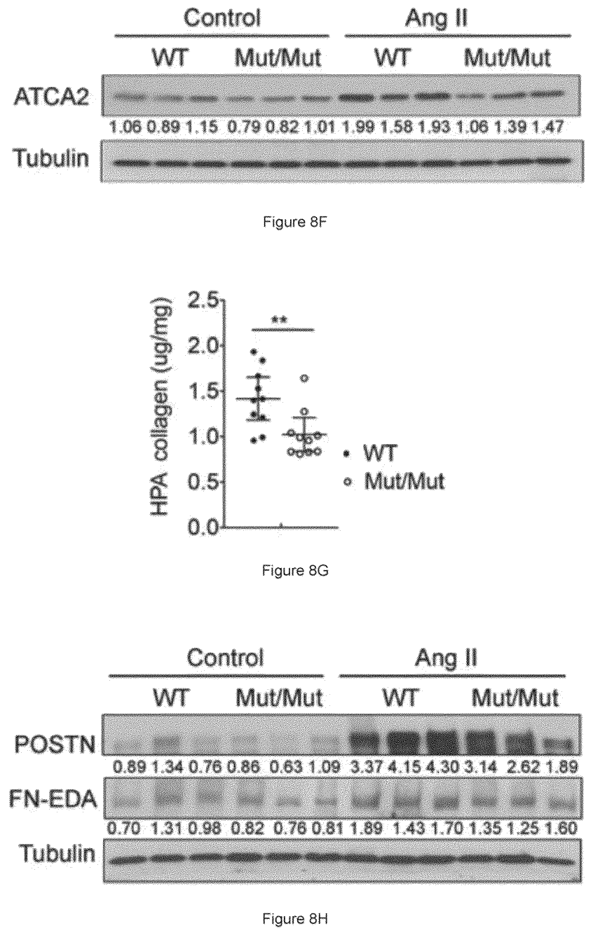

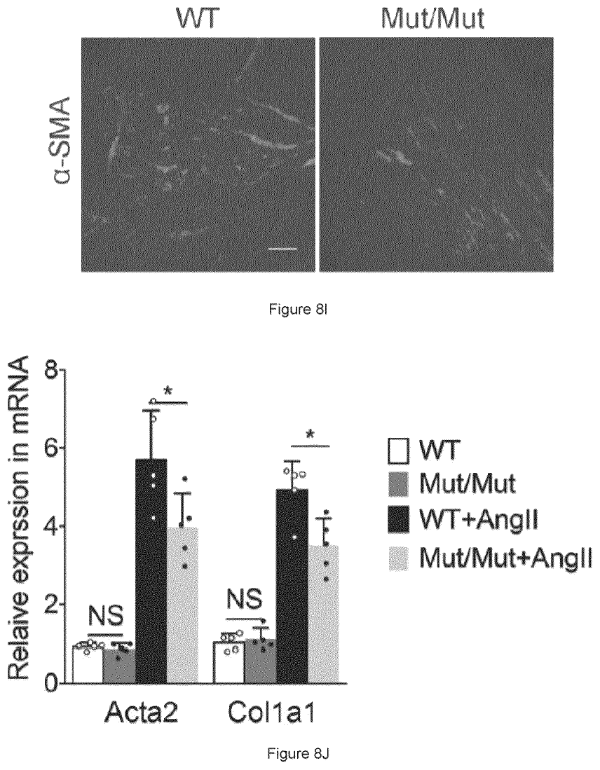

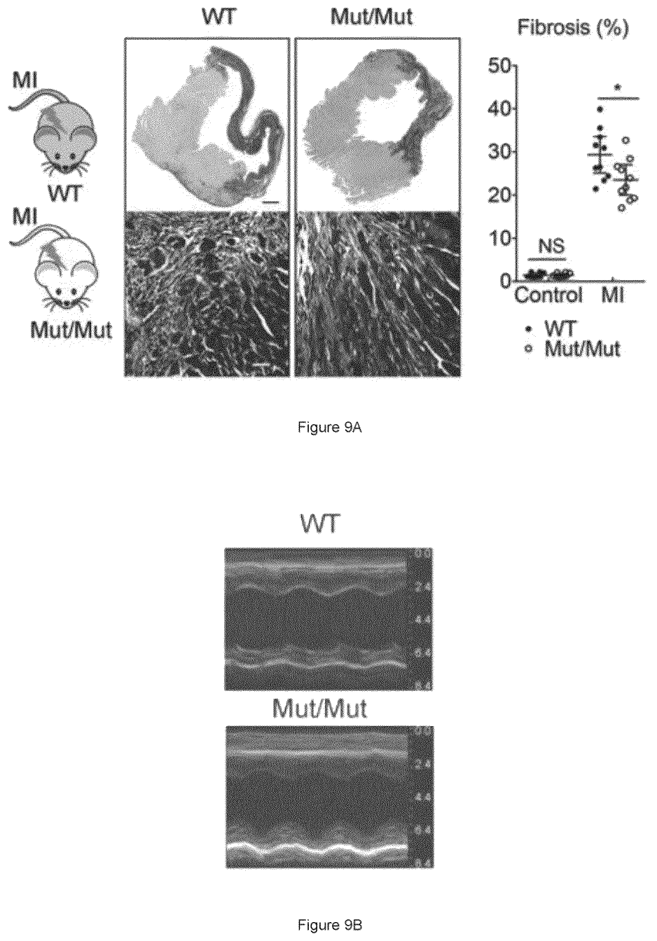

In contrast to WT mice, Ang II-infused Wwp2^{Mut/Mut} mice displayed significantly less fibrosis. LV sections from Ang II-infused Wwp2^{Mut/Mut} mice showed reduced Sirius red and Masson's Trichrome staining relative to Ang II-infused WT mice (FIG. 8A). The Ang II-infused Wwp2^{Mut/Mut} mice also displayed reduced cardiac hypertrophy and less inhibition of cardiac function as compared to Ang II-infused WT mice (FIGS. 8B, 8C and 8D).

Bulk RNA-seq analysis in Ang II-treated WT (n=8) and Wwp2^{Mut/Mut} mice (n=8) revealed that the mouse orthologs of the hECM-network genes detected in DCM heart, showed a different co-expression pattern with increased gene-gene correlation in the LV of WT compared with Wwp2^{Mut/Mut} mice (P=0.003). In Wwp2^{Mut/Mut} mice, this pattern of differential co-expression was remarkably similar to the pattern observed between human fibrotic disease (DCM or rTOF) and controls; the Wwp2^{Mut/Mut} mice showed a hECM-network co-expression pattern similar to control heart. Consistent with human data, the hECM-network was also significantly enriched for differentially expressed genes between Ang II-treated WT and Wwp2^{Mut/Mut} mice. In addition, “TGFβ signaling” and “extracellular matrix” were two of the major downregulated pathways in Wwp2^{Mut/Mut} mice following Ang II-infusion, (Normalized Enrichment Score (NES) of −2.71 and −2.8; see FIG. 8E).

Protein-based assays showed that Wwp2^{Mut/Mut} mice had reduced levels of fibroblast activation and ECM protein markers as marked by α-smooth muscle actin (ACTA2; FIGS. 8F, 8I and 8J), collagen1 (COL1A1; FIG. 8J), fibronectin extracellular domain A (FN-EDA; FIG. 8H) and periostin (POSTN; FIG. 8H) in the heart after Ang II-infusion, as compared to WT mice.

### Myocardial Infarction Model

The inventors next investigated whether WWP2-N/FL LOF had a protective effect on cardiac fibrosis and function post myocardial infarction (MI).

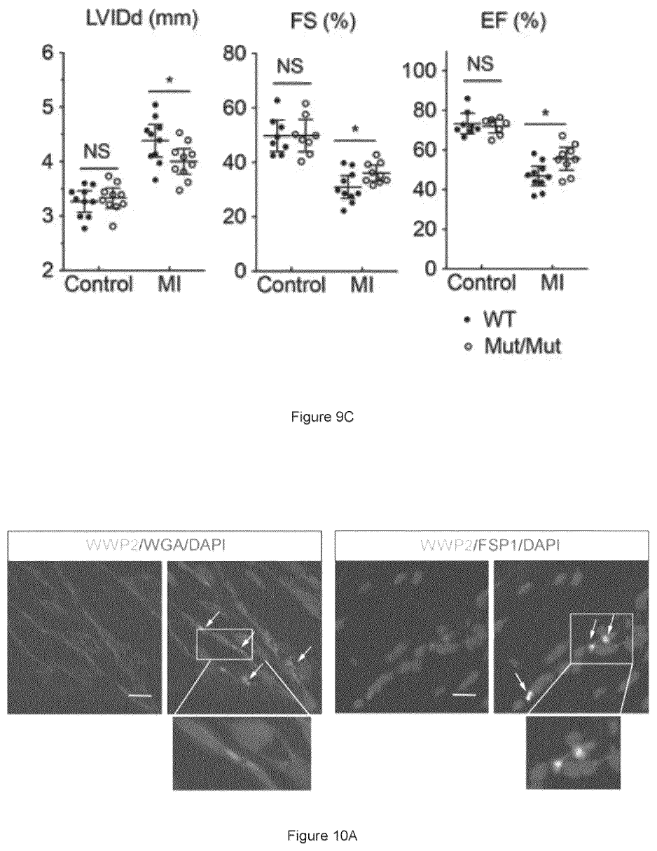

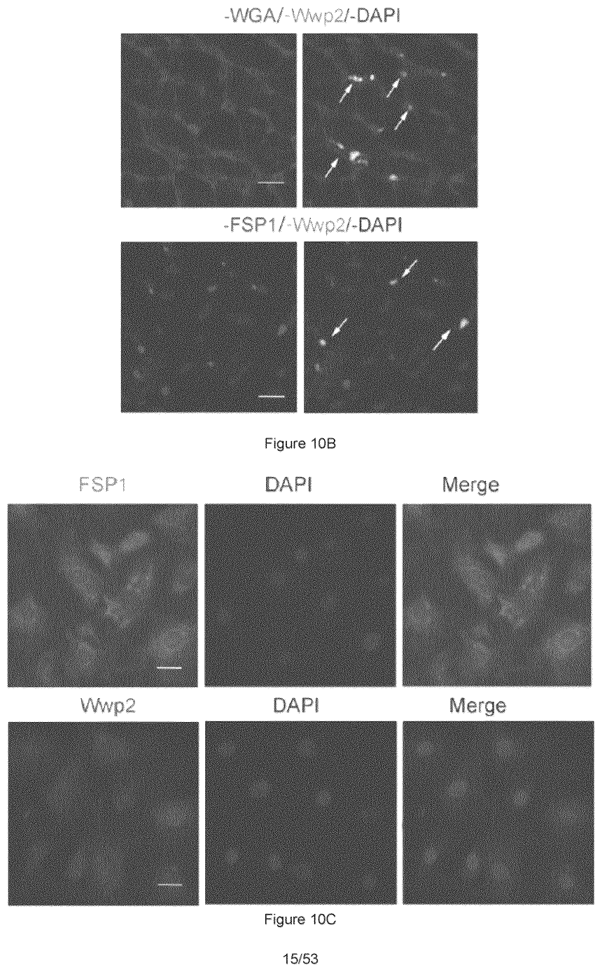

Histological analysis revealed less post-MI fibrotic remodelling in the LV of Wwp2^{Mut/Mut} mice as compared to WT mice (FIG. 9A). This was associated with reduced chamber dilation and greater preservation of contractile function in Wwp2^{Mut/Mut} mice post-MI as compared to WT mice (FIGS. 9B and 9C).

Taken together, the results in the Ang II infusion and MI models suggest that LOF of the WWP2 isoforms containing N-terminal region (i.e. WWP2-FL and WWP2-N) reduces cardiac fibrosis and improves cardiac function following Ang II-treatment or MI. This is in keeping with a role for WWP2 as a positive regulator of fibrosis in the diseased heart.

### Example 4: WWP2 Regulates the TGFβ1-Induced Fibrotic Response in Primary Cardiac Fibroblasts

### 4.1 Analysis of WWP2 Expression in Cardiac Cells

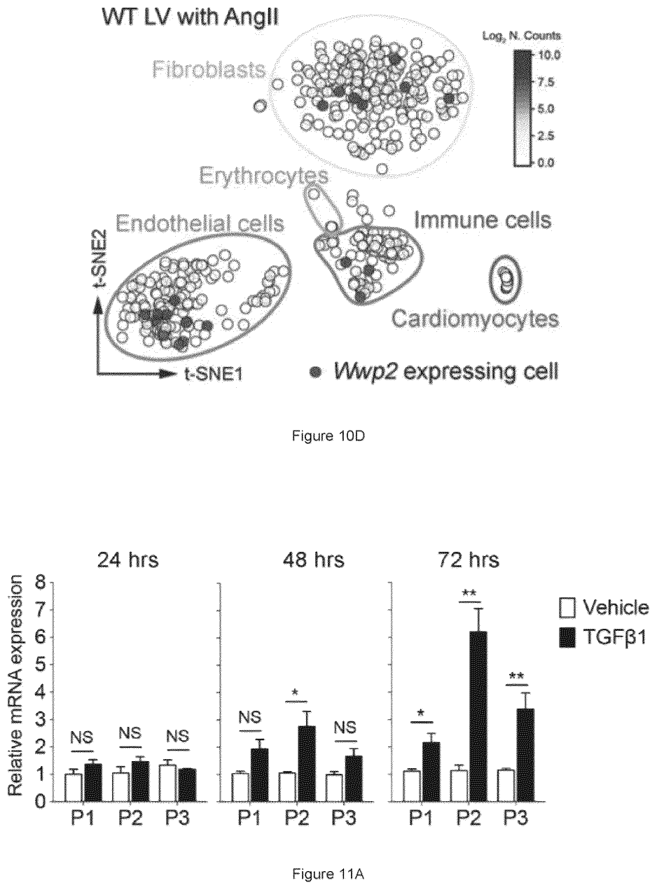

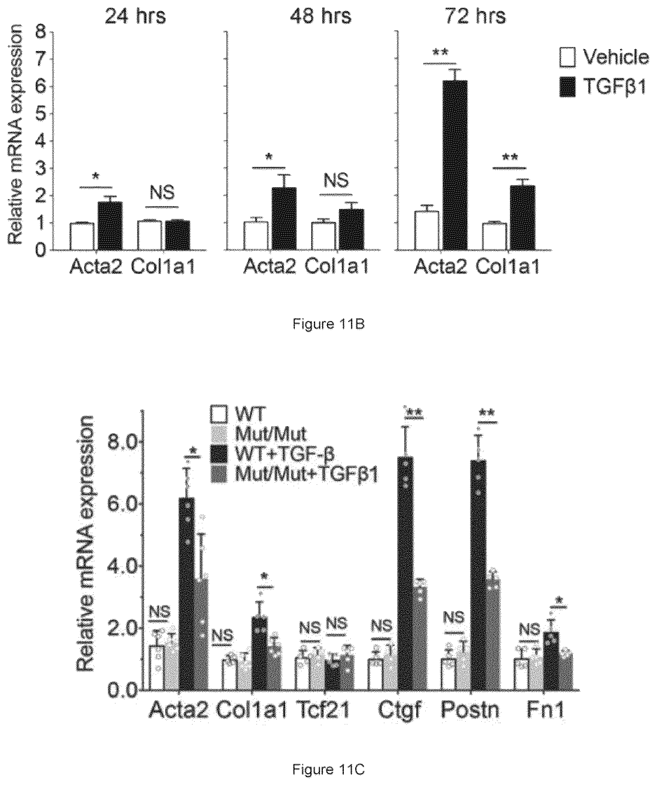

To investigate the regulation of WWP2 in the cardiac cells, the inventors imaged WWP2-expressing cells in heart sections by immunofluorescence. WWP2-positive cells did not show the morphology typical of a sarcomere-containing cardiomyocyte, and some WWP2-positive cells expressed fibroblast-specific protein 1 (FSP1) (FIGS. 10A and 10B). Cultured fibroblasts isolated from the LV of WT mice showed both expression of WWP2 and FSP1 (FIG. 10C). The WWP2 expression data from immunofluorescence studies was further corroborated by results of single-cell RNA-seq analysis in the WT heart following Ang II-infusion, that provide evidence of WWP2 expression in fibroblasts, but also in endothelial cells and immune cell populations (FIG. 10D). In agreement with the immunofluorescence data, WWP2 was not found to be expressed by cardiomyocytes.

### 4.2 Analysis of the Effects of TGFβ1 Stimulation

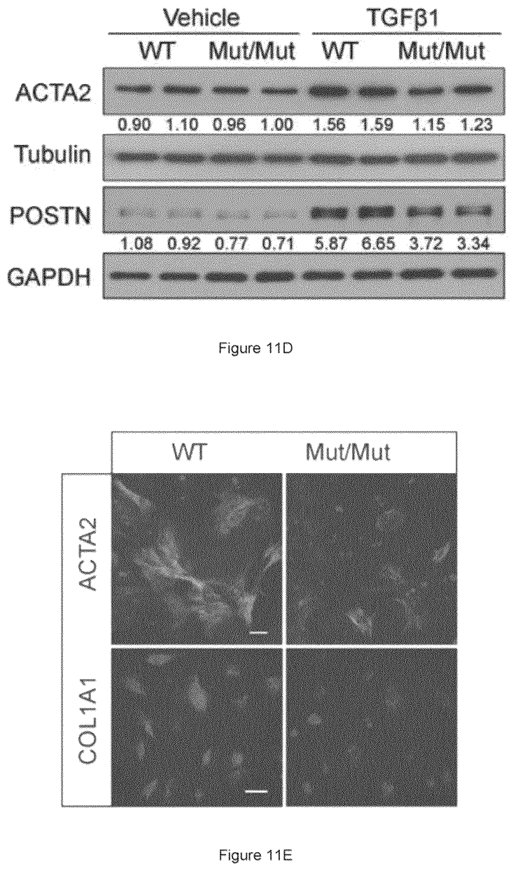

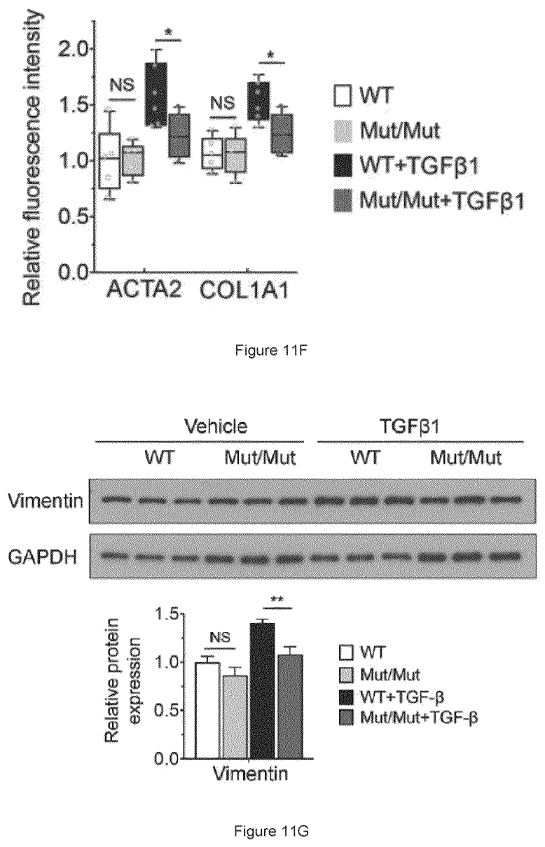

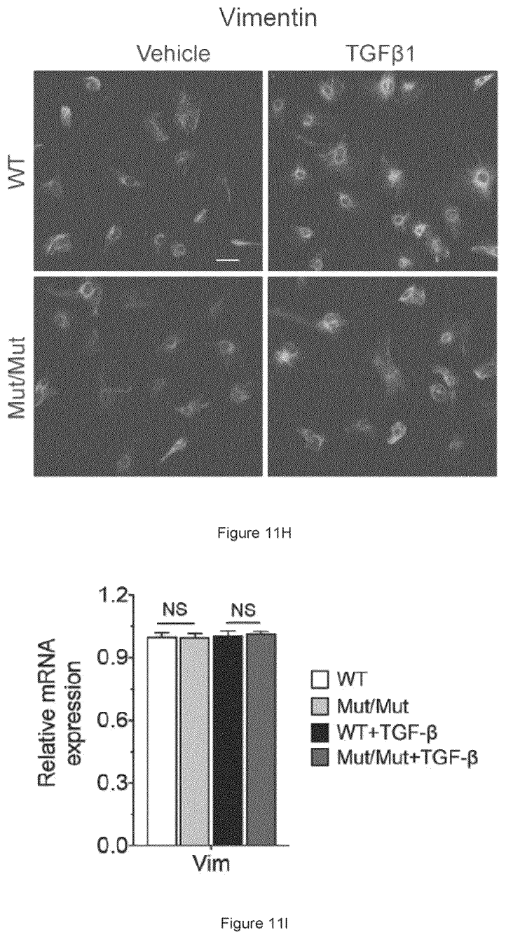

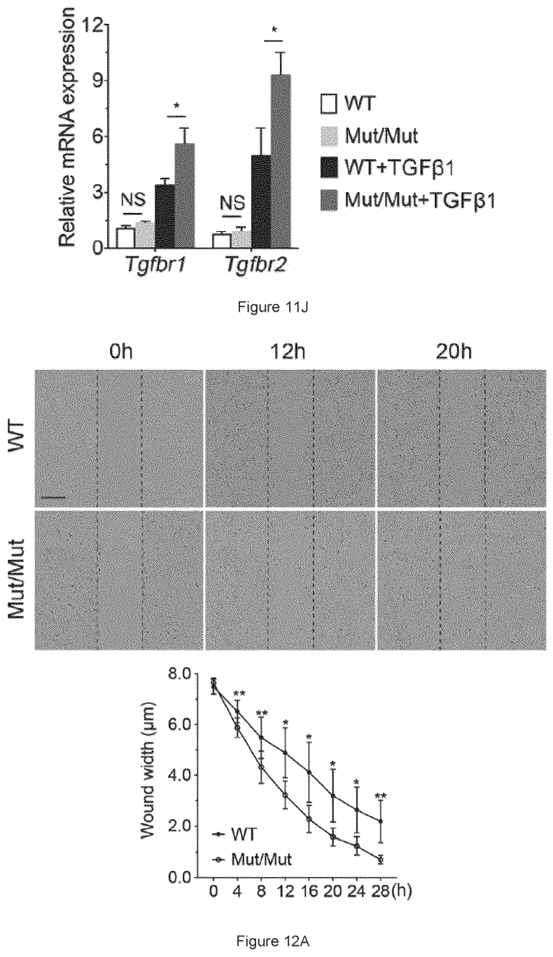

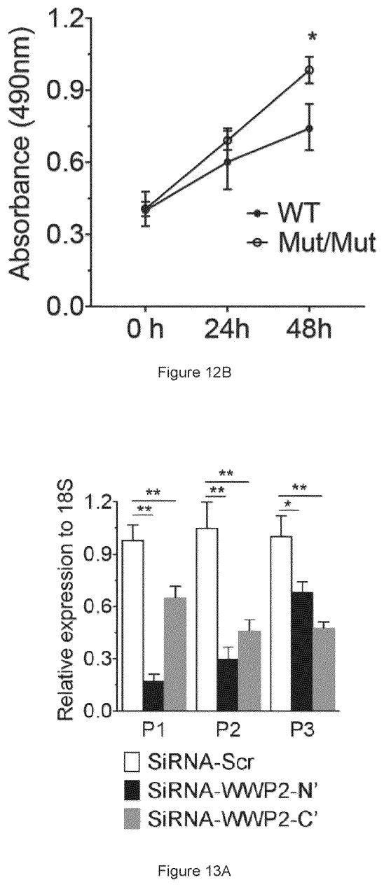

TGFβ1 stimulation of primary murine LV fibroblasts induced robust Wwp2 transcription at 72 hrs of treatment (FIGS. 11A and 11B). The inventors then investigated the impact of WWP2 in the response to prolonged (72 hrs) TGFβ1 treatment in primary murine LV fibroblasts, and found that TGFβ1 stimulation increased fibroblast activity and ECM production (as measured by ACTA2, COL1A1 and POSTN expression) in WT fibroblasts, but the pro-fibrotic TGFβ1-induced expressional changes at both the mRNA and protein levels were largely prevented in Wwp2^{Mut/Mut} fibroblasts (FIGS. 11C and 11D).

TGFβ1-stimulated WT fibroblasts presented a clear organization of ACTA2 into stress fibers, while Wwp2^{Mut/Mut}-derived cells displayed diffuse expression of ACTA2 with rare incorporation into stress fibers (FIGS. 11E and 11F). TGFβ1 also mildly increased vimentin protein expression, which was reduced in Wwp2^{Mut/Mut} cells (FIGS. 11G and 11H). However, no difference in the mRNA level of vimentin and Transcription Factor 21 (Tcf21) expression were observed (FIGS. 11C and 11I). TGFβ1 treatment was also found to induce TGFβ receptor expression (Tgfbr1 and Tgfbr2) in cardiac fibroblasts from Wwp2^{Mut/Mut} mice (FIG. 11J), suggesting a potential compensatory effect of WWP2 on TGFβ-signalling activation.

### 4.3 Analysis of the Functional Cellular Consequences of WWP2 Deficiency

Interestingly, WWP2 heterodimerizes with WWP1 (Chaudhary and Maddika, 2014), another HECT-type E3 ligase, which has been previously reported to induce ubiquitin-dependent degradation of TGFBR1 (Komuro et al., 2004). In addition, Wwp2^{Mut/Mut} cardiac fibroblasts showed higher cell proliferation and migration compared to controls (FIGS. 12A and 12B), which is consistent with reduced conversion of fibroblast to myofibroblasts (Schmidt et al., 2015).

Isoform-eQTL analysis of WWP2 in the heart of DCM patients suggested a regulatory role for the WWP2 isoforms containing N-terminal region in fibrosis, and in agreement with this, murine cardiac fibroblasts responded to TGFβ1 treatment with increased protein levels of the WWP2-FL/N isoforms, but not of WWP2-C. Increased expression of WWP2-FL/N isoforms was consistent with the findings in the heart following Ang II-infusion in vivo. Wwp2^{Mut/Mut} mice lack WWP2-FL and WWP2-N protein isoforms, and this is sufficient to alter the co-regulation of the hECM network genes and reduce cardiac fibrosis in vivo and inhibit conversion of fibroblasts to myofibroblasts in vitro. These data combined suggest a primary role for WWP2 gene isoforms containing N-terminal region of the protein (i.e., WWP2-FL and WWP2-N) in regulating the molecular program associated with cardiac fibrosis.

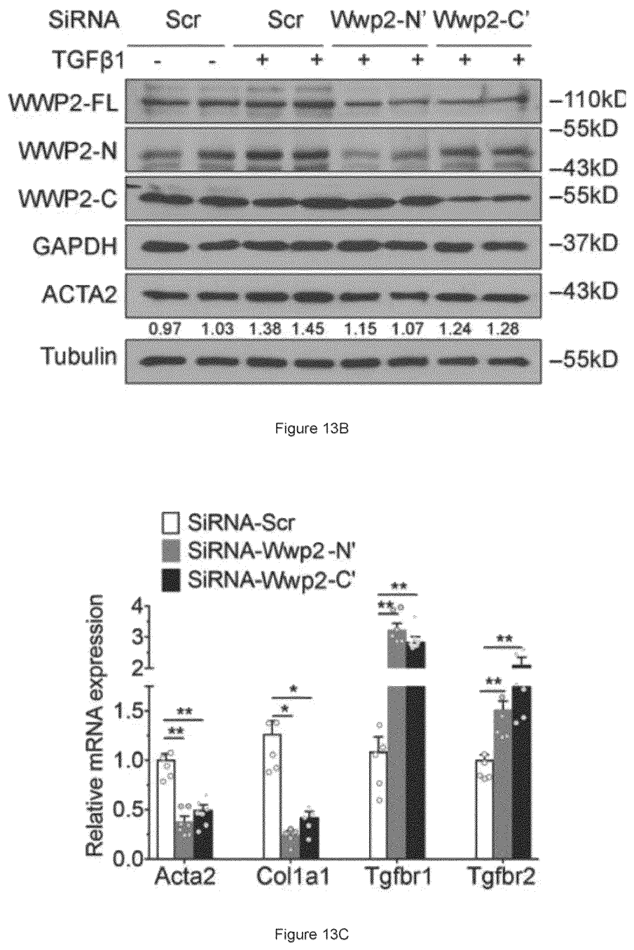

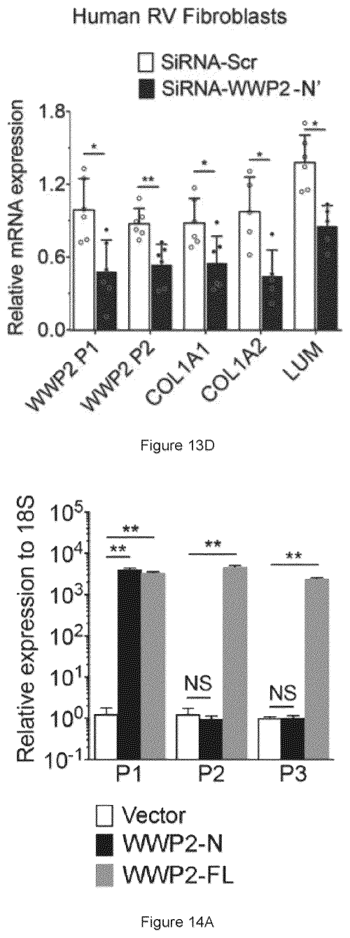

To confirm the differential effects of the WWP2 N-terminal region on cardiac fibroblast activity, siRNA sequences were designed, targeting either the 5′-terminal (siRNA-Wwp2-N′) or 3′-terminal (siRNA-Wwp2-C′) regions of the Wwp2 mRNA. siRNA transfection in human cardiac fibroblast successfully decreased the expression of WWP2-FL/N and WWP2-FL/C isoforms in cardiac fibroblasts, respectively (FIG. 13A). Compared with the scrambled control, both siRNA abrogated the expression of ACTA2 in WT primary cardiac fibroblasts treated with TGFβ1 (FIGS. 13B and 13C). In agreement with the data obtained in mouse Wwp2^{Mut/Mut} cardiac fibroblasts, after WWP2 knockdown, the fibroblasts showed increased mRNA expression of Tgfbr1 and Tgfbr2 following TGFβ1 treatment (FIG. 13C). Additional siRNA targeting 5′-terminal of WWP2 mRNA (siRNA-WWP-N′) experiments in primary human cardiac fibroblasts, confirmed that WWP2-N/FL knockdown reduces expression of pro-fibrotic genes (FIG. 13D).

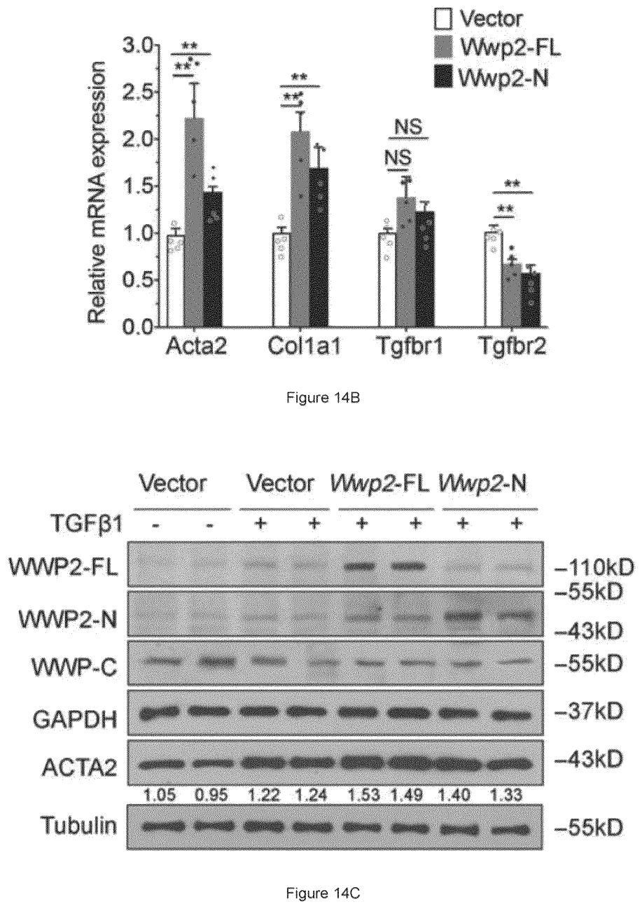

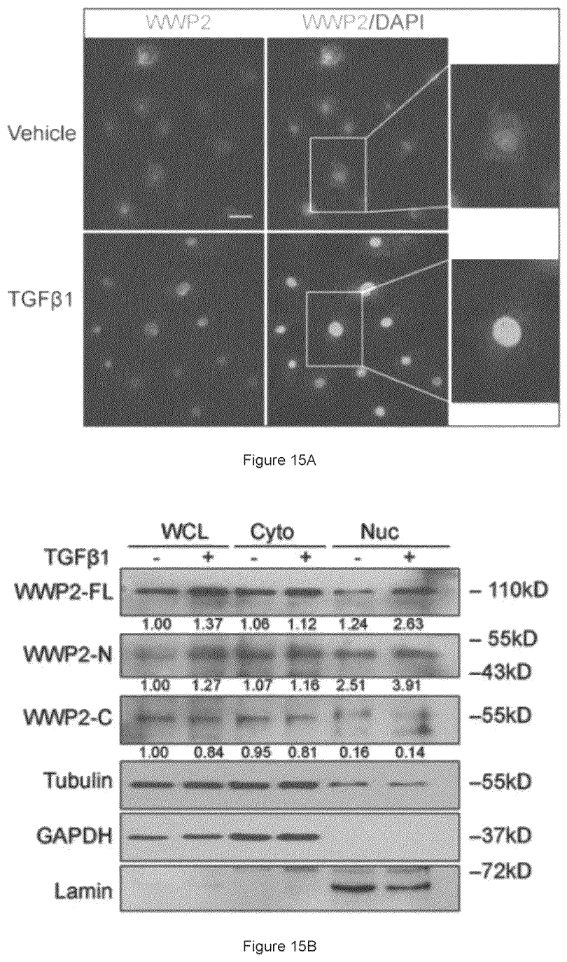

The inventors then carried out a rescue experiment by re-introducing the two isoforms containing WWP2 N-terminal region (WWP2-FL and WWP2-N) separately in primary cardiac fibroblasts from Wwp2^{Mut/Mut} mice (FIG. 14). Both Wwp2-FL and Wwp2-N individually increased the expression of pro-fibrotic genes in the Wwp2^{Mut/Mut} fibroblast cells treated with TGFβ1 (FIGS. 14B and 14C), suggesting that these two WWP2 isoforms are positive regulators of fibrogenic processes.

### Example 5: WWP2 Regulates the Nucleocytoplasmic Shuttling of the TGFβ-Signaling Transducer SMAD2

### Subcellular Localisation of WWP2 Isoforms

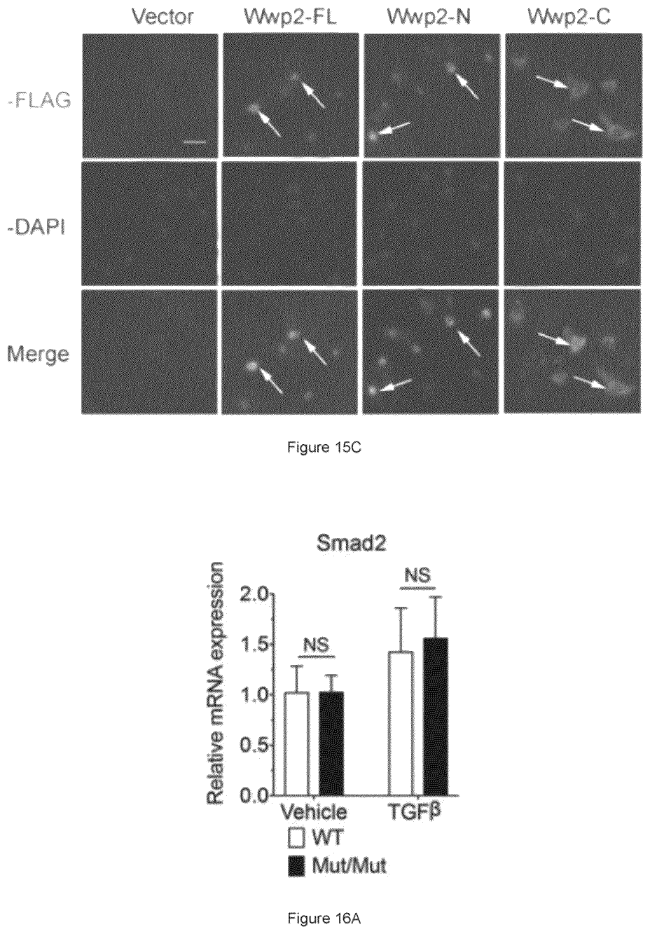

The inventors further investigated the cellular mechanisms through which WWP2 regulates the fibrogenic response downstream of TGFβ signaling activation. WWP2 was found to weakly localize in both the cytoplasm and nuclei in quiescent cardiac fibroblasts; however, upon TGFβ1 simulation (16 hrs), increased WWP2 expression localized predominantly in the nucleus was observed (FIG. 15A). Specifically, increased levels of WWP2-FL and WWP2-N protein isoforms were detected in the nucleus after TGFβ1 stimulation, while the WWP2-C isoform remained in the cytoplasm (FIG. 15B). This was further confirmed by transfecting three FLAG-tagged isoforms into cells; after TGFβ1 stimulation, WWP2-FL and WWP2-N isoforms were restricted to the nucleus, while WWP2-C presented cytoplasmic localization (FIG. 15C).

The presence of WWP2 isoforms in the nucleus makes it plausible that WWP2 may be involved in regulating gene expression, possibly targeting transcription factors for ubiquitination as shown for other E3 ubiquitin ligases (Horwitz et al., 2007; Gao et al., 2016). Examples 3 and 4 herein provide evidence for WWP2 involvement in the downstream regulation of TGFβ-signalling in vivo and in vitro.

WWP2 Isoform Interaction with SMAD Proteins

It has been demonstrated that the pro-fibrotic transcriptional program (i.e., the hECM-network) positively regulated by WWP2 is enriched for transcriptional targets of the TGFβ-signalling transducer SMAD transcription factors. Genes downregulated by WWP2 in vivo in the heart following Ang II-infusion were significantly enriched for “SMAD binding” (FDR=0.003), “SMAD protein signal” (FDR=0.006) and “transcriptional activity of SMAD2/3/4 heterotrimer” (FDR=0.015) by GSEA.

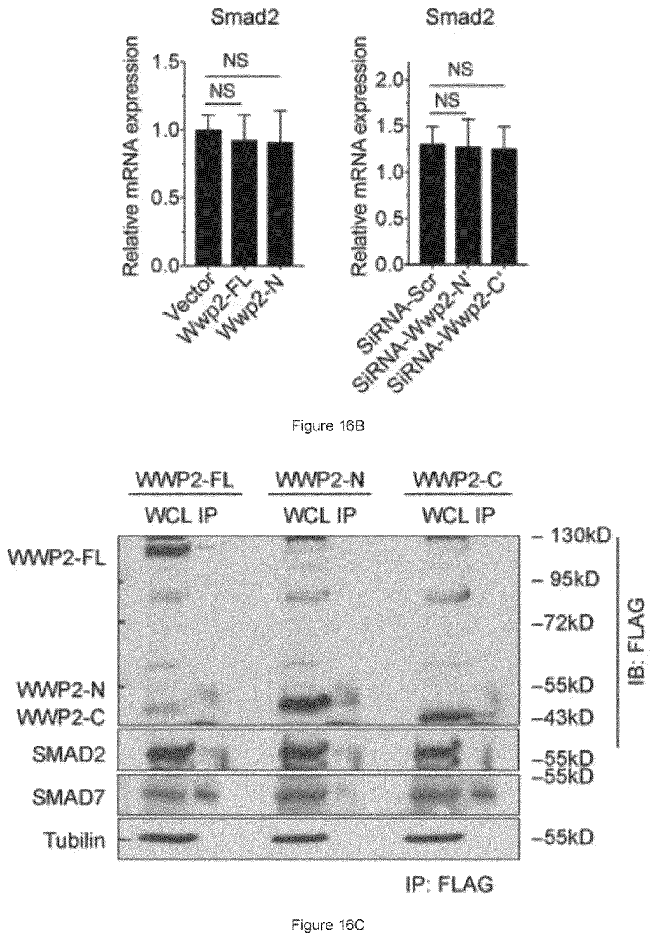

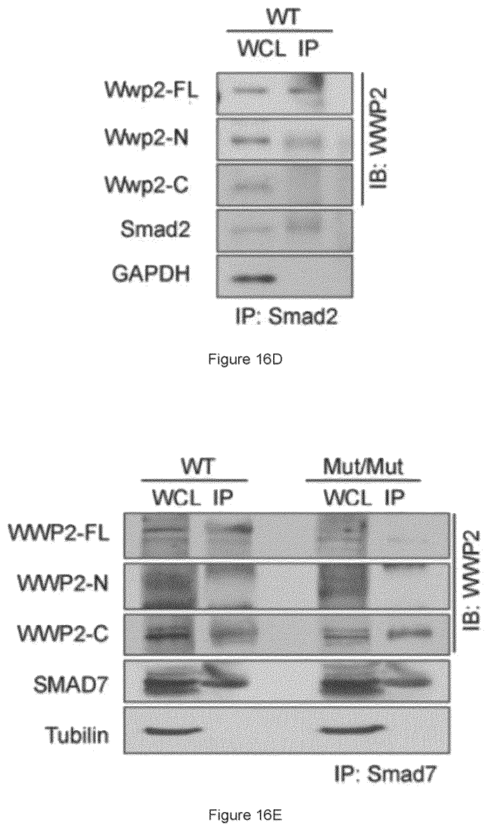

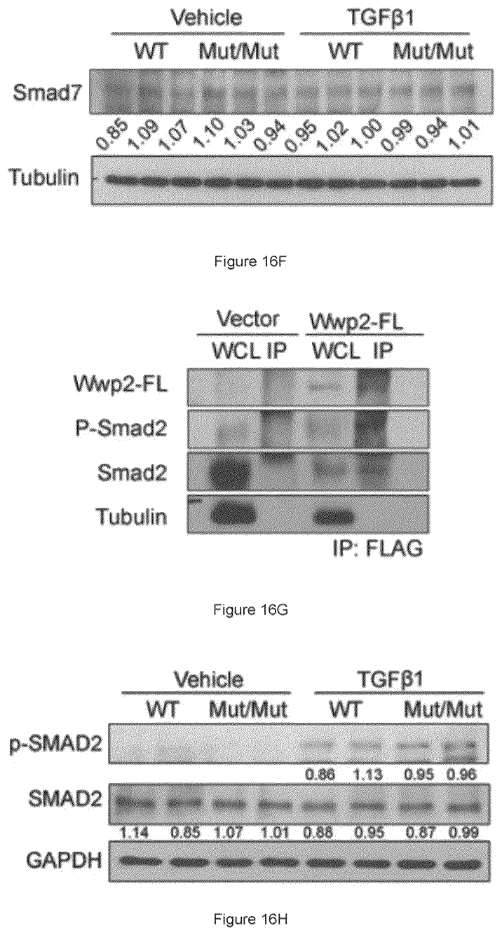

No difference was observed at the mRNA level of SMAD2 transcription factor in Wwp2^{Mut/Mut} cardiac fibroblasts (FIG. 16A) or in cells overexpressing or silencing the WWP2-N/FL isoforms (FIG. 16B), suggesting that WWP2 function on cardiac fibrosis could be exerted through SMAD2 interaction and ubiquitination at the protein level.

FLAG-tagged WWP2-FL and WWP2-N but not WWP2-C expressed in NIH-3T3 mouse embryonic fibroblast cells co-immunoprecipitated with SMAD2 protein (FIG. 16C). Furthermore, mouse WT primary cardiac fibroblasts responded to TGFβ1 stimulation with SMAD2 binding to WWP2-FL and WWP2-N, but not to WWP2-C (FIG. 16D). Analysis of inhibitory SMAD7, a preferred substrate for WWP2-FL and WWP2-C (Soond and Chantry, 2011), showed co-immunoprecipitation mainly with WWP2-FL and WWP2-C following TGFβ1 stimulation (FIG. 16C), which was confirmed in primary fibroblasts (FIGS. 16E and 16F).

These data suggest an endogenous physiological interaction between SMAD2 and WWP2-FL/N protein isoforms.

WWP2 was also found to interact directly with p-SMAD2 (FIG. 16G), and the levels of SMAD2 and p-SMAD2 proteins were similar in the WT and Wwp2^{Mut/Mut} fibroblasts treated with TGFβ1 (FIG. 16H). This is consistent with monoubiquitination of SMAD2 by WWP2, a post-translational modification not affecting SMAD2 protein levels.

### Analysis of WWP2-Mediated Ubiquitination of SMAD Proteins and Nucleocytoplasmic Shuttling

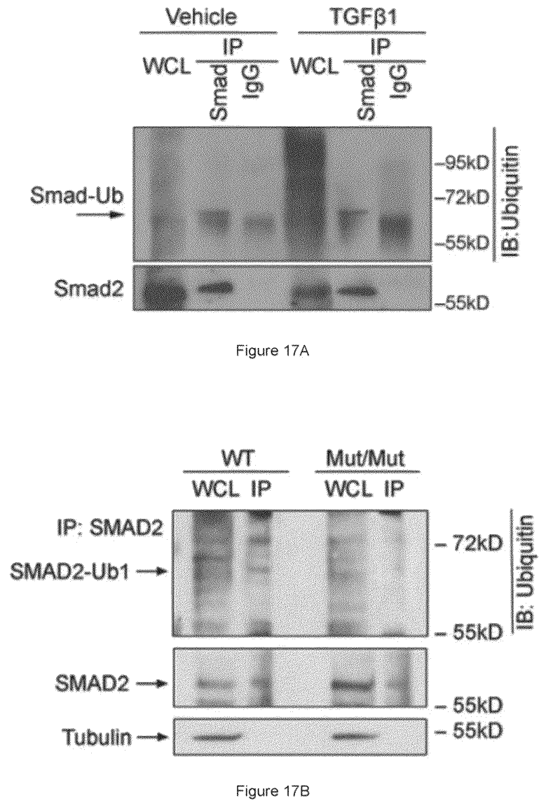

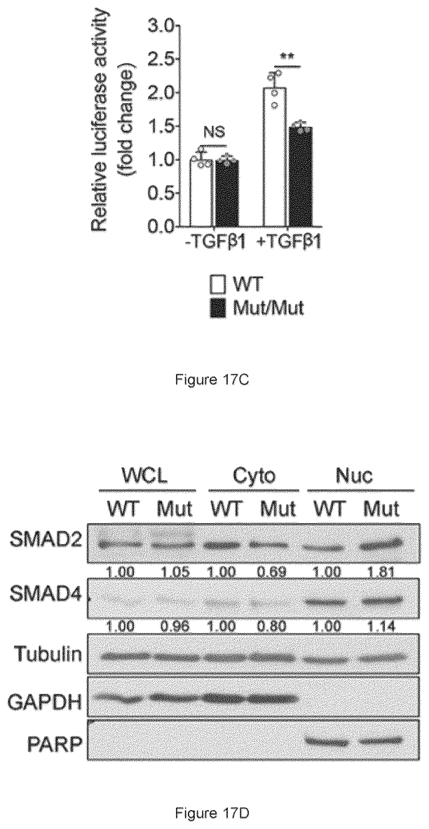

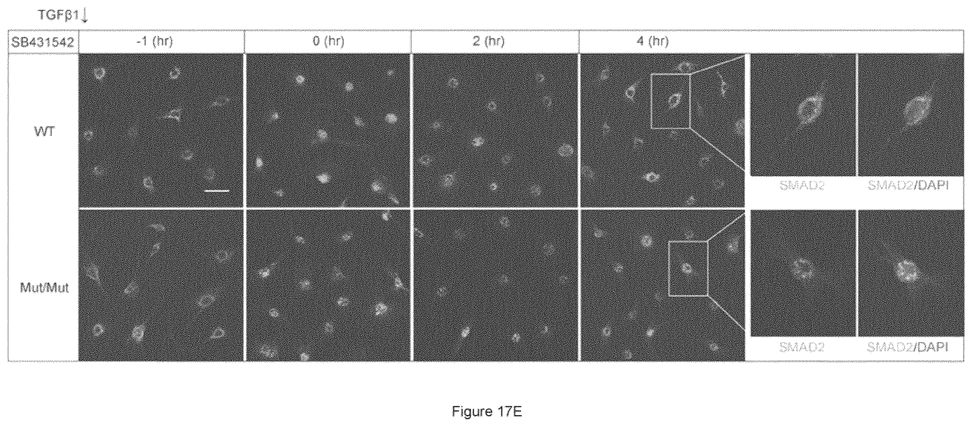

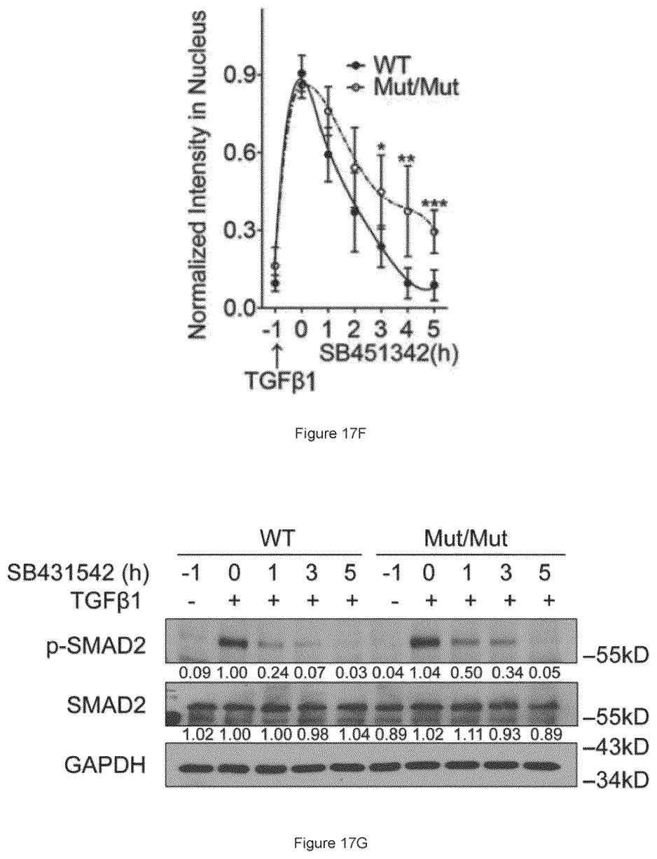

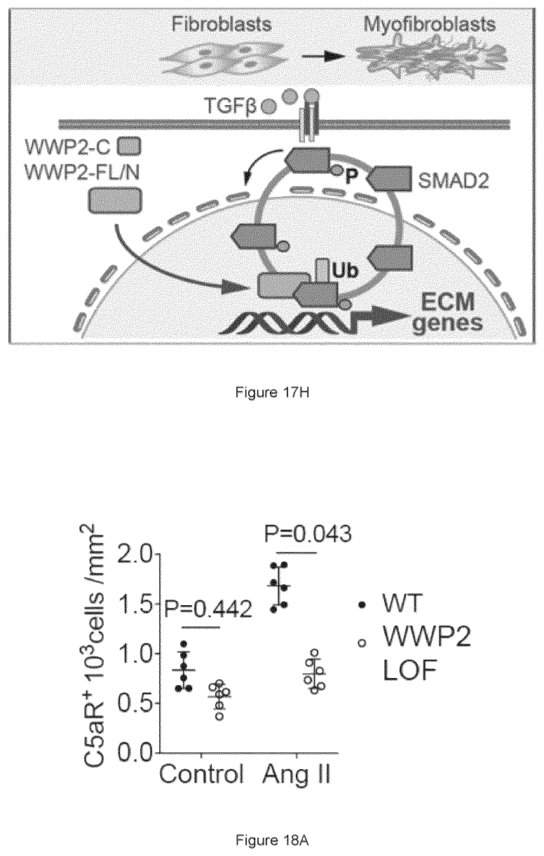

Ubiquitination assays followed by SMAD2 immunoprecipitation detected monoubiquitinated SMAD2 within 16 hours of TGFβ1 stimulation of the mouse NIH-3T3 fibroblast cell line (FIG. 17A). Monoubiquitination of SMAD2 by WWP2 was then confirmed in mouse primary cardiac fibroblasts, and this was reduced in Wwp2^{Mut/Mut} cells (FIG. 17B).

Monoubiquitination of SMADs by ubiquitin E3 ligases reportedly affects their transcriptional activity in primary fibroblasts (Xie et al., 2014; Tang et al., 2011). SMAD2-dependent luciferase reporter activity assays were performed in primary cardiac fibroblasts from WT and Wwp2^{Mut/Mut} mice to investigate whether WWP2 affects the transcriptional activity of SMAD2. TGFβ1-dependent SMAD2 reporter activity was significantly lower in Wwp2^{Mut/Mut} cardiac fibroblasts compared to WT cells (FIG. 17C).

TGFβ-receptor activation promotes the nuclear accumulation of SMAD2/3/4 (Xu and Massague, 2004) and this process is not necessarily accompanied by SMAD degradation in the nucleus, as SMADs are exported out of the nucleus upon dephosphorylation and dissociation of the SMAD complexes (Gareth J. Inman et al., 2002; Lin et al., 2006). Analysis of nuclear and cytoplasmic fractions obtained from cardiac fibroblasts showed nuclear accumulation of SMAD2 upon TGFβ1 stimulation (<16 hr). More SMAD2 was found to be localised to the nucleus in Wwp2^{Mut/Mut} fibroblasts compared to WT cells (FIG. 17D). SMAD4, which forms a heteromeric complex with SMAD2 after TGFβ1 activation, showed similar protein level and subcellular localization in WT and Wwp2^{Mut/Mut} fibroblasts. Despite the fact the SMAD2 protein is more abundant in the nucleus of Wwp2^{Mut/Mut} fibroblasts, lack of WWP2-N/FL was associated with a reduced transcriptional activity of SMAD2 downstream of TGFβ-receptor activation (FIG. 17C).

Monoubiquitination is also important for the proper subcellular localization of SMADs, and in turn might regulate their transcriptional activity in the nucleus (Gareth J Inman et al., 2002; Schmierer and Hill, 2005). SB431542 (a selective inhibitor of TGFβ superfamily type I activin receptor-like kinase (ALK) receptors (Gareth J Inman et al., 2002)) was used to study the differential nuclear export and nucleocytoplasmic shuttling of SMAD2 (Schmierer and Hill, 2005). Using primary fibroblasts from WWP2^{Mut/Mut} and WT mice, a delay in the nuclear export of SMAD2 in Wwp2^{Mut/Mut} mice was observed (FIGS. 17E and 17F). In addition, compared with WT cells, Wwp2^{Mut/Mut} cardiac fibroblasts maintained sizeable levels of p-SMAD2 at 3 hours treatment with SB432542 (FIG. 17G).

Taken together, these data show that in primary cardiac fibroblasts WWP2 interacts with SMAD2, promoting its monoubiquitination, and modulates the nucleocytoplasmic shuttling and transcriptional activity of SMAD2 downstream of TGFβ-signalling activation (FIG. 17G).

### Example 6: Analysis of Immunomodulatory Function of WWP2 in Fibrosis

Bulk RNA-seq analysis in the fibrotic heart (upon AngII-infusion) of Wwp2^{Mut/Mut} and WT mice showed several pathways that are either up- or down-regulated by WWP2. The analysis revealed that both TGFβ-signalling and the coagulation/complement pathway were strongly downregulated by WWP2, the latter being the most significant pathway affected in vivo.

The complement system consists of more than 40 soluble and membrane bound proteins and is activated in several heart diseases. Different parts of the complement system play a role in both stable and unstable coronary heart disease (Oksjoki et al., 2007) and in idiopathic dilated and ischemic cardiomyopathies (Aukrust et al., 2001). One active component is the cleavage product of complement factor 5 (C5), complement factor 5a (C5a), which has chemotactic and inflammatory properties (Iyer et al., 2011).

### C5a Receptor Expression, Fibrosis and the Role of WWP2

Since C5a receptor (C5aR) is widely expressed in several different cell types in the liver, lung, heart, intestine, and kidney tissues (Haviland et al., 1995), the expression of C5aR was analysed in the fibrotic murine heart. The inventors found that C5aR expression was localized to non-myocytes cell in the heart tissues from both WT and Wwp2 LOF mice. AngII-infusion has been reported to activate C5aR signalling in circulating immune cells (Zhang et al., 2014).

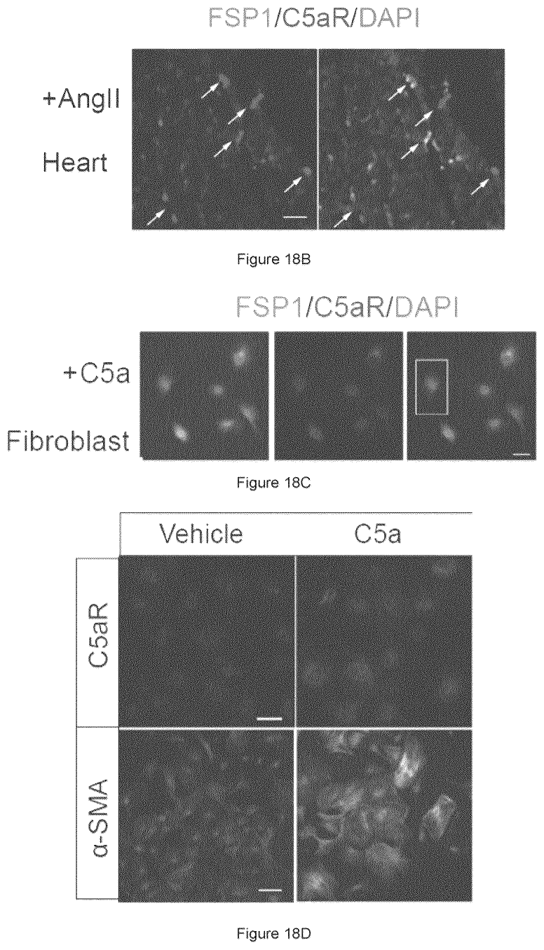

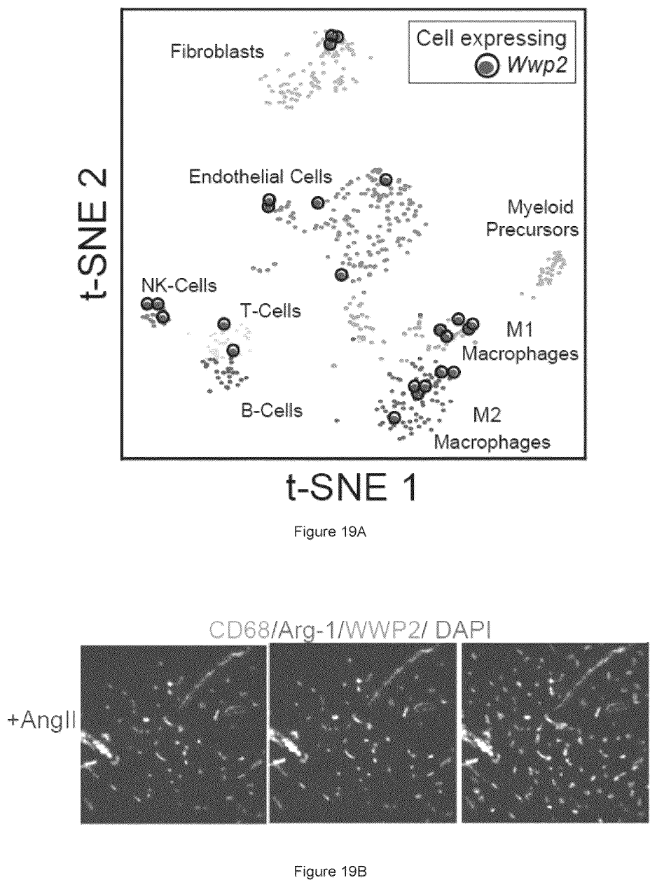

AngII treatment was found to upregulate C5aR expression, and the number of C5aR+ cells induced by AngII-treatment was found to be significantly reduced in Wwp2 LOF mice (FIG. 18A). C5aR activation plays a key role in the triggering of local inflammation, but its effect on cardiac fibrosis is less characterized. Immunofluorescence staining showed that both C5aR and FSP1 are co-localized in the heart sections upon AngII-treatment (FIG. 18B) and that they are expressed in cultured cardiac fibroblasts following C5a stimulation (FIG. 18C). Finally, C5a was found to upregulate C5aR expression in WT fibroblasts and simultaneously induces α-SMA expression (FIG. 18D).

Overall these data suggest that complement activation in heart tissue is involved in the fibrotic response, and that this process is regulated by WWP2 in vivo. Cardiac fibrosis has been proposed as an important therapeutic target in heart failure patients (Segura et al., 2014; Edgley et al., 2012), and so the identification of genes that regulate pathological fibrosis may provide new avenues to control the progression to heart failure.

### WWP2 Expression by Immune Cells

To start exploring the role of WWP2 in regulating immune cell functions in cardiac fibrosis, the inventors first examined the specific immune cells expressing WWP2 in the heart. Preliminary SCS analysis in the heart suggested that WWP2 is expressed by several different types of cardiac cells, including fibroblasts, endothelial cells and various immune cells, such as M1 and M2 macrophages (FIG. 19A). Further analyses of the SCS data, focusing on the resident immune cells in the heart, showed that the majority of WWP2 positive cells were M1 and M2 macrophages (and NK-cells, although to a much less extent). Therefore, SCS analysis revealed that WWP2 is expressed in resident macrophages from WT heart, which was further confirmed by immuno-fluorescence analyses showing WWP2 co-localized with CD-68 and Arg-1, both of which are M2 macrophage markers (FIG. 19B).

SCS data was also analysed to determine whether genes in the complement pathway are transcriptionally co-expressed with WWP2 in individual cardiac cells. Using different immune cell types (i.e. macrophages, T-cells, etc.) expressing WWP2, the expression levels of WWP2 were correlated with the expression levels of genes involved in the complement pathway in each immune cell-type. The strongest positive association between WWP2 and complement pathway genes was found in M2 and M1 macrophages, suggesting that macrophages are a main source contributing to the complement pathway in WT heart (which was most strongly downregulated in the heart of WWP2 LOF mice):

| | Immune cell types expressing | | | |
|---|---|---|---|---|
| | WWP2 in the heart | | | |
| | M2 | M1 | NK | T |
| | macr. | macr. | cells | cells |
| Strength of WWP2 correlation with | 1.32 | 1.28 | 0.70 | 0.46 |
| expression of complement genes | | | | |

The inventors also investigated WWP2 activation in the context of macrophage polarization. Macrophages are a heterogeneous population of tissue-resident professional phagocytes, characterized by phenotypic and functional diversities that play a crucial role in fibrogenesis (Wynn and Vannella, 2016). Since macrophage polarization states (pro-inflammatory M1 and alternatively activated anti-inflammatory M2) are important in mediating cardiac fibrosis (Zhou et al., 2017; Mylonas et al., 2015), bone marrow derived macrophages (BMDMs) were cultured and induced to differentiate to M1 and M2 macrophages by LPS/IFNγ and IL4/IL13 stimulation, respectively.

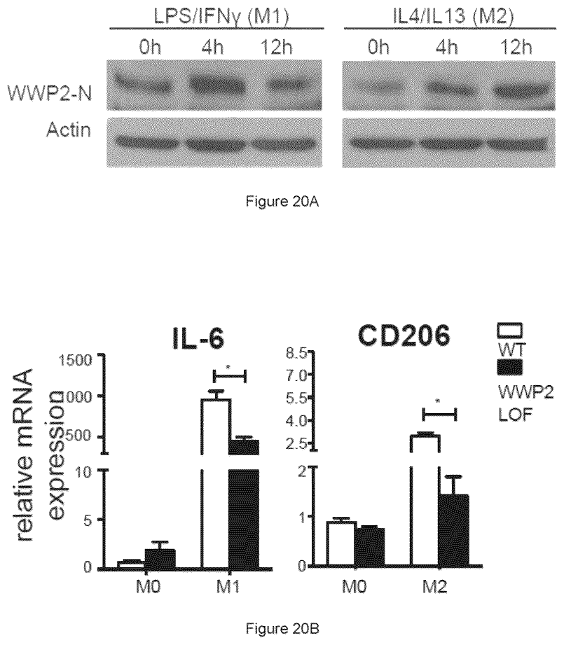

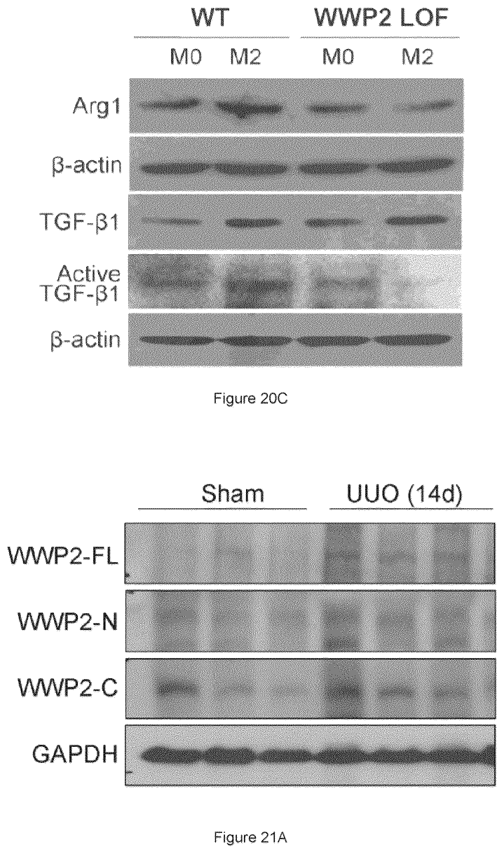

Both M1 and M2 polarization states were associated with increased expression of WWP2 (N-isoform) (FIG. 20A). However, WWP2 expression varied in their temporal dynamics and magnitude.

The inventors further tested the potential regulation of macrophage phenotype/polarization by Wwp2 using cultured BMDMs from both WT and Wwp2 LOF mice. Upon LPS/IFNγ stimulation, BMDMs showed an M1 phenotype, producing pro-inflammatory cytokines such as IL-6. This pro-inflammatory change in M1 macrophages was largely prevented in Wwp2 LOF BMDMs (FIG. 20B). Upon IL4/IL13 stimulation, BMDMs showed M2 phenotype, expressing arginase (Arg-1) and CD206. Compared with WT, BMDMs from W2p2 LOF expressed less Argi and CD206 (MRC1) on M2 polarization (FIGS. 20B and 20C). Although BMDMs from both WT and Wwp2 LOF mice showed similar increase in TGFβ1 protein after IL4/IL13 stimulation, BMDMs from Wwp2 LOF showed significantly reduced expression of active TGFβ1 compared with WT (FIG. 20C). Additional SCS analyses in the murine heart revealed other genes dysregulated by WWP2 LOF, including Fizzi and Cxcl12, which are important for cell migration.

These findings suggest that Wwp2 LOF affects (at least in part) pro-inflammatory and pro-fibrotic phenotypes under M1/M2 polarization.

### Example 7: WWP2 Regulates Kidney Fibrosis In Vivo

Renal fibrosis is a widespread pathological feature of progressive renal disease of virtually any etiology.

The inventors explored whether WWP2 regulates fibrosis in kidney fibrotic disease using a unilateral ureteral obstruction (UUO) model, which generates progressive renal fibrosis (Chevalier et al., 2009), in WT and Wwp2^{Mut/Mut} mice.

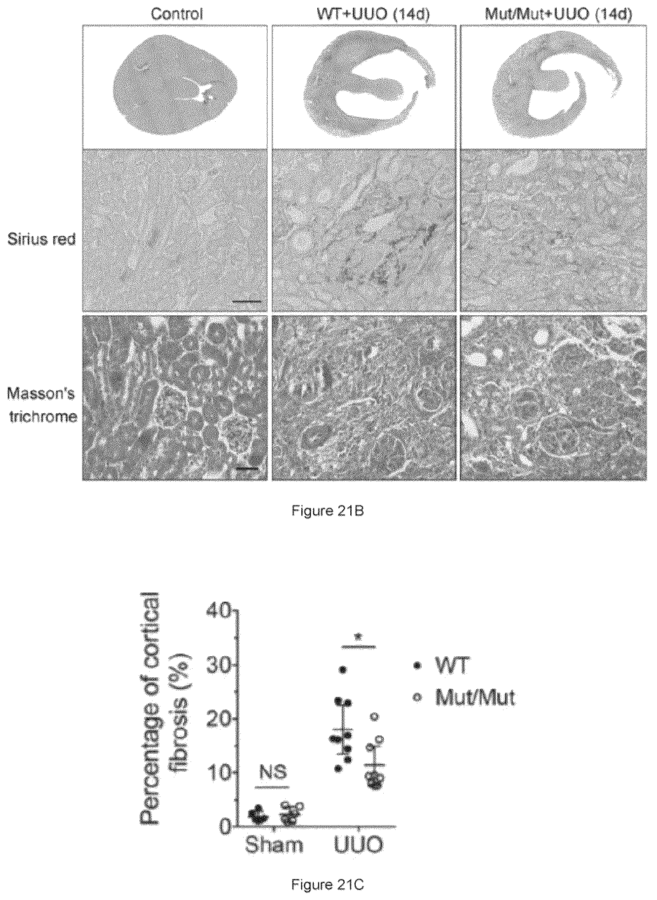

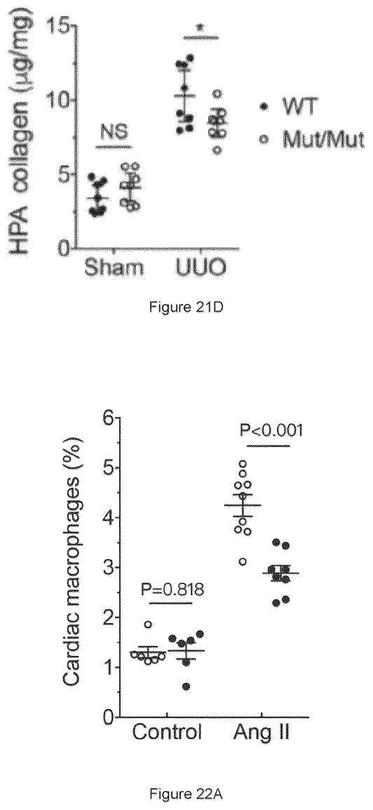

After 14 days, increased levels of all WWP2 protein isoforms (i.e. WWP2-FL, WWP2-N and WWP2-C) was observed (FIG. 21A). After UUO, mice showed increased levels of fibrosis and Wwp2^{Mut/Mut} mice, showed reduced levels of fibrosis and collagen content (HPA assay) as compared to WT mice (FIGS. 21B to 21D).

### Example 8: Discussion

In the present studies, starting from the identification of a pro-fibrotic gene network conserved in rat and in human heart disease characterized by diffuse myocardial remodelling and fibrosis, the inventors identified WWP2 as a regulator of pathological fibrosis. The WWP2-regulated pro-fibrotic gene network was conserved across different fibrotic diseases and cardiac tissues, and was upregulated in dilated cardiomyopathy that progresses to heart failure in mice (Burke et al., 2016). The lack of regulation of the pro-fibrotic gene network by WWP2 in human control heart tissue suggests that WWP2 exerts its regulatory role on cardiac fibrosis upon disease.

WWP2 mRNA expression was only marginally increased in fibrotic heart disease (less than 2 folds in either human DCM, rTOF or mouse HF (Burke et al., 2016)). This might explain why WWP2 passed undetected by GWAS and other eQTL genetic mapping or cellular screening studies of fibrotic diseases. In the heart of DCM patients, isoform-specific eQTL analysis led the inventors to hypothesize that increased expression of the WWP2 N-terminal isoform was associated with the activation of a pro-fibrotic gene program downstream of TGFβ/SMAD signalling activation. This hypothesis was tested in primary cardiac fibroblasts and in vivo models of cardiac fibrosis.

The results provide the first indication of a role for WWP2 in regulating pathophysiological processes in the heart. In detail, the inventors have demonstrated that WWP2-N/FL LOF improves cardiac function and reduces myocardial fibrosis, in two established preclinical models of cardiovascular fibrosis (Example 3.2). Moreover, upon treatment with Ang II, a typical promoter of cardiac fibrosis, Wwp2^{Mut/Mut} mice displayed increased oxidative phosphorylation, and exacerbated cell cycle and IFNα and IFNγ response in the heart. This suggests that depletion of the N-terminal part of WWP2 might have a wider role in myocardial fibrosis, preventing the glycolytic metabolic reprogramming required for myofibroblast differentiation (Bernard et al., 2015; Selvarajah et al., 2016), and potentially blocking the cell cycle arrest that has been shown to be a feature of fibrosis (Lovisa et al., 2015).

Depletion of the N-terminal part of WWP2 additionally increased the expression of IFNα and IFNγ response genes, which have been shown to induce apoptosis of myofibroblasts (Nedelec et al., 2001; Yokozeki et al., 1999).

Working in primary fibroblast cultures, the inventors have shown that WWP2 positively regulates the expression of established pro-fibrotic genes downstream of TGFβ/SMAD signalling activation. However, compared with in vivo results, the pro-fibrotic response to TGFβ1 in quiescent cardiac fibroblasts was more modest, possibly because some of the cultured fibroblasts have been already converted to myofibroblasts before TGFβ1 treatment (Santiago et al., 2010). Despite this, the phenotypic differences if the fibrotic response observed between WT and Wwp2^{Mut/Mut} cardiac fibroblasts were consistent in all WWP2 loss-of-function, gain-of-function and rescue experiments.

Poly-ubiquitination and proteasome-mediated degradation of nuclear SMAD2 has already been described (Lo and Massague, 1999). In addition, monoubiquitination can regulate nuclear accumulation and the nucleocytoplasmic shuttling of SMAD complexes, which are crucial for transduction of TGFβ-superfamily signals (Gareth J. Inman et al., 2002; Hill, 2009), and there is mounting evidence supporting the role of monoubiquitination of SMADs by E3 ubiquitin ligases in this context (Tang et al., 2011; Gareth J. Inman et al., 2002).

The inventors have shown for the first time that TGFβ1 stimulation promotes the translocation of WWP2 to the nucleus, where it interacts directly with SMAD2, possibly promoting its monoubiquitination, as shown for other ubiquitin E3 ligases (Komuro et al., 2004; Tang et al., 2011). The regulatory consequences of E3 ligase-mediated monoubiquitination appear to be complex and context specific. Monoubiquitination can regulate protein location, activity, and protein interactions with binding partners (Schnell and Hicke, 2003). Monoubiquitination has also been shown to be required for the activity and the intrinsic nuclear import of target transcription factor(s) (Zou et al., 2011; Trotman et al., 2007); while in other instances monoubiquitination has been reported to disrupt specific transcription factor interactions and their transcriptional activity (Inui et al., 2011).

In the present studies, the inventors did not observe SMAD2/3 degradation by endogenous WWP2 in primary fibroblasts, suggesting a mechanism of degradation-independent repression of SMAD2 activity downstream of TGFβ-signalling activation. The present data suggest that blocking WWP2 function can delay the TGFβ1-induced nucleocytoplasmic shuttling of SMAD2 in primary fibroblasts.

### Example 9: Conclusion

The present studies contribute to the understanding of the regulation of fibrosis and pathological inflammation, and identify a specific E3 ubiquitin ligase, WWP2, as a regulator of ECM accumulation downstream of TGFβ/SMAD signalling, and of immune cell phenotype.

In summary, using a systems genetics approach followed by functional validation in several cellular and pre-clinical animal model studies, the inventors have first identified and mechanistically explained that WWP2 is a novel and druggable therapeutic target with the potential to control fibrosis in pathological inflammation and tissue remodelling.

### Example 10: References to Examples 2 to 8

Arpino, V. et al. (2015) The role of TIMPs in regulation of extracellular matrix proteolysis. Matrix Biology
Aschner, Y. et al. (2014) Protein tyrosine phosphatase a mediates profibrotic signaling in lung fibroblasts through TGF-β responsiveness. American Journal of Pathology.
Aukrust, P. et al. (2001) Complement activation in patients with congestive heart failure: Effect of high-dose intravenous immunoglobulin treatment. Circulation.
Baum, J. & Duffy, H. S. (2011) Fibroblasts and myofibroblasts: what are we talking about? Journal of cardiovascular pharmacology. 57 (4), 376-379.
Bernard, K. et al. (2015) Metabolic Reprogramming Is Required for Myofibroblast Contractility and Differentiation. The Journal of biological chemistry. 290 (42), 25427-25438.
Bottolo, L. et al. (2011) Bayesian detection of expression quantitative trait loci hot spots. Genetics. 189 (4), 1449-1459.
Brooks, C. L. et al. (2007) Mechanistic studies of MDM2-mediated ubiquitination in p53 regulation. Journal of Biological Chemistry.
Burke, M. A. et al. (2016) Molecular profiling of dilated cardiomyopathy that progresses to heart failure. JCI Insight.
Carvajal, L. A. et al. (2018) Dual inhibition of MDMX and MDM2 as a therapeutic strategy in leukemia. Science Translational Medicine.
Chaudhary, N. & Maddika, S. (2014) WWP2-WWP1 Ubiquitin Ligase Complex Coordinated by PPM1 G Maintains the Balance between Cellular p73 and Np73 Levels. Molecular and Cellular Biology.
Chevalier, R. L. et al. (2009) Ureteral obstruction as a model of renal interstitial fibrosis and obstructive nephropathy. Kidney International. 75 (11), 1145-1152.
Ciftciler, R. et al. (2017) The importance of serum biglycan levels as a fibrosis marker in patients with chronic hepatitis B. Journal of Clinical Laboratory Analysis. 31 (5), e22109.
DeBerge, M. et al. (2019) Macrophages in Heart Failure with Reduced versus Preserved Ejection Fraction. Trends in molecular medicine. 25 (4), 328-340.
Ding, L. et al. (2017) Inhibition of Skp2 suppresses the proliferation and invasion of osteosarcoma cells. Oncology Reports.
Edgley, A. J. et al. (2012) Targeting Fibrosis for the Treatment of Heart Failure: A Role for Transforming Growth Factor-β. Cardiovascular Therapeutics. 30 (1), e30-e40.
Fukumoto, C. et al. (2014) WWP2 is overexpressed in human oral cancer, determining tumor size and poor prognosis in patients: downregulation of WWP2 inhibits the AKT signaling and tumor growth in mice. Oncoscience. 1807.
Gao, R. et al. (2016) E3 ubiquitin ligase RLIM negatively regulates c-Myc transcriptional activity and restrains cell proliferation. PLoS ONE.
Haviland, D. L. et al. (1995) Cellular expression of the C5a anaphylatoxin receptor (C5aR): demonstration of C5aR on nonmyeloid cells of the liver and lung. Journal of immunology (Baltimore, Md.: 1950). 154 (4), 1861-1869.
Heinig, M. et al. (2010) A trans-acting locus regulates an anti-viral expression network and type 1 diabetes risk. Nature. 467 (7314), 460-464.
Heinig, M. et al. (2017) Natural genetic variation of the cardiac transcriptome in non-diseased donors and patients with dilated cardiomyopathy. Genome Biology. 18 (1), 170.
Hill, C. S. (2009) Nucleocytoplasmic shuttling of Smad proteins. Cell Research Horwitz, A. A. et al. (2007) A mechanism for transcriptional repression dependent on the BRCA1 E3 ubiquitin ligase. Proceedings of the National Academy of Sciences.
Hu, H.-H. et al. (2018) New insights into TGF-β/Smad signaling in tissue fibrosis. Chemico-Biological Interactions. 29276-83. Huang, X. & Dixit, V. M. (2016) Drugging the undruggables: exploring the ubiquitin system for drug development. Cell Research. 26 (4), 484-498.
Huang, X. L. et al. (2016) E3 ubiquitin ligase: A potential regulator in fibrosis and systemic sclerosis. Cellular Immunology Hubner, N. et al. (2005) Integrated transcriptional profiling and linkage analysis for identification of genes underlying disease. Nature genetics. 37 (3), 243-253.
Inman, G. J. et al. (2002) Nucleocytoplasmic shuttling of Smads 2, 3, and 4 permits sensing of TGF-β receptor activity. Molecular Cell.
Inman, G. J. et al. (2002) SB-431542 is a potent and specific inhibitor of transforming growth factor-beta superfamily type I activin receptor-like kinase (ALK) receptors ALK4, ALK5, and ALK7. Molecular pharmacology.
Inui, M. et al. (2011) USP15 is a deubiquitylating enzyme for receptor-activated SMADs. Nature Cell Biology.
Iyer, A. et al. (2011) Inhibition of inflammation and fibrosis by a complement C5a receptor antagonist in DOCA-salt hypertensive rats. Journal of Cardiovascular Pharmacology.
Jia, L. & Sun, Y. (2011) SCF E3 Ubiquitin Ligases as Anticancer Targets. Current Cancer Drug Targets.
Johnson, M. R. et al. (2015) Systems genetics identifies Sestrin 3 as a regulator of a proconvulsant gene network in human epileptic hippocampus. Nature communications. 6 (1), 6031.
Kang, H. et al. (2014) Kcnn4 is a regulator of macrophage multinucleation in bone homeostasis and inflammatory disease. Cell reports. 8 (4), 1210-1224.
Khalil, H. et al. (2017) Fibroblast-specific TGF-β-Smad2/3 signaling underlies cardiac fibrosis. Journal of Clinical Investigation. 127 (10), 3770-3783.
Kim, S. et al. (2018) Two Genetic Variants Associated with Plantar Fascial Disorders. International Journal of Sports Medicine. 39 (04), 314-321.
Komuro, A. et al. (2004) Negative regulation of transforming growth factor-β (TGF-φ signaling by WW domain-containing protein 1 (WWP1). Oncogene.
Lachmann, A. et al. (2010) ChEA: transcription factor regulation inferred from integrating genome-wide ChIP-X experiments. Bioinformatics (Oxford, England). 26 (19), 2438-2444.
Langley, S. R. et al. (2013) Systems-level approaches reveal conservation of trans-regulated genes in the rat and genetic determinants of blood pressure in humans. Cardiovascular research. 97 (4), 653-665.
Leach, H. G. et al. (2013) Endothelial Cells Recruit Macrophages and Contribute to a Fibrotic Milieu in Bleomycin Lung Injury. American Journal of Respiratory Cell and Molecular Biology. 49 (6), 1093-1101.
Leask, A. (2015) Getting to the Heart of the Matter: New Insights Into Cardiac Fibrosis. Circulation research. 116 (7), 1269-1276. Lewin, A. et al. (2016) MT-HESS: an efficient Bayesian approach for simultaneous association detection in OMICS datasets, with application to eQTL mapping in multiple tissues. Bioinformatics (Oxford, England). 32 (4), 523-532.
Lin, X. et al. (2006) PPM1A Functions as a Smad Phosphatase to Terminate TGFβ Signaling. Cell.
Lo, R. S. & Massague, J. (1999) Ubiquitin-dependent degradation of TGF-β-activated Smad2. Nature Cell Biology.
Lovisa, S. et al. (2015) Epithelial-to-mesenchymal transition induces cell cycle arrest and parenchymal damage in renal fibrosis. Nature medicine. 21 (9), 998-1009.
Maddika, S. et al. (2011) WWP2 is an E3 ubiquitin ligase for PTEN. Nature cell biology. 13 (6), 728-733.
Mancini, M. et al. (2013) Mapping genetic determinants of coronary microvascular remodeling in the spontaneously hypertensive rat. Basic research in cardiology. 108 (1), 316.
Massague, J. & Wotton, D. (2000) NEW EMBO MEMBERS REVIEW: Transcriptional control by the TGF-beta/Smad signaling system. The EMBO Journal. 19 (8), 1745-1754.
Matsumura, S. I. et al. (2005) Targeted deletion or pharmacological inhibition of MMP-2 prevents cardiac rupture after myocardial infarction in mice. Journal of Clinical Investigation.
McVicker, B. L. & Bennett, R. G. (2017) Novel Anti-fibrotic Therapies. Frontiers in Pharmacology. 8318.
Moustakas, A. et al. (2001) Smad regulation in TGF-beta signal transduction. Journal of cell science. 114 (Pt 24), 4359-4369.
Mylonas, K. J. et al. (2015) The adult murine heart has a sparse, phagocytically active macrophage population that expands through monocyte recruitment and adopts an ‘M2’ phenotype in response to Th2 immunologic challenge. Immunobiology.
Nakamura, Y. et al. (2011) Wwp2 is essential for palatogenesis mediated by the interaction between Sox9 and mediator subunit 25. Nature communications. 2251.
Nedelec, B. et al. (2001) Myofibroblasts and apoptosis in human hypertrophic scars: The effect of interferon-α2b. Surgery. 130 (5), 798-808.
Oksjoki, R. et al. (2007) Receptors for the anaphylatoxins C3a and C5a are expressed in human atherosclerotic coronary plaques. Atherosclerosis.
Peterson, J. T. et al. (2000) Evolution of matrix metalloprotease and tissue inhibitor expression during heart failure progression in the infarcted rat. Cardiovascular Research.
Petretto, E. et al. (2008) Integrated genomic approaches implicate osteoglycin (Ogn) in the regulation of left ventricular mass. Nature genetics. 40 (5), 546-552.
Piersma, B. et al. (2015) Signaling in Fibrosis: TGF-β, WNT, and YAP/TAZ Converge. Frontiers in Medicine. 259.
Pradegan, N. et al. (2016) Myocardial histopathology in late-repaired and unrepaired adults with tetralogy of Fallot. Cardiovascular Pathology.
Pravenec, M. et al. (2008) Identification of renal Cd36 as a determinant of blood pressure and risk for hypertension. Nature genetics. 40 (8), 952-954.
Rockey, D. C. et al. (2015) Fibrosis—A Common Pathway to Organ Injury and Failure. The New England journal of medicine. 372 (12), 1138-1149.
Ross, S. & Hill, C. S. (2008) How the Smads regulate transcription. The International Journal of Biochemistry & Cell Biology. 40 (3), 383-408.
Rotival, M. & Petretto, E. (2014) Leveraging gene co-expression networks to pinpoint the regulation of complex traits and disease, with a focus on cardiovascular traits. Briefings in functional genomics. 13 (1), 66-78.
Santiago, J. J. et al. (2010) Cardiac fibroblast to myofibroblast differentiation in vivo and in vitro: Expression of focal adhesion components in neonatal and adult rat ventricular myofibroblasts. Developmental Dynamics.
Schafer, S. et al. (2017) IL11 is a crucial determinant of cardiovascular fibrosis. Nature. 552 (7683), 110.
Schmidt, M. et al. (2015) Controlling the Balance of Fibroblast Proliferation and Differentiation: Impact of Thy-1. Journal of Investigative Dermatology. 135 (7), 1893-1902.
Schmierer, B. & Hill, C. S. (2005) Kinetic analysis of Smad nucleocytoplasmic shuttling reveals a mechanism for transforming growth factor beta-dependent nuclear accumulation of Smads. Molecular and cellular biology.
Schnell, J. D. & Hicke, L. (2003) Non-traditional Functions of Ubiquitin and Ubiquitin-binding Proteins. Journal of Biological Chemistry.
Schwanekamp, J. A. et al. (2017) TGFBI functions similar to periostin but is uniquely dispensable during cardiac injury. PLoS ONE.
Segura, A. M. et al. (2014) Fibrosis and heart failure. Heart Failure Reviews.
Selvarajah, B. et al. (2016) Metabolic shift during TGF-β induced collagen synthesis. QJM: An International Journal of Medicine. 109 (suppl_1), S3-S3.
Soond, S. M. & Chantry, A. (2011) Selective targeting of activating and inhibitory Smads by distinct WWP2 ubiquitin ligase isoforms differentially modulates TGFβ signalling and EMT. Oncogene. 30 (21), 2451-2462.
Styrkarsdottir, U. et al. (2018) Meta-analysis of Icelandic and UK data sets identifies missense variants in SMO, IL11, COL11 A1 and 13 more new loci associated with osteoarthritis. Nature Genetics. 50 (12), 1681-1687.
Subramanian, A. et al. (2005) Gene set enrichment analysis: a knowledge-based approach for interpreting genome-wide expression profiles. Proceedings of the National Academy of Sciences of the United States of America. 102 (43), 15545-15550.
Tang, L. Y. et al. (2011) Ablation of Smurf2 reveals an inhibition in TGF-β signalling through multiple mono-ubiquitination of Smad3. EMBO Journal.
Trotman, L. C. et al. (2007) Ubiquitination Regulates PTEN Nuclear Import and Tumor Suppression. Cell.
Uderhardt, S. et al. (2019) Resident Macrophages Cloak Tissue Microlesions to Prevent Neutrophil-Driven Inflammatory Damage. Cell. 0 (0).
Villafañe, J. et al. (2013) Hot topics in tetralogy of fallot. Journal of the American College of Cardiology
Wang, Q. et al. (2011) Cooperative interaction of CTGF and TGF-β in animal models of fibrotic disease. Fibrogenesis & tissue repair. 4 (1), 4.
Wynn, T. A. & Vannella, K. M. (2016) Macrophages in Tissue Repair, Regeneration, and Fibrosis. Immunity
Xie, F. et al. (2014) Regulation of TGF-β Superfamily Signaling by SMAD Mono-Ubiquitination. Cells.
Xu, L. & Massague, J. (2004) Nucleocytoplasmic shuttling of signal transducers. Nature Reviews Molecular Cell Biology
Yang, Y. et al. (2013) E3 ligase WWP2 negatively regulates TLRβ-mediated innate immune response by targeting TRIF for ubiquitination and degradation. Proceedings of the National Academy of Sciences. 110 (13), 5115-5120.
Yokozeki, M. et al. (1999) Interferon-gamma inhibits the myofibroblastic phenotype of rat palatal fibroblasts induced by transforming growth factor-beta1 in vitro. FEBS letters. 442 (1), 61-64.
Zhang, C. et al. (2014) Complement 5a receptor mediates angiotensin II-induced cardiac inflammation and remodeling. Arteriosclerosis, Thrombosis, and Vascular Biology.
Zhao, T. et al. (2013) Platelet-derived growth factor-D promotes fibrogenesis of cardiac fibroblasts. American journal of physiology. Heart and circulatory physiology. 304 (12), H1719-26.
Zhou, D. et al. (2017) Macrophage polarization and function: new prospects for fibrotic disease. Immunology and Cell Biology. 95 (10), 864-869.
Zou, W., Chen, X., Shim, J.-H., Huang, Z., Brady, N., Hu, D., Drapp, R., Sigrist, K., Glimcher, L. H., et al. (2011) The E3 ubiquitin ligase Wwp2 regulates craniofacial development through mono-ubiquitylation of Goosecoid. Nature cell biology. 13 (1), 59-65.

### Example 11: Identification of WWP2 Inhibitors

Inhibitors of WWP2 isoforms WWP2-FL and WWP2-N were successfully identified.

### 11.1 Identification of Test Compounds that Bind to WWP2

WWP2-FL, WWP2-N and WWP2-C were expressed as GST fusion proteins in bacteria, and purified using glutathione sepharose 4B resin (GE Healthcare) according to the manufacturer's instructions. Protein levels were determined by Coomasie staining and Western blot prior to use in Octet Binding assays.

Octet binding assays were performed using the Octet RED96e System (ForteBio Inc., Menlo Park, CA). Proteins were biotinylated using NHS-PEO4-biotin (Pierce). Super-streptavidin (SSA) biosensors (ForteBio Inc., Menlo Park, CA) were coated in a solution containing 1 μM of biotinylated protein for 4 hours at 25° C. A duplicate set of sensors was incubated in an assay buffer (1× kinetics buffer of ForteBio Inc.) with 2.5% DMSO without protein for use as a background binding control. Both sets of sensors were blocked with a solution of 10 μg/ml Biocytin for 5 minutes at 25° C. A negative control of 2.5% DMSO was also used.

Binding of test compound samples (250 μM) to coated and uncoated reference sensors was measured over 120 seconds.

Data analysis on the ForteBio Octet RED instrument was performed using a double reference subtraction (sample and sensor references) in the ForteBio data analysis software. The analysis accounts for non-specific binding, background, and signal drift and minimizes well-based and sensor variability.

Test compounds determined to bind to WWP2-N, and determined not to bind to WWP2-C were selected for further characterisation.

### 11.2 Identification of Test Compounds that Inhibit WWP2-Mediated Ubiquitination

In vitro ubiquitination assays are performed. The assays contain 150 ng E1 (BIOMOL, Plymouth Meeting), 150 ng E2 (UbcH7 or UbcH5c, BIOMOL; 500 ng UbcH7 with Smurf2), 10 μg ubiquitin (BIOMOL), and 20 ng E3 (WWP2-FL or WWP2-C).

For a quantitative readout, untagged ubiquitin (10 μg) is mixed with N-terminally biotinylated ubiquitin (1 μg), and 1 μg of GST-tagged SMAD2 substrate is adsorbed to glutathione-coated (or for some experiments, anti-GST coated) ELISA plates (Pierce; maximum capacity, 10 ng protein) for 45 min, followed by incubation with HRP-coupled Streptavidin for 20 min. Ubiquitinated SMAD2 is quantified after addition of 100 μL tetramethylbenzidine solution (Sigma-Aldrich) on a TECAN Infinite F200 microplate reader.

Candidate inhibitor test compounds are pre-incubated with E3 for 10-30 min at a range of concentrations prior to addition to the other components of the ubiquitination assay. Inhibition of WWP2FL/C ubiquitination activity by the test compounds is compared relative to vehicle control conditions.

### 11.3 Evaluation of Test Compounds for Inhibition of Cellular Fibrosis

Primary cultured cardiac fibroblasts are plated at a density of 350 cells per well on poly-D-lysine (PDL, 10 μg/mL)-coated 96-well plates. After 24-h serum starvation, medium is incubated with test compounds (5 μM) for 30 minutes, and which are then added to the cardiac fibroblasts, and the cells are stimulated by culture in the presence of TGF-β1 (5 ng/mL; Sigma).

After 48 h, cells are fixed with a 4% paraformaldehyde solution for 10 min, subsequently blocked for 30 min (5% FBS, 0.3% Triton X-100 in PBS) and then stained overnight at 4° C. with primary mouse anti-αSMA antibody (in 1% FBS, 0.1% Triton X-100 in PBS). After washing with PBS, cells are stained with secondary antibody (donkey anti-mouse Alexa Fluor 488, Invitrogen; 1:500) for 2 h at room temperature in the dark. After three washes with PBS, the plate is sealed for imaging.

High-content imaging is performed with an Operetta High Content Imaging system (PerkinElmer). Four imaging fields are captured per well with a 5× imaging objective, to enable visualization of the entire well.

Relative cell area and relative staining intensity of α-SMA are determined using internal algorithms in the Cellomics Scan software package, and thresholds fitted using multiple wells of positive (SB-431542, 10 μM) and negative (DMSO, 0.1%) controls present in each screening plate.

### Example 12: Treatment of Fibrosis in Various Different Tissues Using WWP2 Inhibitors

The utility of WWP2 inhibitors to treat/prevent fibrosis is demonstrated in in vivo mouse models of fibrosis for various different tissues.

### 12.1 Heart Fibrosis

An AngII infusion model is established as described in Example 1.8. A myocardial infarction model is established as described in Example 1.8.

Mice are treated with WWP2 inhibitor or vehicle (negative control). At the end of the experiment, hearts are harvested and analysed for correlates of fibrosis.

Mice treated with WWP2 inhibitor are found to display a reduced level of heart fibrosis as compared to vehicle-treated controls.

### 12.2 Kidney Fibrosis

A mouse model of kidney fibrosis is established by unilateral ureteral obstruction (UUO), as described in Chevalier et al., Kidney International (2009) 75 (11), 1145-1152.

Mice are treated with WWP2 inhibitor or vehicle (negative control). At the end of the experiment, kidneys are harvested and analysed for correlates of fibrosis.

Mice treated with WWP2 inhibitor are found to display a reduced level of kidney fibrosis as compared to vehicle-treated controls.

### 12.3 Liver Fibrosis

A mouse model of liver fibrosis is established.

Mice are treated with WWP2 inhibitor or vehicle (negative control). At the end of the experiment, livers are harvested and analysed for correlates of fibrosis.

Mice treated with WWP2 inhibitor are found to display a reduced level of liver fibrosis as compared to vehicle-treated controls.

### 12.4 Lung Fibrosis

A mouse model of lung fibrosis is established.

Mice are treated with WWP2 inhibitor or vehicle (negative control). At the end of the experiment, lungs are harvested and analysed for correlates of fibrosis.

Mice treated with WWP2 inhibitor are found to display a reduced level of lung fibrosis as compared to vehicle-treated controls.

### 12.5 Skin Fibrosis

A mouse model of skin fibrosis is established.

Mice are treated with WWP2 inhibitor or vehicle (negative control). At the end of the experiment, the skin is analysed for correlates of fibrosis.

Mice treated with WWP2 inhibitor are found to display a reduced level of skin fibrosis as compared to vehicle-treated controls.

### 12.6 Eye Fibrosis

A mouse model of eye fibrosis is established.

Mice are treated with WWP2 inhibitor or vehicle (negative control). At the end of the experiment, the eyes are analysed for correlates of fibrosis.

Mice treated with WWP2 inhibitor are found to display a reduced level of eye fibrosis as compared to vehicle-treated controls.

### 12.7 Bowel Fibrosis

A mouse model of bowel fibrosis is established.

Mice are treated with WWP2 inhibitor or vehicle (negative control). At the end of the experiment, the bowel is analysed for correlates of fibrosis.

Mice treated with WWP2 inhibitor are found to display a reduced level of bowel fibrosis as compared to vehicle-treated controls.

### Example 13: WWP2 Regulates Immune Function of Cardiac Macrophages in Fibrosis

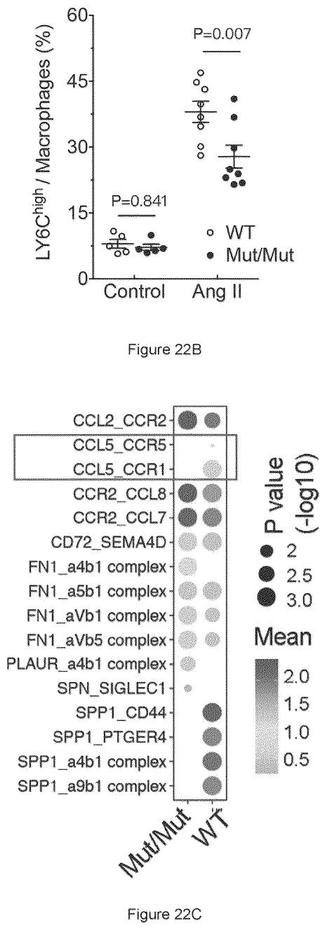

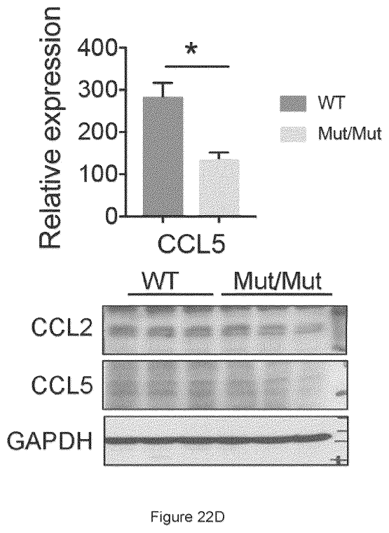

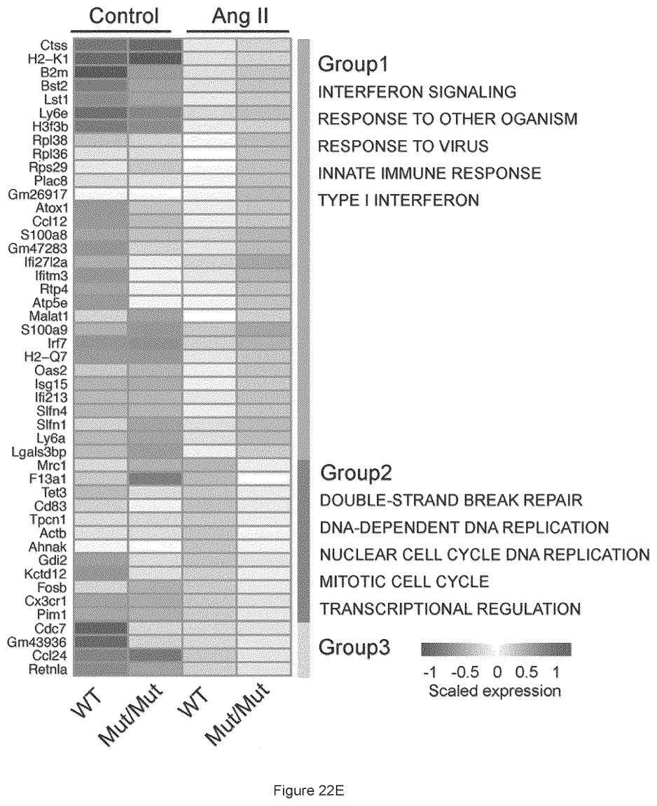

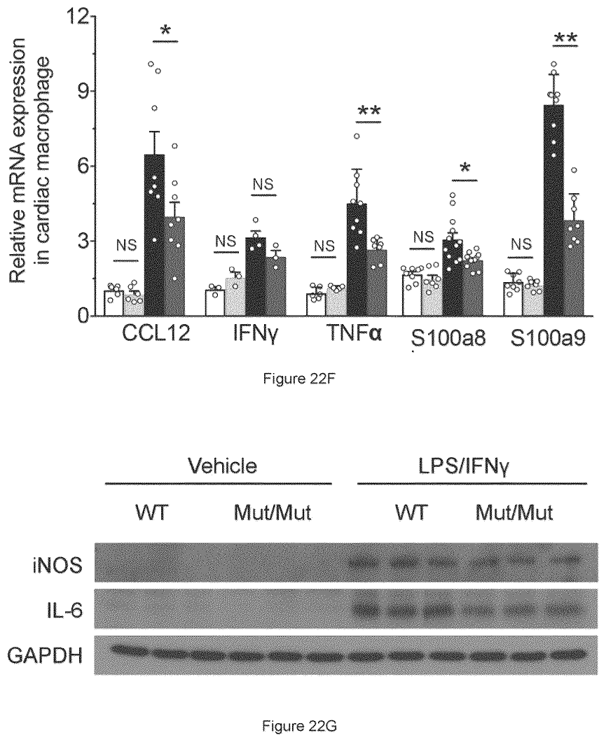

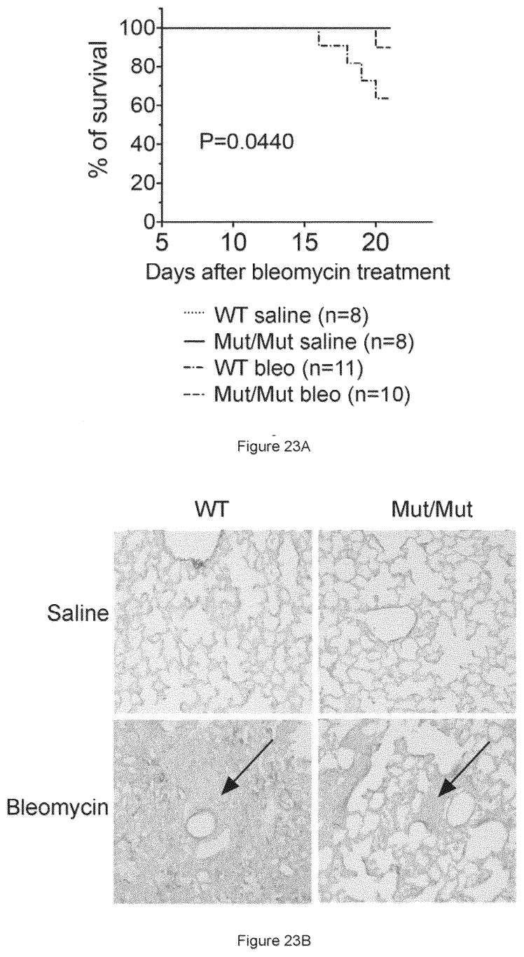

The inventors first assessed the macrophages in the heart following Ang II infusion. Ang II infusion was performed as described in Example 1.8 but the duration of infusion was 7 days. Hearts from sham or AngII-infused animals were analysed by flow cytometry. Ang II induced significant accumulation of macrophages (CD45⁺/CD11b⁺/Ly6G⁻/F4/80⁺/CD64⁺) at 7 days. In contrast to WT mice, Wwp2^{Mut/Mut} mice showed a significant attenuation of Ang II-induced macrophages (FIG. 22A).

Ly6C^{high} cells are often referred to as “inflammatory monocytes”. The inventors observed an increase of Ly6C^{high} macrophages in fibrotic hearts, as well as a suppressing effect of Wwp2 LOF in this macrophage population (FIG. 22B).

Using CellphoneDB (a publicly available repository of curated receptors, ligands and their interactions) on the single-cell RNA-sequencing data, the inventors found significant Ligand-Receptor interactions in inflammatory macrophages for both WT and Wwp2 LOF mice. Wwp2 LOF inhibited activation of the CCL5 pathway (FIG. 22C). This was confirmed by qPCR analysis showing that the levels (relative expression) of CCL5 were decreased in Wwp2 LOF cardiac macrophages as compared with WT cells (FIG. 22D). This finding was further confirmed at the protein level in cultured bone marrow derived-macrophages (BMDM) as shown in FIG. 22D (lower panel).

Differential expression analysis between WT and Wwp2 LOF inflammatory macrophages was performed in order to investigate the regulation of cardiac macrophages by Wwp2 (FIG. 22E). Here, Ang II infusion upregulated a group of genes associated with inflammation (e.g. Interferon signalling, see Group 1 in FIG. 22E), which is expected in the early fibrotic response, while Wwp2 LOF significantly suppressed such an inflammatory response in macrophages from fibrotic heart, and conversely, Wwp2 LOF was associated with upregulation of reparatory pathways in macrophages (e.g. DNA replication, see Group 2 in FIG. 22E).

Analysis of cardiac macrophages sorted from fibrotic heart validated that Wwp2 LOF suppressed the inflammatory response (e.g. CCL12, S100a9) (FIG. 22F). Similar findings were further confirmed in BMDM with LPS/IFNγ stimulation (FIG. 22G).

### Example 14: WWP2 LOF Protects from Lung Fibrosis in Mice

Bleomycin-induced lung fibrosis is a widely used mouse model for human idiopathic pulmonary fibrosis (IPF)—see Liu et al., Methods Mol Biol (2017) 1627:27-42. Briefly, lung fibrosis was induced by oropharyngeal treatment of bleomycin: 8-10 weeks old female mice weighing approximately 20 to 22 g were anesthetized by isoflurane inhalation and then bleomycin (Sigma-Aldrich) was administered oropharyngeal at a dose of 1 mg/kg body weight. Saline administration was treated as sham control. Mice were observed daily for body weight and activity levels and harvested on day 21 post-bleomycin challenge. Administration of bleomycin to the lung was found to cause moderate long-term mortality. The inventors observed that fibrosis is fully developed with extensive and diffuse tissue involvement of the lung at 21 days of bleomycin treatment.

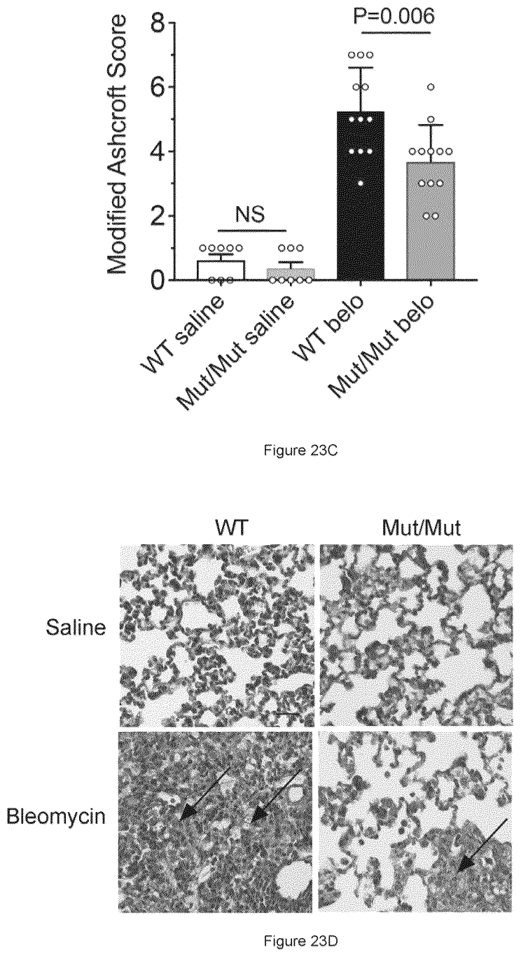

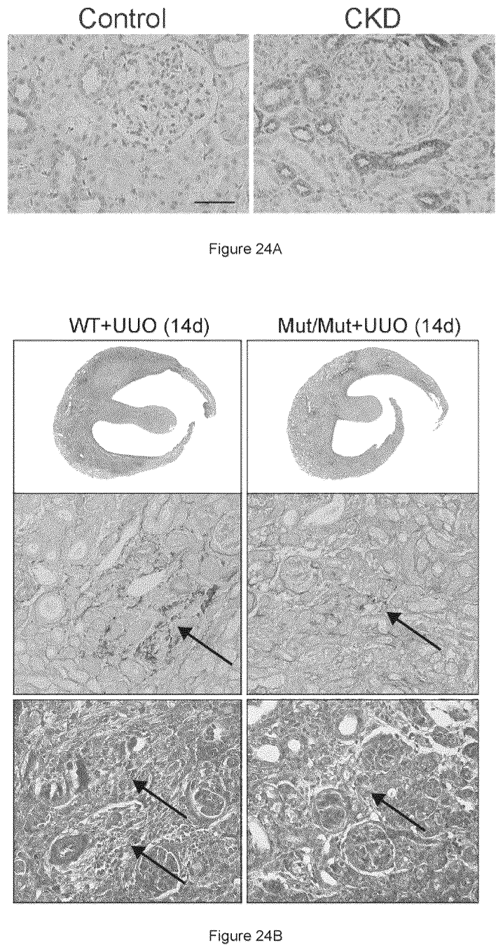

When bleomycin was administered to Wwp2^{Mut/Mut} mice, the animals showed decreased mortality (P=0.044) when compared to WT mice (FIG. 23A).

Further, in Wwp2^{Mut/Mut} mice, histological analysis revealed decreased pulmonary fibrosis (FIG. 23B), and the lesions were significantly attenuated (FIG. 23C), when compared with WT mice.

Masson's Trichrome staining of lung tissue showed that more prominent staining corresponding to mature collagen was distributed in the alveolar septa or interstitial and peribronchial connective tissue in the bleomycin-injured WT lung than that in the Wwp2^{Mut/Mut} lung (FIG. 23D).

### Example 15: WWP2 is Upregulated in Human Chronic Kidney Disease, and WWP2 LOF Protects Mice from Renal Fibrosis

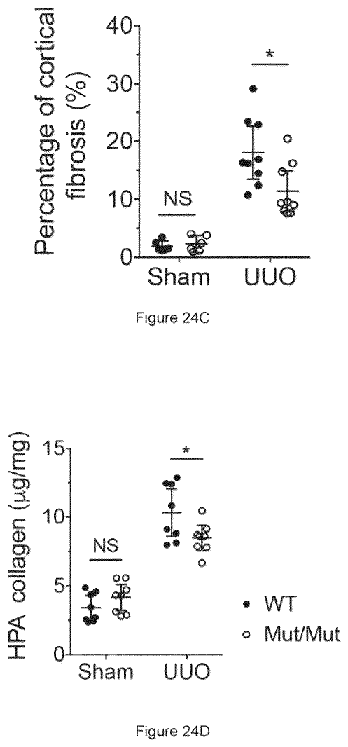

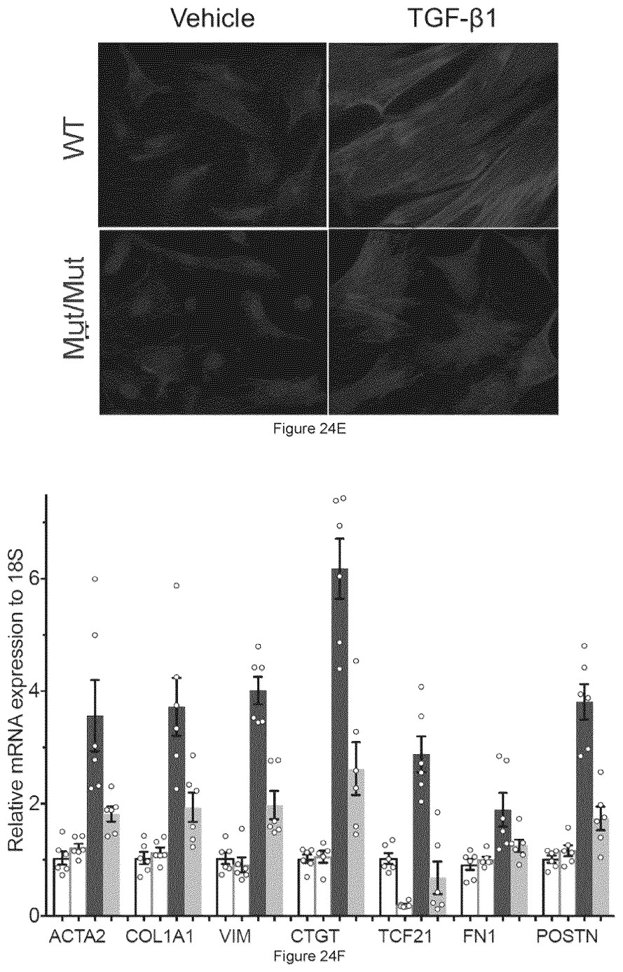

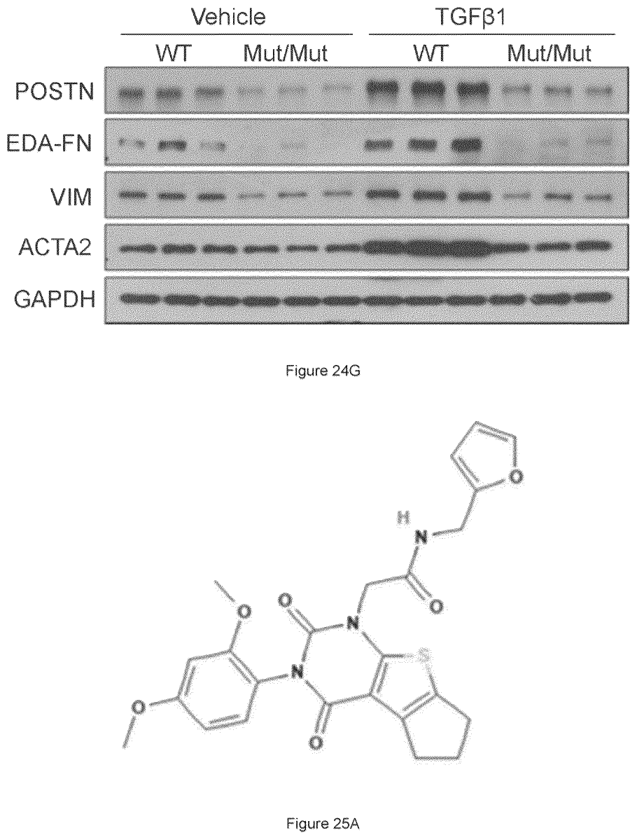

Considering that fibrosis is the common pathway in chronic kidney disease (CKD), the inventors assessed the expression of WWP2 kidney tissue from patients with CKD. Immuno-histological analysis showed that expression of WWP2 was obviously higher in the kidney tissue of patients with CKD than in the healthy control at the tubulointerstitial region (FIG. 24A).

The inventors further investigated the effects of Wwp2 knockout in the unilateral ureteral obstruction (UUO) model, a well-established rodent model of progressive renal fibrosis. The UUO model was established as described in Chevalier et al., Kidney International (2009) 75 (11), 1145-1152, and resulted in diffuse fibrosis in the kidneys of WT mice after 14 days. By contrast, Wwp2^{Mut/Mut} mice had significantly less fibrosis throughout the kidney (FIG. 24B). Quantification of the results showed that the percentage of fibrosis and collagen content in kidneys were significantly reduced in Wwp2^{Mut/Mut} mice as compared to WT mice (FIGS. 24C and 24D).

The inventors also investigated whether inhibiting WWP2 reduced pro-fibrotic phenotypes in renal fibroblasts in vitro. TGFβ1-stimulated renal fibroblasts from WT mice displayed clear organization of ACTA2 into stress fibers, while Wwp2^{Mut/Mut}-derived cells displayed diffuse expression of ACTA2, with rare incorporation into stress fibers (FIG. 24E). Moreover, the pro-fibrotic TGFβ1-induced expressional changes at both the mRNA and protein levels were largely prevented in Wwp2^{Mut/Mut} fibroblasts (FIGS. 24F and 24G, respectively).

### Example 16: Inhibition of WWP2 Using a Small Molecule Inhibitor Reduces Fibrosis

### 16.1 Methods

### 16.1.1 Evaluation of Binding to WWP2 by Octet RED Analysis

The WWP2 isoforms WWP2-FL, WWP2-N and WWP2-C were expressed as a GST fusion protein in bacteria and purified using glutathione sepharose 4B resin (GE Healthcare) according to the manufacturer's instructions. Expression and purity of the expressed WWP2 proteins was analysed by Coomassie Blue staining of the gel after separation by SDS-PAGE and western blotting (WB).

Binding assays were performed using the Octet RED96e System (ForteBio Inc., Menlo Park, CA). WWP2-FL, WWP2-N and WWP2-C proteins were biotinylated using NHS-PEO4-biotin (Pierce). Super-streptavidin (SSA) biosensors (ForteBio Inc., Menlo Park, CA) were coated in a solution containing 1 μM of biotinylated protein for 4 hours at 25° C. A duplicate set of sensors were incubated in assay buffer (1×kinetics buffer of ForteBio Inc.) with 5% DMSO without protein, for use as a background binding control. Both sets of sensors were blocked with a solution of 10 μg/ml Biocytin for 5 minutes at 25° C. A negative control of 5% DMSO was also used.

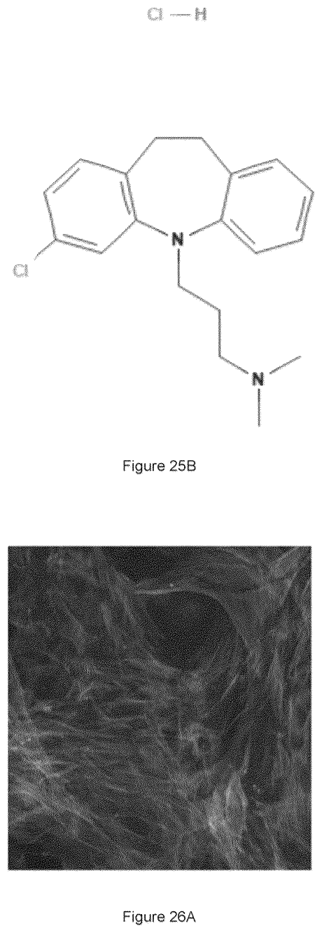

The compounds analysed in the assay were as follows:
(i) 2-[11-(2,4-dimethoxyphenyl)-0,12-dioxo-7-thia-9,11-diazatricyclo[6.4.0.02,6]dodeca-1 (8),2(6)-dien-9-yl]-N-(furan-2-ylmethyl)acetamide (PubChem CID: 3240890; Molecular Formula: C24H23N3O6S), referred to hereafter as EP1. The chemical structure for EP1 is shown in FIG. 25A.
(ii) 3-(2-chloro-5,6-dihydrobenzo[b][1]benzazepin-11-yl)-N,N-dimethylpropan-1-amine hydrochloride (PubChem CID: 68539; Molecular Formula: C19H23ClN2·HCl), referred to hereafter as Clomipramine hydrochloride. The chemical structure for Clomipramine hydrochloride is shown in FIG. 25B.

Clomipramine hydrochloride (Sigma Aldrich, #C7291) was employed as a positive control, having previously been demonstrated to be an inhibitor of HECT domain-containing E3 ligase activity (see e.g. Rossi et al., Cell Death Dis. 2014 May; 5(5): e1203, hereby incorporated by reference in its entirety).

Binding of samples containing the different compounds (at a concentration of 250 μM) to coated and uncoated reference sensors was measured over 120 seconds. Analysis of the data was performed using a double reference subtraction (sample and sensor references) in the ForteBio data analysis software.

The analysis accounts for non-specific binding, background, and signal drift and minimizes well-based and sensor variability.

### 16.1.2 Cellular Fibrosis Assays for the Small Molecules

Primary renal fibroblasts were obtained from the renal cortex of mice. Minced renal cortex pieces (1-3 mm³) were placed in 6 cm dishes with DMEM supplemented with 20% fetal bovine serum for less than 10 days to generate mice renal fibroblasts (P0), and passaged to P1 and P2 in DMEM supplemented with 10% fetal bovine serum for experiments. To mimic the in vivo activation of renal fibroblasts in fibrosis, cells were treated with human transforming growth factor-β1 (TGFβ1) (Sigma Aldrich, #T7039) at a concentration of 5 ng/μl for 72 hours.

To test the anti-fibrotic effect of candidate small-molecules, cells were treated with each compound (0.3 mM) for 1 hour, followed by stimulation with TGFβ1 before harvesting. After fixation with ice-cold acetone and blocking with 1% BSA for 30 min at RT, the slides were incubated with primary anti-smooth muscle a actin (ACTA2) antibody (ACTA2 is a widely recognised myofibroblast marker, indicating fibroblast activation) (Sigma-Aldrich, #A5228, 1:100) overnight at 4° C. Following washing steps, slides were incubated with Bovine Anti-Mouse IgG-CFL 488 secondary antibody (Santacruz Biotechnology, #sc-362256,1:100) for 2 hours at RT. VectaShield Mounting Medium (Vector laboratories, #H-1200) with DAPI was used to stain the nuclei and the slides were covered by coverslip. Slides were imaged on Leica fluorescence microscope (Leica).

### 16.2 Results

Binding of Clomipramine hydrochloride at 250 μM to WWP2-N and WWP2-C was 0.08 nm and 0.0839 nm, respectively. Thus, a binding level of 20.08 nm can be considered to be indicative of binding to WWP2.

EP1 displayed stronger binding to WWP2-N than Clomipramine hydrochloride (0.1253 nm), and weaker binding to WWP2-C than Clomipramine hydrochloride (0.0061 nm) as indicated in the table below:

| | Binding level (nm) | | |
|---|---|---|---|
| | to WWP2 N | to WWP2 C | Anti-fibrotic response |
| Compound name | isoform | isoform | in renal fibroblasts |
| EP1 | 0.1253 | 0.0061 | Reduced expression of |
| | | | ACTA2 in TGFβ1- |
| | | | stimulated fibroblasts |
| Clomipramine HCl | 0.0800 | 0.0839 | Positive control drug |

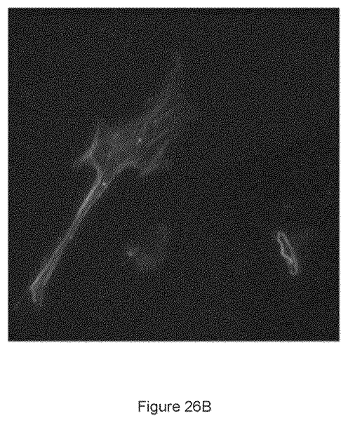

TGFβ1 stimulation of primary renal fibroblasts increased fibrosis as evidenced by increased ACTA2 expression, as determined by fluorescence microscopy (FIG. 26A).

Treatment of the fibroblasts with EP1 prior to stimulation with TGFβ1 was associated with reduced expression of ACTA2 (FIG. 26B).

Thus EP1 is identified as an inhibitor of WWP2, and demonstrates that small molecule inhibitors of WWP2 (in particular, small molecule WWP2 inhibitors which bind to the WWP2 N-terminal isoform comprising the C2 domain) are useful for the treatment/prevention of fibrosis, as evidenced by its ability to antagonise TGFβ1-mediated activation of fibroblasts to myofibroblasts (which are effectors of fibrosis).

1.-12. (canceled)

13. A method of treating or preventing fibrosis or a disease characterised by fibrosis, comprising administering a therapeutically or prophylactically effective amount of a WWP2 inhibitor to a subject in need thereof.

14. The method according to claim 13, wherein the fibrosis is fibrosis of the kidney, lung, liver, heart, skeletal muscle, blood vessels, eye, skin, pancreas, bowel, small intestine, large intestine, colon, brain, and/or bone marrow.

15. The method according to claim 13, wherein the method is a method of treating or preventing a disease characterised by fibrosis, and wherein the disease characterised by fibrosis is selected from the group consisting of: kidney disease, renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus, tubulointerstitial fibrosis, glomerular fibrosis, pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, asthma, chronic liver disease, cirrhosis, non-alcoholic fatty liver disease (NAFLD), non-alcoholic steatohepatitis (NASH), primary biliary cirrhosis (PBC), schistosomal liver disease, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), atherosclerosis, varicose veins, cerebral infarcts, gliosis, Alzheimer's disease, muscular dystrophy, Duchenne muscular dystrophy (DMD), Becker's muscular dystrophy (BMD), Crohn's disease, microscopic colitis, primary sclerosing cholangitis (PSC), scleroderma, nephrogenic systemic fibrosis, cutis keloid, arthrofibrosis, Dupuytren's contracture, mediastinal fibrosis, retroperitoneal fibrosis, myelofibrosis, Peyronie's disease, adhesive capsulitis, progressive systemic sclerosis (PSS), chronic graft versus host disease, Grave's ophthalmopathy, epiretinal fibrosis, retinal fibrosis, subretinal fibrosis, subretinal fibrosis associated with macular degeneration, subretinal fibrosis associated with wet age-related macular degeneration (AMD), diabetic retinopathy, glaucoma, corneal fibrosis, post-surgical fibrosis, post-surgical fibrosis of the posterior capsule following cataract surgery, post-surgical fibrosis of the bleb following trabeculectomy for glaucoma, conjunctival fibrosis, subconjunctival fibrosis, arthritis, fibrotic pre-neoplastic disease, fibrotic neoplastic disease, fibrosis induced by chemical or environmental insult, fibrosis induced by cancer chemotherapy, fibrosis induced by pesticides, and fibrosis induced by radiation or cancer radiotherapy.

16. The method according to claim 13, wherein the disease characterised by fibrosis is selected from the group consisting of: kidney disease, renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus, tubulointerstitial fibrosis, glomerular fibrosis, pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, asthma, chronic liver disease, cirrhosis, non-alcoholic fatty liver disease (NAFLD), non-alcoholic steatohepatitis (NASH), primary biliary cirrhosis (PBC), schistosomal liver disease, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, and arrhythmogenic right ventricular cardiomyopathy (ARVC).

17. The method according to claim 13, wherein the WWP2 inhibitor binds to WWP2 and inhibits interaction between WWP2 and an interaction partner for WWP2.

18. The method according to claim 17, wherein the interaction partner for WWP2 is selected from the group consisting of: a SMAD protein, SMAD2, SMAD3, SMAD7, PTEN, an OCT protein, OCT4, and EGR.

19. The method according to claim 17, wherein the interaction partner for WWP2 is selected from the group consisting of: a SMAD protein, SMAD2, and SMAD3.

20. The method according to claim 13, wherein the WWP2 inhibitor binds to a WW domain and/or the C2 domain of WWP2.

21. The method according to claim 13, wherein the WWP2 inhibitor is a small molecule inhibitor of WWP2.

22. A method of treating or preventing pathological inflammation or a disease characterised by pathological inflammation, comprising administering a therapeutically or prophylactically effective amount of a WWP2 inhibitor to a subject in need thereof.

23. The method according to claim 22, wherein the pathological inflammation is associated with a chronic infection, a cancer, an autoimmune disease, a degenerative disease, or an allergic disease.

24. The method according to claim 22, wherein the method is a method of treating or preventing a disease characterised by pathological inflammation, and wherein the disease characterised by pathological inflammation is selected from the group consisting of: a chronic viral infection, a chronic bacterial infection, a chronic protozoal infection, human immunodeficiency virus (HIV) infection, hepatitis B virus (HBV) infection, hepatitis C virus (HCV) infection, Epstein-Barr Virus (EBV) infection, measles virus (MV) infection, cytomegalovirus (CMV) infection, human T-cell leukemia virus (HTLV) infection, human herpesvirus (HHV) infection, herpes simplex virus (HSV) infection, Varicella-Zoster virus (VZV) infection, human papovavirus infection, JC virus infection, BK virus infection, adenovirus (AdV) infection, parvovirus infection, human papillomaviruses (HPV) infection, Mycobacterium tuberculosis infection, Helicobacter pylori infection, Salmonella Typhi infection, Treponema pallidum infection, Pseudomonas aeruginosa infection, Escherichia coli infection, Staphylococcus aureus infection, Hemophilus influenza infection, Mycobacterium leprae infection, Candida spp infection, Aspergillus infection, Plasmodium spp infection, Babesia spp. infection, Giardia spp. infection, Leishmania spp. infection, Trypanosoma spp. infection, Toxoplasma spp. infection, a cancer, a primary cancer, a metastatic cancer, adrenal gland cancer, adrenal medulla cancer, anal cancer, appendiceal cancer, bladder cancer, a blood cancer, bone cancer, bone marrow cancer, brain cancer, breast cancer, caecal cancer, a central nervous system cancer, cerebellar cancer, cervical cancer, colon cancer, duodenal cancer, endometrial cancer, epithelial cell cancer, renal epithelial cell cancer, gallbladder cancer, oesophageal cancer, glial cell cancer, cardiac cancer, ileal cancer, jejunal cancer, kidney cancer, lacrimal gland cancer, laryngeal cancer, liver cancer, lung cancer, lymphoma, nodal lymphoma, lymphoblastic cancer, maxillary cancer, mediastinal cancer, mesenteric cancer, myometrial cancer, nasopharyngeal cancer, omental cancer, oral cavity cancer, ovarian cancer, pancreatic cancer, parotid gland cancer, peripheral nervous system cancer, peritoneal cancer, pleural cancer, prostate cancer, salivary gland cancer, sigmoid colon cancer, skin cancer, small intestine cancer, soft tissue cancer, splenic cancer, stomach cancer, testicular cancer, thymic cancer, thyroid cancer, tongue cancer, tonsillar cancer, tracheal cancer, uterine cancer, vulvar cancer, white blood cell cancer, diabetes mellitus type 1, diabetes mellitus type 2, coeliac disease, Graves' disease, inflammatory bowel disease, Crohn's disease, multiple sclerosis, psoriasis, rheumatoid arthritis, systemic lupus erythematosus, Alzheimer's disease, amyotrophic lateral sclerosis, Charcot-Marie-Tooth disease, chronic traumatic encephalopathy, cystic fibrosis, degenerative Leigh syndrome, Ehlers-Danlos syndrome, fibrodysplasia ossificans progressiva, Friedreich's ataxia, frontotemporal dementia, atherosclerotic cardiovascular disease, coronary artery disease, aortic stenosis, myocardial infarction, pulmonary arterial hypertension, Huntington's disease, infantile neuroaxonal dystrophy, keratoconus, keratoglobus, leukodystrophies, macular degeneration, Marfan's syndrome, mitochondrial myopathies, mitochondrial DNA depletion syndrome, multiple system atrophy, muscular dystrophies, neuronal ceroid lipofuscinosis, Niemann-Pick disease, osteoarthritis, osteoporosis, Parkinson's disease, pulmonary arterial hypertension, a prion disease, Creutzfeldt-Jakob disease, fatal familial insomnia, progressive supranuclear palsy, retinitis pigmentosa, rheumatoid arthritis, Sandhoff Disease, spinal muscular atrophy, subacute sclerosing panencephalitis, Tay-Sachs disease, vascular dementia, allergic asthma, allergic rhinitis, food allergy, and atopic dermatitis.

25. The method according to claim 22, wherein the WWP2 inhibitor binds to WWP2 and inhibits interaction between WWP2 and an interaction partner for WWP2.

26. The method according to claim 25, wherein the interaction partner for WWP2 is selected from the group consisting of: a SMAD protein, SMAD2, SMAD3, SMAD7, PTEN, an OCT protein, OCT4, and EGR.

27. The method according to claim 25, wherein the interaction partner for WWP2 is selected from the group consisting of: a SMAD protein, SMAD2, and SMAD3.

28. The method according to claim 22, wherein the WWP2 inhibitor binds to a WW domain and/or the C2 domain of WWP2.

29. The method according to claim 22, wherein the WWP2 inhibitor is a small molecule inhibitor of WWP2.
